(19) United States
(12) Patent Application Publication
(10) Pub. No.: US 2025/0004366 A1
(43) Pub. Date: Jan. 2, 2025

(54) RESIST COMPOSITION, RESIST PATTERN FORMATION METHOD, COMPOUND, ACID-GENERATING AGENT, ACID DIFFUSION CONTROL AGENT, AND HIGH-MOLECULAR-WEIGHT COMPOUND

(71) Applicant: TOKYO OHKA KOGYO CO., LTD., Kawasaki-shi (JP)

(72) Inventors: Masatoshi ARAI, Kawasaki-shi (JP); Seiji TODOROKI, Kawasaki-shi (JP)

(21) Appl. No.: 18/684,046

(22) PCT Filed: Aug. 24, 2022
(86) PCT No.: PCT/JP2022/031908

§ 371 Date: Feb. 15, 2024

(30) Foreign Application Priority Data
Aug. 26, 2021 (JP) 2021-138091

(51) Int. Cl. G03F 7/004; G03F 7/038; G03F 7/039

(52) U.S. Cl. CPC G03F 7/0045; G03F 7/0382; G03F 7/0397

(57) ABSTRACT

A resist composition which generates an acid upon light exposure and whose solubility in a developing solution is changed due to an action of an acid, the resist composition including a compound that contains a cation represented by General Formula (c0) in which X⁰¹ represents an electron-withdrawing group, X⁰² represents an electron-withdrawing group different from X⁰¹, R⁰¹ represents an electron-donating group, R⁰² and R⁰³ each independently represents a substituent, nx1 and nx2 each independently represents an integer of 1 to 4, n1 represents an integer of 0 to 3, nx1+nx2+n1≤5 is satisfied, and n2 and n3 each independently represent an integer of 0 to 4

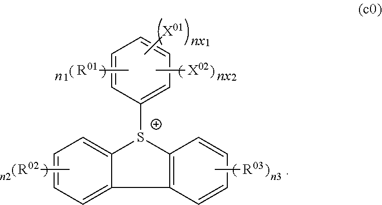

### TECHNICAL FIELD

The present invention relates to a resist composition, a resist pattern formation method, a compound, an acid-generating agent, an acid diffusion control agent, and a high-molecular-weight compound.

Priority is claimed on Japanese Patent Application No. 2021-138091, filed Aug. 26, 2021, the content of which is incorporated herein by reference.

### BACKGROUND ART

In recent years, in the manufacture of semiconductor elements and liquid crystal display elements, advances in lithography technologies have led to rapid progress in the field of pattern miniaturization. These pattern miniaturization techniques typically involve shortening the wavelength (increasing the energy) of the exposure light source.

Resist materials require lithography characteristics such as a high resolution that enables reproduction of patterns with minute dimensions, and a high level of sensitivity to these kinds of exposure light sources.

As a resist material that satisfies these requirements, a chemically amplified resist composition containing a base material component whose solubility in a developing solution is changed due to an action of an acid and an acid-generating agent component that generates an acid upon light exposure has been used.

In the chemically amplified resist composition, a resin having a plurality of constitutional units is typically used in order to improve lithography characteristics and the like.

Further, in the resist pattern formation, the behavior of an acid generated from an acid-generating agent component upon light exposure serves as an element that greatly affects the lithographic characteristics.

Various kinds of acid-generating agents have been suggested as the acid-generating agent used in a chemically amplified resist composition. Examples of such known acid-generating agents include an onium salt-based acid-generating agent such as an iodonium salt or a sulfonium salt, an oxime sulfonate-based acid-generating agent, a diazomethane-based acid-generating agent, a nitrobenzyl sulfonate-based acid-generating agent, an imino sulfonate-based acid-generating agent, and a disulfone-based acid-generating agent.

For example, Patent Document 1 discloses a resist composition that employs, as an acid-generating agent, a compound in which an electron-withdrawing group is introduced into a sulfonium cation.

### CITATION LIST

### Patent Document

### [Patent Document 1]

Japanese Unexamined Patent Application, First Publication No. 2019-207404

### SUMMARY OF INVENTION

### Technical Problem

With the miniaturization of resist patterns, for example, the aim is to form a fine pattern with a size of several tens of nanometers in lithography using EUV and EB. With such miniaturization of resist patterns, an object to be achieved is to improve the sensitivity and reduce defects.

The present invention has been made in consideration of the above-described circumstances, and an object thereof is to provide a resist composition with high sensitivity and satisfactory lithography characteristics such as fewer defects, a resist pattern formation method using the resist composition, and a compound, an acid-generating agent, an acid diffusion control agent, and a high-molecular-weight compound which are used in the resist composition.

### Solution to Problem

In order to achieve the above-described object, the present invention employs the following configurations.

That is, according to a first aspect of the present invention, a resist composition is provided which generates an acid upon light exposure and whose solubility in a developing solution is changed due to an action of the acid, the resist composition including: a compound containing a cation (C0) represented by General Formula (c0).

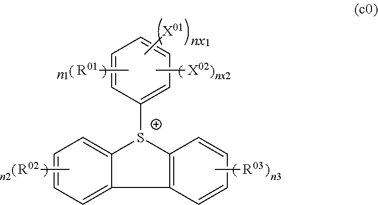

[In the formula, X⁰¹ represents an electron-withdrawing group, X⁰² represents an electron-withdrawing group different from X⁰¹, and R⁰¹ represents an electron-donating group. R⁰² and R⁰³ each independently represents a substituent. nx1 and nx2 each independently represents an integer of 1 to 4, n1 represents an integer of 0 to 3, and nx1+nx2+n1≤5 is satisfied. n2 and n3 each independently represents an integer of 0 to 4. In a case where nx1 represents 2 or greater, a plurality of X¹'s may be the same as or different from each other. In a case where nx2 represents 2 or greater, a plurality of X⁰²'s may be the same as or different from each other. In a case where n1 represents 2 or greater, a plurality of R⁰¹'s may be the same as or different from each other. In a case where n2 represents 2 or greater, a plurality of R⁰²'s may be the same as or different from each other. In a case where n3 represents 2 or greater, a plurality of R⁰³'s may be the same as or different from each other.]

According to a second aspect of the present invention, a resist pattern formation method is provided including a step of forming a resist film on a support using the resist composition according to the first aspect, a step of exposing the resist film to light, and a step of developing the resist film exposed to light to form a resist pattern.

According to a third aspect of the present invention, a compound represented by General Formula (M0) is provided.

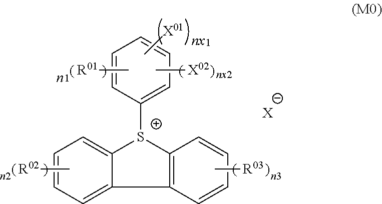

[In the formula, X⁰¹ represents an electron-withdrawing group, X⁰² represents an electron-withdrawing group different from X⁰¹, and R⁰¹ represents an electron-donating group. R⁰² and R⁰³ each independently represents a substituent. nx1 and nx2 each independently represents an integer of 1 to 4, n1 represents an integer of 0 to 3, and nx1+nx2+n1≤5 is satisfied. n2 and n3 each independently represents an integer of 0 to 4. In a case where nx1 represents 2 or greater, a plurality of X⁰¹'s may be the same as or different from each other. In a case where nx2 represents 2 or greater, a plurality of X⁰²'s may be the same as or different from each other. In a case where n1 represents 2 or greater, a plurality of R⁰¹'s may be the same as or different from each other. In a case where n2 represents 2 or greater, a plurality of R⁰²'s may be the same as or different from each other. In a case where n3 represents 2 or greater, a plurality of R⁰³'s may be the same as or different from each other.]

According to the third aspect of the present invention, an acid-generating agent containing a compound represented by General Formula (B0) is provided.

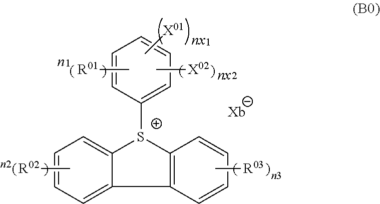

[In the formula, X⁰¹ represents an electron-withdrawing group, X⁰² represents an electron-withdrawing group different from X⁰¹, and R⁰¹ represents an electron-donating group. R⁰² and R⁰³ each independently represents a substituent. nx1 and nx2 each independently represents an integer of 1 to 4, n1 represents an integer of 0 to 3, and nx1+nx2+n1≤5 is satisfied. n2 and n3 each independently represents an integer of 0 to 4. In a case where nx1 represents 2 or greater, a plurality of X⁰¹'s may be the same as or different from each other. In a case where nx2 represents 2 or greater, a plurality of X⁰²'s may be the same as or different from each other. In a case where n1 represents 2 or greater, a plurality of R⁰¹'s may be the same as or different from each other. In a case where n2 represents 2 or greater, a plurality of R⁰²'s may be the same as or different from each other. In a case where n3 represents 2 or greater, a plurality of R⁰³'s may be the same as or different from each other. Xb⁻ represents a counter anion.]

According to a fourth aspect of the present invention, an acid diffusion control agent containing a compound represented by General Formula (D0) is provided.

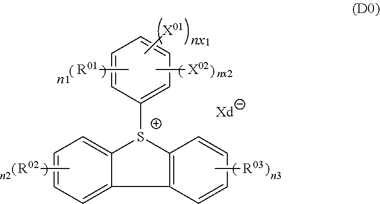

[In the formula, X⁰¹ represents an electron-withdrawing group, X⁰² represents an electron-withdrawing group different from X⁰¹, and R⁰¹ represents an electron-donating group. R⁰² and R⁰³ each independently represents a substituent. nx1 and nx2 each independently represents an integer of 1 to 4, n1 represents an integer of 0 to 3, and nx1+nx2+n1≤5 is satisfied. n2 and n3 each independently represents an integer of 0 to 4. In a case where nx1 represents 2 or greater, a plurality of X⁰¹'s may be the same as or different from each other. In a case where nx2 represents 2 or greater, a plurality of X⁰²'s may be the same as or different from each other. In a case where n1 represents 2 or greater, a plurality of R⁰¹'s may be the same as or different from each other. In a case where n2 represents 2 or greater, a plurality of R⁰²'s may be the same as or different from each other. In a case where n3 represents 2 or greater, a plurality of R⁰³'s may be the same as or different from each other. Xd⁻ represents a counter anion.]

According to a fifth aspect of the present invention, a high-molecular-weight compound having a constitutional unit (a0) represented by General Formula (A0-1) is provided.

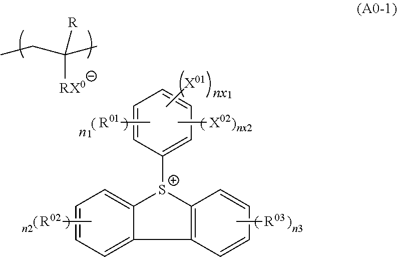

[In the formula, R represents a hydrogen atom, an alkyl group having 1 to 5 carbon atoms, or a halogenated alkyl group having 1 to 5 carbon atoms. Rx⁰ represents a group containing an anion. X⁰¹ represents an electron-withdrawing group, X⁰² represents an electron-withdrawing group different from X⁰¹, and R⁰¹ represents an electron-donating group. R⁰² and R⁰³ each independently represents a substituent. nx1 and nx2 each independently represents an integer of 1 to 4, n1 represents an integer of 0 to 3, and nx1+nx2+n1≤5 is satisfied. n2 and n3 each independently represents an integer of 0 to 4. In a case where nx1 represents 2 or greater, a plurality of X⁰¹'s may be the same as or different from each other. In a case where nx2 represents 2 or greater, a plurality of X⁰²'s may be the same as or different from each other. In a case where n1 represents 2 or greater, a plurality of R⁰¹'s may be the same as or different from each other. In a case where n2 represents 2 or greater, a plurality of R⁰²'s may be the same as or different from each other. In a case where n3 represents 2 or greater, a plurality of R⁰³'s may be the same as or different from each other.]

### Advantageous Effects of Invention

According to the present invention, it is possible to provide a resist composition with high sensitivity and satisfactory lithography characteristics such as fewer defects, a resist pattern formation method using the resist composition, and a compound, an acid-generating agent, an acid diffusion control agent, and a high-molecular-weight compound, which are used in the resist composition.

### DESCRIPTION OF EMBODIMENTS

In the present specification and the scope of the present claims, the term “aliphatic” is a relative concept used in relation to the term “aromatic”, and defines a group or compound that has no aromaticity.

The term “alkyl group” includes a linear, branched, or cyclic monovalent saturated hydrocarbon group unless otherwise specified. The same applies to the alkyl group in an alkoxy group.

The term “alkylene group” includes a linear, branched, or cyclic divalent saturated hydrocarbon group unless otherwise specified.

Examples of “halogen atom” include a fluorine atom, a chlorine atom, a bromine atom, and an iodine atom.

The term “constitutional unit” denotes a monomer unit constituting a high-molecular-weight compound (a resin, a polymer, or a copolymer).

The expression “may have a substituent” includes both a case where a hydrogen atom (—H) is substituted with a monovalent group and a case where a methylene (—CH₂—) group is substituted with a divalent group.

The term “light exposure” is a general concept for irradiation with radiation.

The term “acid-decomposable group” indicates a group having acid decomposability in which at least a part of a bond in the structure of the acid-decomposable group can be cleaved due to the action of an acid.

Examples of the acid-decomposable group whose polarity is increased due to the action of an acid include groups which are decomposed due to the action of an acid to generate a polar group.

Examples of the polar group include a carboxy group, a hydroxyl group, an amino group, and a sulfo group (—SO₃H).

More specific examples of the acid-decomposable group include a group in which the above-described polar group has been protected by an acid-dissociable group (such as a group in which a hydrogen atom of the OH-containing polar group has been protected by an acid-dissociable group).

Here, the term “acid-dissociable group” indicates both a group (i) having an acid dissociation property in which a bond between the acid-dissociable group and an atom adjacent to the acid-dissociable group can be cleaved due to the action of an acid and a group (ii) in which some bonds are cleaved due to the action of an acid, a decarboxylation reaction occurs, and thus the bond between the acid-dissociable group and the atom adjacent to the acid-dissociable group can be cleaved.

It is necessary that the acid-dissociable group that constitutes the acid-decomposable group be a group which exhibits a lower polarity than that of the polar group generated by the dissociation of the acid-dissociable group. Thus, in a case where the acid-dissociable group is dissociated due to the action of an acid, a polar group exhibiting a higher polarity than that of the acid-dissociable group is generated so that the polarity is increased. As a result, the polarity of an entire component (A1) is increased. Due to the increase in the polarity, relatively, the solubility in a developing solution is changed such that the solubility is increased in a case where the developing solution is an alkali developing solution and the solubility is decreased in a case where the developing solution is an organic developing solution.

The term “base material component” denotes an organic compound having a film-forming ability. Organic compounds used as the base material component are classified into non-polymers and polymers. As the non-polymers, those having a molecular weight of 500 or greater and less than 4,000 are typically used. Hereinafter, the term “low-molecular-weight compound” denotes a non-polymer having a molecular weight of 500 or greater and less than 4,000. As the polymer, those having a molecular weight of 1,000 or greater are typically used. Hereinafter, “resin”, “high-molecular-weight compound”, or “polymer” denotes a polymer having a molecular weight of 1,000 or greater. As the molecular weight of the polymer, the weight-average molecular weight in terms of polystyrene according to gel permeation chromatography (GPC) is used.

The expression “constitutional unit derived from” denotes a constitutional unit formed by cleavage of a multiple bond between carbon atoms, for example, an ethylenic double bond.

In “acrylic acid ester”, the hydrogen atom bonded to the carbon atom at the α-position may be substituted with a substituent. The substituent (R^{αx}) that substitutes the hydrogen atom bonded to the carbon atom at the α-position is an atom other than the hydrogen atom or a group. Further, the acrylic acid ester includes itaconic acid diester in which the substituent (R^{αx}) has been substituted with a substituent having an ester bond and α-hydroxyacryl ester in which the substituent (R^{αx}) has been substituted with a hydroxyalkyl group or a group obtained by modifying a hydroxyl group thereof. Further, the carbon atom at the α-position of acrylic acid ester indicates the carbon atom to which the carbonyl group of acrylic acid is bonded, unless otherwise specified.

Hereinafter, acrylic acid ester in which the hydrogen atom bonded to the carbon atom at the α-position has been substituted with a substituent is also referred to as α-substituted acrylic acid ester.

The concept “derivative” includes those obtained by substituting a hydrogen atom at the α-position of a target compound with another substituent such as an alkyl group or a halogenated alkyl group, and derivatives thereof. Examples of the derivatives thereof include those obtained by substituting a hydrogen atom of a hydroxyl group of a target compound, in which the hydrogen atom at the α-position may be substituted with a substituent, with an organic group, and those obtained by bonding a substituent other than a hydroxyl group to a target compound in which the hydrogen atom at the α-position may be substituted with a substituent. Further, the α-position denotes the first carbon atom adjacent to a functional group unless otherwise specified.

Examples of the substituent that substitutes the hydrogen atom at the α-position of hydroxystyrene include those for R^{αx}.

In the present specification and the scope of the present claims, asymmetric carbons may be present and enantiomers or diastereomers may be present depending on the structures of the chemical formulae. In this case, these isomers are represented by one chemical formula. These isomers may be used alone or in the form of a mixture.

### (Resist Composition)

A resist composition according to a first aspect of the present invention is a resist composition which generates an acid upon light exposure and whose solubility in a developing solution is changed due to an action of an acid.

Such a resist composition contains a compound (hereinafter, also referred to as “compound C”) containing a cation (C0) represented by General Formula (c0). The compound (C) may be a base material component (A) (hereinafter, also referred to as “component (A)”) whose solubility in a developing solution is changed due to the action of an acid. The compound (C) may be an acid-generating agent component (B) (hereinafter, also referred to as “component (B)”) that generates an acid upon light exposure. The compound (C) may be an acid diffusion control agent component (D) (hereinafter, also referred to as “component (D)”) that traps an acid generated upon light exposure (that is, controls diffusion of an acid).

The resist composition of the present embodiment has an acid-generating function of generating an acid upon light exposure, and the component (A) may generate an acid upon light exposure or the component (B) may generate an acid upon light exposure.

Specifically, in the resist composition according to the present embodiment, (1) the resist composition may contain the component (B) as a component that generates an acid upon light exposure, (2) the component (A) may be a component that generates an acid upon light exposure, or (3) the component (A) may be a component that generates an acid upon light exposure and the resist composition may contain the component (B).

That is, in cases (2) and (3), the component (A) is “base material component that generates an acid upon light exposure and whose solubility in a developing solution is changed due to the action of an acid”. In a case where the component (A) is a base material component which generates an acid upon light exposure and whose solubility in a developing solution is changed due to the action of the acid, it is preferable that a component (A1) described below be a high-molecular-weight compound which generates an acid upon light exposure and whose solubility in a developing solution is changed due to the action of the acid. As such a high-molecular-weight compound, a resin having a constitutional unit that generates an acid upon light exposure can be used. As the constitutional unit that generates an acid upon light exposure, known constitutional units can be used.

In a case where a resist film is formed using the resist composition of the present embodiment and the formed resist film is subjected to selective light exposure, an acid is generated from the component (A) or the component (B) at an exposed portion of the resist film, and the solubility of the component (A) in a developing solution is not changed at an unexposed portion of the resist film while the solubility of the component (A) in a developing solution is changed due to the action of the acid, and thus a difference occurs in the solubility in a developing solution between the exposed portion and the unexposed portion. Therefore, in a case where the resist film is developed, the exposed portion of the resist film is dissolved and removed to form a positive-tone resist pattern in a case where the resist composition is of a positive-tone, whereas the unexposed portion of the resist film is dissolved and removed to form a negative-tone resist pattern in a case where the resist composition is of a negative tone.

In the present specification, a resist composition which forms a positive-tone resist pattern by dissolving and removing the exposed portion of the resist film is referred to as a positive-tone resist composition, and a resist composition which forms a negative-tone resist pattern by dissolving and removing the unexposed portion of the resist film is referred to as a negative-tone resist composition. The resist composition of the present embodiment may be a positive-tone resist composition or a negative-tone resist composition. Further, the resist composition of the present embodiment may be used in an alkali developing process using an alkali developing solution in the developing treatment in a case of forming a resist pattern or may be used in a solvent developing process using a developing solution containing an organic solvent (organic developing solution) in the developing treatment.

### <Compound (C) Containing Cation (C0)>

The resist composition according to the present embodiment contains a compound (C) containing a cation (C0) represented by General Formula (c0).

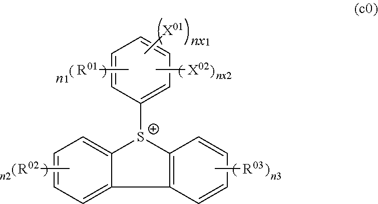

[In the formula, X⁰¹ represents an electron-withdrawing group, X⁰² represents an electron-withdrawing group different from X⁰¹, and R⁰¹ represents an electron-donating group. R⁰² and R⁰³ each independently represents a substituent. nx1 and nx2 each independently represents an integer of 1 to 4, n1 represents an integer of 0 to 3, and nx1+nx2+n1≤5 is satisfied. n2 and n3 each independently represents an integer of 0 to 4. In a case where nx1 represents 2 or greater, a plurality of X¹'s may be the same as or different from each other. In a case where nx2 represents 2 or greater, a plurality of X⁰²'s may be the same as or different from each other. In a case where n1 represents 2 or greater, a plurality of R⁰¹'s may be the same as or different from each other. In a case where n2 represents 2 or greater, a plurality of R⁰²'s may be the same as or different from each other. In a case where n3 represents 2 or greater, a plurality of R⁰³'s may be the same as or different from each other.]

In Formula (c0), X⁰¹ represents an electron-withdrawing group.

Examples of the electron-withdrawing group include an acyl group, a methanesulfonyl group (mesyl group), a halogen atom, a halogenated alkyl group, a halogenated alkoxy group, a halogenated alkylamino group, a halogenated alkylthio group, a cyano group, a nitro group, a dialkylphosphono group, an alkylsulfonyl group, a sulfonyloxy group, an acylthio group, a sulfamoyl group, a thiocyanate group, and a thiocarbonyl group.

Examples of the halogen atom in the halogen atom, the halogenated alkyl group, the halogenated alkoxy group, the halogenated aryloxy group, the halogenated alkylamino group, and the halogenated alkylthio group include a fluorine atom, a bromine atom, and an iodine atom. Among these, a fluorine atom is preferable.

The alkyl group in the halogenated alkyl group, the halogenated alkoxy group, the halogenated alkylamino group, the halogenated alkylthio group, the dialkylphosphono group, and the alkylsulfonyl group has preferably 1 to 5 carbon atoms, more preferably 1 to 3 carbon atoms, and still more preferably 1 or 2 carbon atoms.

Among these, as the electron-withdrawing group, a fluorine atom or a fluorinated alkyl group is preferable, a fluorine atom or a fluorinated alkyl group having 1 to 3 carbon atoms is more preferable, and a fluorine atom or a trifluoromethyl group is still more preferable.

Specific examples of the electron-withdrawing group are shown below.

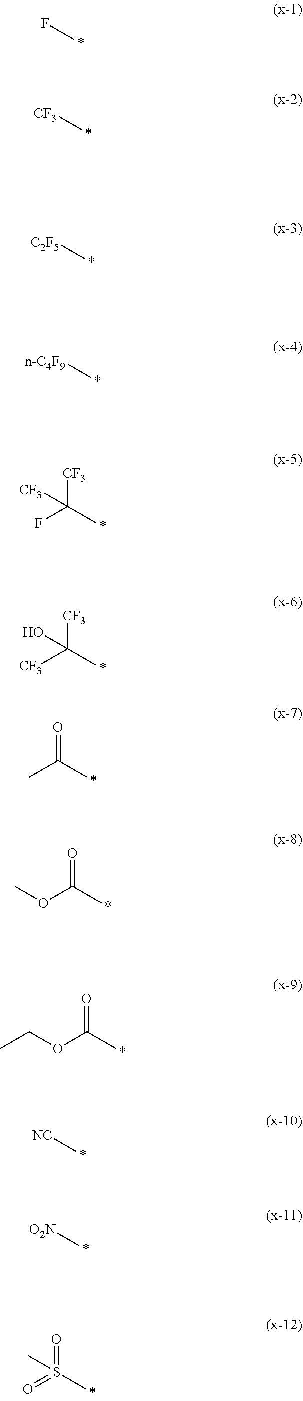

In Formula (c0), X⁰² represents an electron-withdrawing group different from X⁰¹.

Examples of the electron-withdrawing group as X⁰² include the same groups as those for the electron-withdrawing group as X⁰¹. Here, the electron-withdrawing group as X⁰² is different from the electron-withdrawing group as X⁰¹. That is, X⁰¹ and X⁰² do not represent the same electron-withdrawing group.

Examples of a combination of X⁰¹ and X⁰² include a combination of a halogen atom and a halogenated alkyl group, a combination of a halogen atom and a methanesulfonyl group, a combination of a halogenated alkyl group and a methanesulfonyl group, a combination of a halogen atom and a cyano group, and a combination of a halogenated alkyl group and a cyano group. Among these, a combination of a fluorine atom and a fluorinated alkyl group is preferable as the combination of X⁰¹ and X⁰².

In Formula (c0), nx1 and nx2 each independently represents an integer of 1 to 4. In a case where nx1 represents 2 or greater, a plurality of X⁰¹'s may be the same as or different from each other. In a case where nx2 represents 2 or greater, a plurality of X⁰²'s may be the same as or different from each other.

nx1 and nx2 represent preferably an integer of 1 to 3, more preferably an integer of 1 or 2, and still more preferably 1. From the viewpoint that the hydrophobicity is not extremely high, it is preferable that nx1 and nx2 represent 1.

Since the cation (C0) has two or more kinds of electron-withdrawing groups represented by X⁰¹ and X⁰² in the phenyl group bonded to the sulfur atom of the dibenzothiophene skeleton, the symmetry of the electron density of the phenyl group is disturbed. In the cation (C0), the phenyl group bonded to the sulfur atom of the dibenzothiophene skeleton has a high rotational degree of freedom. The solubility in a solvent is considered to be improved due to the disturbance of the electron density of the phenyl group having a high rotational degree of freedom. Therefore, the compound (C) containing the cation (C0) has improved solubility in an organic solvent used for preparing the resist composition. In addition, the cation (C0) decomposed upon light exposure has improved solubility in a developing solution. As a result, defects are reduced.

The bonding positions of X⁰¹ and X⁰² in the phenyl group are not particularly limited. From the viewpoint that the symmetry of the electron density in the phenyl group is not disturbed, it is preferable that X⁰¹ and X⁰² be bonded to the phenyl group such that the balance of electron-withdrawing groups in the phenyl group is asymmetric. For example, it is preferable that the same electron-withdrawing groups not be bonded to two ortho positions of the phenyl group. It is preferable that the same electron-withdrawing groups not be bonded to the two meta positions of the phenyl group. Examples of a combination of bonding positions of X⁰¹ and X⁰² to the phenyl group include a combination of the meta position and the meta position, a combination of the para position and the meta position, a combination of the meta position and the ortho position, and a combination of the ortho position and the para position. Examples of a combination of the meta position and the ortho position include a combination of the 1-position and the 2-position, a combination of the 1-position and the 4-position, a combination of the 2-position and the 5-position, and a combination of the 4-position and the 5-position. As the combination of the meta position and the ortho position, a combination of the 1-position and the 4-position or a combination of the 2-position and the 5-position is preferable.

In Formula (c0), R⁰¹ represents an electron-donating group. The electron-donating group as R⁰¹ is not particularly limited. Examples of the electron-donating group include an alkyl group and an alkoxy group. The alkyl group and the alkoxy group have preferably 1 to 5 carbon atoms, more preferably 1 to 3 carbon atoms, and still more preferably 1 or 2 carbon atoms. Specific examples of the electron-donating group include a methyl group, an ethyl group, a tert-butyl group, an n-butyl group, and a methoxy group.

In Formula (c0), n1 represents an integer of 0 to 3. n1 represents preferably 0 to 2 and more preferably 0 or 1. In a case where n1 represents 2 or greater, a plurality of R⁰¹'s may be the same as or different from each other.

nx1+nx2+n1≤5 is satisfied.

In Formula (c0), R⁰² and R⁰³ each independently represents a substituent. The substituent as R⁰² and R⁰³ may be an electron-withdrawing group or an electron-donating group. Examples of the substituent as R⁰² and R⁰³ include the same substituents as those for the electron-withdrawing group and the electron-donating group.

In Formula (c0), n2 and n3 each independently represents an integer of 0 to 4. n2 and n3 represent preferably 0 to 3, more preferably 0 to 2, and still more preferably 0 or 1. In a case where n2 represents 2 or greater, a plurality of R⁰²'s may be the same as or different from each other. In a case where n3 represents 2 or greater, a plurality of R⁰³'s may be the same as or different from each other.

Specific examples of the cation (C0) are shown below, but the present invention is not limited thereto.

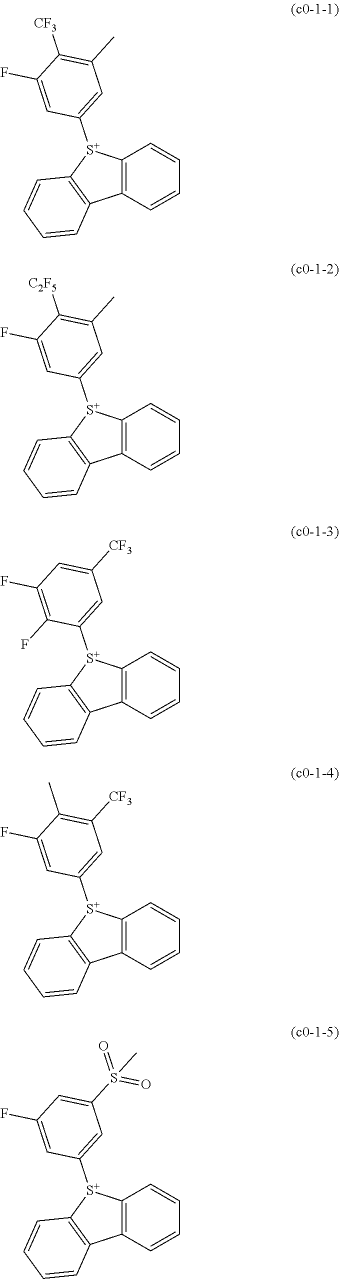

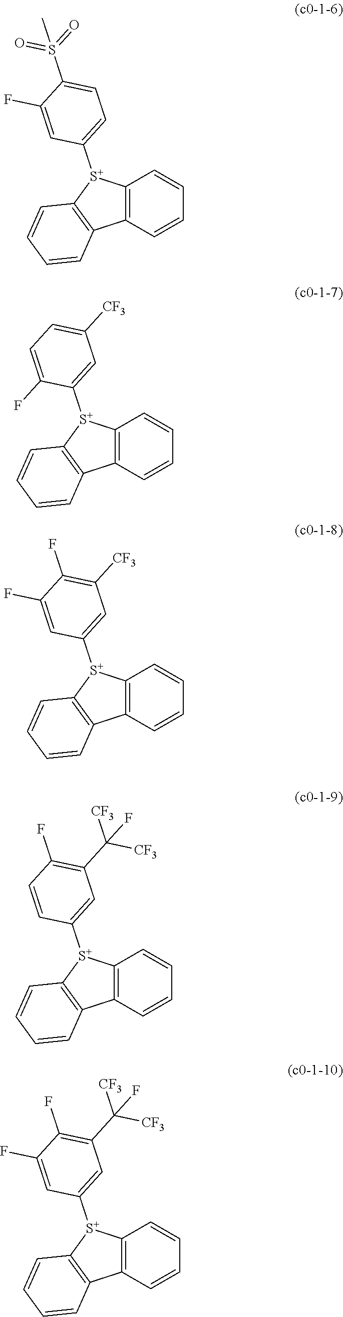 [SMILES: CS(=O)(=O)c1ccc(-[s+]2c3ccccc3c3ccccc32)cc1F ; Fc1ccc(C(F)(F)F)cc1-[s+]1c2ccccc2c2ccccc21 ; Fc1cc(-[s+]2c3ccccc3c3ccccc32)cc(C(F)(F)F)c1F ; Fc1ccc(-[s+]2c3ccccc3c3ccccc32)cc1C(F)(C(F)(F)F)C(F)(F)F ; Fc1cc(-[s+]2c3ccccc3c3ccccc32)cc(C(F)(C(F)(F)F)C(F)(F)F)c1F]

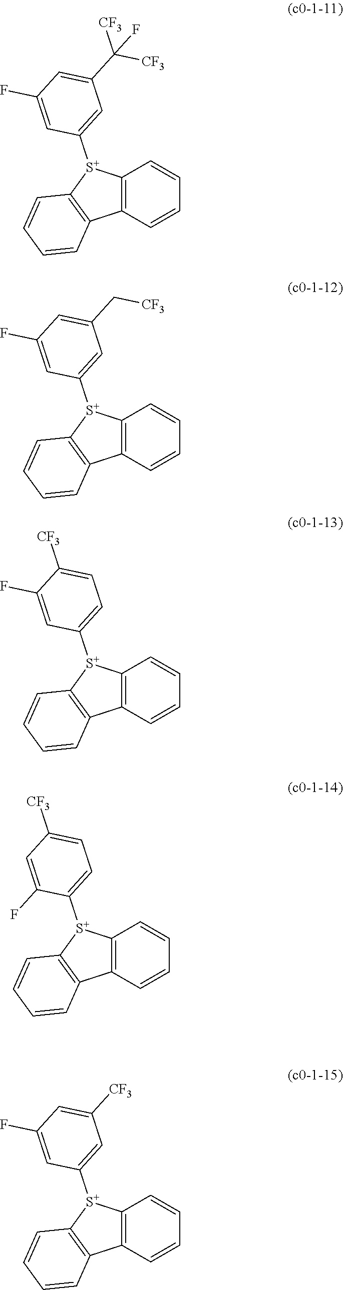 [SMILES: Fc1cc(-[s+]2c3ccccc3c3ccccc32)cc(C(F)(C(F)(F)F)C(F)(F)F)c1 ; Fc1cc(CC(F)(F)F)cc(-[s+]2c3ccccc3c3ccccc32)c1 ; Fc1cc(-[s+]2c3ccccc3c3ccccc32)ccc1C(F)(F)F ; Fc1cc(C(F)(F)F)ccc1-[s+]1c2ccccc2c2ccccc21 ; Fc1cc(-[s+]2c3ccccc3c3ccccc32)cc(C(F)(F)F)c1]

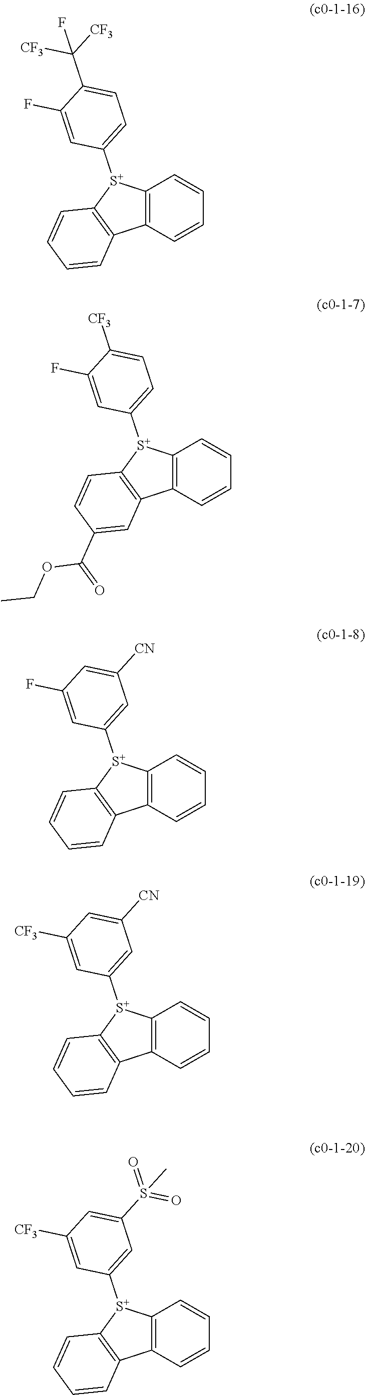 [SMILES: Fc1cc(-[s+]2c3ccccc3c3ccccc32)ccc1C(F)(C(F)(F)F)C(F)(F)F ; CCOC(=O)c1ccc2c(c1)c1ccccc1[s+]2-c1ccc(C(F)(F)F)c(F)c1 ; N#Cc1cc(F)cc(-[s+]2c3ccccc3c3ccccc32)c1 ; N#Cc1cc(-[s+]2c3ccccc3c3ccccc32)cc(C(F)(F)F)c1 ; CS(=O)(=O)c1cc(-[s+]2c3ccccc3c3ccccc32)cc(C(F)(F)F)c1]

### First Embodiment

The resist composition according to the first embodiment contains the compound (C) as the component (B). The resist composition according to the first embodiment contains the component (A) which generates an acid upon light exposure and whose solubility in a developing solution is changed due to the action of an acid, and the acid-generating agent component (B) that generates an acid upon light exposure. The acid-generating agent component (B) contains a compound represented by General Formula (b0).

M^{b⊕}Xb^{⊖} (b0)

[In the formula, M^{b+} represents the cation (C0). Xb⁻ represents a counter anion.]

### <Component (A)>

In the resist composition of the present embodiment, it is preferable that the component (A) have a resin component (A1) whose solubility in a developing solution is changed due to the action of an acid (hereinafter, also referred to as “component (A1)”). Since the polarity of the base material component before and after the light exposure is changed by using the component (A1), an excellent development contrast can be obtained not only in an alkali developing process but also in a solvent developing process.

As the component (A), at least the component (A1) is used, and other high-molecular-weight compounds and/or low-molecular-weight compounds may be used in combination with the component (A1).

In a case of applying an alkali developing process, the base material component having the component (A1) is insoluble in an alkali developing solution before light exposure, but in a case where an acid is generated from the component (B) upon light exposure, the polarity of the component (A1) is increased due to the action of the acid, thereby increasing the solubility of the component (A1) in an alkali developing solution. Therefore, in a case where selective light exposure is performed on a resist film formed by coating a support with the resist composition in the resist pattern formation, the exposed portion of the resist film is changed from being insoluble to being soluble in an alkali developing solution, whereas the unexposed portion of the resist film remains insoluble in an alkali developing solution, and thus a positive-tone resist pattern is formed by performing alkali development.

Meanwhile, in a case of a solvent developing process, the base material component having the component (A1) exhibits high solubility in an organic developing solution before light exposure. For example, in a case where an acid is generated from the component (B) upon light exposure, the polarity of the component (A1) is increased due to the action of the acid, thereby decreasing the solubility of the component (A1) in an organic developing solution. Therefore, in a case where selective light exposure is performed on a resist film formed by coating a support with the resist composition in the resist pattern formation, the exposed portion of the resist film is changed from being soluble to being insoluble in an organic developing solution, whereas the unexposed portion of the resist film remains soluble in an organic developing solution. Therefore, a negative-tone resist pattern is formed by performing development using an organic developing solution so that a contrast is imparted between the exposed portion and the unexposed portion.

In the resist composition according to the present embodiment, the component (A) may be used alone or in combination of two or more kinds thereof.

### Regarding Component (A1)

The component (A1) is a resin component whose solubility in a developing solution is changed due to the action of an acid.

As the component (A1), those having a constitutional unit (a1) containing an acid-decomposable group whose polarity is increased due to the action of an acid are preferable.

Further, the component (A1) may have other constitutional units as necessary in addition to the constitutional unit (a1).

<<Constitutional Unit (a1)>>

The constitutional unit (a1) is a constitutional unit that contains an acid-decomposable group whose polarity is increased due to the action of an acid.

Examples of the acid-dissociable group are the same as those which have been suggested as the acid-dissociable groups of the base resin for a chemically amplified resist composition.

Specific examples of the acid-dissociable group of the base resin for a chemically amplified resist composition include an “acetal type acid-dissociable group”, a “tertiary alkyl ester type acid-dissociable group”, and a “tertiary alkyloxycarbonyl acid-dissociable group” described below.

Acetal type acid-dissociable group:

Examples of the acid-dissociable group that protects a carboxy group or a hydroxyl group in the polar groups include an acid-dissociable group represented by General Formula (a1-r-1) (hereinafter, also referred to as “acetal type acid-dissociable group”).

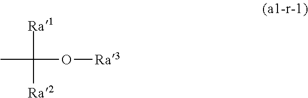

[In the formula, Ra′¹ and Ra′² represent a hydrogen atom or an alkyl group. Ra′³ represents a hydrocarbon group, and Ra′³ may be bonded to any of Ra′¹ and Ra′² to form a ring.]

In Formula (a1-r-1), it is preferable that at least one of Ra′¹ or Ra′² represent a hydrogen atom and more preferable that both Ra′¹ and Ra′² represent a hydrogen atom. In a case where Ra′¹ or Ra′² represents an alkyl group, examples of the alkyl group include the same alkyl groups described as the substituent which may be bonded to the carbon atom at the α-position in the description of the α-substituted acrylic acid ester. Among these, an alkyl group having 1 to 5 carbon atoms is preferable. Specific preferred examples thereof include linear or branched alkyl groups. More specific examples thereof include a methyl group, an ethyl group, a propyl group, an isopropyl group, an n-butyl group, an isobutyl group, a tert-butyl group, a pentyl group, an isopentyl group, and a neopentyl group. Among these, a methyl group or an ethyl group is more preferable, and a methyl group is particularly preferable.

In Formula (a1-r-1), examples of the hydrocarbon group as Ra′³ include a linear or branched alkyl group and a cyclic hydrocarbon group.

The linear alkyl group has preferably 1 to 5 carbon atoms, more preferably 1 to 4 carbon atoms, and still more preferably 1 or 2 carbon atoms. Specific examples thereof include a methyl group, an ethyl group, an n-propyl group, an n-butyl group, and an n-pentyl group. Among these, a methyl group, an ethyl group, or an n-butyl group is preferable, and a methyl group or an ethyl group is more preferable.

The branched alkyl group has preferably 3 to 10 carbon atoms and more preferably 3 to 5 carbon atoms. Specific examples thereof include an isopropyl group, an isobutyl group, a tert-butyl group, an isopentyl group, a neopentyl group a 1,1-diethylpropyl group, and a 2,2-dimethylbutyl group. Among these, an isopropyl group is preferable.

In a case where Ra′³ represents a cyclic hydrocarbon group, the hydrocarbon group may be an aliphatic hydrocarbon group or an aromatic hydrocarbon group and may be a polycyclic group or a monocyclic group.

As the aliphatic hydrocarbon group which is a monocyclic group, a group in which one hydrogen atom has been removed from a monocycloalkane is preferable. The monocycloalkane has preferably 3 to 6 carbon atoms, and specific examples thereof include cyclopentane and cyclohexane.

As the aliphatic hydrocarbon group which is a polycyclic group, a group in which one hydrogen atom has been removed from a polycycloalkane is preferable. As the polycycloalkane, a group having 7 to 12 carbon atoms is preferable, and specific examples thereof include adamantane, norbornane, isobornane, tricyclodecane, and tetracyclododecane.

In a case where the cyclic hydrocarbon group as Ra′³ is an aromatic hydrocarbon group, the aromatic hydrocarbon group is a hydrocarbon group having at least one aromatic ring.

The aromatic ring is not particularly limited as long as the aromatic ring is a cyclic conjugated system having (4n+2) π electrons and may be monocyclic or polycyclic. The aromatic ring has preferably 5 to 30 carbon atoms, more preferably 5 to 20 carbon atoms, still more preferably 6 to 15 carbon atoms, and particularly preferably 6 to 12 carbon atoms.

Specific examples of the aromatic ring include aromatic hydrocarbon rings such as benzene, naphthalene, anthracene, and phenanthrene; and aromatic heterocyclic rings in which some carbon atoms constituting the above-described aromatic hydrocarbon rings have been substituted with hetero atoms. Examples of the hetero atom in the aromatic heterocyclic rings include an oxygen atom, a sulfur atom, and a nitrogen atom. Specific examples of the aromatic heterocyclic ring include a pyridine ring and a thiophene ring.

Specific examples of the aromatic hydrocarbon group as Ra′³ include a group in which one hydrogen atom has been removed from the above-described aromatic hydrocarbon ring or aromatic heterocyclic ring (such as an aryl group or a heteroaryl group); a group in which one hydrogen atom has been removed from an aromatic compound having two or more aromatic rings (such as biphenyl or fluorene); and a group in which one hydrogen atom of the above-described aromatic hydrocarbon ring or aromatic heterocyclic ring has been substituted with an alkylene group (for example, an arylalkyl group such as a benzyl group, a phenethyl group, a 1-naphthylmethyl group, a 2-naphthylmethyl group, a 1-naphthylethyl group, or a 2-naphthylethyl group). The number of carbon atoms in the alkylene group bonded to the aromatic hydrocarbon ring or aromatic heterocyclic ring is preferably in a range of 1 to 4, more preferably 1 or 2, and particularly preferably 1.

The cyclic hydrocarbon group as Ra′³ may include a substituent. Examples of the substituent include —R^{P1}, —R^{P2}—O—R^{P1}, —R^{P2}—CO—R^{P1}, —R^{P2}—CO—OR^{P1}, —R^{P2}—O—CO—R^{P1}, —R^{P2}—OH, —R^{P2}—CN, and —R^{P2}—COOH (hereinafter, these substituents will also be collectively referred to as “Ra^{x5}”).

Here, R^{P1} represents a chain-like monovalent saturated hydrocarbon group having 1 to 10 carbon atoms, a monovalent aliphatic cyclic saturated hydrocarbon group having 3 to 20 carbon atoms, or a monovalent aromatic hydrocarbon group having 6 to 30 carbon atoms. Further, R^{P2} represents a single bond, a chain-like divalent saturated hydrocarbon group having 1 to 10 carbon atoms, a divalent aliphatic cyclic saturated hydrocarbon group having 3 to 20 carbon atoms, or a divalent aromatic hydrocarbon group having 6 to 30 carbon atoms. Here, a part or all of the hydrogen atoms in the chain-like saturated hydrocarbon group, the aliphatic cyclic saturated hydrocarbon group, and the aromatic hydrocarbon group as R^{P1} and R^{P2} may be substituted with fluorine atoms. The aliphatic cyclic hydrocarbon group may have one or more of a single kind of substituents or one or more of each of plural kinds of the substituents.

Examples of the chain-like monovalent saturated hydrocarbon group having 1 to 10 carbon atoms include a methyl group, an ethyl group, a propyl group, a butyl group, a pentyl group, a hexyl group, a heptyl group, an octyl group, and a decyl group.

Examples of the monovalent aliphatic cyclic saturated hydrocarbon group having 3 to 20 carbon atoms include a monocyclic aliphatic saturated hydrocarbon group such as a cyclopropyl group, a cyclobutyl group, a cyclopentyl group, a cyclohexyl group, a cycloheptyl group, a cyclooctyl group, a cyclodecyl group, or a cyclododecyl group; and a polycyclic aliphatic saturated hydrocarbon group such as a bicyclo[2.2.2]octanyl group, a tricyclo[5.2.1.02,6]decanyl group, a tricyclo[3.3.1.13,7]decanyl group, a tetracyclo[6.2.1.13,6.02,7] dodecanyl group, or an adamantyl group.

Examples of the monovalent aromatic hydrocarbon group having 6 to 30 carbon atoms include a group formed by removing one hydrogen atom from an aromatic hydrocarbon ring such as benzene, biphenyl, fluorene, naphthalene, anthracene, or phenanthrene.

In a case where Ra′³ is bonded to any of Ra′¹ and Ra′² to form a ring, the cyclic group is preferably a 4- to 7-membered ring and more preferably a 4- to 6-membered ring. Specific examples of the cyclic group include a tetrahydropyranyl group and a tetrahydrofuranyl group.

### Tertiary Alkyl Ester Type Acid-Dissociable Group:

Examples of the acid-dissociable group that protects a carboxy group among the polar groups include an acid-dissociable group represented by General Formula (a1-r-2).

Among examples of the acid-dissociable group represented by Formula (a1-r-2), a group formed of an alkyl group is referred to as “tertiary alkyl ester type acid-dissociable group” for convenience.

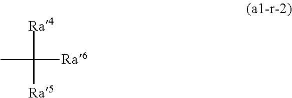

[In the formula, Ra′⁴ to Ra′⁶ each represents a hydrocarbon group, and Ra′⁵ and Ra′⁶ may be bonded to each other to form a ring.]

Examples of the hydrocarbon group as Ra′⁴ include a linear or branched alkyl group, a chain-like or cyclic alkenyl group, and a cyclic hydrocarbon group.

Examples of the linear or branched alkyl group and the cyclic hydrocarbon group (an aliphatic hydrocarbon group which is a monocyclic group, an aliphatic hydrocarbon group which is a polycyclic group, or an aromatic hydrocarbon group) as Ra′⁴ include the same groups as those for Ra′³.

As the chain-like or cyclic alkenyl group as Ra′⁴, an alkenyl group having 2 to 10 carbon atoms is preferable.

Examples of the hydrocarbon group as Ra′⁵ or Ra′⁶ include the same groups as those for Ra′³.

In a case where Ra′⁵ and Ra′⁶ are bonded to each other to form a ring, suitable examples thereof include a group represented by General Formula (a1-r2-1), a group represented by General Formula (a1-r2-2), and a group represented by General Formula (a1-r2-3).

Meanwhile, in a case where Ra′⁴ to Ra′⁶ independently represent a hydrocarbon group without being bonded to one another, suitable examples thereof include a group represented by General Formula (a1-r2-4).

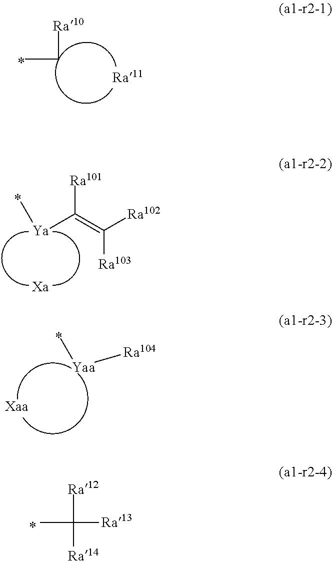

[In Formula (a1-r2-1), Ra′¹⁰ represents a linear or branched alkyl group having to 12 carbon atoms, in which a part thereof may be substituted with a halogen atom or a hetero atom-containing group. Ra′¹¹ represents a group that forms an aliphatic cyclic group with the carbon atom to which Ra′¹⁰ has been bonded. In Formula (a1-r2-2), Ya represents a carbon atom. Xa represents a group that forms a cyclic hydrocarbon group with Ya. A part or all of the hydrogen atoms in this cyclic hydrocarbon group may be substituted. Ra¹⁰¹ to Ra¹⁰³ each independently represents a hydrogen atom, a chain-like monovalent saturated hydrocarbon group having 1 to 10 carbon atoms, or a monovalent aliphatic cyclic saturated hydrocarbon group having 3 to 20 carbon atoms. A part or all of the hydrogen atoms in the chain-like saturated hydrocarbon group and the aliphatic cyclic saturated hydrocarbon group may be substituted. Two or more of Ra′⁰¹ to Ra¹⁰³ may be bonded to each other to form a cyclic structure. In Formula (a1-r2-3), Yaa represents a carbon atom. Xaa represents a group that forms an aliphatic cyclic group with Yaa. Ra¹⁰⁴ represents an aromatic hydrocarbon group which may have a substituent. In Formula (a1-r2-4), Ra′¹² and Ra′¹³ each independently represents a chain-like monovalent saturated hydrocarbon group having 1 to 10 carbon atoms. A part or all of the hydrogen atoms in this chain-like saturated hydrocarbon group may be substituted. Ra′¹⁴ represents a hydrocarbon group which may have a substituent. * represents a bonding site.]

In Formula (a1-r2-1), Ra′¹⁰ represents a linear or branched alkyl group having 1 to 12 carbon atoms, in which a part thereof may be substituted with a halogen atom or a hetero atom-containing group.

The linear alkyl group as Ra′¹⁰ has 1 to 12 carbon atoms, preferably 1 to 10 carbon atoms, and particularly preferably 1 to 5 carbon atoms.

Examples of the branched alkyl group as Ra′¹⁰ include those for Ra′³ described above.

The alkyl group in Ra′¹⁰ may be partially substituted with a halogen atom or a hetero atom-containing group. For example, some hydrogen atoms constituting the alkyl group may be substituted with a halogen atom or a hetero atom-containing group. Further, some carbon atoms (methylene group or the like) constituting the alkyl group may be substituted with a hetero atom-containing group.

Examples of the hetero atoms here include an oxygen atom, a nitrogen atom, and a sulfur atom. Examples of the hetero atom-containing group include (—O—), —C(═O)—O—, —O—C(═O)—, —C(═O)—, —O—C(═O)—O—, —C(═O)—NH—, —NH—, —S—, —S(═O)₂—, and —S(═O)₂—O—.

In Formula (a1-r2-1), preferred examples of Ra′¹¹ (aliphatic cyclic group that is formed together with a carbon atom to which Ra′¹⁰ is bonded) include the groups described as the aliphatic hydrocarbon group (alicyclic hydrocarbon group) which is a monocyclic group or a polycyclic group as Ra′³ in Formula (a1-r-1). Among these, a monocyclic alicyclic hydrocarbon group is preferable, specifically, a cyclopentyl group or a cyclohexyl group is more preferable, and a cyclopentyl group is still more preferable.

In Formula (a1-r2-2), examples of the cyclic hydrocarbon group that is formed by Xa together with Ya include a group in which one or more hydrogen atoms have been further removed from the cyclic monovalent hydrocarbon group (aliphatic hydrocarbon group) as Ra′³ in Formula (a1-r-1).

The cyclic hydrocarbon group that is formed by Xa together with Ya may have a substituent. Examples of the substituent include those described as the substituents that the cyclic hydrocarbon group as Ra′³ may have.

In Formula (a1-r2-2), examples of the chain-like monovalent saturated hydrocarbon group having 1 to 10 carbon atoms as Ra¹⁰¹ to Ra¹⁰³ include a methyl group, an ethyl group, a propyl group, a butyl group, a pentyl group, a hexyl group, a heptyl group, an octyl group, and a decyl group.

Examples of the monovalent aliphatic cyclic saturated hydrocarbon group having 3 to 20 carbon atoms as Ra¹⁰¹ to Ra¹⁰³ include a monocyclic aliphatic saturated hydrocarbon group such as a cyclopropyl group, a cyclobutyl group, a cyclopentyl group, a cyclohexyl group, a cycloheptyl group, a cyclooctyl group, a cyclodecyl group, or a cyclododecyl group; and a polycyclic aliphatic saturated hydrocarbon group such as a bicyclo[2.2.2] octanyl group, a tricyclo[5.2.1.02,6]decanyl group, a tricyclo[3.3.1.13,7]decanyl group, a tetracyclo[6.2.1.13,6.02,7] dodecanyl group, or an adamantyl group. From the viewpoint of ease of synthesis, Ra¹⁰¹ to Ra¹⁰³ represent preferably a hydrogen atom or a chain-like monovalent saturated hydrocarbon group having 1 to 10 carbon atoms, more preferably a hydrogen atom, a methyl group, or an ethyl group, and particularly preferably a hydrogen atom.

Examples of the substituent included in the chain-like saturated hydrocarbon group or the aliphatic cyclic saturated hydrocarbon group represented by Ra¹⁰¹ to Ra¹⁰³ include the same substituents as those for Ra^{x5}.

Examples of the group having a carbon-carbon double bond generated by two or more of Ra¹⁰¹ to Ra¹⁰³ being bonded to each other to form a cyclic structure include a cyclopentenyl group, a cyclohexenyl group, a methylcyclopentenyl group, a methylcyclohexenyl group, a cyclopentylidenethenyl group, and a cyclohexylidenethenyl group. Among these, from the viewpoint of ease of synthesis, a cyclopentenyl group, a cyclohexenyl group, or a cyclopentylidenethenyl group is preferable.

In Formula (a1-r2-3), as the aliphatic cyclic group that is formed by Xaa together with Yaa, the group described as the aliphatic hydrocarbon group which is a monocyclic group or a polycyclic group as Ra′³ in Formula (a1-r-1) is preferable.

In Formula (a1-r2-3), examples of the aromatic hydrocarbon group as Ra¹⁰⁴ include a group in which one or more hydrogen atoms have been removed from an aromatic hydrocarbon ring having 5 to 30 carbon atoms. Among the examples, Ra¹⁰⁴ represents preferably a group in which one or more hydrogen atoms have been removed from an aromatic hydrocarbon ring having 6 to 15 carbon atoms, more preferably a group in which one or more hydrogen atoms have been removed from benzene, naphthalene, anthracene, or phenanthrene, still more preferably a group in which one or more hydrogen atoms have been removed from benzene, naphthalene, or anthracene, particularly preferably a group in which one or more hydrogen atoms have been removed from benzene or naphthalene, and most preferably a group in which one or more hydrogen atoms have been removed from benzene.

Examples of the substituent that Ra¹⁰⁴ in Formula (a1-r2-3) may have include a methyl group, an ethyl group, a propyl group, a hydroxy group, a carboxy group, a halogen atom, an alkoxy group (such as a methoxy group, an ethoxy group, a propoxy group, or a butoxy group), and an alkyloxycarbonyl group.

In Formula (a1-r2-4), Ra′¹² and Ra′¹³ each independently represents a chain-like monovalent saturated hydrocarbon group having 1 to 10 carbon atoms. Examples of the chain-like monovalent saturated hydrocarbon group having 1 to 10 carbon atoms as Ra′12 and Ra′¹³ include those described as the chain-like monovalent saturated hydrocarbon group having 1 to 10 carbon atoms as Ra¹⁰¹ to Ra¹⁰³. A part or all of the hydrogen atoms in this chain-like saturated hydrocarbon group may be substituted.

Ra′¹² and Ra′¹³ represent preferably an alkyl group having 1 to 5 carbon atoms, more preferably an alkyl group having 1 to 5 carbon atoms, still more preferably a methyl group or an ethyl group, and particularly preferably a methyl group.

In a case where the chain-like saturated hydrocarbon group represented by Ra′¹² and Ra′¹³ is substituted, examples of the substituent thereof include the same substituents as those for Ra^{x5}.

In Formula (a1-r2-4), Ra′¹⁴ represents a hydrocarbon group which may have a substituent. Examples of the hydrocarbon group as Ra′¹⁴ include a linear or branched alkyl group and a cyclic hydrocarbon group.

The linear alkyl group as Ra′¹⁴ has preferably 1 to 5 carbon atoms, more preferably 1 to 4 carbon atoms, and still more preferably 1 or 2 carbon atoms. Specific examples thereof include a methyl group, an ethyl group, an n-propyl group, an n-butyl group, and an n-pentyl group. Among these, a methyl group, an ethyl group, or an n-butyl group is preferable, and a methyl group or an ethyl group is more preferable.

The branched alkyl group as Ra′¹⁴ has preferably 3 to 10 carbon atoms and more preferably 3 to 5 carbon atoms. Specific examples thereof include an isopropyl group, an isobutyl group, a tert-butyl group, an isopentyl group, a neopentyl group a 1,1-diethylpropyl group, and a 2,2-dimethylbutyl group. Among these, an isopropyl group is preferable.

In a case where Ra′¹⁴ represents a cyclic hydrocarbon group, the hydrocarbon group may be an aliphatic hydrocarbon group or an aromatic hydrocarbon group and may be a polycyclic group or a monocyclic group.

As the aliphatic hydrocarbon group which is a monocyclic group, a group in which one hydrogen atom has been removed from a monocycloalkane is preferable. The monocycloalkane has preferably 3 to 6 carbon atoms, and specific examples thereof include cyclopentane and cyclohexane.

As the aliphatic hydrocarbon group which is a polycyclic group, a group in which one hydrogen atom has been removed from a polycycloalkane is preferable. As the polycycloalkane, a group having 7 to 12 carbon atoms is preferable, and specific examples thereof include adamantane, norbornane, isobornane, tricyclodecane, and tetracyclododecane.

Examples of the aromatic hydrocarbon group as Ra′¹⁴ include the same groups as those for the aromatic hydrocarbon group as Ra¹⁰⁴. Among these, Ra′¹⁴ represents preferably a group in which one or more hydrogen atoms have been removed from an aromatic hydrocarbon ring having 6 to 15 carbon atoms, more preferably a group in which one or more hydrogen atoms have been removed from benzene, naphthalene, anthracene, or phenanthrene, still more preferably a group in which one or more hydrogen atoms have been removed from benzene, naphthalene, or anthracene, particularly preferably a group in which one or more hydrogen atoms have been removed from naphthalene or anthracene, and most preferably a group in which one or more hydrogen atoms have been removed from naphthalene.

Examples of the substituent that Ra′¹⁴ may have include the same groups as those for the substituent that Ra¹⁰⁴ may have.

In a case where Ra′¹⁴ in Formula (a1-r2-4) represents a naphthyl group, the position bonded to the tertiary carbon atom in Formula (a1-r2-4) may be the 1-position or the 2-position of the naphthyl group.

In a case where Ra′¹⁴ in Formula (a1-r2-4) represents an anthryl group, the position bonded to the tertiary carbon atom in Formula (a1-r2-4) may be the 1-position, the 2-position, or the 9-position of the anthryl group.

Specific examples of the group represented by Formula (a1-r2-1) are shown below.

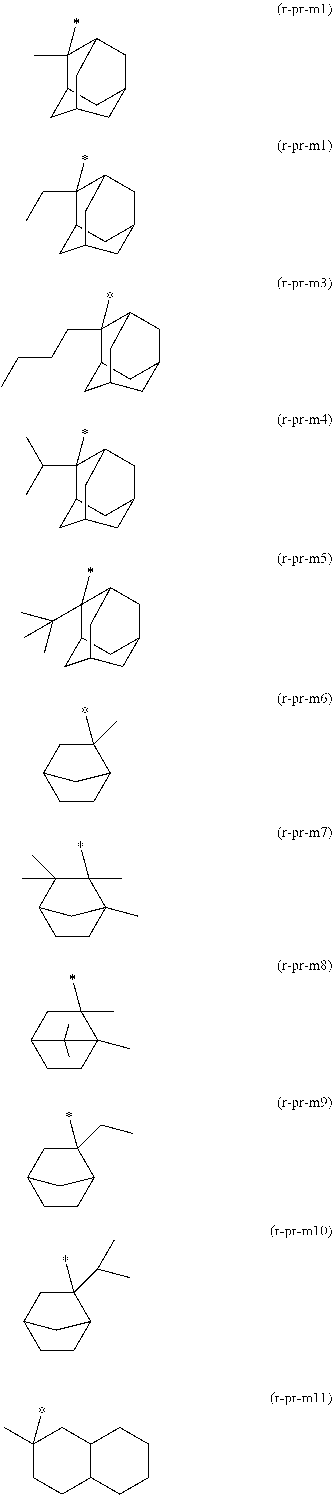

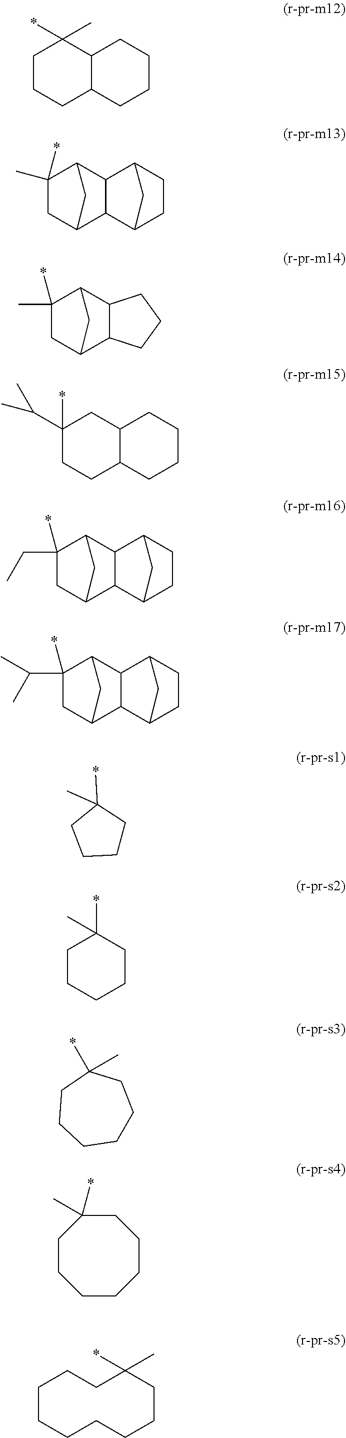

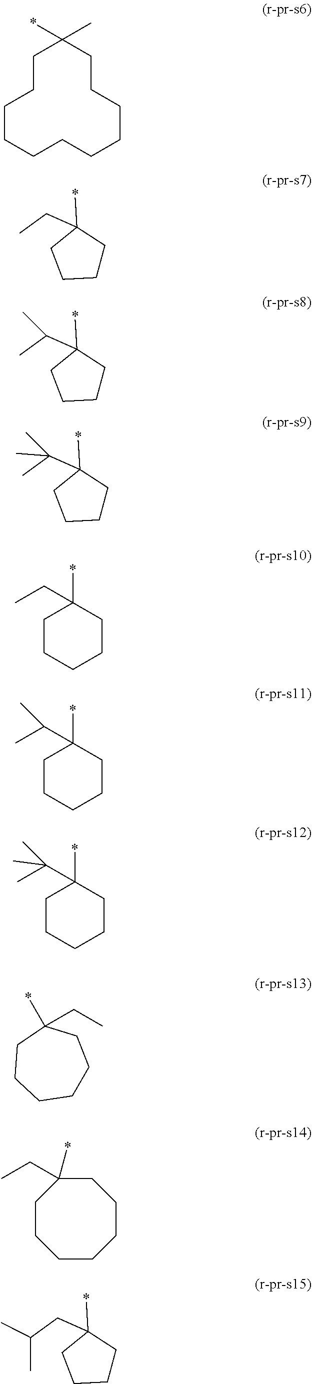

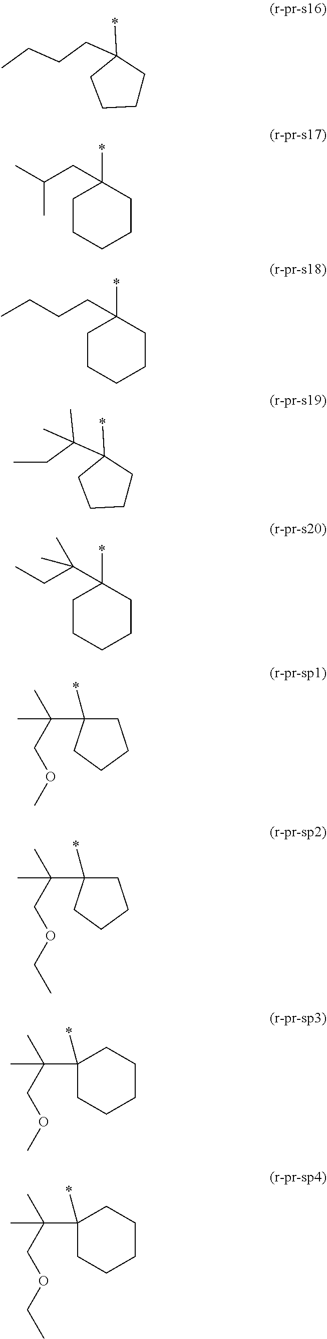

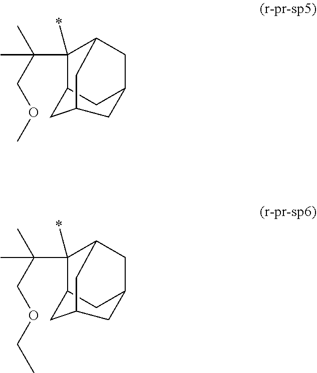 [SMILES: *C1(C(C)(C)COC)C2CC3CC(C2)CC1C3 ; *C1(C(C)(C)COCC)C2CC3CC(C2)CC1C3]

Specific examples of the group represented by Formula (a1-r2-2) are shown below.

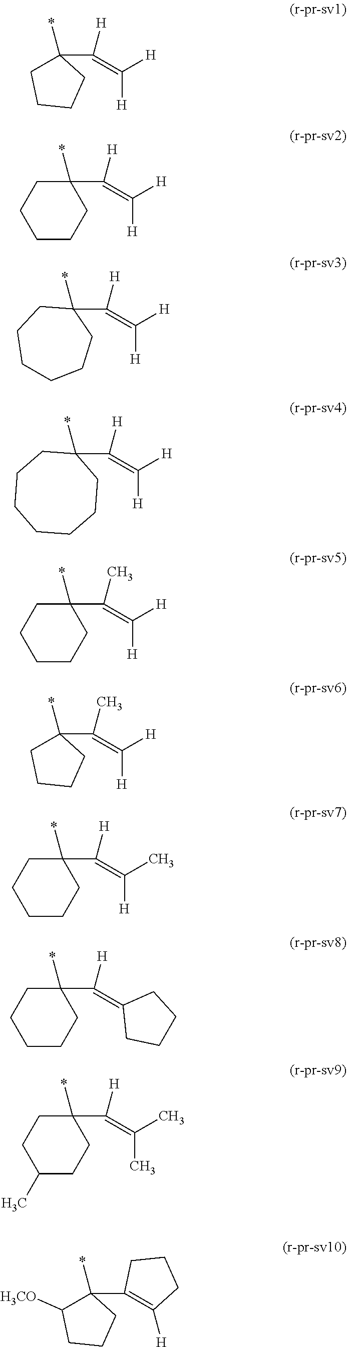

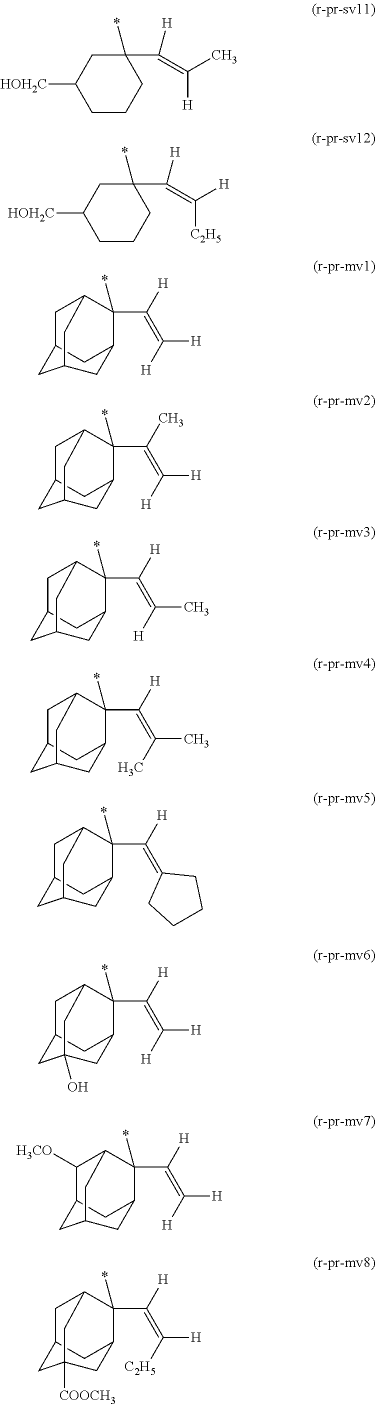

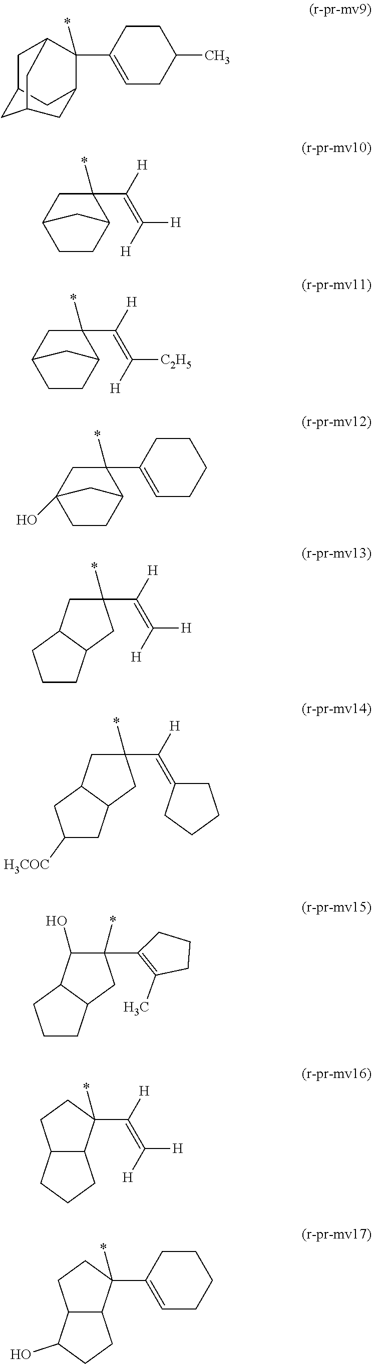

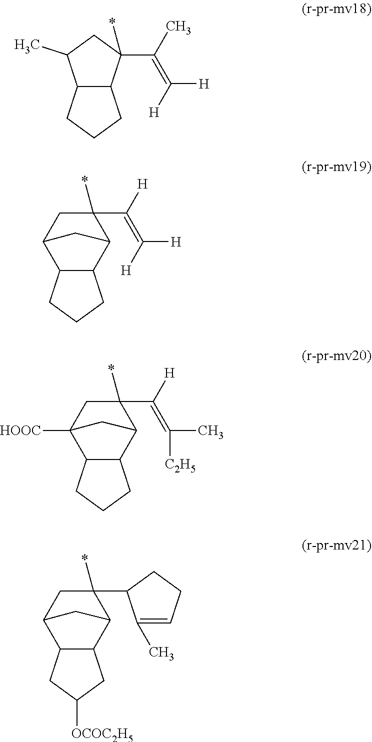

Specific examples of the group represented by Formula (a1-r2-3) are shown below.

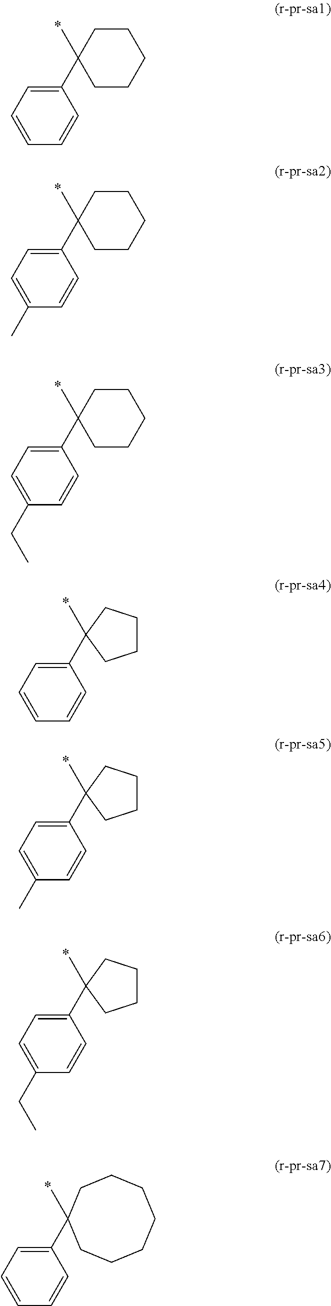

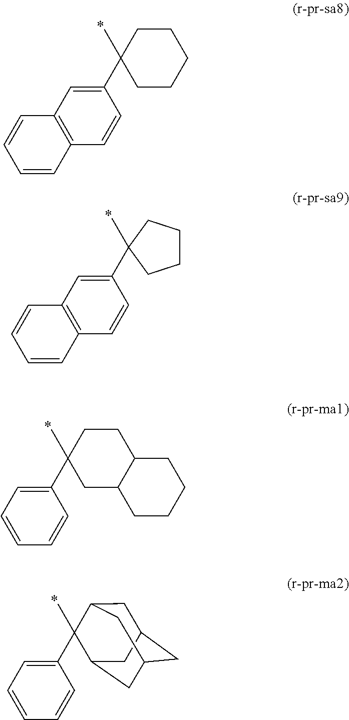

Specific examples of the group represented by Formula (a1-r2-4) are shown below.

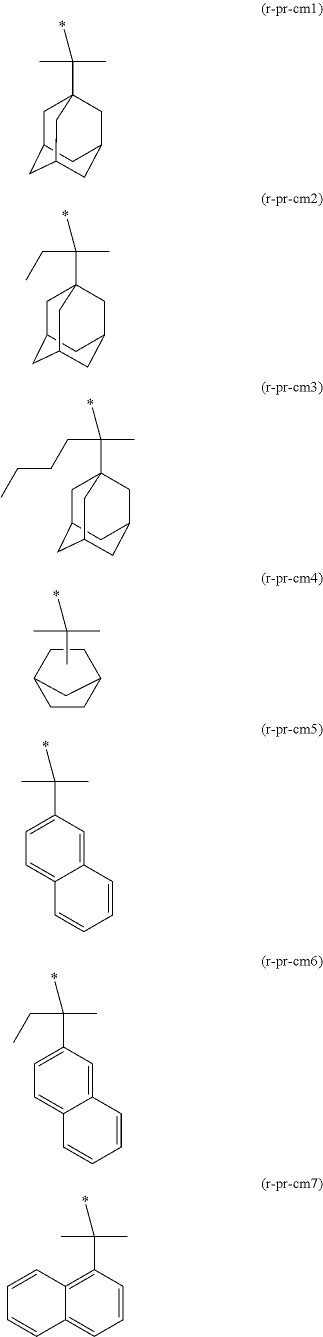

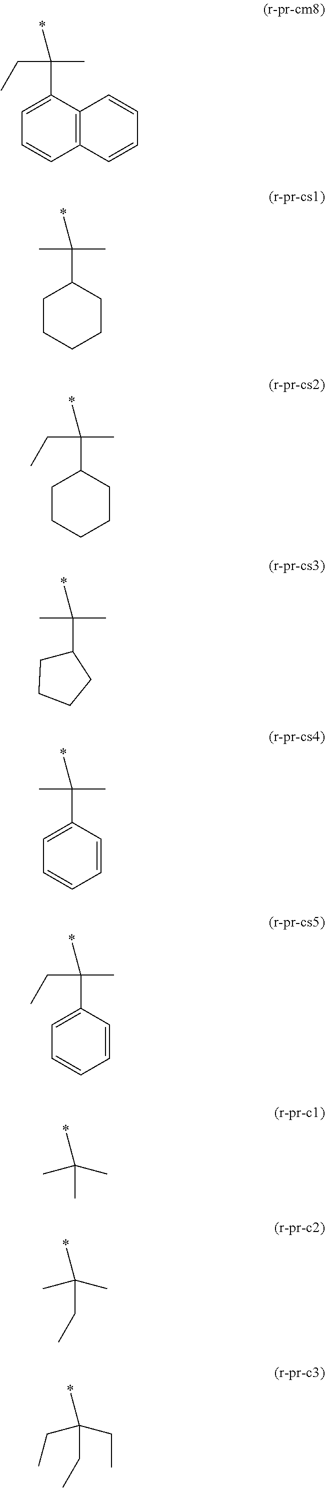

### Tertiary Alkyloxycarbonyl Acid-Dissociable Group:

Examples of the acid-dissociable group that protects a hydroxyl group among the polar groups include an acid-dissociable group (hereinafter, also referred to as “tertiary alkyloxycarbonyl acid-dissociable group” for convenience) represented by General Formula (a1-r-3).

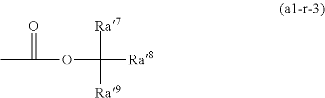

[In the formula, Ra′⁷ to Ra′⁹ each represents an alkyl group.]

In Formula (a1-r-3), Ra′⁷ to Ra′⁹ each represents preferably an alkyl group having 1 to 5 carbon atoms and more preferably an alkyl group having 1 to 3 carbon atoms.

Further, the total number of carbon atoms in each alkyl group is preferably in a range of 3 to 7, more preferably in a range of 3 to 5, and most preferably 3 or 4.

Examples of the constitutional unit (a1) include a constitutional unit derived from acrylic acid ester in which the hydrogen atom bonded to the carbon atom at the α-position may be substituted with a substituent; a constitutional unit derived from acrylamide; a constitutional unit in which at least some hydrogen atoms in a hydroxyl group of a constitutional unit derived from hydroxystyrene or a hydroxystyrene derivative are protected by a substituent containing the acid-decomposable group; and a constitutional unit in which at least some hydrogen atoms in —C(═O)—OH of a constitutional unit derived from vinylbenzoic acid or a vinylbenzoic acid derivative are protected by a substituent containing the acid-decomposable group.

Among the examples, as the constitutional unit (a1), a constitutional unit derived from acrylic acid ester in which the hydrogen atom bonded to the carbon atom at the α-position may be substituted with a substituent is preferable.

Specific preferred examples of such a constitutional unit (a1) include constitutional units represented by General Formula (a1-1) or (a1-2) shown below.

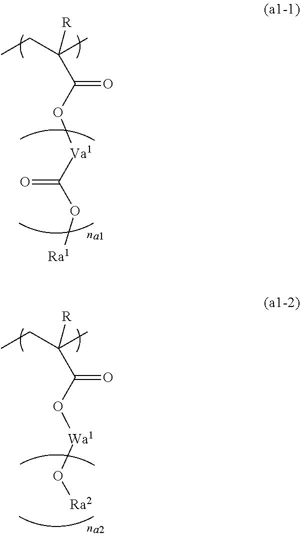

[In the formula, R represents a hydrogen atom, an alkyl group having 1 to 5 carbon atoms, or a halogenated alkyl group having 1 to 5 carbon atoms. Va¹ represents a divalent hydrocarbon group which may have an ether bond. nₐ₁ represents an integer of 0 to 2. Ra¹ represents an acid-dissociable group represented by General Formula (a1-r-1) or (a1-r-2). Wa¹ represents a (nₐ₂+1)-valent hydrocarbon group, nₐ₂ represents an integer of 1 to 3, and Ra² represents an acid-dissociable group represented by General Formula (a1-r-1) or (a1-r-3).]

In Formula (a1-1), as the alkyl group having 1 to 5 carbon atoms as R, a linear or branched alkyl group having 1 to 5 carbon atoms is preferable, and specific examples thereof include a methyl group, an ethyl group, a propyl group, an isopropyl group, an n-butyl group, an isobutyl group, a tert-butyl group, a pentyl group, an isopentyl group, and a neopentyl group. The halogenated alkyl group having 1 to 5 carbon atoms is a group in which a part or all of the hydrogen atoms in the alkyl group having 1 to 5 carbon atoms have been substituted with halogen atoms. As the halogen atom, a fluorine atom is particularly preferable.

R preferably represents a hydrogen atom, an alkyl group having 1 to 5 carbon atoms, or a fluorinated alkyl group having 1 to 5 carbon atoms and most preferably a hydrogen atom or a methyl group from the viewpoint of the industrial availability.

In Formula (a1-1), the divalent hydrocarbon group as Va¹ may be an aliphatic hydrocarbon group or an aromatic hydrocarbon group.

The aliphatic hydrocarbon group as the divalent hydrocarbon group represented by Va¹ may be saturated or unsaturated. In general, it is preferable that the aliphatic hydrocarbon group be saturated.

More specific examples of the aliphatic hydrocarbon group include a linear or branched aliphatic hydrocarbon group and an aliphatic hydrocarbon group having a ring in the structure thereof.

The linear aliphatic hydrocarbon group has preferably 1 to 10 carbon atoms, more preferably 1 to 6 carbon atoms, still more preferably 1 to 4 carbon atoms, and most preferably 1 to 3 carbon atoms.

As the linear aliphatic hydrocarbon group, a linear alkylene group is preferable, and specific examples thereof include a methylene group [—CH₂—], an ethylene group [—(CH₂)₂—], a trimethylene group [—(CH₂)₃—], a tetramethylene group [—(CH₂)₄—], and a pentamethylene group [—(CH₂)₅—].

The branched aliphatic hydrocarbon group has preferably 2 to 10 carbon atoms, more preferably 3 to 6 carbon atoms, still more preferably 3 or 4 carbon atoms, and most preferably 3 carbon atoms.

As the branched aliphatic hydrocarbon group, a branched alkylene group is preferable, and specific examples thereof include alkylalkylene groups, for example, alkylmethylene groups such as —CH(CH₃)—, —CH(CH₂CH₃)—, —C(CH₃)₂—, —C(CH₃)(CH₂CH₃)—, —C(CH₃)(CH₂CH₂CH₃)—, and —C(CH₂CH₃)₂—; alkylethylene groups such as —CH(CH₃)CH₂—, —CH(CH₃)CH(CH₃)—, —C(CH₃)₂CH₂—, —CH(CH₂CH₃)CH₂—, and —C(CH₂CH₃)₂—CH₂—; alkyltrimethylene groups such as —CH(CH₃)CH₂CH₂— and —CH₂CH(CH₃)CH₂—; and alkyltetramethylene groups such as —CH(CH₃)CH₂CH₂CH₂— and —CH₂CH(CH₃)CH₂CH₂—. As the alkyl group in the alkylalkylene group, a linear alkyl group having 1 to 5 carbon atoms is preferable.

Examples of the aliphatic hydrocarbon group having a ring in the structure thereof include an alicyclic hydrocarbon group (a group in which two hydrogen atoms have been removed from an aliphatic hydrocarbon ring), a group in which the alicyclic hydrocarbon group is bonded to the terminal of the linear or branched aliphatic hydrocarbon group, and a group in which the alicyclic hydrocarbon group is interposed in the middle of the linear or branched aliphatic hydrocarbon group. Examples of the linear or branched aliphatic hydrocarbon group include the same groups as those for the linear aliphatic hydrocarbon group or the branched aliphatic hydrocarbon group.

The alicyclic hydrocarbon group has preferably 3 to 20 carbon atoms and more preferably 3 to 12 carbon atoms.

The alicyclic hydrocarbon group may be monocyclic or polycyclic. As the monocyclic alicyclic hydrocarbon group, a group in which two hydrogen atoms have been removed from a monocycloalkane is preferable. The monocycloalkane has preferably 3 to 6 carbon atoms, and specific examples thereof include cyclopentane and cyclohexane. As the polycyclic alicyclic hydrocarbon group, a group in which two hydrogen atoms have been removed from a polycycloalkane is preferable. As the polycycloalkane, a group having 7 to 12 carbon atoms is preferable. Specific examples thereof include adamantane, norbornane, isobornane, tricyclodecane, and tetracyclododecane.

The aromatic hydrocarbon group as the divalent hydrocarbon group represented by Va¹ is a hydrocarbon group having an aromatic ring.

The aromatic hydrocarbon group has preferably 3 to 30 carbon atoms, more preferably 5 to 30 carbon atoms, still more preferably 5 to 20 carbon atoms, particularly preferably 6 to 15 carbon atoms, and most preferably 6 to 12 carbon atoms. Here, the number of carbon atoms in a substituent is not included in the above number of carbon atoms.

Specific examples of the aromatic ring contained in the aromatic hydrocarbon group include aromatic hydrocarbon rings such as benzene, biphenyl, fluorene, naphthalene, anthracene, and phenanthrene; and aromatic heterocyclic rings in which some carbon atoms constituting the above-described aromatic hydrocarbon rings have been substituted with hetero atoms. Examples of the hetero atom in the aromatic heterocyclic rings include an oxygen atom, a sulfur atom, and a nitrogen atom.

Specific examples of the aromatic hydrocarbon group include a group in which two hydrogen atoms have been removed from the above-described aromatic hydrocarbon ring (an arylene group); and a group in which one hydrogen atom of a group (an aryl group) formed by removing one hydrogen atom from the aromatic hydrocarbon ring has been substituted with an alkylene group (for example, a group formed by further removing one more hydrogen atom from an aryl group in an arylalkyl group such as a benzyl group, a phenethyl group, a 1-naphthylmethyl group, a 2-naphthylmethyl group, a 1-naphthylethyl group, or a 2-naphthylethyl group). The alkylene group (alkyl chain in the arylalkyl group) has preferably 1 to 4 carbon atoms, more preferably 1 or 2 carbon atoms, and particularly preferably 1 carbon atom.

In Formula (a1-1), Ra¹ represents an acid-dissociable group represented by Formula (a1-r-1) or (a1-r-2).

In Formula (a1-2), the (nₐ₂+1)-valent hydrocarbon group as Wa¹ may be an aliphatic hydrocarbon group or an aromatic hydrocarbon group. The aliphatic hydrocarbon group indicates a hydrocarbon group that has no aromaticity and may be saturated or unsaturated. In general, it is preferable that the aliphatic hydrocarbon group be saturated. Examples of the aliphatic hydrocarbon group include a linear or branched aliphatic hydrocarbon group, an aliphatic hydrocarbon group having a ring in the structure thereof, and a group obtained by combining the linear or branched aliphatic hydrocarbon group and the aliphatic hydrocarbon group having a ring in the structure thereof.

The valency of nₐ₂+1 is preferably divalent to tetravalent and more preferably divalent or trivalent.

In the Formula (a1-2), Ra² represents an acid-dissociable group represented by General Formula (a1-r-1) or (a1-r-3).

Specific examples of the constitutional unit represented by Formula (a1-1) are shown below. In the formulae shown below, R^{α} represents a hydrogen atom, a methyl group, or a trifluoromethyl group.

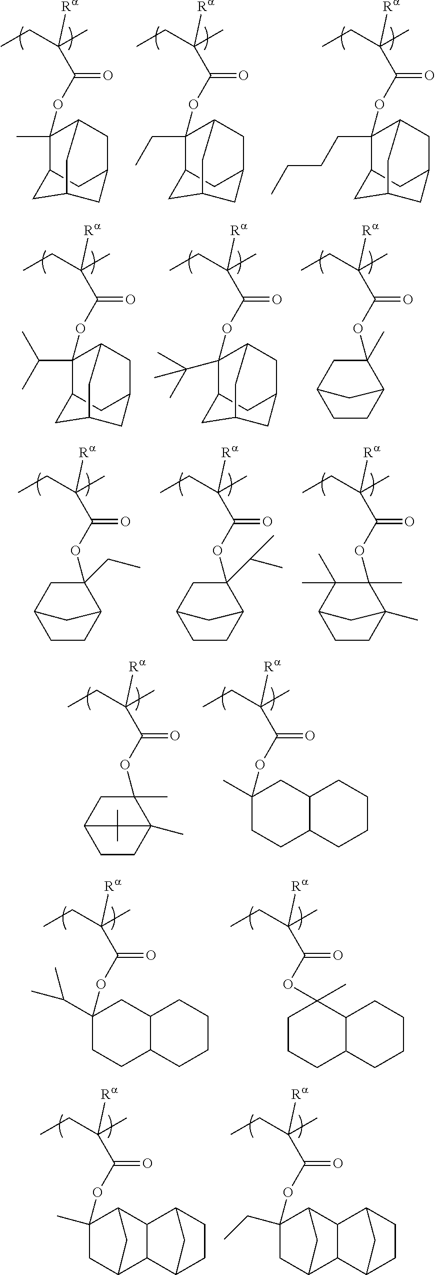

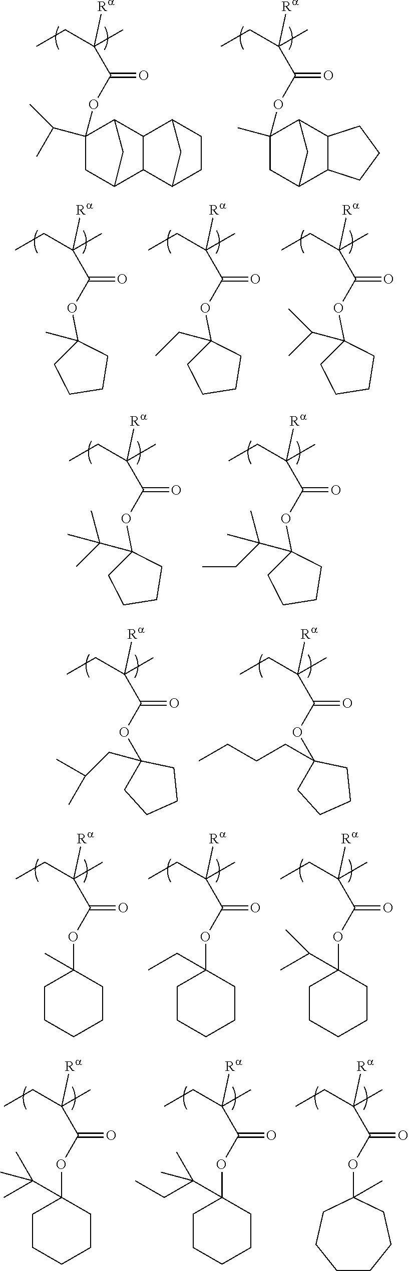

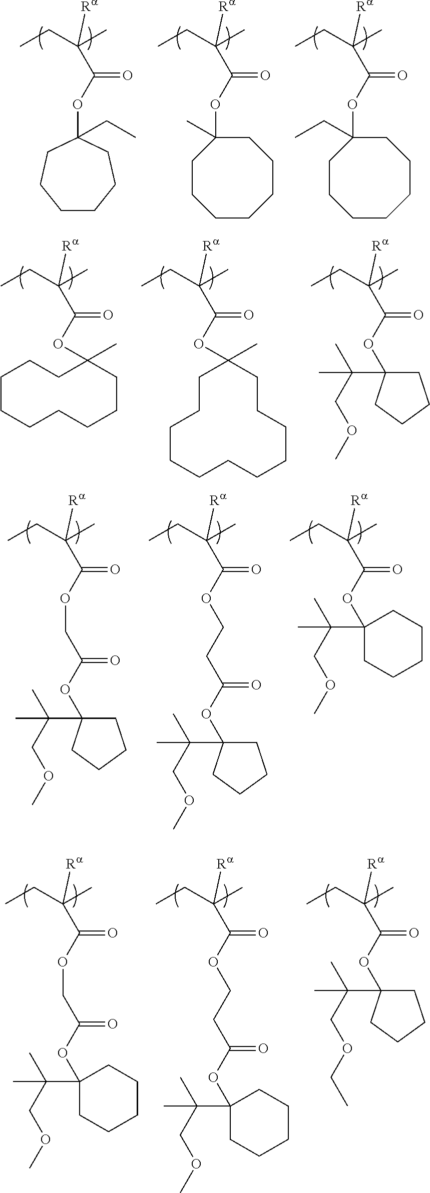

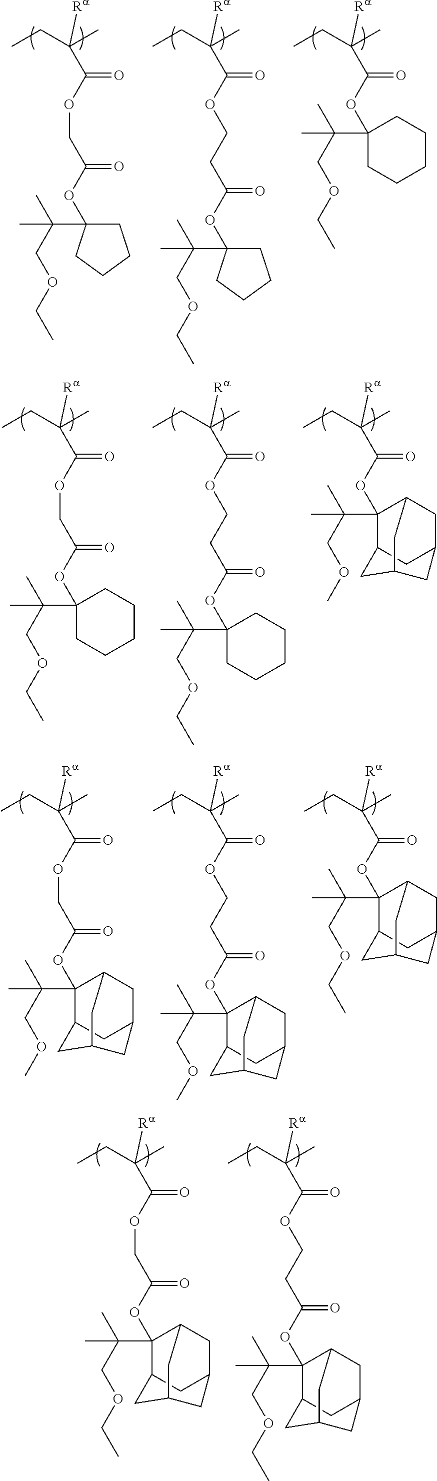

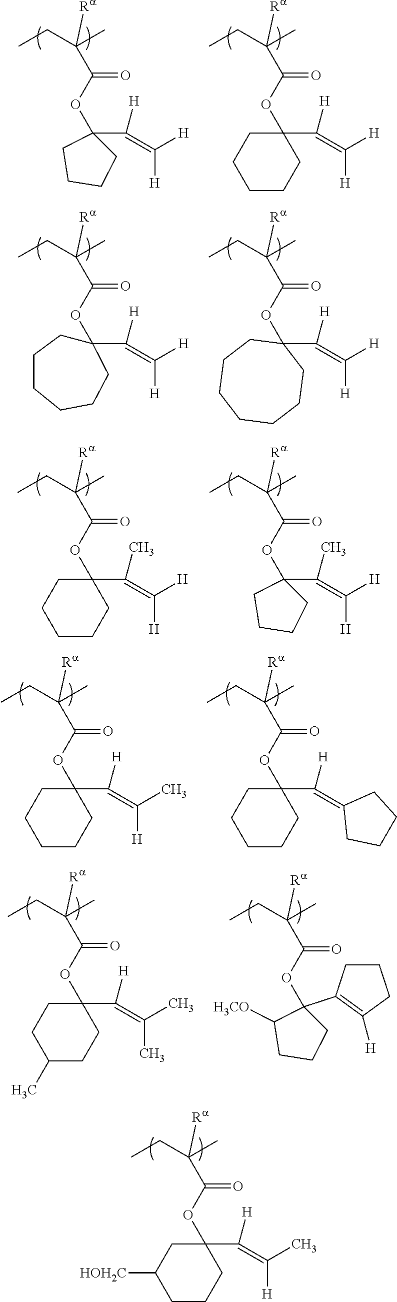

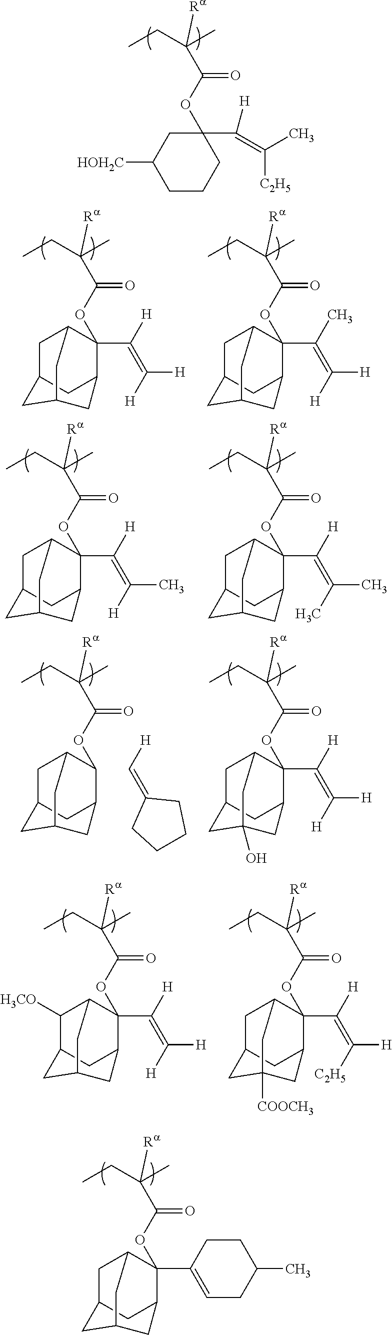

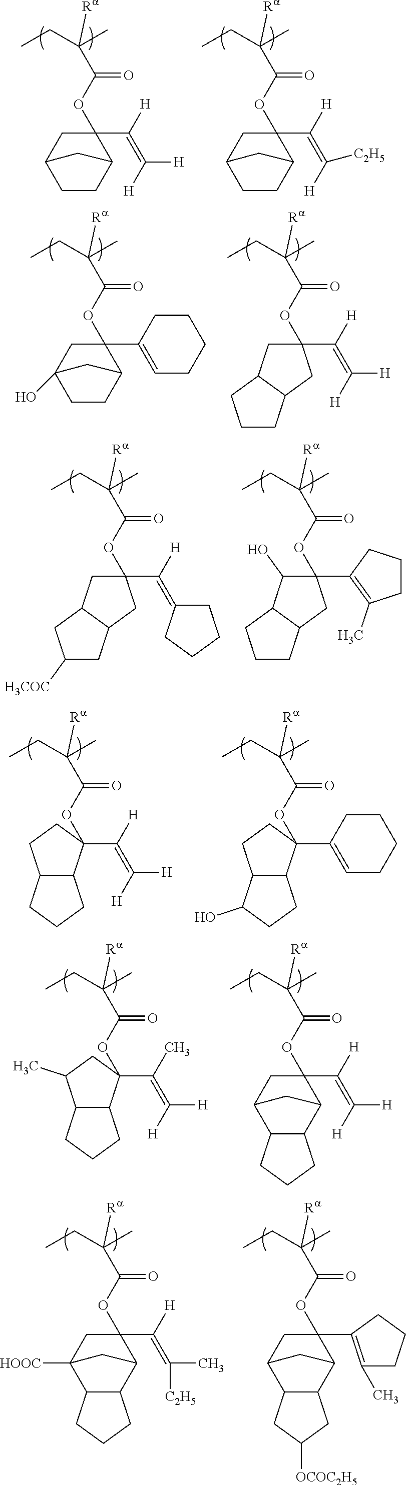

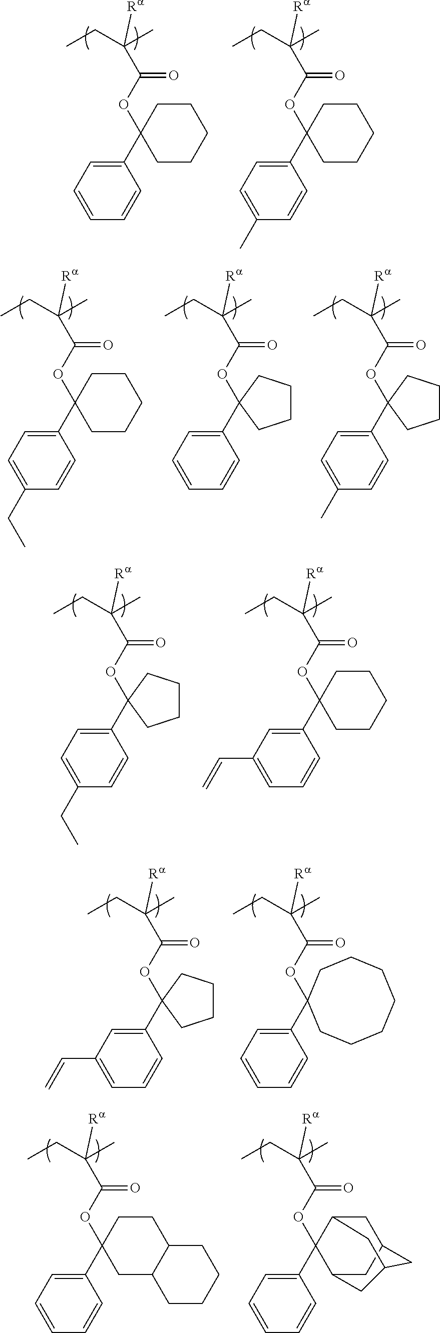

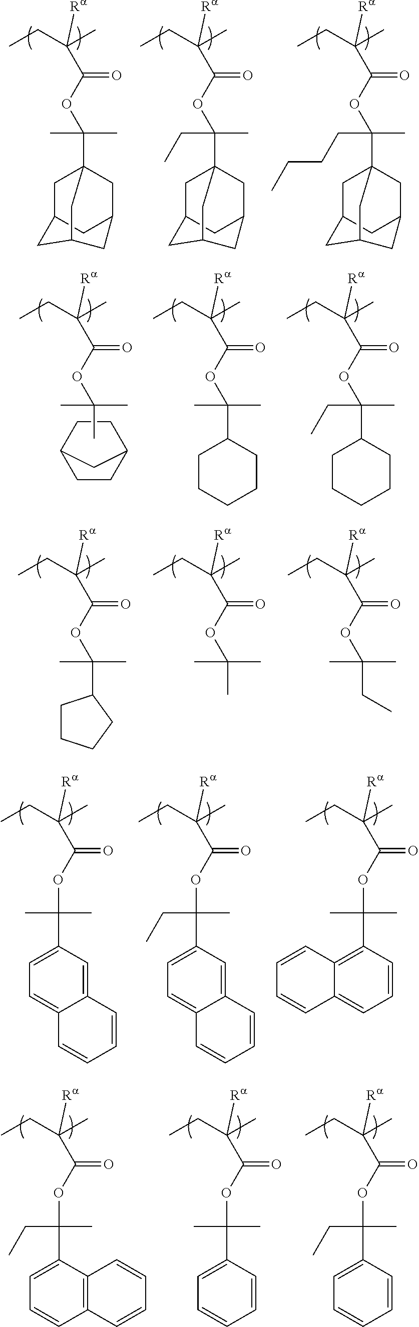

The constitutional unit (a1) included in the component (A1) may be used alone or two or more kinds thereof.

Since the lithography characteristics (the sensitivity, the shape, and the like) are easily improved using electron beams or EUV, a constitutional unit represented by Formula (a1-1) is preferable as the constitutional unit (a1).

Among the examples, as the constitutional unit (a1), those having a constitutional unit represented by General Formula (a1-1-1) are particularly preferable.

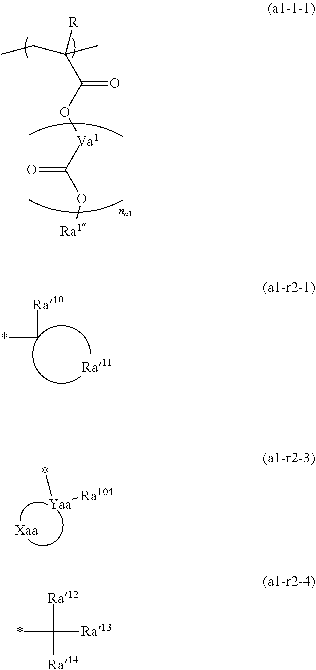

[In the formulae, Ra¹″ represents an acid-dissociable group represented by General Formula (a1-r2-1), (a1-r2-3), or (a1-r2-4).]

R, Va¹, and nₐ₁ in Formula (a1-1-1) each is the same as that for R, Va¹, and nₐ₁ in Formula (a1-1).

The description of the acid-dissociable group represented by General Formula (a1-r2-1), (a1-r2-3), or (a1-r2-4) is the same as described above. Among these, it is preferable to select those in which the acid-dissociable group is a cyclic group because the reactivity is enhanced for EB or EUV, which is preferable.

In Formula (a1-1-1), it is preferable that Ra¹″ represent an acid-dissociable group represented by General Formula (a1-r2-1) among the examples described above.

The proportion of the constitutional unit (a1) in the component (A1) is preferably in a range of 5% to 80% by mole, more preferably in a range of 10% to 75% by mole, still more preferably in a range of 30% to 70% by mole, and particularly preferably in a range of 40% to 70% by mole with respect to the total amount (100% by mole) of all constitutional units constituting the component (A1).

By setting the proportion of the constitutional unit (a1) to be greater than or equal to the lower limits of the above-described preferable ranges, improved lithography characteristics such as sensitivity, resolution, and roughness are achieved. Further, in a case where the proportion of the constitutional unit (a1) is less than or equal to the upper limits of the above-described preferable ranges, the constitutional unit (a1) and other constitutional units can be balanced, and the lithography characteristics are improved.

### <<Other Constitutional Units>>

Further, the component (A1) may have other constitutional units as necessary in addition to the constitutional unit (a1).

Examples of other constitutional units include a constitutional unit (a2) that contains a lactone-containing cyclic group, a —SO₂-containing cyclic group, or a carbonate-containing cyclic group; a constitutional unit (a3) that contains a polar group-containing aliphatic hydrocarbon group; a constitutional unit (a4) that contains an acid non-dissociable aliphatic cyclic group; a constitutional unit (a10) represented by General Formula (a10-1); a constitutional unit (st) derived from styrene or a styrene derivative; and a constitutional unit derived from a hydroxystyrene or a hydroxystyrene derivative.

### Regarding Constitutional Unit (a2):

The component (A1) may further have a constitutional unit (a2) (here, those corresponding to the constitutional unit (a1) are excluded) containing a lactone-containing cyclic group, a —SO₂-containing cyclic group, or a carbonate-containing cyclic group, in addition to the constitutional unit (a1).

In a case where the component (A1) is used for forming a resist film, the lactone-containing cyclic group, the —SO₂-containing cyclic group, or the carbonate-containing cyclic group in the constitutional unit (a2) is effective for improving the adhesiveness of the resist film to the substrate. Further, in a case where the component (A1) contains the constitutional unit (a2), the lithography characteristics and the like are improved due to the effects of appropriately adjusting the acid diffusion length, increasing the adhesiveness of the resist film to the substrate, and appropriately adjusting the solubility during the development.

The term “lactone-containing cyclic group” indicates a cyclic group that has a ring (lactone ring) containing —O—C(═O)— in the ring skeleton. In a case where the lactone ring is counted as the first ring and the group contains only the lactone ring, the group is referred to as a monocyclic group. Further, in a case where the group has other ring structures, the group is referred to as a polycyclic group regardless of the structures. The lactone-containing cyclic group may be a monocyclic group or a polycyclic group.

The lactone-containing cyclic group in the constitutional unit (a2) is not particularly limited, and any constitutional unit can be used. Specific examples thereof include groups each represented by General Formulae (a2-r-1) to (a2-r-7).

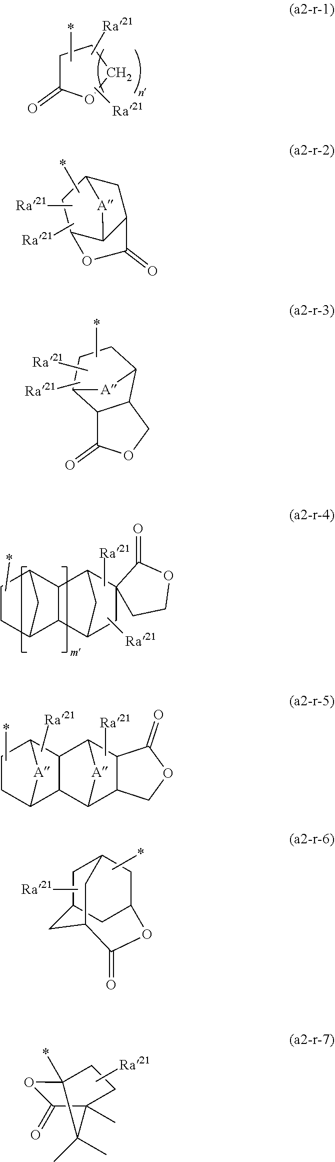

[In the formulae, Ra′²¹'s each independently represents a hydrogen atom, an alkyl group, an alkoxy group, a halogen atom, a halogenated alkyl group, a hydroxyl group, —COOR″, —OC(═O)R″, a hydroxyalkyl group, or a cyano group; R″ represents a hydrogen atom, an alkyl group, a lactone-containing cyclic group, a carbonate-containing cyclic group, or a —SO₂-containing cyclic group; A″ represents an alkylene group having 1 to 5 carbon atoms which may have an oxygen atom (—O—) or a sulfur atom (—S—), an oxygen atom, or a sulfur atom; n′ represents an integer of 0 to 2; and m′ represents 0 or 1.]

In General Formulae (a2-r-1) to (a2-r-7), it is preferable that the alkyl group as Ra′²¹ be an alkyl group having 1 to 6 carbon atoms. Further, it is preferable that the alkyl group be linear or branched. Specific examples thereof include a methyl group, an ethyl group, a propyl group, an isopropyl group, an n-butyl group, an isobutyl group, a tert-butyl group, a pentyl group, an isopentyl group, a neopentyl group, and a hexyl group. Among these, a methyl group or ethyl group is preferable, and a methyl group is particularly preferable.

It is preferable that the alkoxy group as Ra′²¹ be an alkoxy group having 1 to 6 carbon atoms. Further, it is preferable that the alkoxy group be linear or branched. Specific examples of the alkoxy groups include a group formed by linking the above-described alkyl group described as the alkyl group represented by Ra′²¹ to an oxygen atom (—O—).

As the halogen atom as Ra′²¹, a fluorine atom is preferable.

Examples of the halogenated alkyl group as Ra′²¹ include groups in which a part or all of the hydrogen atoms in the alkyl group as Ra′²¹ have been substituted with the halogen atoms. As the halogenated alkyl group, a fluorinated alkyl group is preferable, and a perfluoroalkyl group is particularly preferable.

In —COOR″ and —OC(═O)R″ as Ra′²¹, each R″ represents a hydrogen atom, an alkyl group, a lactone-containing cyclic group, a carbonate-containing cyclic group, or a —SO₂-containing cyclic group.

The alkyl group as R″ may be linear, branched, or cyclic and has preferably 1 to 15 carbon atoms.

In a case where R″ represents a linear or branched alkyl group, an alkyl group having 1 to 10 carbon atoms is preferable, an alkyl group having 1 to 5 carbon atoms is more preferable, and a methyl group or an ethyl group is particularly preferable.

In a case where R″ represents a cyclic alkyl group, the number of carbon atoms thereof is preferably in a range of 3 to 15, more preferably in a range of 4 to 12, and most preferably in a range of 5 to 10. Specific examples thereof include groups in which one or more hydrogen atoms have been removed from a monocycloalkane, which may or may not be substituted with a fluorine atom or a fluorinated alkyl group; and groups in which one or more hydrogen atoms have been removed from a polycycloalkane such as bicycloalkane, tricycloalkane, or tetracycloalkane. More specific examples thereof include groups in which one or more hydrogen atoms have been removed from a monocycloalkane such as cyclopentane or cyclohexane; and groups in which one or more hydrogen atoms have been removed from a polycycloalkane such as adamantane, norbornane, isobornane, tricyclodecane, or tetracyclododecane.

Examples of the lactone-containing cyclic group as R″ include the same groups as those for the groups each represented by General Formulae (a2-r-1) to (a2-r-7).

The carbonate-containing cyclic group as R″ is the same as that for the carbonate-containing cyclic group described below. Specific examples of the carbonate-containing cyclic group include groups each represented by General Formulae (ax3-r-1) to (ax3-r-3).

The —SO₂-containing cyclic group as R″ is the same as that for the —SO₂-containing cyclic group described below, and specific examples thereof include a group represented by any of General Formulae (a5-r-1) to (a5-r-4).

As the hydroxyalkyl group as Ra′²¹, a hydroxyalkyl group having 1 to 6 carbon atoms is preferable, and specific examples thereof include a group in which at least one hydrogen atom in the alkyl group as Ra′²¹ has been substituted with a hydroxyl group.

Among the examples, it is preferable that Ra′²¹'s each independently represents a hydrogen atom or a cyano group.

In General Formulae (a2-r-2), (a2-r-3) and (a2-r-5), as the alkylene group having 1 to 5 carbon atoms as A″, a linear or branched alkylene group is preferable, and examples thereof include a methylene group, an ethylene group, an n-propylene group, and an isopropylene group. In a case where the alkylene group has an oxygen atom or a sulfur atom, specific examples thereof include groups in which —O— or —S— is interposed between the terminals or carbon atoms of the alkylene group. Further, examples thereof include —O—CH₂—, —CH₂—O—CH₂—, —S—CH₂—, and —CH₂—S—CH₂—. A″ preferably represents an alkylene group having 1 to 5 carbon atoms or —O—, more preferably an alkylene group having 1 to 5 carbon atoms, and most preferably a methylene group.

Specific examples of the groups each represented by General Formulae (a2-r-1) to (a2-r-7) are shown below.

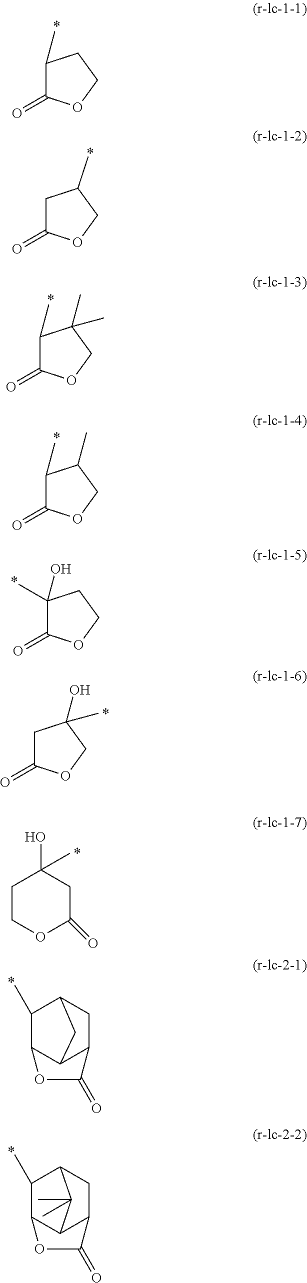

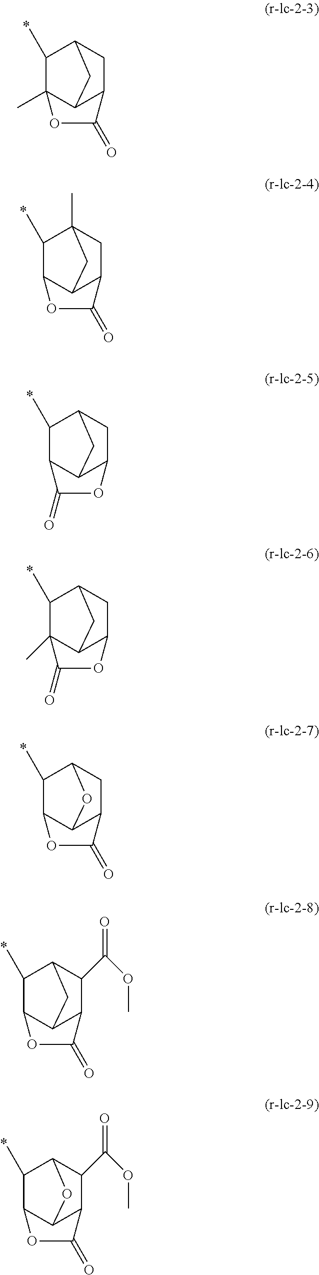

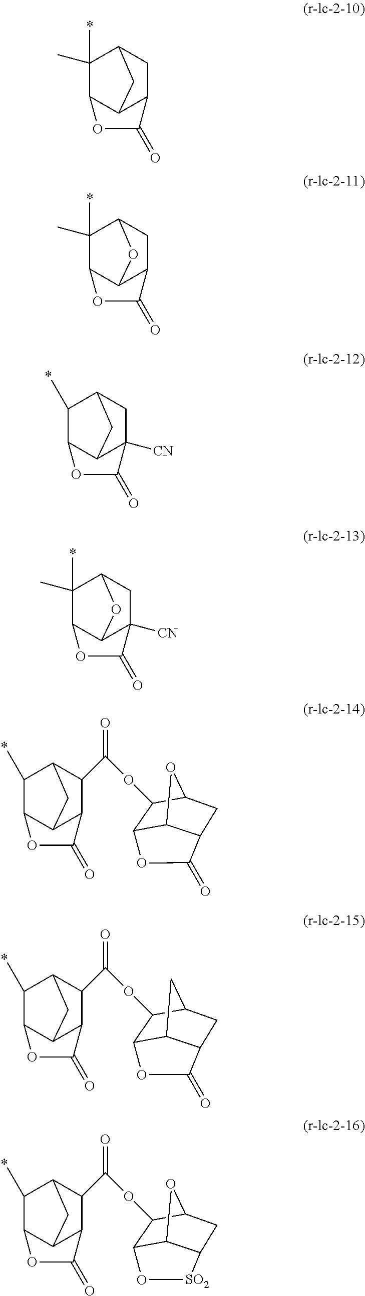

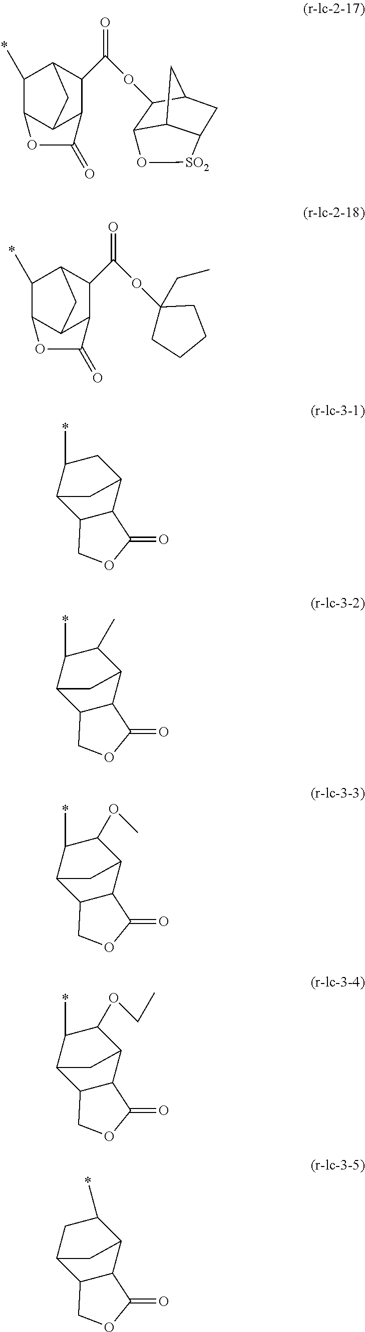

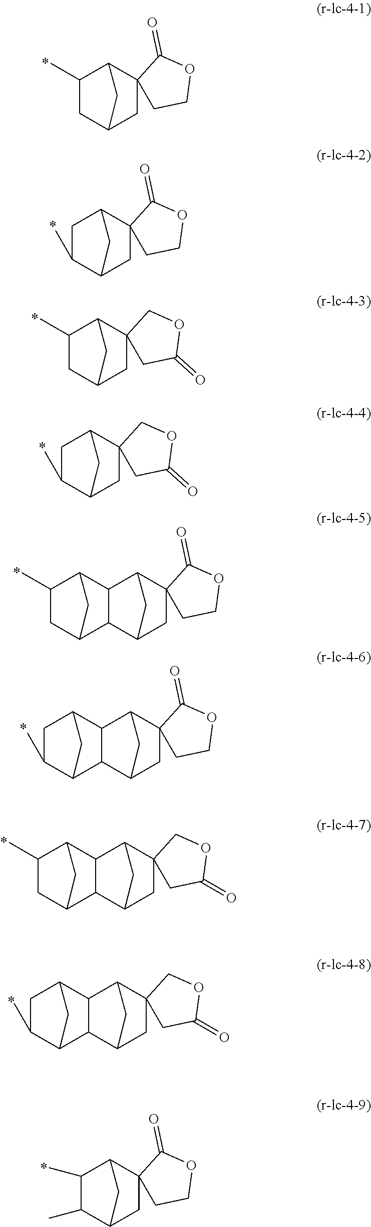

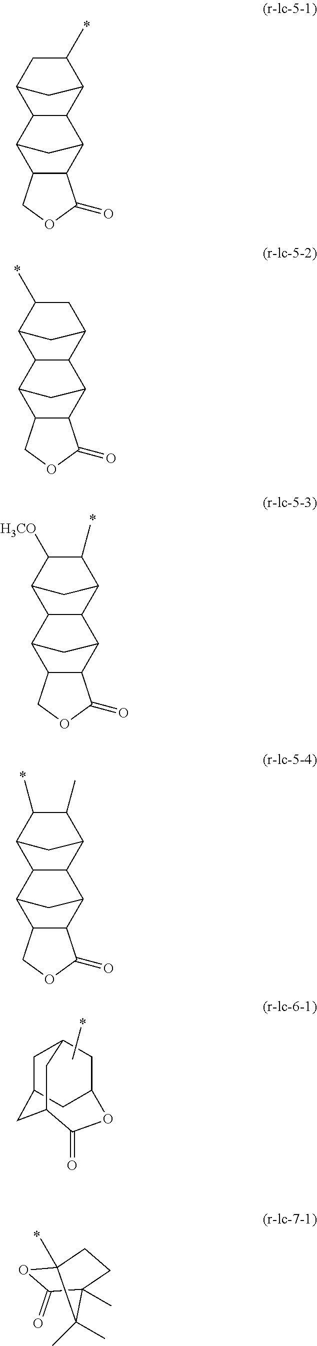

The term “—SO₂-containing cyclic group” denotes a cyclic group having a ring containing —SO₂— in the ring skeleton thereof. Specifically, the —SO₂-containing cyclic group is a cyclic group in which the sulfur atom (S) in —SO₂— forms a part of the ring skeleton of the cyclic group. In a case where the ring containing —SO₂— in the ring skeleton thereof is counted as the first ring and the group has only the ring, the group is referred to as a monocyclic group. In a case where the group further has other ring structures, the group is referred to as a polycyclic group regardless of the structures. The —SO₂-containing cyclic group may be a monocyclic group or a polycyclic group.

The —SO₂-containing cyclic group is particularly preferably a cyclic group containing —O—SO₂— in the ring skeleton thereof, that is, a cyclic group containing a sultone ring in which —O—S— in —O—SO₂— forms a part of the ring skeleton thereof.

More specific examples of the —SO₂-containing cyclic group include groups each represented by General Formulae (a5-r-1) to (a5-r-4).

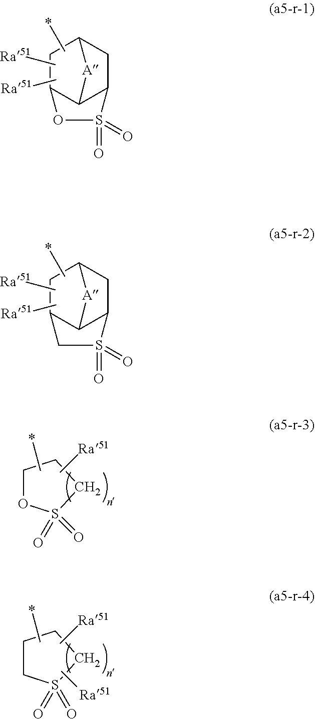

[In the formulae, Ra′⁵¹'s each independently represents a hydrogen atom, an alkyl group, an alkoxy group, a halogen atom, a halogenated alkyl group, a hydroxyl group, —COOR″, —OC(═O)R″, a hydroxyalkyl group, or a cyano group, R″ represents a hydrogen atom, an alkyl group, a lactone-containing cyclic group, a carbonate-containing cyclic group, or a —SO₂-containing cyclic group, A″ represents an alkylene group having 1 to 5 carbon atoms which may have an oxygen atom or a sulfur atom, an oxygen atom, or a sulfur atom, and n′ represents an integer of 0 to 2.]

In General Formulae (a5-r-1) and (a5-r-2), A″ is the same as that for A″ in Formulae (a2-r-2), (a2-r-3) and (a2-r-5).

Examples of the alkyl group, the alkoxy group, the halogen atom, the halogenated alkyl group, —COOR″, —OC(═O)R″, and the hydroxyalkyl group as Ra′⁵¹ include the same groups as those for Ra′²¹ in General Formulae (a2-r-1) to (a2-r-7).

Specific examples of the groups each represented by General Formulae (a5-r-1) to (a5-r-4) are shown below. In the formulae shown below, “Ac” represents an acetyl group.

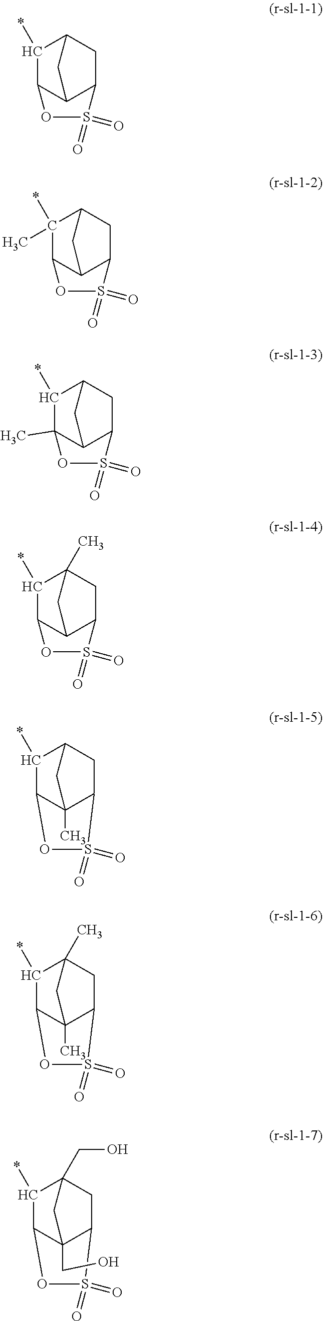

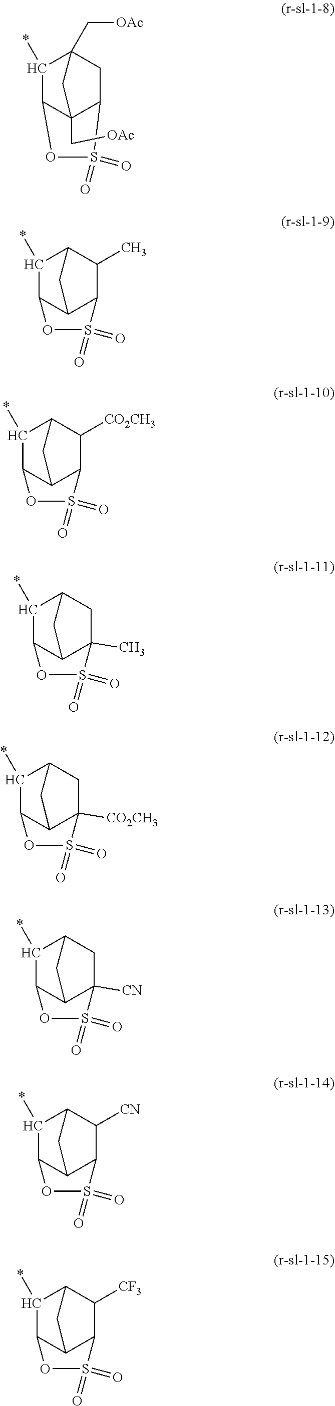

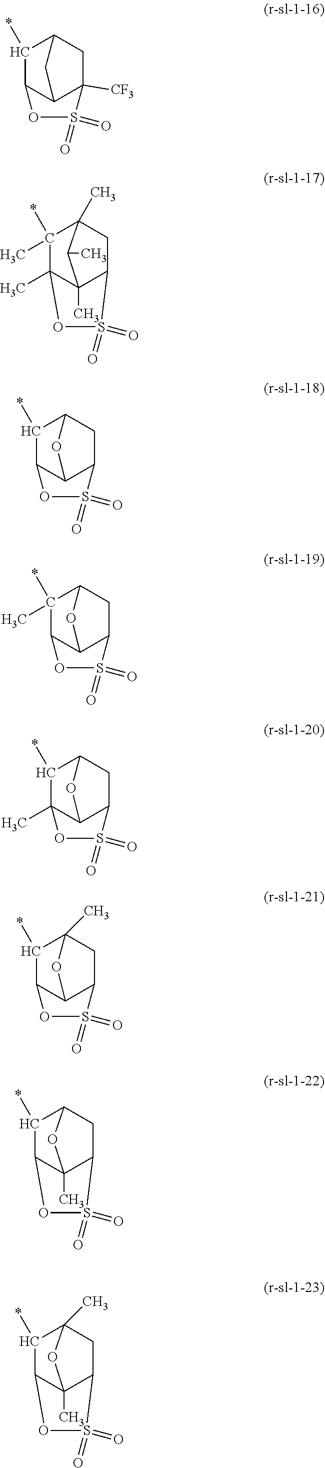

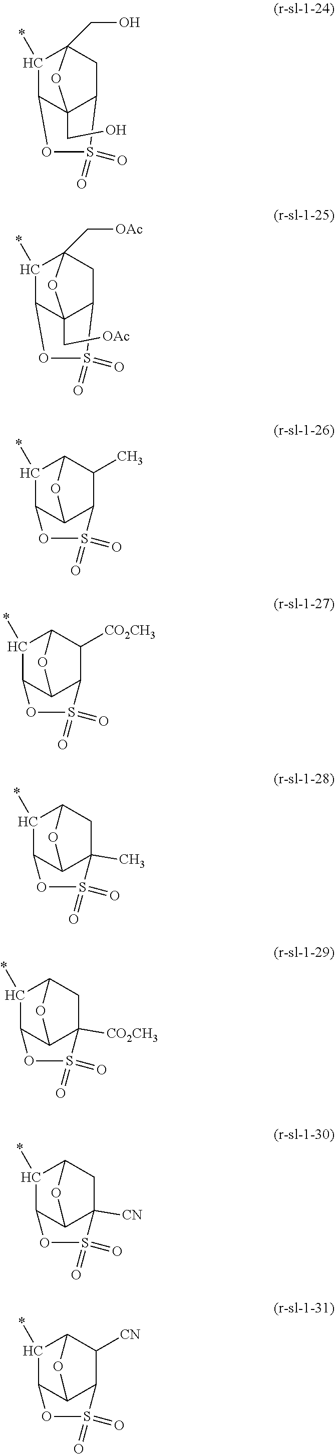

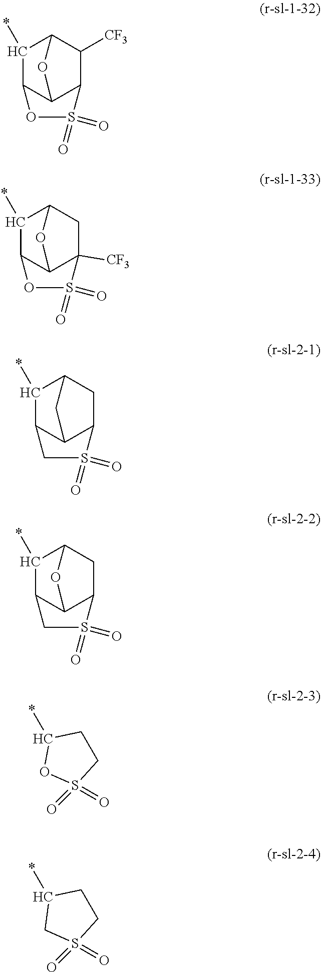

The term “carbonate-containing cyclic group” indicates a cyclic group that has a ring (a carbonate ring) containing —O—C(═O)—O— in the ring skeleton thereof. In a case where the carbonate ring is counted as the first ring and the group has only the carbonate ring, the group is referred to as a monocyclic group. Further, in a case where the group has other ring structures, the group is referred to as a polycyclic group regardless of the structures. The carbonate-containing cyclic group may be a monocyclic group or a polycyclic group.

The carbonate-containing cyclic group is not particularly limited, and any group can be used. Specific examples thereof include groups each represented by General Formulae (ax3-r-1) to (ax3-r-3) shown below.

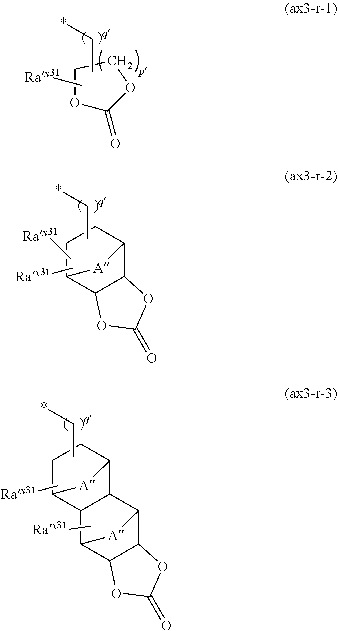

[In the formulae, Ra′^{x31}'s each independently represents a hydrogen atom, an alkyl group, an alkoxy group, a halogen atom, a halogenated alkyl group, a hydroxyl group, —COOR″, —OC(═O)R″, a hydroxyalkyl group, or a cyano group, R″ represents a hydrogen atom, an alkyl group, a lactone-containing cyclic group, a carbonate-containing cyclic group, or a —SO₂-containing cyclic group, A″ represents an alkylene group having 1 to 5 carbon atoms which may have an oxygen atom or a sulfur atom, an oxygen atom, or a sulfur atom, p′ represents an integer of 0 to 3, and q′ represents 0 or 1.]

In General Formulae (ax3-r-2) and (ax3-r-3), A″ is the same as that for A″ in Formulae (a2-r-2), (a2-r-3) and (a2-r-5).

Examples of the alkyl group, the alkoxy group, the halogen atom, the halogenated alkyl group, —COOR″, —OC(═O)R″, and the hydroxyalkyl group as Ra′³¹ include the same groups as those for Ra′²¹ in General Formulae (a2-r-1) to (a2-r-7).

Specific examples of the groups each represented by General Formulae (ax3-r-1) to (ax3-r-3) are shown below.

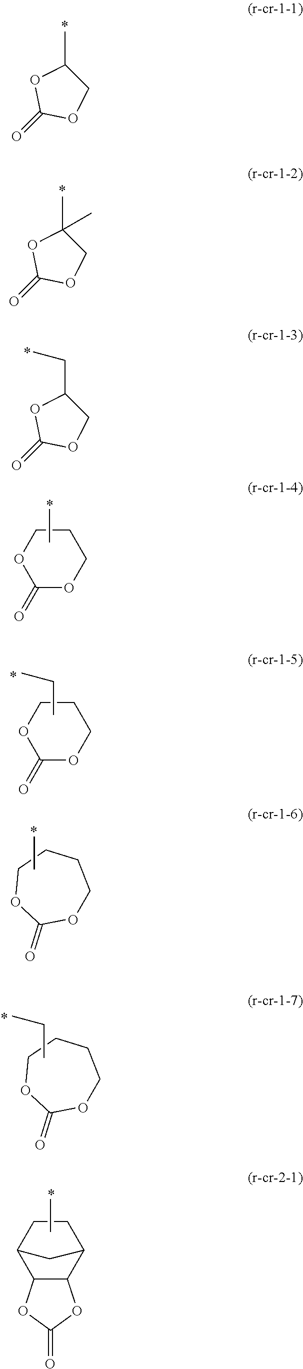

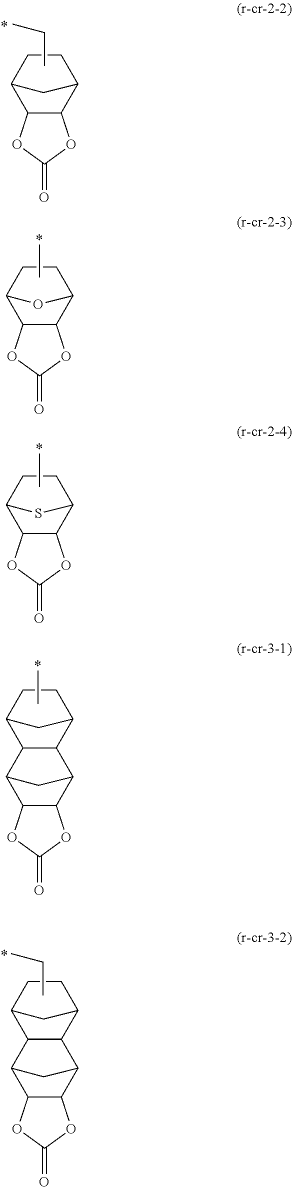

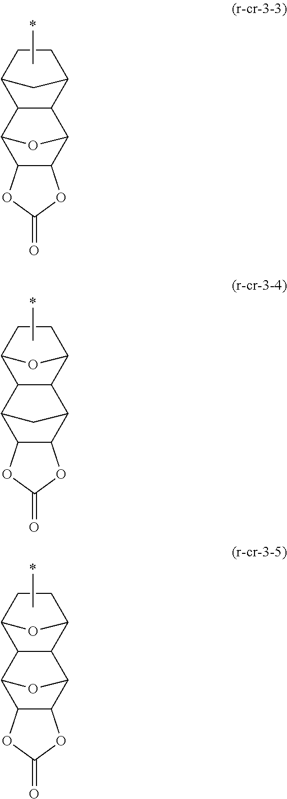

As the constitutional unit (a2), a constitutional unit derived from acrylic acid ester in which the hydrogen atom bonded to the carbon atom at the α-position may be substituted with a substituent is preferable.

It is preferable that such a constitutional unit (a2) be a constitutional unit represented by General Formula (a2-1).

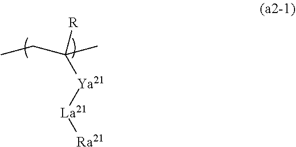

[In the formula, R represents a hydrogen atom, an alkyl group having 1 to 5 carbon atoms, or a halogenated alkyl group having 1 to 5 carbon atoms. Ya²¹ represents a single bond or a divalent linking group. La²¹ represents —O—, —COO—, —CON(R′)—, —OCO—, —CONHCO—, or —CONHCS—, and R′ represents a hydrogen atom or a methyl group. In a case where La²¹ represents —O—, Ya²¹ does not represent —CO—. Ra²¹ represents a lactone-containing cyclic group, a carbonate-containing cyclic group, or a —SO₂-containing cyclic group.]

In Formula (a2-1), R is the same as described above. R preferably represents a hydrogen atom, an alkyl group having 1 to 5 carbon atoms, or a fluorinated alkyl group having 1 to 5 carbon atoms and particularly preferably a hydrogen atom or a methyl group from the viewpoint of the industrial availability.

In Formula (a2-1), the divalent linking group as Ya²¹ is not particularly limited, and suitable examples thereof include a divalent hydrocarbon group which may have a substituent and a divalent linking group having a hetero atom.

Divalent Hydrocarbon Group which May have Substituent:

In a case where Ya²¹ represents a divalent hydrocarbon group which may have a substituent, the hydrocarbon group may be an aliphatic hydrocarbon group or an aromatic hydrocarbon group.

### Aliphatic Hydrocarbon Group as Ya²¹

The aliphatic hydrocarbon group indicates a hydrocarbon group that has no aromaticity. The aliphatic hydrocarbon group may be saturated or unsaturated. In general, it is preferable that the aliphatic hydrocarbon group be saturated.

Examples of the aliphatic hydrocarbon group include a linear or branched aliphatic hydrocarbon group and an aliphatic hydrocarbon group having a ring in the structure thereof.

### Linear or Branched Aliphatic Hydrocarbon Group

The linear aliphatic hydrocarbon group has preferably 1 to 10 carbon atoms, more preferably 1 to 6 carbon atoms, still more preferably 1 to 4 carbon atoms, and most preferably 1 to 3 carbon atoms.

As the linear aliphatic hydrocarbon group, a linear alkylene group is preferable, and specific examples thereof include a methylene group [—CH₂—], an ethylene group [—(CH₂)₂—], a trimethylene group [—(CH₂)₃—], a tetramethylene group [—(CH₂)₄—], and a pentamethylene group [—(CH₂)₅—].

The branched aliphatic hydrocarbon group has preferably 2 to 10 carbon atoms, more preferably 3 to 6 carbon atoms, still more preferably 3 or 4 carbon atoms, and most preferably 3 carbon atoms.

As the branched aliphatic hydrocarbon group, a branched alkylene group is preferable, and specific examples thereof include alkylalkylene groups, for example, alkylmethylene groups such as —CH(CH₃)—, —CH(CH₂CH₃)—, —C(CH₃)₂—, —C(CH₃)(CH₂CH₃)—, —C(CH₃)(CH₂CH₂CH₃)—, and —C(CH₂CH₃)₂—; alkylethylene groups such as —CH(CH₃)CH₂—, —CH(CH₃)CH(CH₃)—, —C(CH₃)₂CH₂—, —CH(CH₂CH₃)CH₂—, and —C(CH₂CH₃)₂—CH₂—; alkyltrimethylene groups such as —CH(CH₃)CH₂CH₂— and —CH₂CH(CH₃)CH₂—; and alkyltetramethylene groups such as —CH(CH₃)CH₂CH₂CH₂— and —CH₂CH(CH₃)CH₂CH₂—. As the alkyl group in the alkylalkylene group, a linear alkyl group having 1 to 5 carbon atoms is preferable.

The linear or branched aliphatic hydrocarbon group may or may not have a substituent. Examples of the substituent include a fluorine atom, a fluorinated alkyl group having 1 to 5 carbon atoms which has been substituted with a fluorine atom, and a carbonyl group.

### Aliphatic Hydrocarbon Group Having Ring in Structure Thereof

Examples of the aliphatic hydrocarbon group having a ring in the structure thereof include a cyclic aliphatic hydrocarbon group which may have a substituent having a hetero atom in the ring structure thereof (a group in which two hydrogen atoms have been removed from an aliphatic hydrocarbon ring), a group in which the cyclic aliphatic hydrocarbon group is bonded to the terminal of a linear or branched aliphatic hydrocarbon group, and a group in which the cyclic aliphatic hydrocarbon group is interposed in the middle of a linear or branched aliphatic hydrocarbon group. Examples of the linear or branched aliphatic hydrocarbon group include the same groups as those described above.

The cyclic aliphatic hydrocarbon group has preferably 3 to 20 carbon atoms and more preferably 3 to 12 carbon atoms.

The cyclic aliphatic hydrocarbon group may be a polycyclic group or a monocyclic group. As the monocyclic alicyclic hydrocarbon group, a group in which two hydrogen atoms have been removed from a monocycloalkane is preferable. The monocycloalkane has preferably 3 to 6 carbon atoms, and specific examples thereof include cyclopentane and cyclohexane. As the polycyclic alicyclic hydrocarbon group, a group in which two hydrogen atoms have been removed from a polycycloalkane is preferable. As the polycycloalkane, a group having 7 to 12 carbon atoms is preferable. Specific examples of the polycycloalkane include adamantane, norbornane, isobornane, tricyclodecane, and tetracyclododecane.

The cyclic aliphatic hydrocarbon group may or may not have a substituent. Examples of the substituent include an alkyl group, an alkoxy group, a halogen atom, a halogenated alkyl group, a hydroxyl group, and a carbonyl group.

As the alkyl group as the substituent, an alkyl group having 1 to 5 carbon atoms is preferable, and a methyl group, an ethyl group, a propyl group, an n-butyl group, or a tert-butyl group is more preferable.

As the alkoxy group as the substituent, an alkoxy group having 1 to 5 carbon atoms is preferable, a methoxy group, an ethoxy group, an n-propoxy group, an iso-propoxy group, an n-butoxy group, or a tert-butoxy group is more preferable, and a methoxy group or an ethoxy group is still more preferable.

As the halogen atom as the substituent, a fluorine atom is preferable.

Examples of the halogenated alkyl group as the substituent include groups in which a part or all of the hydrogen atoms in the above-described alkyl groups have been substituted with the above-described halogen atoms.

In the cyclic aliphatic hydrocarbon group, some carbon atoms constituting the ring structure thereof may be substituted with a substituent having a hetero atom. As the substituent having a hetero atom, —O—, —C(═O)—O—, —S—, —S(═O)₂—, or —S(═O)₂—O— is preferable.

### Aromatic Hydrocarbon Group as Ya²¹

The aromatic hydrocarbon group is a hydrocarbon group having at least one aromatic ring.

The aromatic ring is not particularly limited as long as the aromatic ring is a cyclic conjugated system having (4n+2) π electrons and may be monocyclic or polycyclic. The aromatic ring has preferably 5 to 30 carbon atoms, more preferably 5 to 20 carbon atoms, still more preferably 6 to 15 carbon atoms, and particularly preferably 6 to 12 carbon atoms. Here, the number of carbon atoms in a substituent is not included in the above number of carbon atoms.

Specific examples of the aromatic ring include aromatic hydrocarbon rings such as benzene, naphthalene, anthracene, and phenanthrene; and aromatic heterocyclic rings in which some carbon atoms constituting the above-described aromatic hydrocarbon rings have been substituted with hetero atoms. Examples of the hetero atom in the aromatic heterocyclic rings include an oxygen atom, a sulfur atom, and a nitrogen atom. Specific examples of the aromatic heterocyclic ring include a pyridine ring and a thiophene ring.

Specific examples of the aromatic hydrocarbon group include a group in which two hydrogen atoms have been removed from the above-described aromatic hydrocarbon ring or aromatic heterocyclic ring (an arylene group or a heteroarylene group); a group in which two hydrogen atoms have been removed from an aromatic compound having two or more aromatic rings (such as biphenyl or fluorene); and a group in which one hydrogen atom of a group (an aryl group or a heteroaryl group) obtained by removing one hydrogen atom from the above-described aromatic hydrocarbon ring or aromatic heterocyclic ring has been substituted with an alkylene group (for example, a group obtained by further removing one hydrogen atom from an aryl group in an arylalkyl group such as a benzyl group, a phenethyl group, a 1-naphthylmethyl group, a 2-naphthylmethyl group, a 1-naphthylethyl group, or a 2-naphthylethyl group). The number of carbon atoms in the alkylene group bonded to the aryl group or the heteroaryl group is preferably in a range of 1 to 4, more preferably 1 or 2, and particularly preferably 1.

In the aromatic hydrocarbon group, the hydrogen atom in the aromatic hydrocarbon group may be substituted with a substituent. For example, the hydrogen atom bonded to the aromatic ring in the aromatic hydrocarbon group may be substituted with a substituent. Examples of substituents include an alkyl group, an alkoxy group, a halogen atom, a halogenated alkyl group, and a hydroxyl group.

As the alkyl group as the substituent, an alkyl group having 1 to 5 carbon atoms is preferable, and a methyl group, an ethyl group, a propyl group, an n-butyl group, or a tert-butyl group is more preferable.

As the alkoxy group, the halogen atom, and the halogenated alkyl group as the substituents, the groups described as the substituents that substitute a hydrogen atom in the cyclic aliphatic hydrocarbon group are exemplary examples.

### Divalent Linking Group Having Hetero Atom:

In a case where Ya²¹ represents a divalent linking group having a hetero atom, preferred examples of the linking group include —O—, —C(═O)—O—, —O—C(═O)—, —C(═O)—, —O—C(═O)—O—, —C(═O)—NH—, —NH—, —NH—C(═NH)— (H may be substituted with a substituent such as an alkyl group or an acyl group), —S—, —S(═O)₂—, —S(═O)₂—O—, and a group represented by General Formula: —Y²¹—O—Y²²—, —Y²¹—O—, —Y²¹—C(═O)—O—, —C(═O)—O—Y²¹—, —[Y²¹—C(═O)—O]_{m″}—Y²²—, —Y²¹—O—C(═O)—Y²²—, or —Y²¹—S(═O)₂—O—Y²²— [in the formulae, Y²¹ and Y²² each independently represents a divalent hydrocarbon group which may have a substituent, O represents an oxygen atom, and m″ represents an integer of 0 to 3].

In a case where the divalent linking group containing a hetero atom is —C(═O)—NH—, —C(═O)—NH—C(═O)—, —NH—, or —NH—C(═NH)—, H may be substituted with a substituent such as an alkyl group, an acyl group, or the like. The substituent (an alkyl group, an acyl group, or the like) has preferably 1 to 10 carbon atoms, more preferably 1 to 8 carbon atoms, and particularly preferably 1 to 5 carbon atoms.

In General Formula —Y²¹—O—Y²²—, —Y²¹—O—, —Y²¹—C(═O)—O—, —C(═O)—O—Y²¹—, —[Y²¹—C(═O)—O]_{m″}—Y²²—, —Y²¹—O—C(═O)—Y²²—, or —Y²¹—S(═O)₂—O—Y²²—, Y²¹ and Y²² each independently represents a divalent hydrocarbon group which may have a substituent. Examples of the divalent hydrocarbon group include the same groups as those for the divalent linking group which may have a substituent, as described above for the divalent linking group as Ya²¹.

Y²¹ represents preferably a linear aliphatic hydrocarbon group, more preferably a linear alkylene group, still more preferably a linear alkylene group having 1 to 5 carbon atoms, and particularly preferably a methylene group or an ethylene group.

Y²² represents preferably a linear or branched aliphatic hydrocarbon group and more preferably a methylene group, an ethylene group, or an alkylmethylene group. The alkyl group in the alkylmethylene group is preferably a linear alkyl group having 1 to 5 carbon atoms, more preferably a linear alkyl group having 1 to 3 carbon atoms, and most preferably a methyl group.

In the group represented by Formula —[Y²¹—C(═O)—O]_{m″}—Y²²—, m″ represents an integer of 0 to 3, preferably an integer of 0 to 2, more preferably 0 or 1, and particularly preferably 1. That is, a group represented by Formula —Y²¹—C(═O)—O—Y²²— is particularly preferable as the group represented by Formula —[Y²¹—C(═O)—O]_{m′}—Y²²—. Among these, a group represented by Formula —(CH₂)_{a′}—C(═O)—O—(CH₂)_{b′}— is preferable. In the formula, a′ represents an integer of 1 to 10, preferably an integer of 1 to 8, more preferably an integer of 1 to 5, still more preferably 1 or 2, and most preferably 1. b′ represents an integer of 1 to 10, preferably an integer of 1 to 8, more preferably an integer of 1 to 5, still more preferably 1 or 2, and most preferably 1.

Among the examples, it is preferable that Ya²¹ represent a single bond, an ester bond [—C(═O)—O—], an ether bond (—O—), a linear or branched alkylene group, or a combination thereof.

In Formula (a2-1), Ra²¹ represents a lactone-containing cyclic group, a —SO₂— containing cyclic group, or a carbonate-containing cyclic group.

Suitable examples of the lactone-containing cyclic group, the —SO₂-containing cyclic group, and the carbonate-containing cyclic group as Ra²¹ include groups each represented by General Formulae (a2-r-1) to (a2-r-7), groups each represented by General Formulae (a5-r-1) to (a5-r-4), and groups each represented by General Formulae (ax3-r-1) to (ax3-r-3).

Among the examples, the lactone-containing cyclic group or the —SO₂-containing cyclic group is preferable, a group represented by any of General Formulae (a2-r-1), (a2-r-2), (a2-r-6), and (a5-r-1) is more preferable, and a group represented by any of General Formulae (a2-r-2) and (a5-r-1) is still more preferable. Specifically, a group represented by any of Chemical Formulae (r-lc-1-1) to (r-lc-1-7), (r-lc-2-1) to (r-lc-2-18), (r-lc-6-1), (r-sl-1-1), and (r-sl-1-18) is preferable, a group represented by any of Chemical Formulae (r-lc-2-1) to (r-lc-2-18) and (r-sl-1-1) is more preferable, and a group represented by any of Chemical Formulae (r-lc-2-1), (r-lc-2-12), and (r-sl-1-1) is still more preferable.

The constitutional unit (a2) included in the component (A1) may be used alone or two or more kinds thereof.

In a case where the component (A1) has the constitutional unit (a2), the proportion of the constitutional unit (a2) in the component (A1) is preferably in a range of 5% to 60% by mole, more preferably in a range of 10% to 60% by mole, still more preferably in a range of 20% to 60% by mole, and particularly preferably in a range of 30% to 60% by mole with respect to the total amount (100% by mole) of all constitutional units constituting the component (A1).

In a case where the proportion of the constitutional unit (a2) is set to be greater than or equal to the lower limits of the above-described preferable ranges, the effect to be obtained by allowing the component (A1) to have the constitutional unit (a2) is sufficiently obtained by the above-described effects. Further, in a case where the proportion thereof is set to be less than or equal to the upper limits of the above-described preferable ranges, the constitutional unit (a2) and other constitutional units can be balanced, and the lithography characteristics are improved.

### Regarding Constitutional Unit (a3):

The component (A1) may further have a constitutional unit (a3) containing a polar group-containing aliphatic hydrocarbon group (here, those corresponding to the constitutional unit (a1) or the constitutional unit (a2) are excluded) in addition to the constitutional unit (a1). In a case where the component (A1) has the constitutional unit (a3), the hydrophilicity of the component (A) is increased, which contributes to improvement of the resolution. Further, the acid diffusion length can be appropriately adjusted.

Examples of the polar group include a hydroxyl group, a cyano group, a carboxy group, or a hydroxyalkyl group in which some hydrogen atoms in the alkyl group have been substituted with fluorine atoms. Among these, a hydroxyl group is particularly preferable.

Examples of the aliphatic hydrocarbon group include a linear or branched hydrocarbon group (preferably an alkylene group) having 1 to 10 carbon atoms and a cyclic aliphatic hydrocarbon group (cyclic group). The cyclic group may be a monocyclic group or a polycyclic group. For example, the cyclic group can be appropriately selected from the plurality of groups that have been proposed in the resins for resist compositions for ArF excimer lasers.

In a case where the cyclic group is a monocyclic group, the number of carbon atoms is more preferably in a range of 3 to 10. Among the examples, constitutional units derived from acrylic acid ester that include an aliphatic monocyclic group containing a hydroxyl group, a cyano group, a carboxy group, or a hydroxyalkyl group in which some hydrogen atoms in the alkyl group have been substituted with fluorine atoms are more preferable. Examples of the monocyclic group include groups obtained by removing two or more hydrogen atoms from a monocycloalkane. Specific examples include groups obtained by removing two or more hydrogen atoms from monocycloalkanes such as cyclopentane, cyclohexane, and cyclooctane. Among these monocyclic groups, a group obtained by removing two or more hydrogen atoms from cyclopentane and a group obtained by removing two or more hydrogen atoms from cyclohexane are industrially preferable.

In a case where the cyclic group is a polycyclic group, the number of carbon atoms in the polycyclic group is more preferably in a range of 7 to 30. Among the examples, constitutional units derived from acrylic acid ester that include an aliphatic polycyclic group containing a hydroxyl group, a cyano group, a carboxy group, or a hydroxyalkyl group in which some hydrogen atoms in the alkyl group have been substituted with fluorine atoms are more preferable. Examples of the polycyclic group include groups in which two or more hydrogen atoms have been removed from bicycloalkane, tricycloalkane, tetracycloalkane, or the like. Specific examples thereof include groups in which two or more hydrogen atoms have been removed from a polycycloalkane such as adamantane, norbornane, isobornane, tricyclodecane, or tetracyclododecane. Among these polycyclic groups, a group in which two or more hydrogen atoms have been removed from adamantane, a group in which two or more hydrogen atoms have been removed from norbornane, or a group in which two or more hydrogen atoms have been removed from tetracyclododecane is industrially preferable.

The constitutional unit (a3) is not particularly limited as long as the constitutional unit contains a polar group-containing aliphatic hydrocarbon group, and any constitutional unit can be used.

As the constitutional unit (a3), a constitutional unit derived from acrylic acid ester in which the hydrogen atom bonded to the carbon atom at the α-position may be substituted with a substituent, which is a constitutional unit containing a polar group-containing aliphatic hydrocarbon group is preferable.

In a case where the hydrocarbon group in the polar group-containing aliphatic hydrocarbon group is a linear or branched hydrocarbon group having 1 to 10 carbon atoms, a constitutional unit derived from hydroxyethyl ester of acrylic acid is preferable as the constitutional unit (a3).

Further, in a case where the hydrocarbon group in the polar group-containing aliphatic hydrocarbon group is a polycyclic group, a constitutional unit represented by Formula (a3-1), a constitutional unit represented by Formula (a3-2), or a constitutional unit represented by Formula (a3-3) is preferable as the constitutional unit (a3). Further, in a case where the hydrocarbon group in the polar group-containing aliphatic hydrocarbon group is a monocyclic group, a constitutional unit represented by Formula (a3-4) is preferable as the constitutional unit (a3).

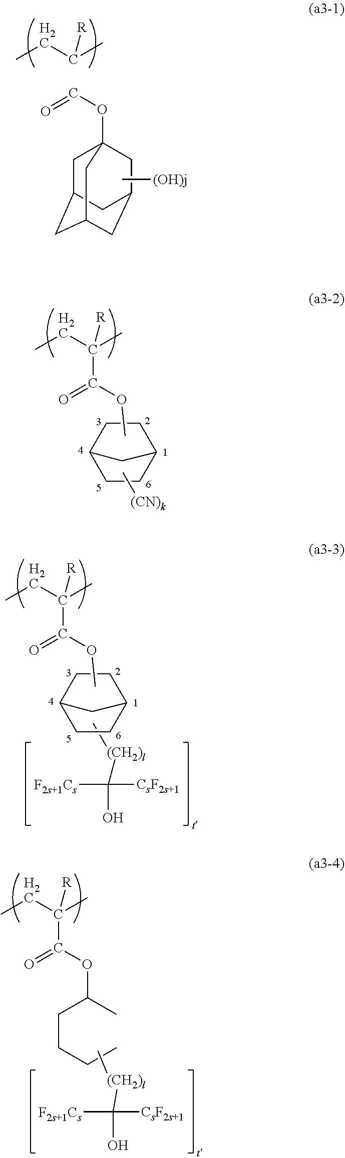

[In the formulae, R is the same as described above, j represents an integer of 1 to 3, k represents an integer of 1 to 3, t′ represents an integer of 1 to 3, 1 represents an integer of 0 to 5, and s represents an integer of 1 to 3.]

In Formula (a3-1), j represents preferably 1 or 2 and more preferably 1. In a case where j represents 2, it is preferable that the hydroxyl groups be bonded to the 3- and 5-positions of the adamantyl group. In a case where j represents 1, it is preferable that the hydroxyl group be bonded to the 3-position of the adamantyl group. It is preferable that j represent 1, and it is particularly preferable that the hydroxyl group be bonded to the 3-position of the adamantyl group.

In Formula (a3-2), it is preferable that k represent 1. It is preferable that the cyano group be bonded to the 5- or 6-position of the norbornyl group.

In Formula (a3-3), it is preferable that t′ represent 1. It is preferable that 1 represent 1. It is preferable that s represent 1. Further, it is preferable that a 2-norbornyl group or 3-norbornyl group be bonded to the terminal of the carboxy group of the acrylic acid. It is preferable that the fluorinated alkyl alcohol be bonded to the 5- or 6-position of the norbornyl group.

In Formula (a3-4), it is preferable that t′ represent 1 or 2. It is preferable that 1 represent 0 or 1. It is preferable that s represent 1. It is preferable that the fluorinated alkyl alcohol be bonded to the 3- or 5-position of the cyclohexyl group.

The constitutional unit (a3) included in the component (A1) may be used alone or two or more kinds thereof.

In a case where the component (A1) has the constitutional unit (a3), the proportion of the constitutional unit (a3) is preferably in a range of 1% to 30% by mole, more preferably in a range of 2% to 25% by mole, and still more preferably in a range of 5% to 20% by mole with respect to the total amount (100% by mole) of all constitutional units constituting the component (A1).

In a case where the proportion of the constitutional unit (a3) is set to be greater than or equal to the lower limits of the above-described preferable ranges, the effect to be obtained by allowing the component (A1) to have the constitutional unit (a3) is sufficiently obtained by the above-described effects. Further, in a case where the proportion thereof is set to be less than or equal to the upper limits of the above-described preferable ranges, the constitutional unit (a3) and other constitutional units can be balanced, and the lithography characteristics are improved.

### Regarding Constitutional Unit (a4):

The component (A1) may further have a constitutional unit (a4) containing an acid non-dissociable aliphatic cyclic group in addition to the constitutional unit (a1).

In a case where the component (A1) has the constitutional unit (a4), the dry etching resistance of a resist pattern to be formed is improved. Further, the hydrophobicity of the component (A) is increased. The improvement of the hydrophobicity contributes to improvement of the resolution, the resist pattern shape, and the like particularly in a case of the solvent developing process.

The term “acid non-dissociable cyclic group” in the constitutional unit (a4) denotes a cyclic group remaining in the constitutional unit without being dissociated due to the action of an acid in a case of generation of an acid in the resist composition upon light exposure (for example, an acid is generated from the component (B) or a constitutional unit that generates an acid upon light exposure).

As the constitutional unit (a4), for example, a constitutional unit derived from acrylic acid ester containing an acid non-dissociable aliphatic cyclic group or the like is preferable. As the cyclic group, a plurality of cyclic groups which have been known in the related art as those used for resin components of resist compositions for an ArF excimer laser, a KrF excimer laser (preferably an ArF excimer laser), and the like can be used.

It is particularly preferable that the cyclic group be at least one selected from a tricyclodecyl group, an adamantyl group, a tetracyclododecyl group, an isobornyl group, and a norbornyl group from the viewpoint of the industrial availability or the like. These polycyclic groups may have a linear or branched alkyl group having 1 to 5 carbon atoms as a substituent.

Specific examples of the constitutional unit (a4) include constitutional units each represented by General Formulae (a4-1) to (a4-7).

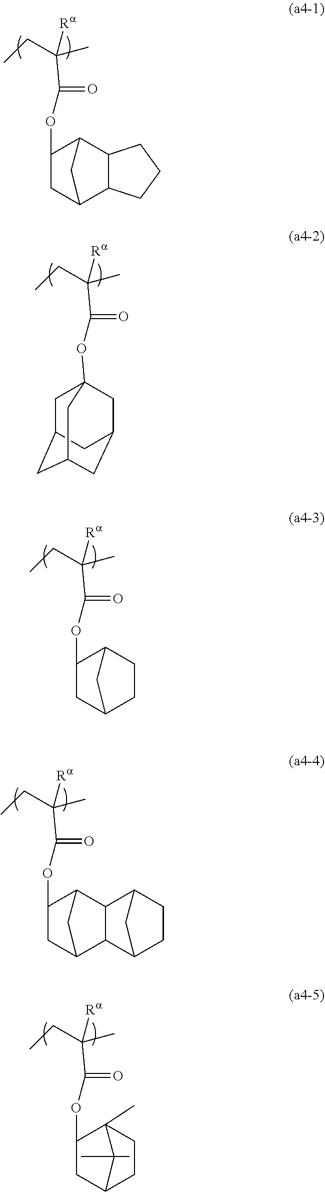

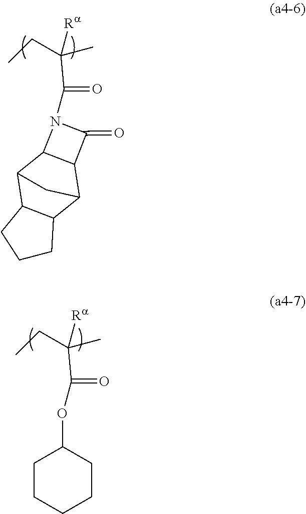

[In the formulae, R^{α} is the same as described above.]

The constitutional unit (a4) included in the component (A1) may be used alone or two or more kinds thereof.

In a case where the component (A1) contains the constitutional unit (a4), the proportion of the constitutional unit (a4) is preferably in a range of 1% to 40% by mole and more preferably in a range of 5% to 20% by mole with respect to the total amount (100% by mole) of all constitutional units constituting the component (A1).

In a case where the proportion of the constitutional unit (a4) is set to be greater than or equal to the lower limits of the above-described preferable ranges, the effect to be obtained by allowing the component (A1) to have the constitutional unit (a4) is sufficiently obtained by the above-described effects. Further, in a case where the proportion thereof is set to be less than or equal to the upper limits of the above-described preferable ranges, the constitutional unit (a4) and other constitutional units are likely to be balanced.

Regarding Constitutional Unit (a10):

The constitutional unit (a10) is a constitutional unit represented by General Formula (a10-1).

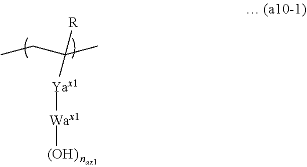

[In the formula, R represents a hydrogen atom, an alkyl group having 1 to 5 carbon atoms, or a halogenated alkyl group having 1 to 5 carbon atoms. Ya^{x1} represents a single bond or a divalent linking group. Wa^{x1} represents an aromatic hydrocarbon group which may have a substituent. nₐₓ₁ represents an integer of 1 or greater.]

In the Formula (a10-1), R represents a hydrogen atom, an alkyl group having 1 to 5 carbon atoms, or a halogenated alkyl group having 1 to 5 carbon atoms. R is the same as that for R in Formula (a0-1-1).

In Formula (a10-1), Ya^{x1} represents a single bond or a divalent linking group. In the chemical formula, the divalent linking group as Ya^{x1} is not particularly limited, and suitable examples thereof include a divalent hydrocarbon group which may have a substituent and a divalent linking group having a hetero atom. Examples of the divalent linking group as Ya^{x1} include the same groups as those for the divalent linking group as Ya²¹ in Formula (a2-1).

Among these, Ya^{x1} represents preferably a single bond, an ester bond [—C(═O)—O— or —O—C(═O)—], an ether bond (—O—), a linear or branched alkylene group, or a combination thereof and more preferably a single bond or an ester bond [—C(═O)—O— or —O—C(═O)—].

In Formula (a10-1), Wa^{x1} represents an aromatic hydrocarbon group which may have a substituent.

Examples of the aromatic hydrocarbon group as Wa^{x1} include a group in which (nₐₓ₁+1) hydrogen atoms have been removed from an aromatic ring which may have a substituent. The aromatic ring is not particularly limited as long as the aromatic ring is a cyclic conjugated system having (4n+2) π electrons and may be monocyclic or polycyclic. The aromatic ring has preferably 5 to 30 carbon atoms, more preferably 5 to 20 carbon atoms, still more preferably 6 to 15 carbon atoms, and particularly preferably 6 to 12 carbon atoms. Specific examples of the aromatic ring include aromatic hydrocarbon rings such as benzene, naphthalene, anthracene, and phenanthrene; and an aromatic heterocyclic ring obtained by substituting some carbon atoms constituting the above-described aromatic hydrocarbon ring with a hetero atom. Examples of the hetero atom in the aromatic heterocyclic rings include an oxygen atom, a sulfur atom, and a nitrogen atom. Specific examples of the aromatic heterocyclic ring include a pyridine ring and a thiophene ring.

Examples of the aromatic hydrocarbon group as Wa^{x1} include a group in which (nₐₓ₁+1) hydrogen atoms have been removed from an aromatic compound (such as biphenyl or fluorene) having an aromatic ring which may have two or more substituents. Among these, Wa^{x1} represents preferably a group in which (nₐₓ₁+1) hydrogen atoms have been removed from benzene, naphthalene, anthracene, or biphenyl, more preferably a group in which (nₐₓ₁+1) hydrogen atoms have been removed from benzene or naphthalene, and still more preferably a group in which (nₐₓ₁+1) hydrogen atoms have been removed from benzene.

The aromatic hydrocarbon group as Wa^{x1} may or may not have a substituent. Examples of substituents include an alkyl group, an alkoxy group, a halogen atom, and a halogenated alkyl group. Examples of the alkyl group, the alkoxy group, the halogen atom, and the halogenated alkyl group as the substituent include those described as the substituent of the cyclic aliphatic hydrocarbon group as Ya^{x1}. The substituent is preferably a linear or branched alkyl group having 1 to 5 carbon atoms, more preferably a linear or branched alkyl group having 1 to 3 carbon atoms, still more preferably an ethyl group or a methyl group, and particularly preferably a methyl group. It is preferable that the aromatic hydrocarbon group as Wa^{x1} have no substituent.

In Formula (a10-1), nₐₓ₁ represents an integer of 1 or greater, preferably an integer of 1 to 10, more preferably an integer of 1 to 5, still more preferably 1, 2, or 3, and particularly preferably 1 or 2.

Specific examples of the constitutional unit (a10) represented by Formula (a10-1) are described below.

In the formulae shown below, R^{α} represents a hydrogen atom, a methyl group, or a trifluoromethyl group.

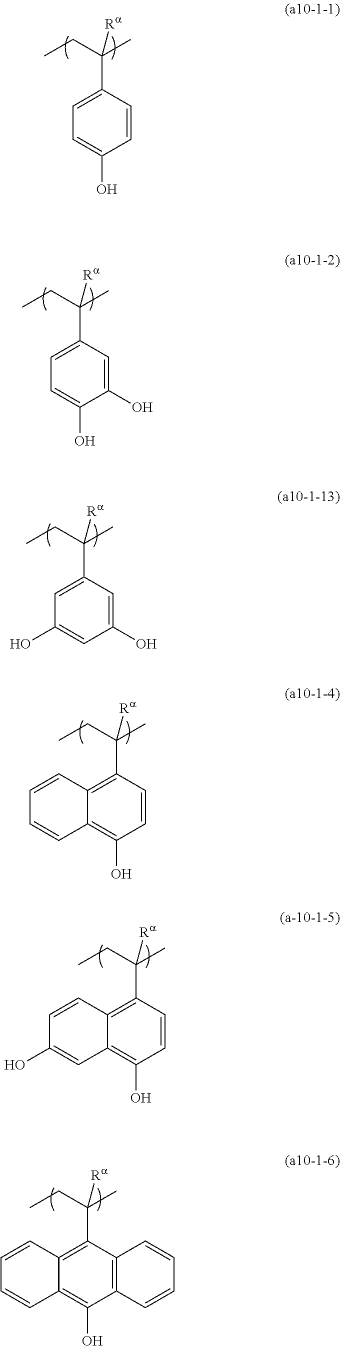

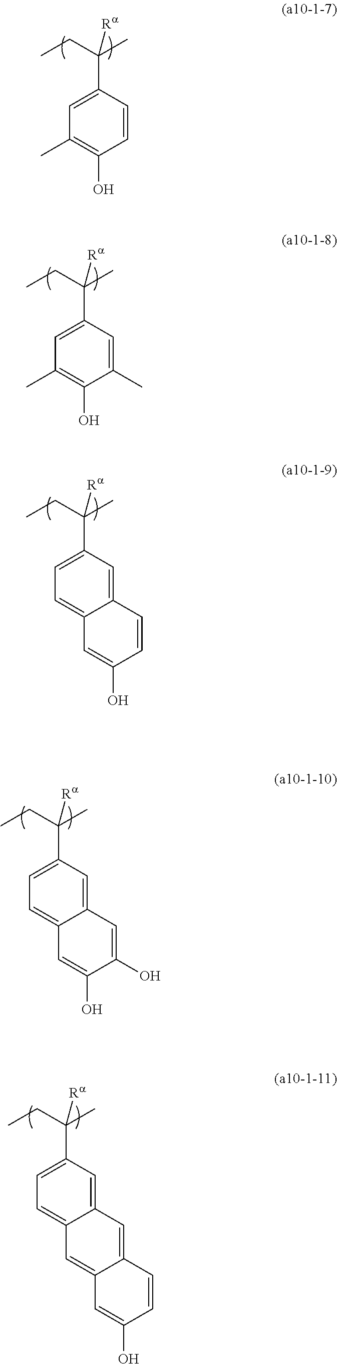

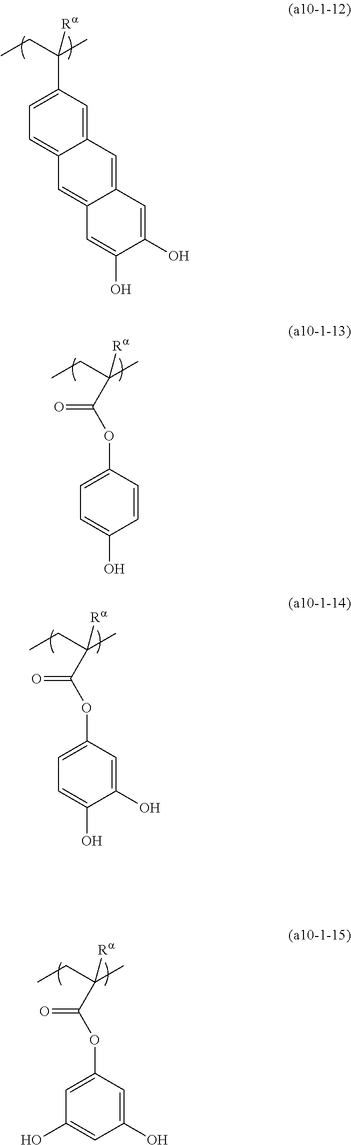

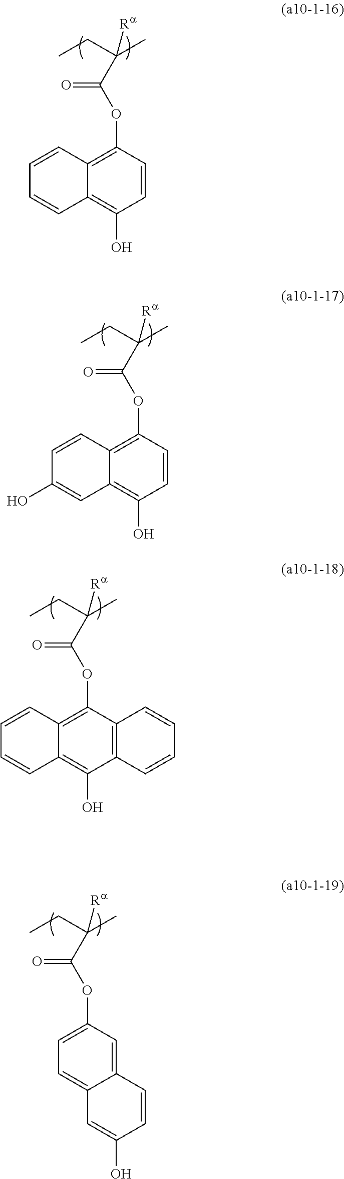

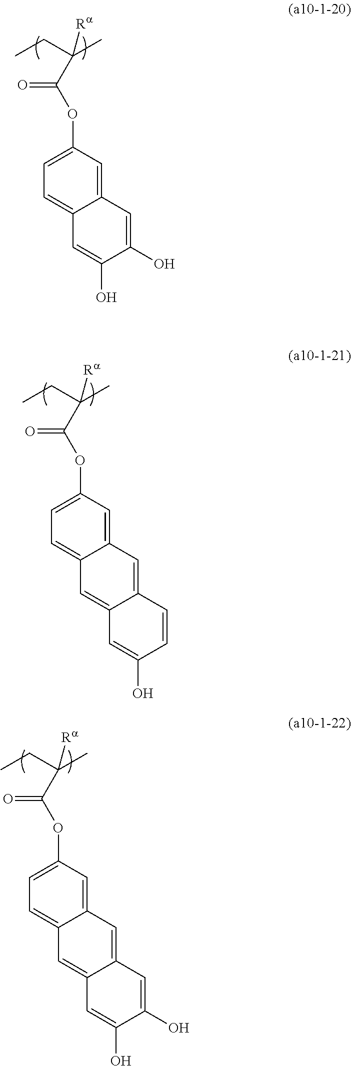

The constitutional unit (a10) included in the component (A1) may be used alone or two or more kinds thereof.

In a case where the component (A1) has the constitutional unit (a10), the proportion of the constitutional unit (a10) in the component (A1) is preferably in a range of 10% to 80% by mole, more preferably in a range of 20% to 70% by mole, still more preferably in a range of 30% to 60% by mole, and particularly preferably in a range of 30% to 50% by mole with respect to the total amount (100% by mole) of all constitutional units constituting the component (A1).

In a case where the proportion of the constitutional unit (a10) is greater than or equal to the lower limits of the above-described preferable ranges, the sensitivity is more likely to be increased. In a case where the proportion of the constitutional unit (a10) is less than or equal to the upper limits of the above-described preferable ranges, the balance between the constitutional unit (a10) and other constitutional units is likely to be achieved.

### Regarding Constitutional Unit (St):

The constitutional unit (st) is a constitutional unit derived from styrene or a styrene derivative thereof. The phrase “constitutional unit derived from styrene” means a constitutional unit that is formed by the cleavage of an ethylenic double bond of styrene. The expression “constitutional unit derived from a styrene derivative” denotes a constitutional unit formed by cleavage of an ethylenic double bond of a styrene derivative (here, those corresponding to the constitutional unit (a10) are excluded).

The term “styrene derivative” denotes a compound in which at least some hydrogen atoms of styrene are substituted with a substituent. Examples of the styrene derivative include those in which the hydrogen atom at the α-position of styrene is substituted with a substituent, those in which one or more hydrogen atoms in the benzene ring of styrene are substituted with a substituent, and those in which the hydrogen atom at the α-position of styrene and one or more hydrogen atoms of the benzene ring are substituted with a substituent.

Examples of the substituent that substitutes the hydrogen atom at the α-position of styrene include an alkyl group having 1 to 5 carbon atoms and a halogenated alkyl group having 1 to 5 carbon atoms.

As the alkyl group having 1 to 5 carbon atoms, a linear or branched alkyl group having 1 to 5 carbon atoms is preferable, and specific examples thereof include a methyl group, an ethyl group, a propyl group, an isopropyl group, an n-butyl group, an isobutyl group, a tert-butyl group, a pentyl group, an isopentyl group, and a neopentyl group. The halogenated alkyl group having 1 to 5 carbon atoms is a group in which a part or all of the hydrogen atoms in the alkyl group having 1 to 5 carbon atoms have been substituted with halogen atoms. As the halogen atom, a fluorine atom is particularly preferable.

As the substituent that substitutes the hydrogen atom at the α-position of styrene, an alkyl group having 1 to 5 carbon atoms or a fluorinated alkyl group having 1 to 5 carbon atoms is preferable, an alkyl group having 1 to 3 carbon atoms or a fluorinated alkyl group having 1 to 3 carbon atoms is more preferable, and a methyl group is still more preferable from the viewpoint of industrial availability.

Examples of substituents that substitute the hydrogen atom of the benzene ring of styrene include an alkyl group, an alkoxy group, a halogen atom, and a halogenated alkyl group.

As the alkyl group as the substituent, an alkyl group having 1 to 5 carbon atoms is preferable, and a methyl group, an ethyl group, a propyl group, an n-butyl group, or a tert-butyl group is more preferable.

As the alkoxy group as the substituent, an alkoxy group having 1 to 5 carbon atoms is preferable, a methoxy group, an ethoxy group, an n-propoxy group, an iso-propoxy group, an n-butoxy group, or a tert-butoxy group is more preferable, and a methoxy group or an ethoxy group is still more preferable.

As the halogen atom as the substituent, a fluorine atom is preferable. Examples of the halogenated alkyl group as the substituent include groups in which a part or all of the hydrogen atoms in the above-described alkyl groups have been substituted with the above-described halogen atoms.

As the substituent that substitutes the hydrogen atom of the benzene ring of styrene, an alkyl group having 1 to 5 carbon atoms is preferable, a methyl group or an ethyl group is more preferable, and a methyl group is still more preferable.

As the constitutional unit (st), a constitutional unit derived from styrene or a constitutional unit derived from a styrene derivative in which the hydrogen atom at the α-position of styrene is substituted with an alkyl group having 1 to 5 carbon atoms or a halogenated alkyl group having 1 to 5 carbon atoms is preferable, a constitutional unit derived from styrene or a constitutional unit derived from a styrene derivative in which the hydrogen atom at the α-position of styrene is substituted with a methyl group is more preferable, and a constitutional unit derived from styrene is still more preferable.

The constitutional unit (st) included in the component (A1) may be used alone or two or more kinds thereof.

In a case where the component (A1) has the constitutional unit (st), the proportion of the constitutional unit (st) is preferably in a range of 1% to 30% by mole and more preferably in a range of 3% to 20% by mole with respect to the total amount (100% by mole) of all constitutional units constituting the component (A1).

The component (A1) contained in the resist composition may be used alone or in combination of two or more kinds thereof.

In the resist composition according to the present embodiment, examples of the component (A1) include a high-molecular-weight compound having a repeating structure of the constitutional unit (a1), and preferred examples thereof include a high-molecular-weight compound having a repeating structure of the constitutional unit (a1) and the constitutional unit (a10).

Among these, suitable examples of the component (A1) include a high-molecular-weight compound consisting of a repeating structure of a constitutional unit (a1) and a constitutional unit (a10); and a high-molecular-weight compound consisting of a repeating structure of a constitutional unit (a1), a constitutional unit (a10), and a constitutional unit (a3).

In the high-molecular-weight compound having a repeating structure of the constitutional unit (a1) and the constitutional unit (a10), the proportion of the constitutional unit (a1) is preferably in a range of 10% to 90% by mole, more preferably in a range of 20% to 80% by mole, still more preferably in a range of 30% to 70% by mole, and particularly preferably in a range of 40% to 70% by mole with respect to the total amount (100% by mole) of all constitutional units constituting the high-molecular-weight compound.

In the high-molecular-weight compound having a repeating structure of the constitutional unit (a1) and the constitutional unit (a10), the proportion of the constitutional unit (a10) is preferably in a range of 10% to 90% by mole, more preferably in a range of 20% to 80% by mole, still more preferably in a range of 30% to 70% by mole, and particularly preferably in a range of 30% to 60% by mole with respect to the total amount (100% by mole) of all constitutional units constituting the high-molecular-weight compound.

In the high-molecular-weight compound having a repeating structure of the constitutional unit (a1), the constitutional unit (a10), and the constitutional unit (a3), the proportion of the constitutional unit (a1) is preferably in a range of 20% to 80% by mole, more preferably in a range of 30% to 70% by mole, still more preferably in a range of 40% to 70% by mole, and particularly preferably in a range of 45% to 65% by mole with respect to the total amount (100% by mole) of all constitutional units constituting the high-molecular-weight compound.

In the high-molecular-weight compound having a repeating structure of the constitutional unit (a1), the constitutional unit (a10), and the constitutional unit (a3), the proportion of the constitutional unit (a10) is preferably in a range of 10% to 70% by mole, more preferably in a range of 20% to 60% by mole, still more preferably in a range of 20% to 50% by mole, and particularly preferably in a range of 25% to 45% by mole with respect to the total amount (100% by mole) of all constitutional units constituting the high-molecular-weight compound.

Further, in the high-molecular-weight compound having a repeating structure of the constitutional unit (a1), the constitutional unit (a10), and the constitutional unit (a3), the proportion of the constitutional unit (a3) is preferably in a range of 1% to 30% by mole, more preferably in a range of 5% to 25% by mole, still more preferably in a range of 5% to 20% by mole, and particularly preferably in a range of 5% to 15% by mole with respect to the total amount (100% by mole) of all constitutional units constituting the high-molecular-weight compound.

The molar ratio (constitutional unit (a1): constitutional unit (a10)) of the constitutional unit (a1) to the constitutional unit (a10) in the high-molecular-weight compound is preferably in a range of 2:8 to 8:2, more preferably in a range of 3:7 to 7:3, and still more preferably in a range of 4:6 to 6:4.

Such a component (A1) can be produced by dissolving a monomer, from which each constitutional unit is derived, in a polymerization solvent and adding a radical polymerization initiator such as azobisisobutylonitrile (AIBN) or dimethyl azobisisobutyrate (for example, V-601) to the solution so that the polymerization is carried out.

Alternatively, such a component (A1) can be produced by dissolving a monomer from which the constitutional unit (a1) is derived and a monomer from which constitutional units (for example, the constitutional unit (a10)) other than the constitutional unit (a1) are derived as necessary in a polymerization solvent, adding the above-described radical polymerization initiator to the solution, and performing polymerization.

Further, a —C(CF₃)₂—OH group may be introduced to the terminal during the polymerization using a combination of chain transfer agents such as HS—CH₂—CH₂—CH₂—C(CF₃)₂—OH. As described above, a copolymer into which a hydroxyalkyl group, formed by substitution of some hydrogen atoms in the alkyl group with fluorine atoms, has been introduced is effective for reducing development defects and reducing line edge roughness (LER: uneven irregularities of a line side wall).

The weight-average molecular weight (Mw) (in terms of polystyrene according to gel permeation chromatography (GPC)) of the component (A1) is not particularly limited, but is preferably in a range of 1,000 to 50,000, more preferably in a range of 2,000 to 30,000, and still more preferably in a range of 3,000 to 20,000. In a case where the Mw of the component (A1) is less than or equal to the upper limits of the above-described preferable ranges, the resist composition exhibits sufficient solubility in a resist solvent for a resist enough to be used as a resist. Meanwhile, in a case where the Mw of the component (A1) is greater than or equal to the lower limits of the above-described preferable ranges, the dry etching resistance and the cross-sectional shape of the resist pattern are enhanced.

Further, the dispersity (Mw/Mn) of the component (A1) is not particularly limited, but is preferably in a range of 1.0 to 4.0, more preferably in a range of 1.0 to 3.0, and particularly preferably in a range of 1.0 to 2.0. Further, Mn represents the number average molecular weight.

### Regarding Component (A2)

In the resist composition of the present embodiment, a base material component (hereinafter, also referred to as “component (A2)”) which does not correspond to the component (A1) and whose solubility in a developing solution is changed due to the action of an acid may be used in combination as the component (A).

The component (A2) is not particularly limited and may be arbitrarily selected from a plurality of components of the related art which have been known as base material components for a chemically amplified resist composition and used.

As the component (A2), a high-molecular-weight compound or a low-molecular-weight compound may be used alone or in combination of two or more kinds thereof.

The proportion of the component (A1) in the component (A) is preferably 25% by mass or greater, more preferably 50% by mass or greater, and still more preferably 75% by mass or greater, and may be 100% by mass with respect to the total mass of the component (A). In a case where the proportion thereof is 25% by mass or greater, a resist pattern having excellent various lithography characteristics such as high sensitivity, high resolution, and improved roughness is likely to be formed.

In the resist composition of the present embodiment, the content of the component (A) may be adjusted according to the thickness of the resist film intended to be formed.

### <Acid-Generating Agent Component (B)>

The resist composition according to the present embodiment contains an acid-generating agent component (B) generating acid upon light exposure. The component (B) contains a compound (B0) (hereinafter, also referred to as “component (B0)”) represented by General Formula (b0).

### <<Component (B0)>>

The component (B0) is a compound represented by General Formula (b0).

M^{b⊕}Xb^{⊖} (b0)

[In the formula, M^{b+} represents the cation (C0). Xb⁻ represents a counter anion.]

### {Cation Moiety}

In General Formula (b0), M^{b+} represents the cation (C0).

### {Anion Moiety}

In General Formula (b0), Xb⁻ represents a counter anion. Xb⁻ is not particularly limited, and those which have been suggested as an anion moiety of an acid-generating agent for a chemically amplified resist composition can be used.

Examples of Xb⁻ include an anion represented by General Formula (b-1-an), General Formula (b-2-an), or General Formula (b-3-an).

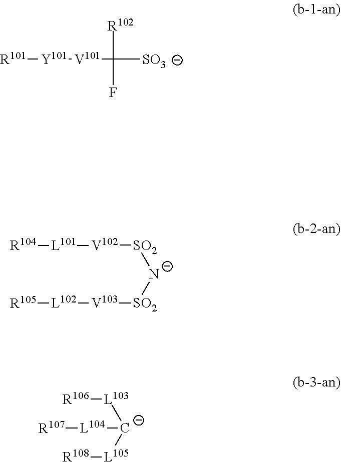

[In the formulae, R¹⁰¹ and R¹⁰⁴ to R¹⁰⁸ each independently represents a cyclic group which may have a substituent, a chain-like alkyl group which may have a substituent, or a chain-like alkenyl group which may have a substituent. R¹⁰⁴ and R¹⁰⁵ may be bonded to each other to form a ring structure. R¹⁰² represents a fluorinated alkyl group having 1 to 5 carbon atoms or a fluorine atom. Y¹⁰¹ represents a divalent linking group having an oxygen atom or a single bond. V¹⁰¹ to V¹⁰³ each independently represents a single bond, an alkylene group, or a fluorinated alkylene group. L¹⁰¹ and L¹⁰² each independently represents a single bond or an oxygen atom. L¹⁰³ to L¹⁰⁵ each independently represents a single bond, —CO—, or —SO₂—.

Anion Represented by Formula (b-1-an)

In Formula (b-1-an), R¹⁰¹ represents a cyclic group which may have a substituent, a chain-like alkyl group which may have a substituent, or a chain-like alkenyl group which may have a substituent.

Cyclic Group which May have Substituent:

The cyclic group is preferably a cyclic hydrocarbon group, and the cyclic hydrocarbon group may be an aromatic hydrocarbon group or an aliphatic hydrocarbon group. The aliphatic hydrocarbon group indicates a hydrocarbon group that has no aromaticity. Further, the aliphatic hydrocarbon group may be saturated or unsaturated. In general, it is preferable that the aliphatic hydrocarbon group be saturated.

The aromatic hydrocarbon group as R¹⁰¹ is a hydrocarbon group having an aromatic ring. The aromatic hydrocarbon group has preferably 3 to 30 carbon atoms, more preferably 5 to 30 carbon atoms, still more preferably 5 to 20 carbon atoms, particularly preferably 6 to 15 carbon atoms, and most preferably 6 to 10 carbon atoms. Here, the number of carbon atoms in a substituent is not included in the above number of carbon atoms.

Specific examples of the aromatic ring of the aromatic hydrocarbon group as R¹⁰¹ include benzene, fluorene, naphthalene, anthracene, phenanthrene, biphenyl, and an aromatic heterocyclic ring in which some carbon atoms constituting any of these aromatic rings have been substituted with hetero atoms. Examples of the hetero atom in the aromatic heterocyclic rings include an oxygen atom, a sulfur atom, and a nitrogen atom.

Specific examples of the aromatic hydrocarbon group as R¹⁰¹ include a group in which one hydrogen atom has been removed from the aromatic ring (an aryl group such as a phenyl group or a naphthyl group) and a group in which one hydrogen atom in the aromatic ring has been substituted with an alkylene group (for example, an arylalkyl group such as a benzyl group, a phenethyl group, a 1-naphthylmethyl group, a 2-naphthylmethyl group, 1-naphthylethyl group, or a 2-naphthylethyl group). The alkylene group (alkyl chain in the arylalkyl group) has preferably 1 to 4 carbon atoms, more preferably 1 or 2 carbon atoms, and particularly preferably 1 carbon atom.

Examples of the cyclic aliphatic hydrocarbon group as R¹⁰¹ include an aliphatic hydrocarbon group having a ring in the structure thereof. The number of carbon atoms of the cyclic aliphatic hydrocarbon group as R¹⁰¹ is preferably in a range of 3 to 50, preferably in a range of 4 to 45, and more preferably in a range of 5 to 40.

Examples of the aliphatic hydrocarbon group having a ring in the structure thereof include an alicyclic hydrocarbon group (a group in which one hydrogen atom has been removed from an aliphatic hydrocarbon ring), a group in which the alicyclic hydrocarbon group is bonded to the terminal of a linear or branched aliphatic hydrocarbon group, and a group in which the alicyclic hydrocarbon group is interposed in the middle of a linear or branched aliphatic hydrocarbon group.

The alicyclic hydrocarbon group has preferably 3 to 20 carbon atoms and more preferably 3 to 12 carbon atoms.

The alicyclic hydrocarbon group may be a polycyclic group or a monocyclic group. As the monocyclic alicyclic hydrocarbon group, a group in which one or more hydrogen atoms have been removed from a monocycloalkane is preferable. The monocycloalkane has preferably 3 to 6 carbon atoms, and specific examples thereof include cyclopentane and cyclohexane. As the polycyclic alicyclic hydrocarbon group, a group in which one or more hydrogen atoms have been removed from a polycycloalkane is preferable, and the number of carbon atoms of the polycycloalkane is preferably in a range of 7 to 30. Among these, a polycycloalkane having a crosslinked ring polycyclic skeleton such as adamantane, norbornane, isobornane, tricyclodecane, or tetracyclododecane; and a polycycloalkane having a fused ring polycyclic skeleton such as a cyclic group having a steroid skeleton are preferable as the polycycloalkane.

Among these examples, as the cyclic aliphatic hydrocarbon group as R¹⁰¹, a group in which one or more hydrogen atoms have been removed from a monocycloalkane or a polycycloalkane is preferable, a group in which one hydrogen atom has been removed from a polycycloalkane is more preferable, an adamantyl group or a norbornyl group is still more preferable, and an adamantyl group is particularly preferable.

The linear aliphatic hydrocarbon group which may be bonded to the alicyclic hydrocarbon group has preferably 1 to 10 carbon atoms, more preferably 1 to 6 carbon atoms, still more preferably 1 to 4 carbon atoms, and most preferably 1 to 3 carbon atoms. As the linear aliphatic hydrocarbon group, a linear alkylene group is preferable, and specific examples thereof include a methylene group [—CH₂—], an ethylene group [—(CH₂)₂—], a trimethylene group [—(CH₂)₃—], a tetramethylene group [—(CH₂)₄—], and a pentamethylene group [—(CH₂)₅—].

The branched aliphatic hydrocarbon group which may be bonded to the alicyclic hydrocarbon group has preferably 2 to 10 carbon atoms, more preferably 3 to 6 carbon atoms, still more preferably 3 or 4 carbon atoms, and most preferably 3 carbon atoms. As the branched aliphatic hydrocarbon group, a branched alkylene group is preferable, and specific examples thereof include alkylalkylene groups, for example, alkylmethylene groups such as —CH(CH₃)—, —CH(CH₂CH₃)—, —C(CH₃)₂—, —C(CH₃)(CH₂CH₃)—, —C(CH₃)(CH₂CH₂CH₃)—, and —C(CH₂CH₃)₂—; alkylethylene groups such as —CH(CH₃)CH₂—, —CH(CH₃)CH(CH₃)—, —C(CH₃)₂CH₂—, —CH(CH₂CH₃)CH₂—, and —C(CH₂CH₃)₂—CH₂—; alkyltrimethylene groups such as —CH(CH₃)CH₂CH₂— and —CH₂CH(CH₃)CH₂—; and alkyltetramethylene groups such as —CH(CH₃)CH₂CH₂CH₂— and —CH₂CH(CH₃)CH₂CH₂—. As the alkyl group in the alkylalkylene group, a linear alkyl group having 1 to 5 carbon atoms is preferable.

Further, the cyclic hydrocarbon group as R¹⁰¹ may have a hetero atom such as a heterocyclic ring. Specific examples thereof include lactone-containing cyclic groups each represented by General Formulae (a2-r-1) to (a2-r-7), —SO₂-containing cyclic groups each represented by General Formulae (a5-r-1) to (a5-r-4), and other heterocyclic groups each represented by Chemical Formulae (r-hr-1) to (r-hr-16). In the formulae, * represents a bonding site bonded to Y¹⁰¹ in Formula (b-1-an).

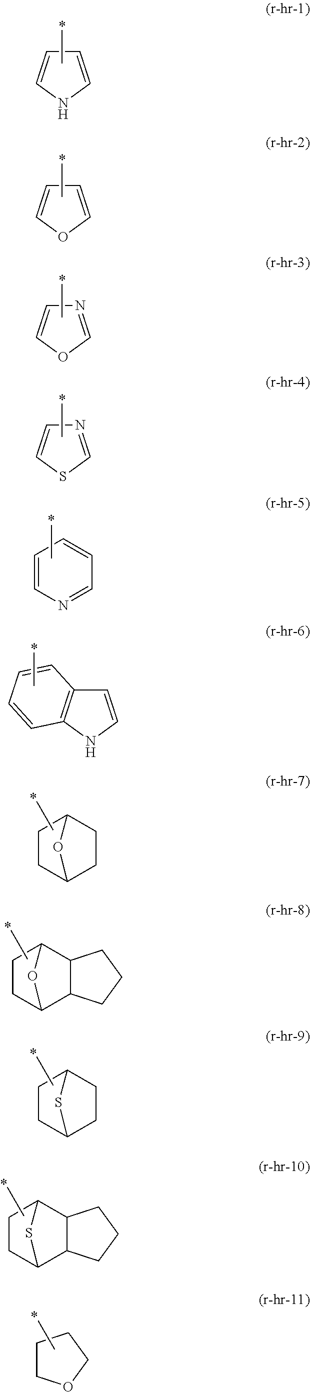

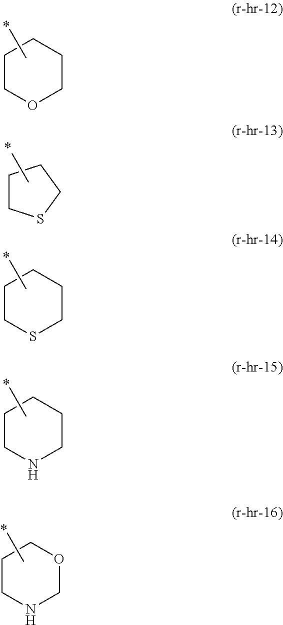

Examples of the substituent for the cyclic group as R¹⁰¹ include an alkyl group, an alkoxy group, a halogen atom, a halogenated alkyl group, a hydroxyl group, a carbonyl group, and a nitro group.

As the alkyl group as the substituent, an alkyl group having 1 to 5 carbon atoms is preferable, and a methyl group, an ethyl group, a propyl group, an n-butyl group, or a tert-butyl group is most preferable.

As the alkoxy group as the substituent, an alkoxy group having 1 to 5 carbon atoms is preferable, a methoxy group, an ethoxy group, an n-propoxy group, an iso-propoxy group, an n-butoxy group, or a tert-butoxy group is more preferable, and a methoxy group or an ethoxy group is most preferable.

Examples of the halogen atom as the substituent include a fluorine atom, a chlorine atom, a bromine atom, and an iodine atom. Among these, a fluorine atom is preferable.

Example of the above-described halogenated alkyl group as the substituent includes a group in which a part or all of the hydrogen atoms in an alkyl group having 1 to 5 carbon atoms such as a methyl group, an ethyl group, a propyl group, an n-butyl group, or a tert-butyl group have been substituted with the above-described halogen atoms.

The carbonyl group as the substituent is a group that substitutes a methylene group (—CH₂—) constituting the cyclic hydrocarbon group.

The cyclic hydrocarbon group as R¹⁰¹ may be a fused cyclic group having a fused ring in which an aliphatic hydrocarbon ring and an aromatic ring are fused. Examples of the fused ring include those obtained by fusing one or more aromatic rings with a polycycloalkane having a crosslinked ring-based polycyclic skeleton. Specific examples of the crosslinked ring-based polycycloalkane include a bicycloalkane such as bicyclo[2.2.1]heptane (norbornane) and bicyclo[2.2.2]octane. As the fused cyclic group, a group having a fused ring in which two or three aromatic rings are fused with a bicycloalkane is preferable, and a group having a fused ring in which two or three aromatic rings are fused with bicyclo[2.2.2]octane is more preferable. Specific examples of the fused cyclic group as R¹⁰¹ include those represented by Formulae (r-br-1) and (r-br-2). In the formulae, * represents a bonding site bonded to Y¹⁰¹ in Formula (b-1-an).

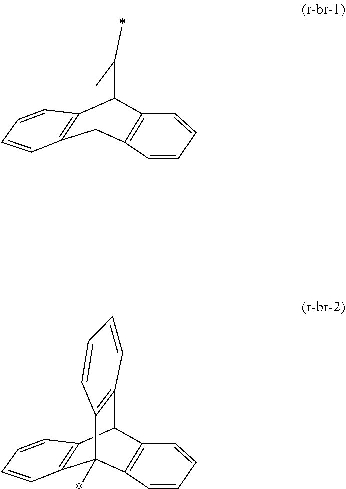 [SMILES: *C(C)C1c2ccccc2Cc2ccccc21 ; *C12c3ccccc3C(c3ccccc31)c1ccccc12]

Examples of the substituent that the fused cyclic group as R¹⁰¹ may have include an alkyl group, an alkoxy group, a halogen atom, a halogenated alkyl group, a hydroxyl group, a carbonyl group, a nitro group, an aromatic hydrocarbon group, and an alicyclic hydrocarbon group.

Examples of the alkyl group, the alkoxy group, the halogen atom, and the halogenated alkyl group as the substituent of the fused cyclic group include those described as the substituent of the cyclic group as R¹⁰¹.

Examples of the aromatic hydrocarbon group as the substituent of the fused cyclic group include a group in which one hydrogen atom has been removed from the aromatic ring (an aryl group such as a phenyl group or a naphthyl group), a group in which one hydrogen atom in the aromatic ring has been substituted with an alkylene group (for example, an arylalkyl group such as a benzyl group, a phenethyl group, a 1-naphthylmethyl group, a 2-naphthylmethyl group, 1-naphthylethyl group, or a 2-naphthylethyl group), and a heterocyclic group represented by any of Formulae (r-hr-1) to (r-hr-6).

Examples of the alicyclic hydrocarbon group as the substituent of the fused cyclic group include a group in which one hydrogen atom has been removed from a monocycloalkane such as cyclopentane or cyclohexane; a group in which one hydrogen atom has been removed from a polycycloalkane such as adamantane, norbornane, isobornane, tricyclodecane, or tetracyclododecane; a lactone-containing cyclic group represented by any of General Formulae (a2-r-1) to (a2-r-7); a —SO₂-containing cyclic group represented by any of General Formulae (a5-r-1) to (a5-r-4); and a heterocyclic group represented by any of Formulae (r-hr-7) to (r-hr-16).

The cyclic hydrocarbon group as R¹⁰¹ may be a group in which two or more aliphatic rings and/or aromatic rings are linked to each other with a linear or branched aliphatic hydrocarbon group which may have a substituent. The linear or branched aliphatic hydrocarbon group that links an alicyclic hydrocarbon group may be formed such that a methylene group (—CH₂—) constituting the aliphatic hydrocarbon chain is substituted with a divalent group having a hetero atom. Examples of the divalent group having a hetero atom include (—O—), —C(═O)—O—, —O—C(═O)—, —C(═O)—, —O—C(═O)—O—, —C(═O)—NH—, —NH—, —S—, —S(═O)₂—, and —S(═O)₂—O—.

Chain-Like Alkyl Group which May have Substituent:

The chain-like alkyl group as R¹⁰¹ may be linear or branched.

The linear alkyl group has preferably 1 to 20 carbon atoms, more preferably 1 to 15 carbon atoms, and most preferably 1 to 10 carbon atoms.

The branched alkyl group has preferably 3 to 20 carbon atoms, more preferably 3 to 15 carbon atoms, and most preferably 3 to 10 carbon atoms. Specific examples thereof include a 1-methylethyl group, a 1-methylpropyl group, a 2-methylpropyl group, a 1-methylbutyl group, a 2-methylbutyl group, a 3-methylbutyl group, a 1-ethylbutyl group, a 2-ethylbutyl group, a 1-methylpentyl group, a 2-methylpentyl group, a 3-methylpentyl group, and a 4-methylpentyl group.

Chain-Like Alkenyl Group which May have Substituent:

The chain-like alkenyl group as R¹⁰¹ may be linear or branched, and the number of carbon atoms thereof is preferably in a range of 2 to 10, more preferably in a range of 2 to 5, still more preferably in a range of 2 to 4, and particularly preferably 3. Examples of the linear alkenyl group include a vinyl group, a propenyl group (an allyl group), and a butynyl group. Examples of the branched alkenyl group include a 1-methylvinyl group, a 2-methylvinyl group, a 1-methylpropenyl group, and a 2-methylpropenyl group.

Among the examples, as the chain-like alkenyl group, a linear alkenyl group is preferable, a vinyl group or a propenyl group is more preferable, and a vinyl group is particularly preferable.

Examples of the substituent for the chain-like alkyl group or alkenyl group as R¹⁰¹ include an alkoxy group, a halogen atom, a halogenated alkyl group, a hydroxyl group, a carbonyl group, a nitro group, an amino group, and a cyclic group as R¹⁰¹.

Among the examples, R¹⁰¹ represents preferably a cyclic group which may have a substituent and more preferably a cyclic hydrocarbon group which may have a substituent. More specifically, as the cyclic hydrocarbon group, a phenyl group, a naphthyl group, or a group in which one or more hydrogen atoms have been removed from a polycycloalkane, a lactone-containing cyclic group represented by any of General Formulae (a2-r-1) to (a2-r-7), or a —SO₂-containing cyclic group represented by any of General Formulae (a5-r-1) to (a5-r-4) is preferable, a group in which one or more hydrogen atoms have been removed from a polycycloalkane or a —SO₂-containing cyclic group represented by any of General Formulae (a5-r-1) to (a5-r-4) is more preferable, and an adamantyl group or a —SO₂-containing cyclic group represented by General Formula (a5-r-1) is still more preferable.

In a case where the cyclic hydrocarbon group has a substituent, it is preferable that the substituent be a hydroxyl group.

In Formula (b-1-an), Y¹⁰¹ represents a single bond or a divalent linking group containing an oxygen atom.

In a case where Y¹⁰¹ represents a divalent linking group containing an oxygen atom, Y¹⁰¹ may contain an atom other than the oxygen atom. Examples of atoms other than an oxygen atom include a carbon atom, a hydrogen atom, a sulfur atom, and a nitrogen atom.

Examples of the divalent linking group having an oxygen atom include a non-hydrocarbon oxygen atom-containing linking group such as an oxygen atom (an ether bond: —O—), an ester bond (—C(═O)—O—), an oxycarbonyl group (—O—C(═O)—), an amide bond (—C(═O)—NH—), a carbonyl group (—C(═O)—), or a carbonate bond (—O—C(═O)—O—); and combinations of the above-described non-hydrocarbon oxygen atom-containing linking groups with an alkylene group. Further, a sulfonyl group (—SO₂—) may be further linked to the combination. Examples of the divalent linking group having an oxygen atom include linking groups each represented by General Formulae (y-al-1) to (y-al-7). Further, in General Formulae (y-al-1) to (y-al-7), V′¹⁰¹ in General Formulae (y-al-1) to (y-al-7) is bonded to R¹⁰¹ in Formula (b-1-an).

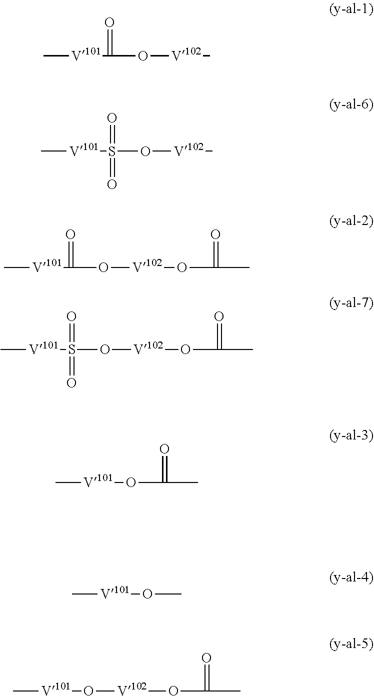

[In the formulae, V′⁰¹ represents a single bond or an alkylene group having 1 to 5 carbon atoms, and V′¹⁰² represents a divalent saturated hydrocarbon group having 1 to 30 carbon atoms.]

As the divalent saturated hydrocarbon group as V′¹⁰², an alkylene group having 1 to 30 carbon atoms is preferable, an alkylene group having 1 to 10 carbon atoms is more preferable, and an alkylene group having 1 to 5 carbon atoms is still more preferable.

The alkylene group as V′¹⁰¹ and V′¹⁰² may be a linear alkylene group or a branched alkylene group, and a linear alkylene group is preferable.

Specific examples of the alkylene group as V′¹⁰¹ and V′¹⁰² include a methylene group [—CH₂—]; an alkylmethylene group such as —CH(CH₃)—, —CH(CH₂CH₃)—, —C(CH₃)₂—, —C(CH₃)(CH₂CH₃)—, —C(CH₃)(CH₂CH₂CH₃)—, or —C(CH₂CH₃)₂—; an ethylene group [—CH₂CH₂—]; an alkylethylene group such as —CH(CH₃)CH₂—, —CH(CH₃)CH(CH₃)—, —C(CH₃)₂CH₂—, or —CH(CH₂CH₃)CH₂—; a trimethylene group (n-propylene group) [—CH₂CH₂CH₂—]; an alkyltrimethylene group such as —CH(CH₃)CH₂CH₂— or —CH₂CH(CH₃)CH₂—; a tetramethylene group [—CH₂CH₂CH₂CH₂—]; an alkyltetramethylene group such as —CH(CH₃)CH₂CH₂CH₂— or —CH₂CH(CH₃)CH₂CH₂—; and a pentamethylene group [—CH₂CH₂CH₂CH₂CH₂—].

Further, a part of methylene group in the alkylene group as V′¹⁰¹ and V′¹⁰² may be substituted with a divalent aliphatic cyclic group having 5 to 10 carbon atoms. As the aliphatic cyclic group, a divalent group in which one hydrogen atom has been further removed from the cyclic aliphatic hydrocarbon group (a monocyclic aliphatic hydrocarbon group or a polycyclic aliphatic hydrocarbon group) as Ra′³ in Formula (a1-r-1) is preferable, and a cyclohexylene group, a 1,5-adamantylene group, or a 2,6-adamantylene group is more preferable.

Y¹⁰¹ represents preferably a divalent linking group having an ester bond or a divalent linking group having an ether bond and more preferably a linking group represented by any of Formulae (y-al-1) to (y-al-5).

In Formula (b-1-an), V¹¹⁰ represents a single bond, an alkylene group, or a fluorinated alkylene group. It is preferable that the alkylene group and the fluorinated alkylene group as V¹⁰¹ have 1 to 4 carbon atoms. Examples of the fluorinated alkylene group as V¹¹⁰ include a group in which a part or all of the hydrogen atoms in the alkylene group as V¹¹⁰ have been substituted with fluorine atoms. Among these examples, it is preferable that V¹¹ represent a single bond or a fluorinated alkylene group having 1 to 4 carbon atoms.

In Formula (b-1-an), R¹⁰² represents a fluorine atom or a fluorinated alkyl group having 1 to 5 carbon atoms. R¹⁰² represents preferably a fluorine atom or a perfluoroalkyl group having 1 to 5 carbon atoms and more preferably a fluorine atom.

Specific examples of the anion moiety represented by Formula (b-1-an) include a fluorinated alkyl sulfonate anion such as a trifluoromethanesulfonate anion or a perfluorobutanesulfonate anion in a case where Y¹⁰¹ represents a single bond; and specific examples thereof include an anion represented by any of Formulae (an-1) to (an-3) in a case where Y¹⁰¹ represents a divalent linking group containing an oxygen atom.

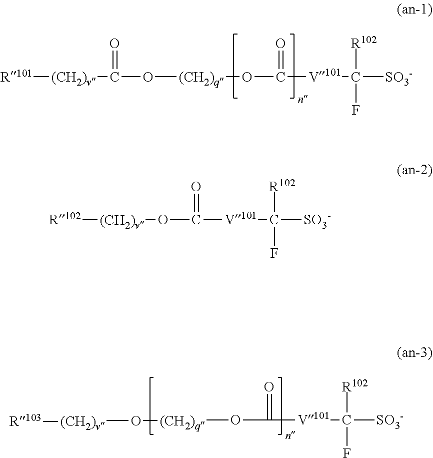

[In the formulae, R″¹⁰¹ represents an aliphatic cyclic group which may have a substituent, a monovalent heterocyclic group represented by any of Chemical Formulae (r-hr-1) to (r-hr-6), a fused cyclic group represented by Formula (r-br-1) or (r-br-2), a chain-like alkyl group which may have a substituent, or an aromatic cyclic group which may have a substituent. R″¹⁰² represents an aliphatic cyclic group which may have a substituent, a fused cyclic group represented by Formula (r-br-1) or (r-br-2), a lactone-containing cyclic group represented by any of General Formulae (a2-r-1) and (a2-r-3) to (a2-r-7), or a —SO₂-containing cyclic group represented by any of General Formulae (a5-r-1) to (a5-r-4). R″¹⁰³ represents an aromatic cyclic group which may have a substituent, an aliphatic cyclic group which may have a substituent, or a chain-like alkenyl group which may have a substituent. V″¹⁰¹ represents a single bond, an alkylene group having 1 to 4 carbon atoms, or a fluorinated alkylene group having 1 to 4 carbon atoms. R¹⁰² represents a fluorine atom or a fluorinated alkyl group having 1 to 5 carbon atoms. Each v″ independently represents an integer of 0 to 3, each q″ independently represents an integer of 0 to 20, and n″ represents 0 or 1.]

As the aliphatic cyclic group as R″¹⁰¹, R″¹⁰², and R″¹⁰³ which may have a substituent, the same groups as those for the cyclic aliphatic hydrocarbon group as R¹⁰¹ in Formula (b-1-an) are preferable. Examples of the substituent include the same groups as those for the substituent that may substitute the cyclic aliphatic hydrocarbon group as R¹⁰¹ in Formula (b-1-an).

As the aromatic cyclic group as R″¹⁰¹ and R″¹⁰³ which may have a substituent, the same groups as those for the aromatic hydrocarbon group in the cyclic hydrocarbon group as R¹⁰¹ in Formula (b-1-an) are preferable. Examples of the substituent include the same groups as those for the substituent that may substitute the aromatic hydrocarbon group as R¹⁰¹ in Formula (b-1-an).

As the chain-like alkyl group as R″¹⁰¹ which may have a substituent, the same groups as those for the chain-like alkyl group as R¹⁰¹ in Formula (b-1-an) are preferable. As the chain-like alkenyl group as R″¹⁰³ which may have a substituent, the same groups as those for the chain-like alkenyl group as R¹⁰¹ in Formula (b-1-an) are preferable.

Specific examples of the anion moiety represented by Formula (b-1-an) are shown below, but the present invention is not limited thereto.

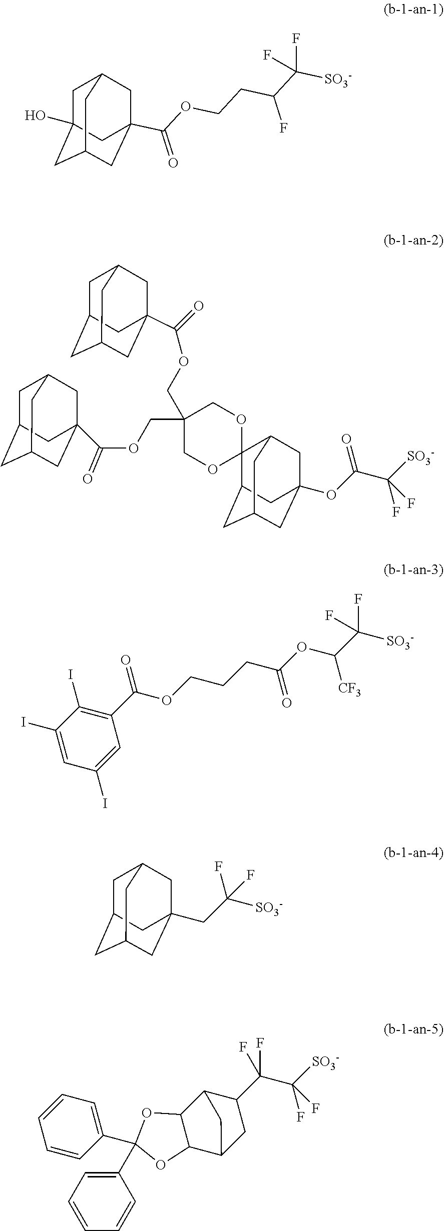

Anion Represented by Formula (b-2-an)

In Formula (b-2-an), R¹⁰⁴ and R¹⁰⁵ each independently represents a cyclic group which may have a substituent, a chain-like alkyl group which may have a substituent, or a chain-like alkenyl group which may have a substituent, and examples thereof each include the same groups as those for R¹⁰¹ in Formula (b-1-an). Here, R¹⁰⁴ and R¹⁰⁵ may be bonded to each other to form a ring.

R¹⁰⁴ and R¹⁰⁵ represent preferably a chain-like alkyl group which may have a substituent and more preferably a linear or branched alkyl group or a linear or branched fluorinated alkyl group.

The chain-like alkyl group has preferably 1 to 10 carbon atoms, more preferably 1 to 7 carbon atoms, and still more preferably 1 to 3 carbon atoms. It is preferable that the number of carbon atoms in the chain-like alkyl group as R¹⁰⁴ and R¹⁰⁵ decrease within the range of the number of carbon atoms from the viewpoint that the solubility in a solvent for a resist is also satisfactory. Further, in the chain-like alkyl group as R¹⁰⁴ and R⁰⁵, it is preferable that the number of hydrogen atoms substituted with fluorine atoms be as large as possible from the viewpoint that the acid strength increases and the transparency to high energy light or electron beams having a wavelength of 250 nm or less is improved. The proportion of fluorine atoms in the chain-like alkyl group, that is, the fluorination ratio is preferably in a range of 70% to 100% and more preferably in a range of 90% to 100%, and it is most preferable that the chain-like alkyl group be a perfluoroalkyl group in which all hydrogen atoms are substituted with fluorine atoms.

In Formula (b-2-an), V¹⁰² and V¹⁰³ each independently represents a single bond, an alkylene group, or a fluorinated alkylene group, and examples thereof each include the same groups as those for V¹¹¹ in Formula (b-1-an).

In Formula (b-2-an), L¹⁰¹ and L¹⁰² each independently represents a single bond or an oxygen atom.

Anion Represented by Formula (b-3-an)

In Formula (b-3-an), R¹⁰⁶ to R¹⁰⁸ each independently represents a cyclic group which may have a substituent, a chain-like alkyl group which may have a substituent, or a chain-like alkenyl group which may have a substituent, and examples thereof each include the same groups as those for R¹⁰¹ in Formula (b-1-an).

In Formula (b-3-an), L¹⁰³ to L¹⁰⁵ each independently represents a single bond, —CO—, or —SO₂—.

Among the examples, it is preferable that Xb⁻ represent an anion represented by Formula (b-1-an). Among the examples, an anion represented by any of General Formulae (an-1) to (an-3) is more preferable, an anion represented by General Formula (an-1) or (an-2) is still more preferable, and an anion represented by General Formula (an-1) is particularly preferable.

Specific examples of the component (B0) are shown below but are not limited thereto.

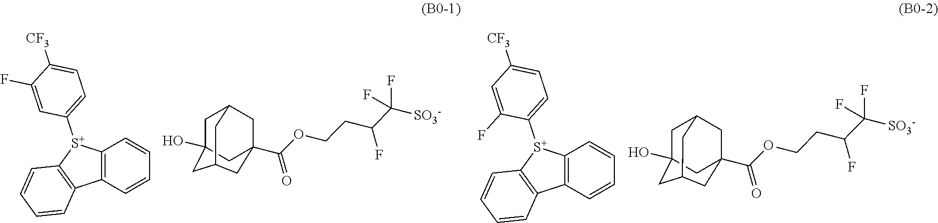

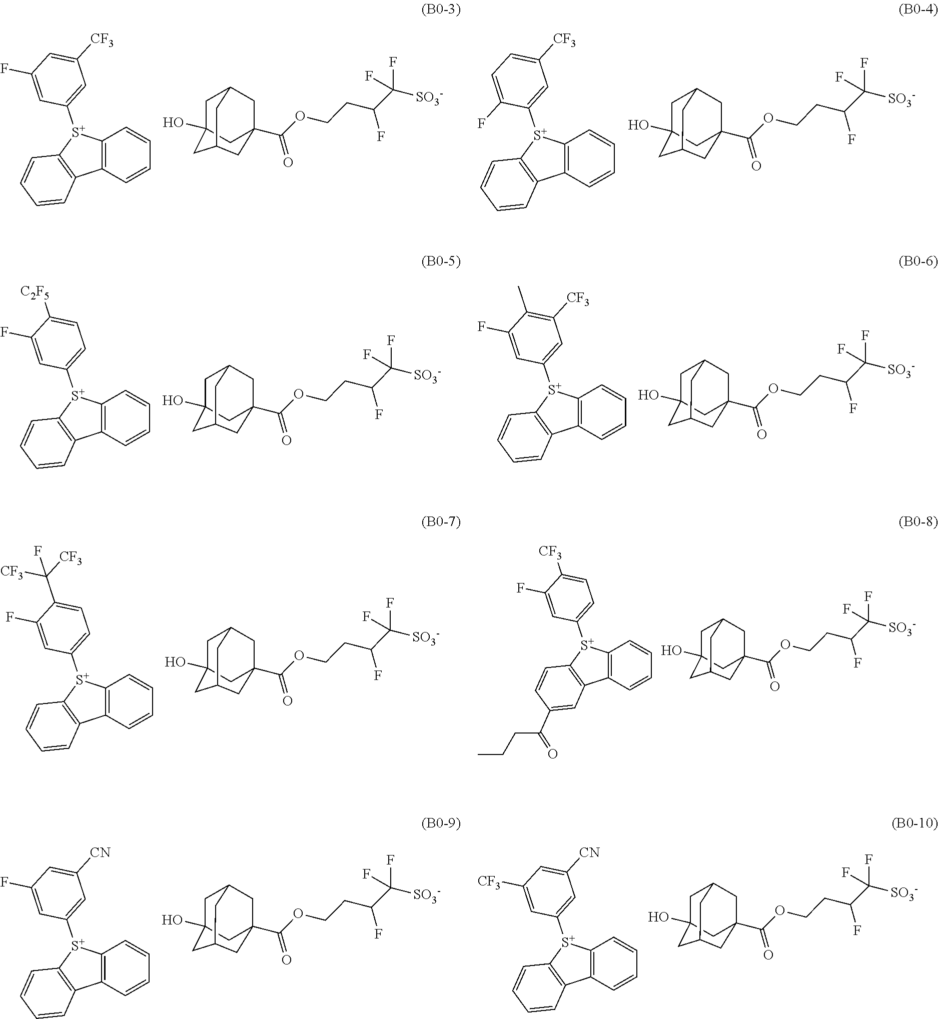

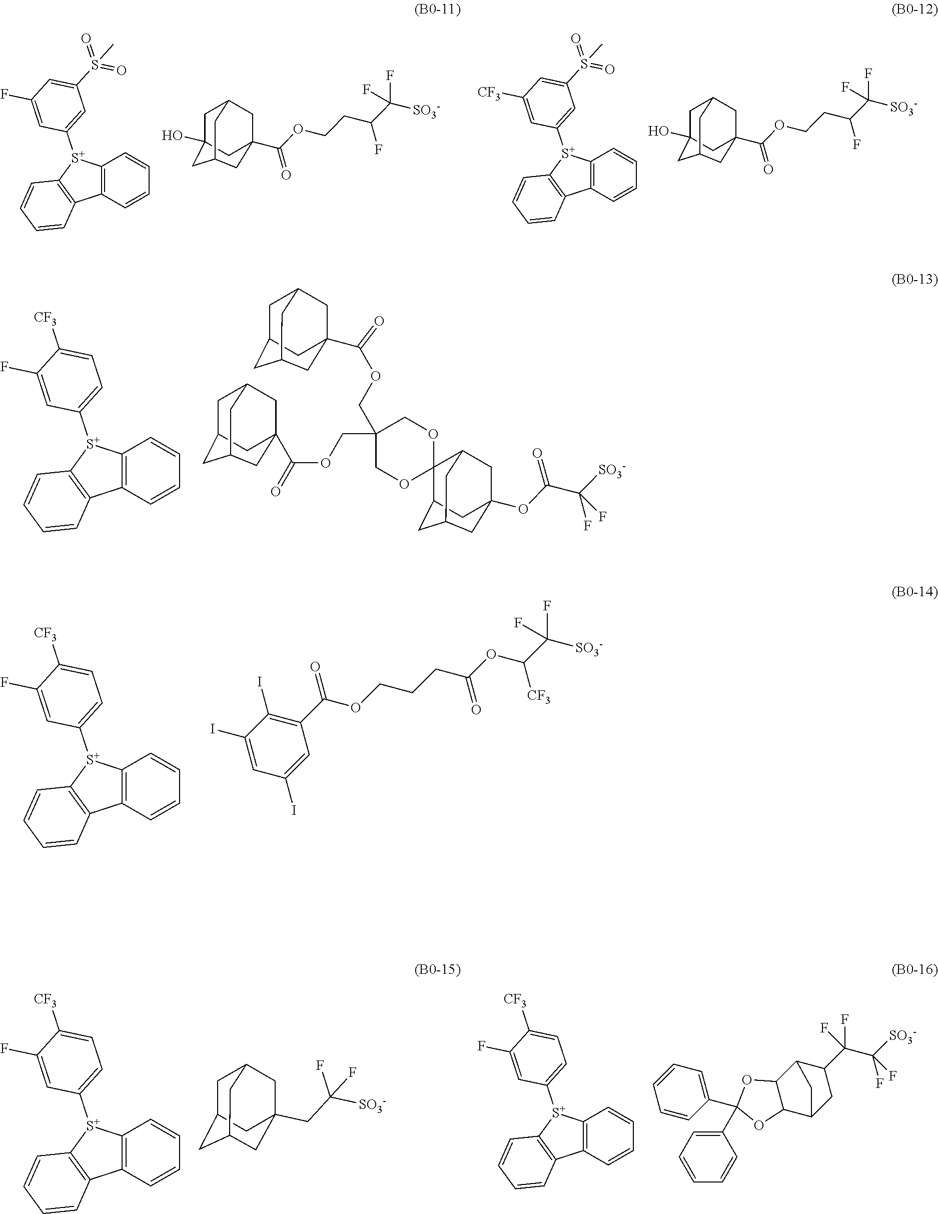

In the resist composition according to the present embodiment, the component (B0) may be used alone or may be used in a combination of two or more kinds thereof.

In the resist composition according to the present embodiment, the content of the component (B0) is preferably in a range of 5 to 40 parts by mass, more preferably in a range of 10 to 40 parts by mass, still more preferably in a range of 12 to 40 parts by mass, and particularly preferably in a range of 15 to 35 parts by mass with respect to 100 parts by mass of the component (A).

In a case where the content of the component (B0) is greater than or equal to the lower limits of the above-described preferable ranges, lithography characteristics such as the sensitivity, reduction of the line width roughness (LWR), the pattern shape, and reduction of defects in the resist pattern formation are further improved. Meanwhile, in a case where the content thereof is less than or equal to the upper limits of the above-described preferable ranges, a uniform solution is easily obtained, and the storage stability of the resist composition is further improved in a case of dissolving each component of the resist composition in an organic solvent.

The proportion of the component (B0) in the total component (B) contained in the resist composition according to the present embodiment is, for example, 50% by mass or greater, preferably 70% by mass or greater, and more preferably 95% by mass or greater. The proportion of the component (B0) in the total component (B) may be 100% by mass.

The component (B) in the resist composition according to the present embodiment may contain an acid-generating agent component (B1) other than the above-described component (B0) (hereinafter, also referred to as “component (B1)”).

### <<Component (B1)>>

Examples of the component (B1) include various compounds such as onium salt-based acid-generating agents (here, a component corresponding to the component (B0) is excluded) such as an iodonium salt and a sulfonium salt; an oxime sulfonate-based acid-generating agent; diazomethane-based acid-generating agents such as bisalkyl or bisaryl sulfonyl diazomethanes and poly(bis-sulfonyl)diazomethanes; nitrobenzyl sulfonate-based acid-generating agents, iminosulfonate-based acid-generating agents, and disulfone-based acid-generating agents.

Examples of the onium salt-based acid-generating agents include a compound represented by General Formula (b-1) (hereinafter, also referred to as “component (b-1)”), a compound represented by General Formula (b-2) (hereinafter, also referred to as “component (b-2)”), and a compound represented by General Formula (b-3) (hereinafter, also referred to as “component (b-3)”).

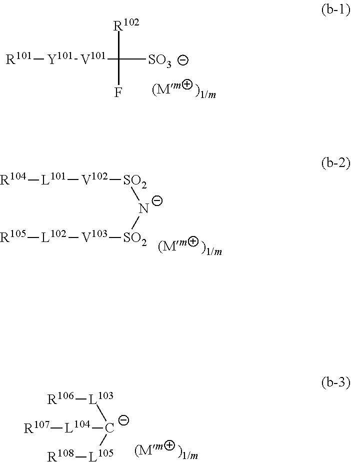

[In the formulae, R¹⁰¹ and R¹⁰⁴ to R¹⁰⁸ each independently represents a cyclic group which may have a substituent, a chain-like alkyl group which may have a substituent, or a chain-like alkenyl group which may have a substituent. R¹⁰⁴ and R¹⁰⁵ may be bonded to each other to form a ring structure. R¹⁰² represents a fluorinated alkyl group having 1 to 5 carbon atoms or a fluorine atom. Y¹⁰¹ represents a divalent linking group having an oxygen atom or a single bond. V¹⁰¹ to V¹⁰³ each independently represents a single bond, an alkylene group, or a fluorinated alkylene group. L¹⁰¹ and L¹⁰² each independently represents a single bond or an oxygen atom. L¹⁰³ to L¹⁰⁵ each independently represents a single bond, —CO—, or —SO₂—. m represents an integer of 1 or greater, and M′^{m+} represents an m-valent onium cation.]

### {Anion Moiety}

Anions in Component (b-1)

The anion moiety in the component (b-1) is an anion represented by Formula (b-1-an).

Anions in Component (b-2)

The anion moiety in the component (b-2) is an anion represented by Formula (b-2-an).

Anions in Component (b-3)

The anion moiety in the component (b-3) is an anion represented by Formula (b-3-an).

Among the examples, an anion represented by Formula (b-1-an) is preferable as the anion moiety of the component (B1). Among the examples, an anion represented by any of General Formulae (an-1) to (an-3) is more preferable, an anion represented by General Formula (an-1) or (an-2) is still more preferable, and an anion represented by General Formula (an-2) is particularly preferable.

### {Cation Moiety}

In Formulae (b-1), (b-2), and (b-3), M^{m+} represents an m-valent onium cation. Among these, a sulfonium cation and an iodonium cation are preferable.

m represents an integer of 1 or greater.

Preferred examples of the cation moiety ((M^{m+})_{1/m}) include organic cations each represented by General Formulae (ca-1) to (ca-5) (here, a cation corresponding to the cation (C0) is excluded).

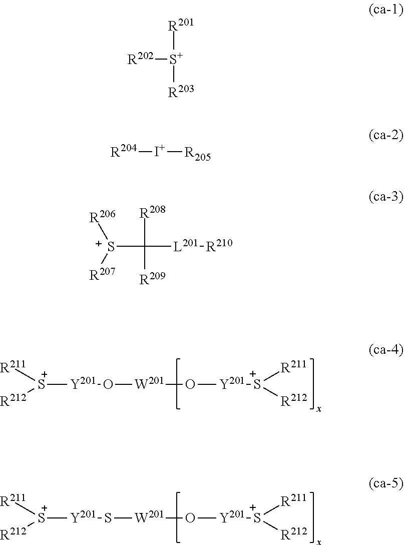

[In the formulae, R²⁰¹ to R²⁰⁷, R²¹¹, and R²¹² each independently represents an aryl group, an alkyl group, or an alkenyl group, which may have a substituent. R²⁰¹ to R²⁰³, R²⁰⁶ and R²⁰⁷, and R²¹¹ and R²¹² may be bonded to each other to form a ring with the sulfur atom in the formula. R²⁰⁸ and R²⁰⁹ each independently represents a hydrogen atom or an alkyl group having 1 to 5 carbon atoms. R²¹⁰ represents an aryl group which may have a substituent, an alkyl group which may have a substituent, an alkenyl group which may have a substituent, or a SO₂-containing cyclic group which may have a substituent. L²⁰¹ represents —C(═O)— or —C(═O)—O—. Y²⁰¹'s each independently represents an arylene group, an alkylene group, or an alkenylene group. x represents 1 or 2. W²⁰¹ represents an (x+1)-valent linking group.]

In General Formulae (ca-1) to (ca-5), examples of the aryl group as R²⁰¹ to R²⁰⁷, R²¹¹, and R²¹² include an unsubstituted aryl group having 6 to 20 carbon atoms. Among these, a phenyl group or a naphthyl group is preferable.

The alkyl group as R²⁰¹ to R²⁰⁷, R²¹¹, and R²¹² is a chain-like or cyclic alkyl group, and the number of carbon atoms thereof is preferably in a range of 1 to 30. It is preferable that the alkenyl group as R²⁰¹ to R²⁰⁷, R²¹¹, and R²¹² have 2 to 10 carbon atoms.

Examples of the substituent that R²⁰¹ to R²⁰⁷ and R²¹⁰ to R²¹² may have include an alkyl group, a halogen atom, a halogenated alkyl group, a carbonyl group, a cyano group, an amino group, an aryl group, and groups each represented by General Formulae (ca-r-1) to (ca-r-7).

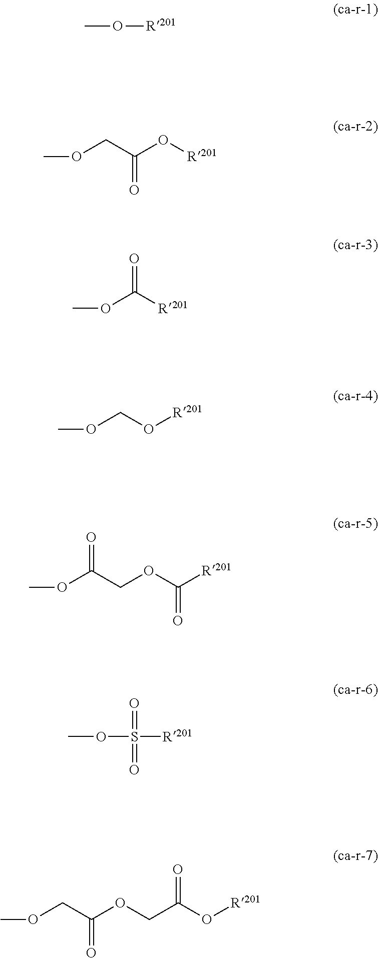

[In the formulae, R′²⁰¹'s each independently represents a hydrogen atom, a cyclic group which may have a substituent, a chain-like alkyl group which may have a substituent, or a chain-like alkenyl group which may have a substituent.]

Cyclic Group which May have Substituent:

The cyclic group is preferably a cyclic hydrocarbon group, and the cyclic hydrocarbon group may be an aromatic hydrocarbon group or an aliphatic hydrocarbon group. The aliphatic hydrocarbon group indicates a hydrocarbon group that has no aromaticity. Further, the aliphatic hydrocarbon group may be saturated or unsaturated. In general, it is preferable that the aliphatic hydrocarbon group be saturated.

The aromatic hydrocarbon group as R′²⁰¹ is a hydrocarbon group having an aromatic ring. The aromatic hydrocarbon group preferably has 3 to 30 carbon atoms, more preferably 5 to 30 carbon atoms, still more preferably 5 to 20 carbon atoms, particularly preferably 6 to 15 carbon atoms, and most preferably 6 to 10 carbon atoms. Here, the number of carbon atoms in a substituent is not included in the above number of carbon atoms.

Specific examples of the aromatic ring contained in the aromatic hydrocarbon group as R′²⁰¹ include benzene, fluorene, naphthalene, anthracene, phenanthrene, biphenyl, or an aromatic heterocyclic ring in which some carbon atoms constituting any of these aromatic rings have been substituted with hetero atoms. Examples of the hetero atom in the aromatic heterocyclic rings include an oxygen atom, a sulfur atom, and a nitrogen atom.

Specific examples of the aromatic hydrocarbon group as R′²⁰¹ include a group in which one hydrogen atom has been removed from the aromatic ring (an aryl group such as a phenyl group or a naphthyl group), and a group in which one hydrogen atom in the aromatic ring has been substituted with an alkylene group (for example, an arylalkyl group such as a benzyl group, a phenethyl group, a 1-naphthylmethyl group, a 2-naphthylmethyl group, 1-naphthylethyl group, or a 2-naphthylethyl group). The alkylene group (alkyl chain in the arylalkyl group) preferably has 1 to 4 carbon atoms, more preferably 1 or 2 carbon atoms, and particularly preferably 1 carbon atom.

Examples of the cyclic aliphatic hydrocarbon group as R′²⁰¹ include an aliphatic hydrocarbon group having a ring in the structure thereof.

Examples of the aliphatic hydrocarbon group having a ring in the structure thereof include an alicyclic hydrocarbon group (a group in which one hydrogen atom has been removed from an aliphatic hydrocarbon ring), a group in which the alicyclic hydrocarbon group is bonded to the terminal of a linear or branched aliphatic hydrocarbon group, and a group in which the alicyclic hydrocarbon group is interposed in the middle of a linear or branched aliphatic hydrocarbon group.

The alicyclic hydrocarbon group has preferably 3 to 20 carbon atoms and more preferably 3 to 12 carbon atoms.

The alicyclic hydrocarbon group may be a polycyclic group or a monocyclic group. As the monocyclic alicyclic hydrocarbon group, a group in which one or more hydrogen atoms have been removed from a monocycloalkane is preferable. The monocycloalkane has preferably 3 to 6 carbon atoms, and specific examples thereof include cyclopentane and cyclohexane. As the polycyclic alicyclic hydrocarbon group, a group in which one or more hydrogen atoms have been removed from a polycycloalkane is preferable, and the number of carbon atoms of the polycycloalkane is preferably in a range of 7 to 30. Among these, a polycycloalkane having a crosslinked ring polycyclic skeleton such as adamantane, norbornane, isobornane, tricyclodecane, or tetracyclododecane; and a polycycloalkane having a fused ring polycyclic skeleton such as a cyclic group having a steroid skeleton are preferable as the polycycloalkane.

Among these examples, as the cyclic aliphatic hydrocarbon group as R′²⁰¹, a group in which one or more hydrogen atoms have been removed from a monocycloalkane or a polycycloalkane is preferable, a group in which one hydrogen atom has been removed from a polycycloalkane is more preferable, an adamantyl group or a norbornyl group is particularly preferable, and an adamantyl group is most preferable.

The linear or branched aliphatic hydrocarbon group which may be bonded to the alicyclic hydrocarbon group has preferably 1 to 10 carbon atoms, more preferably 1 to 6 carbon atoms, still more preferably 1 to 4 carbon atoms, and particularly preferably 1 to 3 carbon atoms.

As the linear aliphatic hydrocarbon group, a linear alkylene group is preferable, and specific examples thereof include a methylene group [—CH₂—], an ethylene group [—(CH₂)₂—], a trimethylene group [—(CH₂)₃—], a tetramethylene group [—(CH₂)₄—], and a pentamethylene group [—(CH₂)₅—].

As the branched aliphatic hydrocarbon group, a branched alkylene group is preferable, and specific examples thereof include alkylalkylene groups, for example, alkylmethylene groups such as —CH(CH₃)—, —CH(CH₂CH₃)—, —C(CH₃)₂—, —C(CH₃)(CH₂CH₃)—, —C(CH₃)(CH₂CH₂CH₃)—, and —C(CH₂CH₃)₂—; alkylethylene groups such as —CH(CH₃)CH₂—, —CH(CH₃)CH(CH₃)—, —C(CH₃)₂CH₂—, —CH(CH₂CH₃)CH₂—, and —C(CH₂CH₃)₂—CH₂—; alkyltrimethylene groups such as —CH(CH₃)CH₂CH₂— and —CH₂CH(CH₃)CH₂—; and alkyltetramethylene groups such as —CH(CH₃)CH₂CH₂CH₂— and —CH₂CH(CH₃)CH₂CH₂—. As the alkyl group in the alkylalkylene group, a linear alkyl group having 1 to 5 carbon atoms is preferable.

Further, the cyclic hydrocarbon group as R′²⁰¹ may have a hetero atom such as a heterocyclic ring. Specific examples thereof include lactone-containing cyclic groups each represented by General Formulae (a2-r-1) to (a2-r-7), —SO₂-containing cyclic groups each represented by General Formulae (a5-r-1) to (a5-r-4), and other heterocyclic groups each represented by Chemical Formulae (r-hr-1) to (r-hr-16).

Examples of the substituent for the cyclic group as R′²⁰¹ include an alkyl group, an alkoxy group, a halogen atom, a halogenated alkyl group, a hydroxyl group, a carbonyl group, and a nitro group.

As the alkyl group as the substituent, an alkyl group having 1 to 5 carbon atoms is preferable, and a methyl group, an ethyl group, a propyl group, an n-butyl group, or a tert-butyl group is most preferable.

As the alkoxy group as the substituent, an alkoxy group having 1 to 5 carbon atoms is preferable, a methoxy group, an ethoxy group, an n-propoxy group, an iso-propoxy group, an n-butoxy group, or a tert-butoxy group is more preferable, and a methoxy group or an ethoxy group is most preferable.

As the halogen atom as a substituent, a fluorine atom is preferable.

Example of the above-described halogenated alkyl group as the substituent includes a group in which a part or all of the hydrogen atoms in an alkyl group having 1 to 5 carbon atoms such as a methyl group, an ethyl group, a propyl group, an n-butyl group, or a tert-butyl group have been substituted with the above-described halogen atoms.

The carbonyl group as the substituent is a group that substitutes a methylene group (—CH₂—) constituting the cyclic hydrocarbon group.

Chain-Like Alkyl Group which May have Substituent:

The chain-like alkyl group as R′²⁰¹ may be linear or branched.

The linear alkyl group has preferably 1 to 20 carbon atoms, more preferably 1 to 15 carbon atoms, and most preferably 1 to 10 carbon atoms.

The branched alkyl group has preferably 3 to 20 carbon atoms, more preferably 3 to 15 carbon atoms, and most preferably 3 to 10 carbon atoms. Specific examples thereof include a 1-methylethyl group, a 1-methylpropyl group, a 2-methylpropyl group, a 1-methylbutyl group, a 2-methylbutyl group, a 3-methylbutyl group, a 1-ethylbutyl group, a 2-ethylbutyl group, a 1-methylpentyl group, a 2-methylpentyl group, a 3-methylpentyl group, and a 4-methylpentyl group.

Chain-Like Alkenyl Group which May have Substituent:

The chain-like alkenyl group as R′²⁰¹ may be linear or branched, and the number of carbon atoms thereof is preferably in a range of 2 to 10, more preferably in a range of 2 to 5, still more preferably in a range of 2 to 4, and particularly preferably 3. Examples of the linear alkenyl group include a vinyl group, a propenyl group (an allyl group), and a butynyl group. Examples of the branched alkenyl group include a 1-methylvinyl group, a 2-methylvinyl group, a 1-methylpropenyl group, and a 2-methylpropenyl group.

Among the examples, as the chain-like alkenyl group, a linear alkenyl group is preferable, a vinyl group or a propenyl group is more preferable, and a vinyl group is particularly preferable.

Examples of the substituent for the chain-like alkyl group or alkenyl group as R′²⁰¹ include an alkoxy group, a halogen atom, a halogenated alkyl group, a hydroxyl group, a carbonyl group, a nitro group, an amino group, and a cyclic group as R′²⁰¹.

Examples of the cyclic group which may have a substituent, the chain-like alkyl group which may have a substituent, and the chain-like alkenyl group which may have a substituent as R′²⁰¹ include those for the acid-dissociable group represented by Formula (a1-r-2) which are the exemplary examples of the cyclic group which may have a substituent and the chain-like alkyl group which may have a substituent, in addition to those described above.

Among the examples, R′²⁰¹ represents preferably a cyclic group which may have a substituent and more preferably a cyclic hydrocarbon group which may have a substituent. More specific preferred examples thereof include a phenyl group, a naphthyl group, a group in which one or more hydrogen atoms have been removed from a polycycloalkane, a lactone-containing cyclic group represented by any of General Formulae (a2-r-1) to (a2-r-7), and a —SO₂-containing cyclic group represented by any of General Formulae (a5-r-1) to (a5-r-4).

In General Formulae (ca-1) to (ca-5), in a case where R²⁰¹ to R²⁰³, R²⁰⁶ and R²⁰⁷, and R²¹¹ and R²¹² are bonded to each other to form a ring with a sulfur atom in the formula, these groups may be bonded to each other via a hetero atom such as a sulfur atom, an oxygen atom, or a nitrogen atom, or a functional group such as a carbonyl group, —SO—, —SO₂—, —SO₃—, —COO—, —CONH— or —N(R_{N})— (here, R_{N} represents an alkyl group having 1 to 5 carbon atoms). As a ring to be formed, a ring containing the sulfur atom in the formula in the ring skeleton thereof is preferably a 3- to 10-membered ring and particularly preferably a 5- to 7-membered ring containing the sulfur atom. Specific examples of the ring to be formed include a thiophene ring, a thiazole ring, a benzothiophene ring, a dibenzothiophene ring, a 9H-thioxanthene ring, a thioxanthone ring, a thianthrene ring, a phenoxathiin ring, a tetrahydrothiophenium ring, and a tetrahydrothiopyranium ring.

R²⁰⁸ and R²⁰⁹ each independently represents a hydrogen atom or an alkyl group having 1 to 5 carbon atoms and preferably a hydrogen atom or an alkyl group having 1 to 3 carbon atoms. In a case where R²⁰⁸ and R²⁰⁹ represent an alkyl group, R²⁰⁸ and R²⁰⁹ may be bonded to each other to form a ring.

R²¹⁰ represents an aryl group which may have a substituent, an alkyl group which may have a substituent, an alkenyl group which may have a substituent, or a SO₂-containing cyclic group which may have a substituent.

Examples of the aryl group as R²¹⁰ include an unsubstituted aryl group having 6 to 20 carbon atoms. Among these, a phenyl group or a naphthyl group is preferable.

As the alkyl group as R²¹⁰, a chain-like or cyclic alkyl group having 1 to 30 carbon atoms is preferable.

It is preferable that the alkenyl group as R²¹⁰ have 2 to 10 carbon atoms.

As the SO₂-containing cyclic group as R²¹⁰ which may have a substituent, “—SO₂-containing polycyclic group” is preferable, and a group represented by General Formula (a5-r-1) is more preferable.

Y²⁰¹'s each independently represents an arylene group, an alkylene group, or an alkenylene group.

Examples of the arylene group as Y²⁰¹ include a group in which one hydrogen atom has been removed from an aryl group described as the aromatic hydrocarbon group as R¹⁰¹ in Formula (b-1-an).

Examples of the alkylene group and alkenylene group as Y²⁰¹ include a group in which one hydrogen atom has been removed from the chain-like alkyl group or the chain-like alkenyl group as R¹⁰¹ in Formula (b-1-an).

In Formula (ca-4), x represents 1 or 2.

W²⁰¹ represents an (x+1)-valent linking group, that is, a divalent or trivalent linking group.

As the divalent linking group represented by W²⁰¹, a divalent hydrocarbon group which may have a substituent is preferable, and examples thereof include the same divalent hydrocarbon groups which may have a substituent as those for Ya²¹ in General Formula (a2-1). The divalent linking group as W²⁰¹ may be any of linear, branched, or cyclic and is preferably cyclic. Among these, a group in which two carbonyl groups are combined with both ends of the arylene group is preferable. Examples of the arylene group include a phenylene group and a naphthylene group. Among these, a phenylene group is particularly preferable.

Examples of the trivalent linking group as W²⁰¹ include a group in which one hydrogen atom has been removed from the above-described divalent linking group as W²⁰¹ and a group obtained by bonding the divalent linking group to another divalent linking group described above. As the trivalent linking group as W²⁰¹, a group obtained by bonding two carbonyl groups to an arylene group is preferable.

Specific examples of suitable cations represented by Formula (ca-1) include cations each represented by Chemical Formulae (ca-1-1) to (ca-1-70).

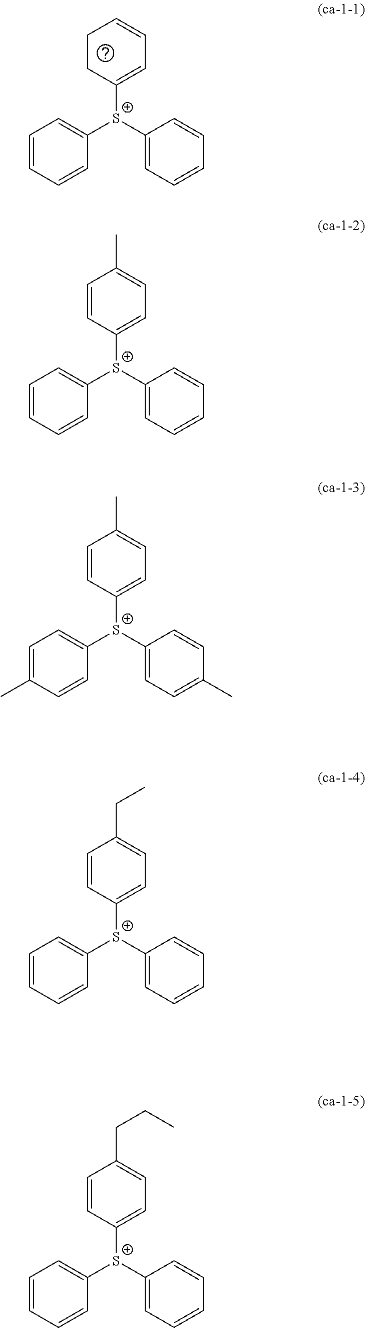

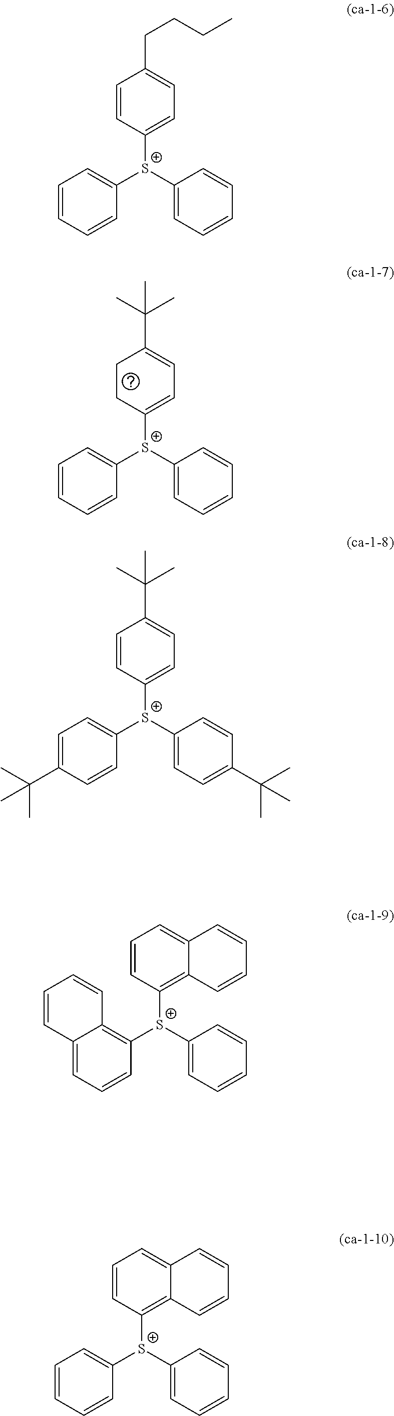 [SMILES: CCCCc1ccc([S+](c2ccccc2)c2ccccc2)cc1 ; CC(C)(C)C1=CC=C([S+](c2ccccc2)c2ccccc2)CC1 ; CC(C)(C)c1ccc([S+](c2ccc(C(C)(C)C)cc2)c2ccc(C(C)(C)C)cc2)cc1 ; c1ccc([S+](c2cccc3ccccc23)c2cccc3ccccc23)cc1 ; c1ccc([S+](c2ccccc2)c2cccc3ccccc23)cc1]

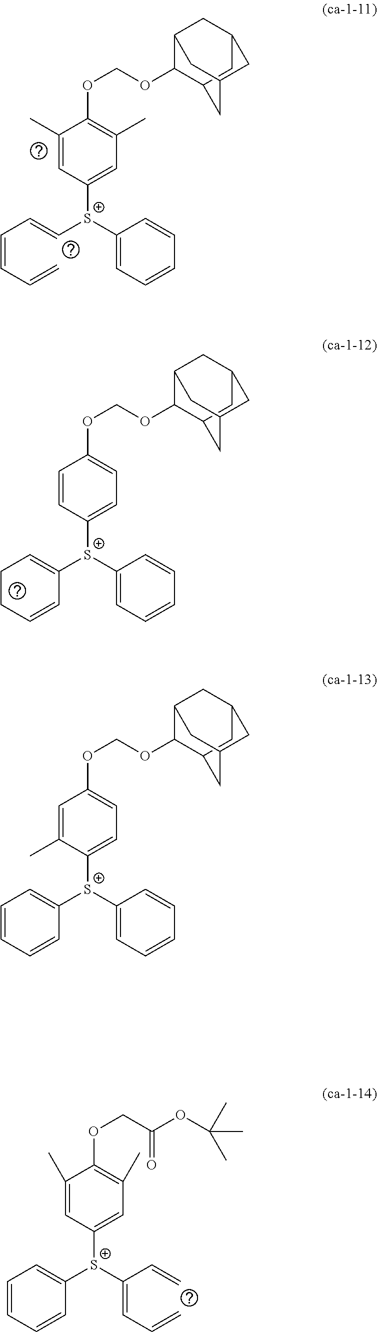 [SMILES: C=C/C=C\C=C\[S+](c1ccccc1)c1cc(C)c(OCOC2C3CC4CC(C3)CC2C4)c(C)c1 ; C1=CC([S+](c2ccccc2)c2ccc(OCOC3C4CC5CC(C4)CC3C5)cc2)=CCC1 ; Cc1cc(OCOC2C3CC4CC(C3)CC2C4)ccc1[S+](c1ccccc1)c1ccccc1 ; C=C/C=C(\C=C)[S+](c1ccccc1)c1cc(C)c(OCC(=O)OC(C)(C)C)c(C)c1]

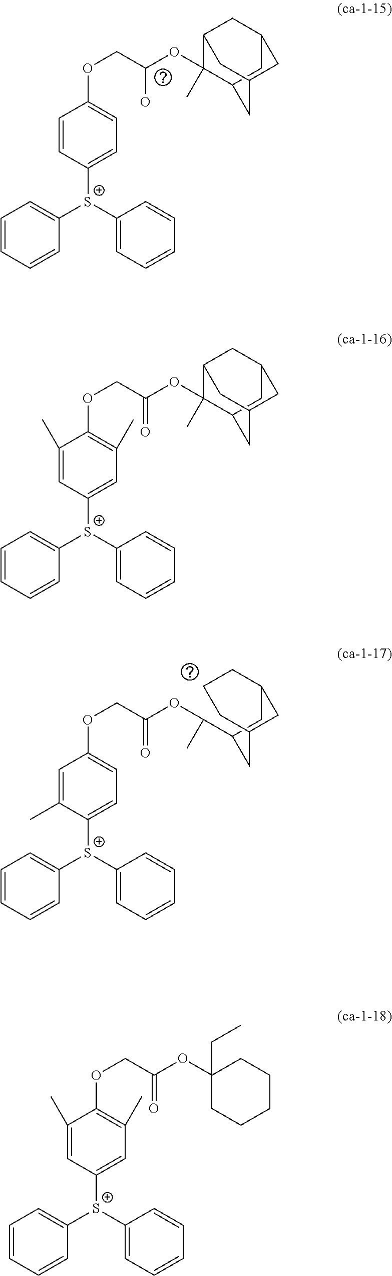 [SMILES: CC1(OC(O)COc2ccc([S+](c3ccccc3)c3ccccc3)cc2)C2CC3CC(C2)CC1C3 ; Cc1cc([S+](c2ccccc2)c2ccccc2)cc(C)c1OCC(=O)OC1(C)C2CC3CC(C2)CC1C3 ; Cc1cc(OCC(=O)OC(C)C2CC3CCCC(C3)C2)ccc1[S+](c1ccccc1)c1ccccc1 ; CCC1(OC(=O)COc2c(C)cc([S+](c3ccccc3)c3ccccc3)cc2C)CCCCC1]

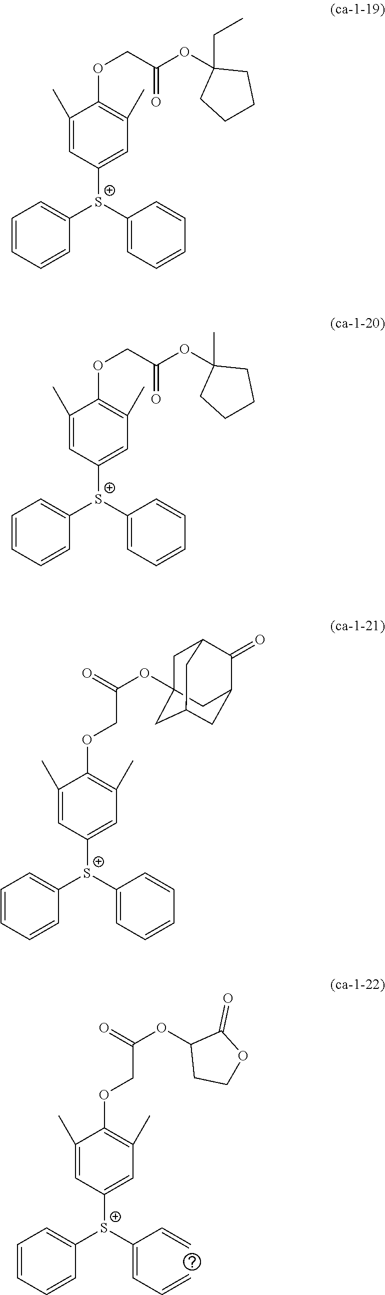 [SMILES: CCC1(OC(=O)COc2c(C)cc([S+](c3ccccc3)c3ccccc3)cc2C)CCCC1 ; Cc1cc([S+](c2ccccc2)c2ccccc2)cc(C)c1OCC(=O)OC1(C)CCCC1 ; Cc1cc([S+](c2ccccc2)c2ccccc2)cc(C)c1OCC(=O)OC12CC3CC(C1)C(=O)C(C3)C2 ; C=C/C=C(\C=C)[S+](c1ccccc1)c1cc(C)c(OCC(=O)OC2CCOC2=O)c(C)c1]

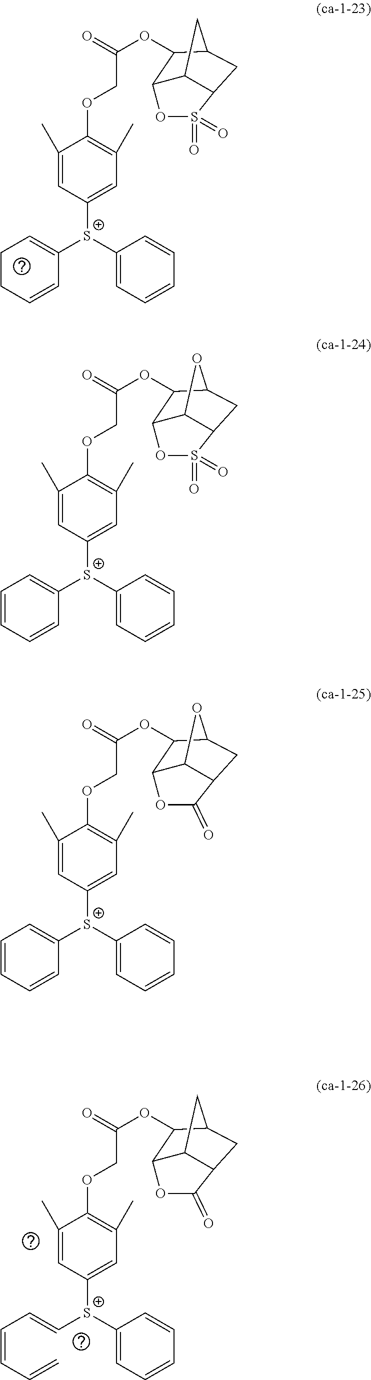 [SMILES: Cc1cc([S+](C2=CCCC=C2)c2ccccc2)cc(C)c1OCC(=O)OC1C2CC3C1OS(=O)(=O)C3C2 ; Cc1cc([S+](c2ccccc2)c2ccccc2)cc(C)c1OCC(=O)OC1C2CC3C(O2)C1OS3(=O)=O ; Cc1cc([S+](c2ccccc2)c2ccccc2)cc(C)c1OCC(=O)OC1C2CC3C(=O)OC1C3O2 ; C=C/C=C\C=C\[S+](c1ccccc1)c1cc(C)c(OCC(=O)OC2C3CC4C(=O)OC2C4C3)c(C)c1]

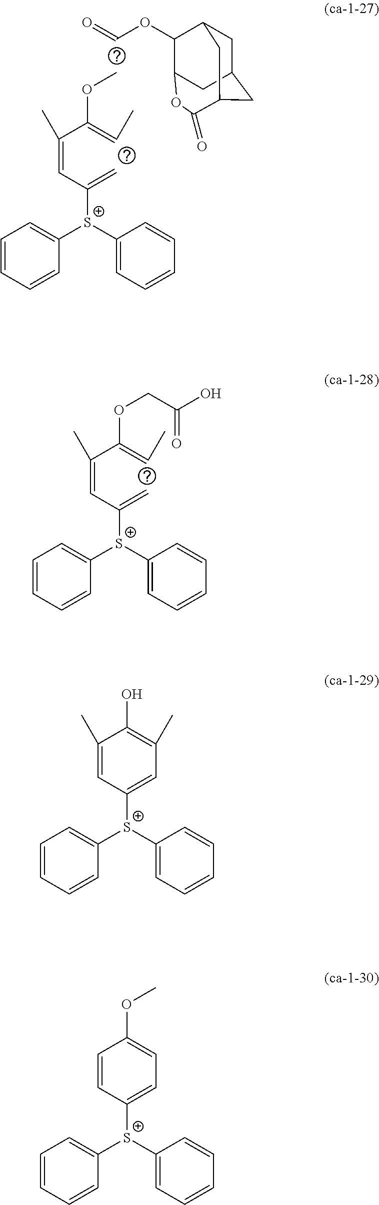 [SMILES: C=C(/C=C(C)\C(=C\C)OC)[S+](c1ccccc1)c1ccccc1 ; O=COC1C2CC3CC(C2)C(=O)OC1C3 ; C=C(/C=C(C)\C(=C\C)OCC(=O)O)[S+](c1ccccc1)c1ccccc1 ; Cc1cc([S+](c2ccccc2)c2ccccc2)cc(C)c1O ; COc1ccc([S+](c2ccccc2)c2ccccc2)cc1]

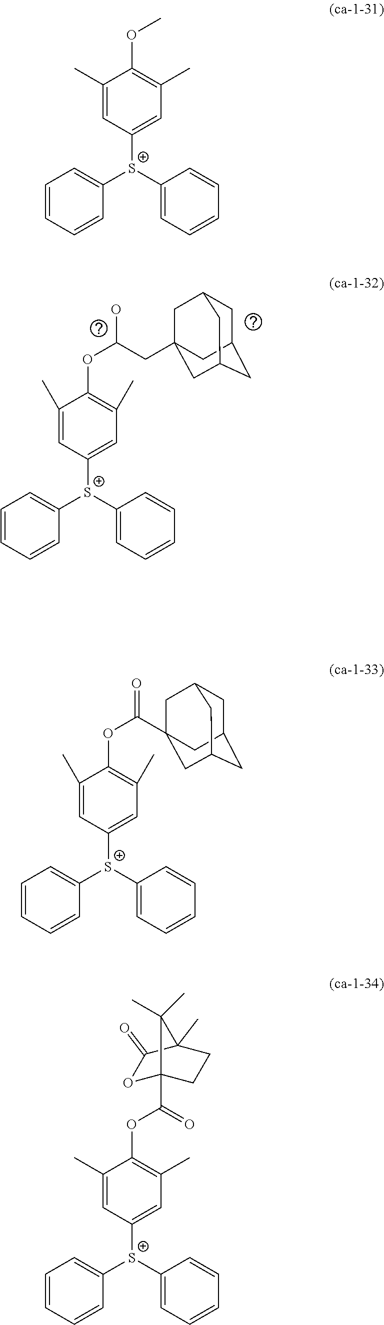 [SMILES: COc1c(C)cc([S+](c2ccccc2)c2ccccc2)cc1C ; Cc1cc([S+](c2ccccc2)c2ccccc2)cc(C)c1OC(O)CC12CC3CC(CC(C3)C1)C2 ; Cc1cc([S+](c2ccccc2)c2ccccc2)cc(C)c1OC(=O)C12CC3CC(CC(C3)C1)C2 ; Cc1cc([S+](c2ccccc2)c2ccccc2)cc(C)c1OC(=O)C12CCC(C)(C(=O)O1)C2(C)C]

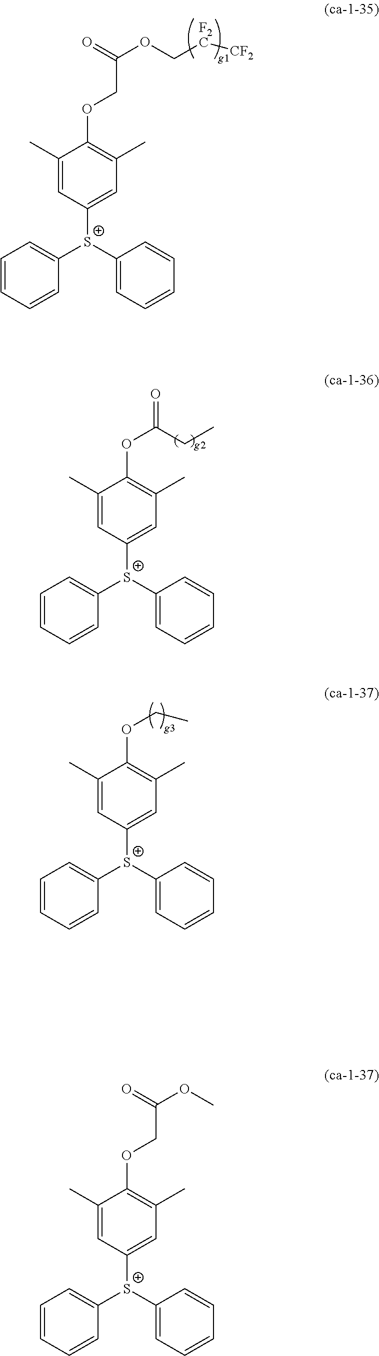

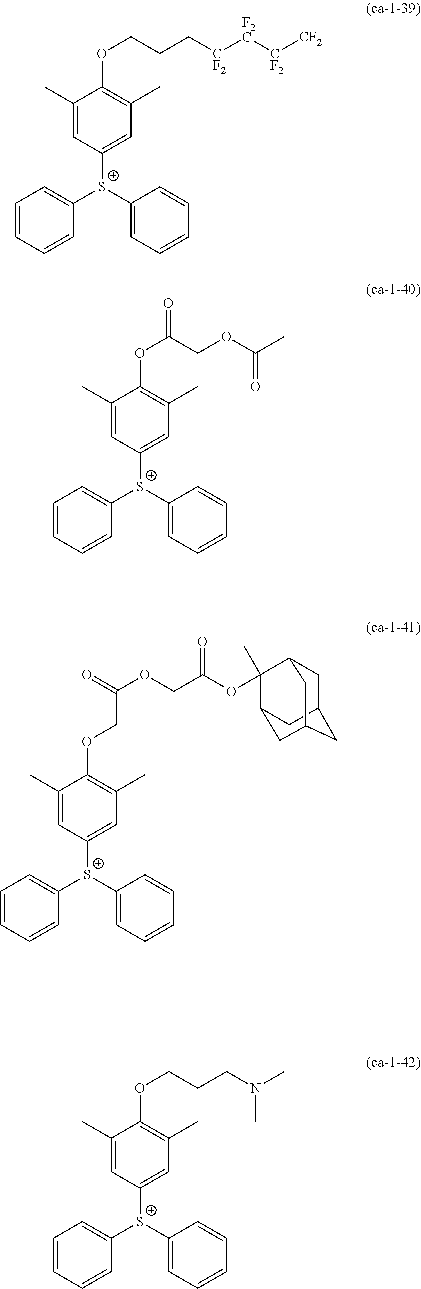

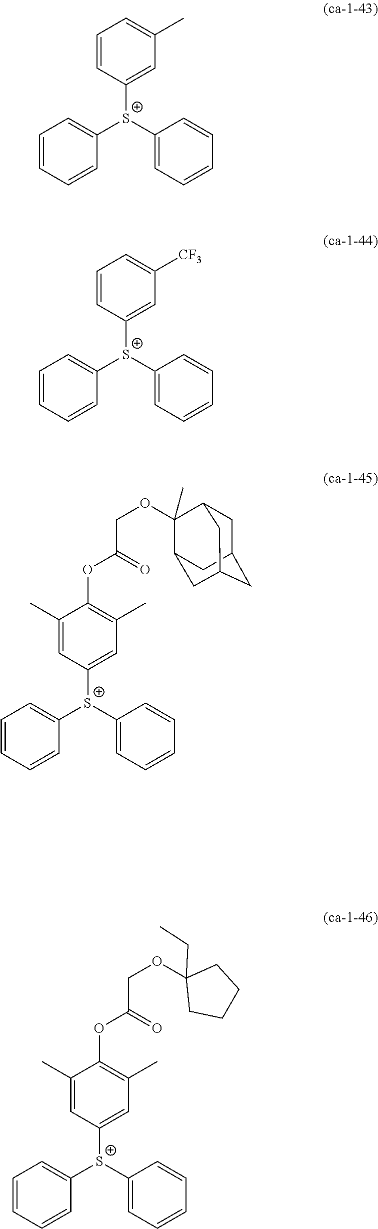 [SMILES: Cc1cccc([S+](c2ccccc2)c2ccccc2)c1 ; FC(F)(F)c1cccc([S+](c2ccccc2)c2ccccc2)c1 ; Cc1cc([S+](c2ccccc2)c2ccccc2)cc(C)c1OC(=O)COC1(C)C2CC3CC(C2)CC1C3 ; CCC1(OCC(=O)Oc2c(C)cc([S+](c3ccccc3)c3ccccc3)cc2C)CCCC1]

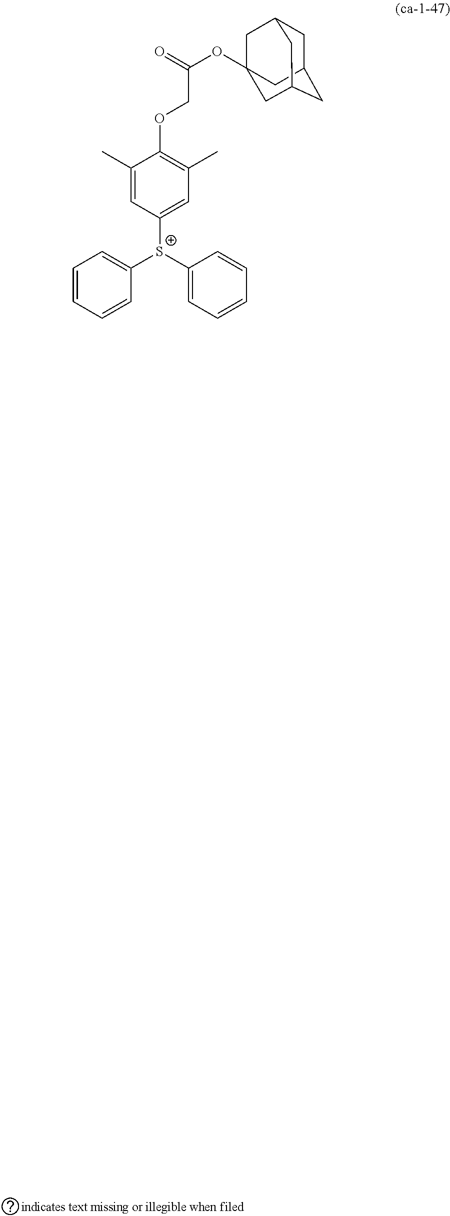 [SMILES: Cc1cc([S+](c2ccccc2)c2ccccc2)cc(C)c1OCC(=O)OC12CC3CC(CC(C3)C1)C2]

[In the formulae, g1, g2, and g3 represent a repeating number, g1 represents an integer of 1 to 5, g2 represents an integer of 0 to 20, and g3 represents an integer of 0 to 20.]

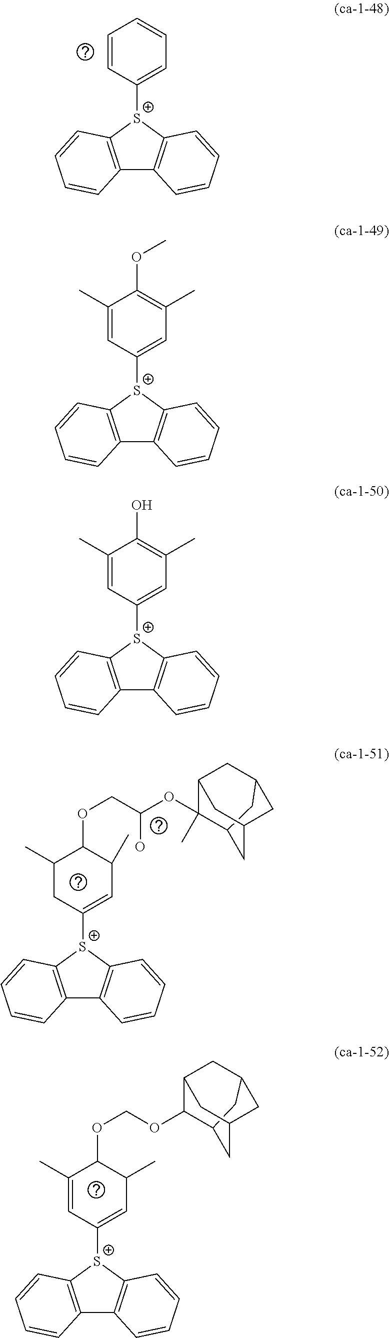 [SMILES: c1ccc(-[s+]2c3ccccc3c3ccccc32)cc1 ; COc1c(C)cc(-[s+]2c3ccccc3c3ccccc32)cc1C ; Cc1cc(-[s+]2c3ccccc3c3ccccc32)cc(C)c1O ; CC1C=C([s+]2c3ccccc3c3ccccc32)CC(C)C1OCC(O)OC1(C)C2CC3CC(C2)CC1C3 ; CC1=CC([s+]2c3ccccc3c3ccccc32)=CC(C)C1OCOC1C2CC3CC(C2)CC1C3]

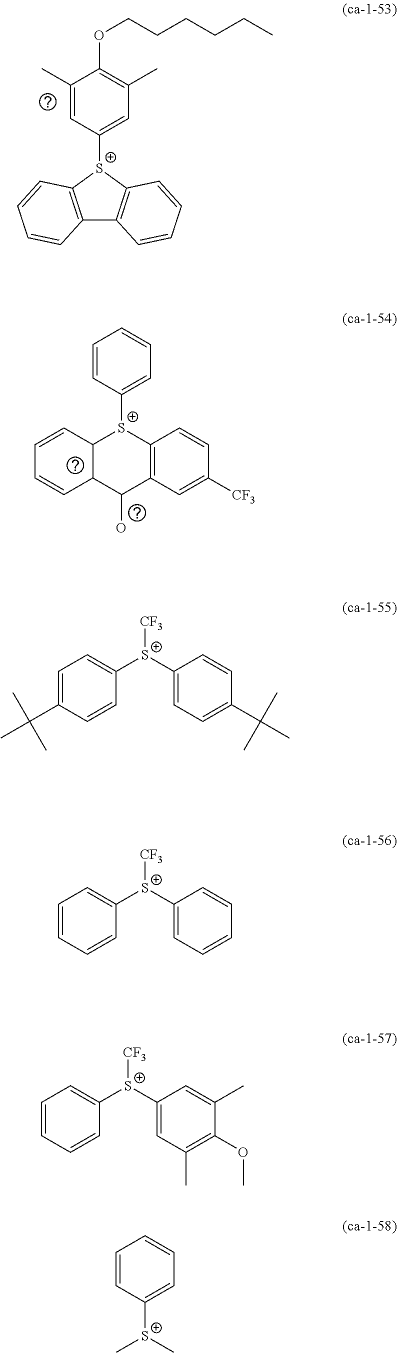

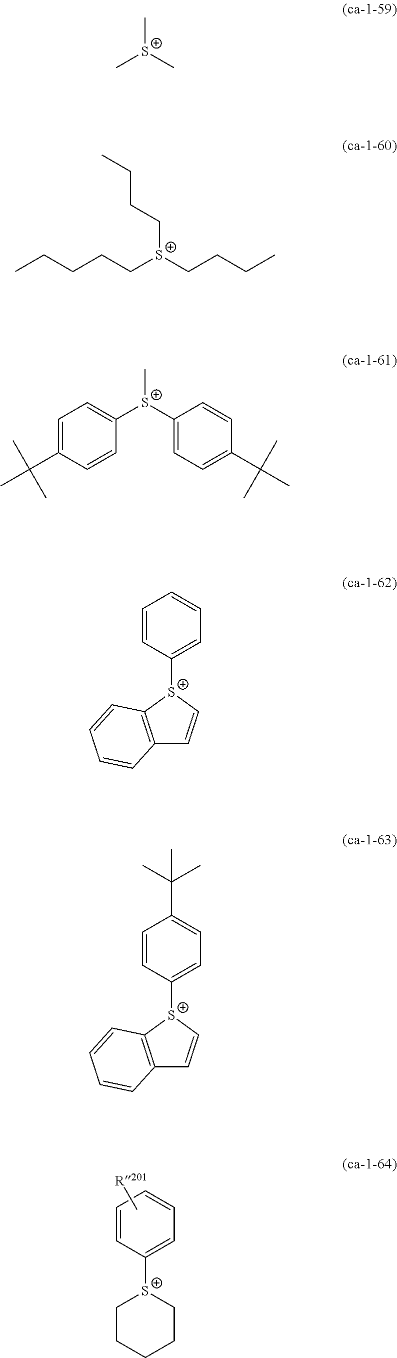

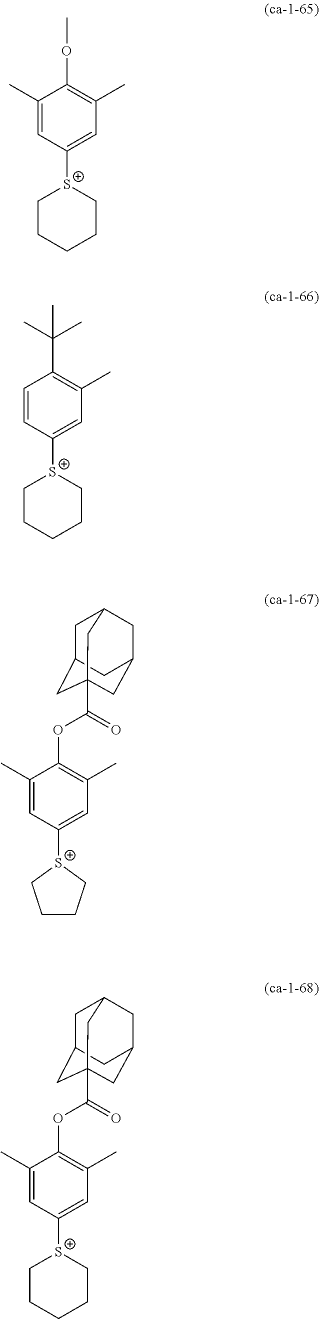

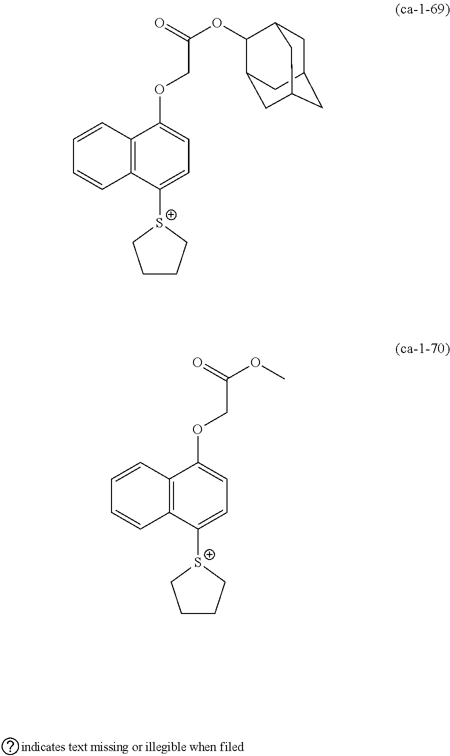 [SMILES: O=C(COc1ccc([S+]2CCCC2)c2ccccc12)OC1C2CC3CC(C2)CC1C3 ; COC(=O)COc1ccc([S+]2CCCC2)c2ccccc12]

[In the formulae, R″²⁰¹ represents a hydrogen atom or a substituent, and examples of the substituent include the same groups as those for the substituents that R²⁰¹ to R²⁰⁷ and R²¹⁰ to R²¹² may have.]

Specific examples of suitable cations represented by Formula (ca-2) include a diphenyliodonium cation and a bis(4-tert-butylphenyl)iodonium cation.

Specific examples of suitable cations represented by Formula (ca-3) include cations each represented by Formulae (ca-3-1) to (ca-3-6).

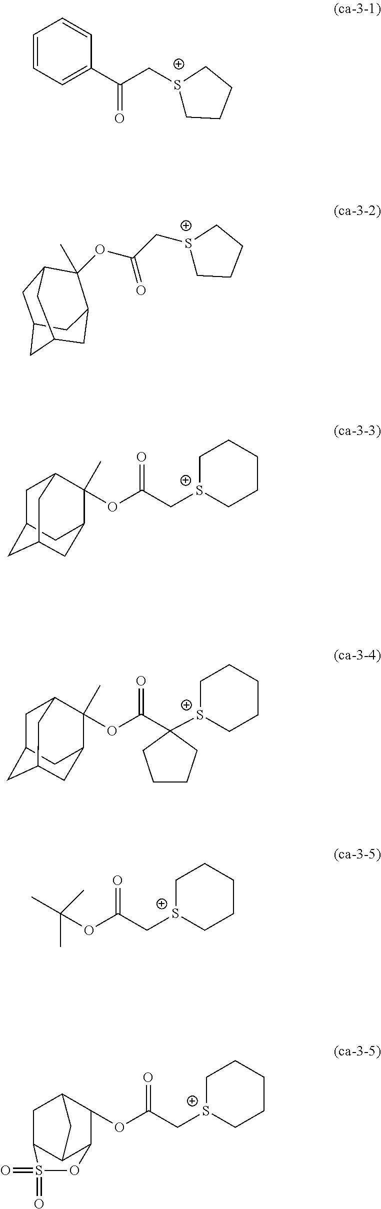 [SMILES: O=C(C[S+]1CCCC1)c1ccccc1 ; CC1(OC(=O)C[S+]2CCCC2)C2CC3CC(C2)CC1C3 ; CC1(OC(=O)C[S+]2CCCCC2)C2CC3CC(C2)CC1C3 ; CC1(OC(=O)C2([S+]3CCCCC3)CCCC2)C2CC3CC(C2)CC1C3 ; CC(C)(C)OC(=O)C[S+]1CCCCC1 ; O=C(C[S+]1CCCCC1)OC1C2CC3C1OS(=O)(=O)C3C2]

Specific examples of suitable cations represented by Formula (ca-4) include cations each represented by Formulae (ca-4-1) and (ca-4-2).

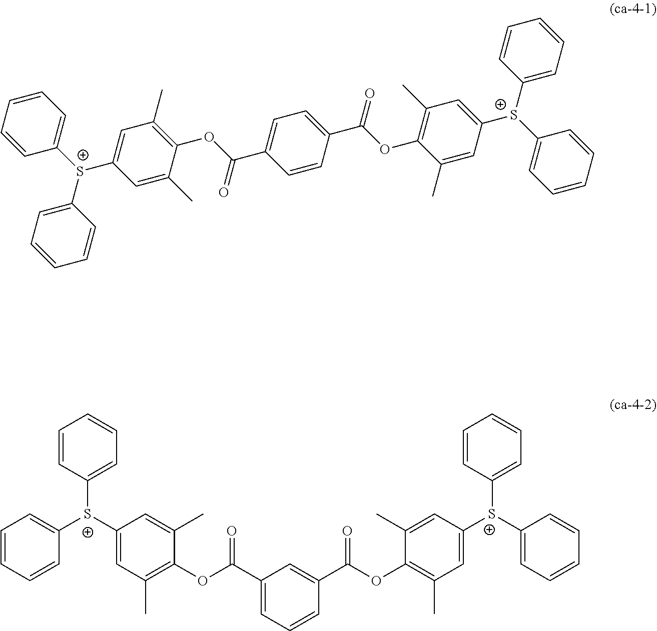 [SMILES: Cc1cc([S+](c2ccccc2)c2ccccc2)cc(C)c1OC(=O)c1ccc(C(=O)Oc2c(C)cc([S+](c3ccccc3)c3ccccc3)cc2C)cc1 ; Cc1cc([S+](c2ccccc2)c2ccccc2)cc(C)c1OC(=O)c1cccc(C(=O)Oc2c(C)cc([S+](c3ccccc3)c3ccccc3)cc2C)c1]

Specific examples of suitable cations represented by Formula (ca-5) include cations each represented by General Formulae (ca-5-1) and (ca-5-3).

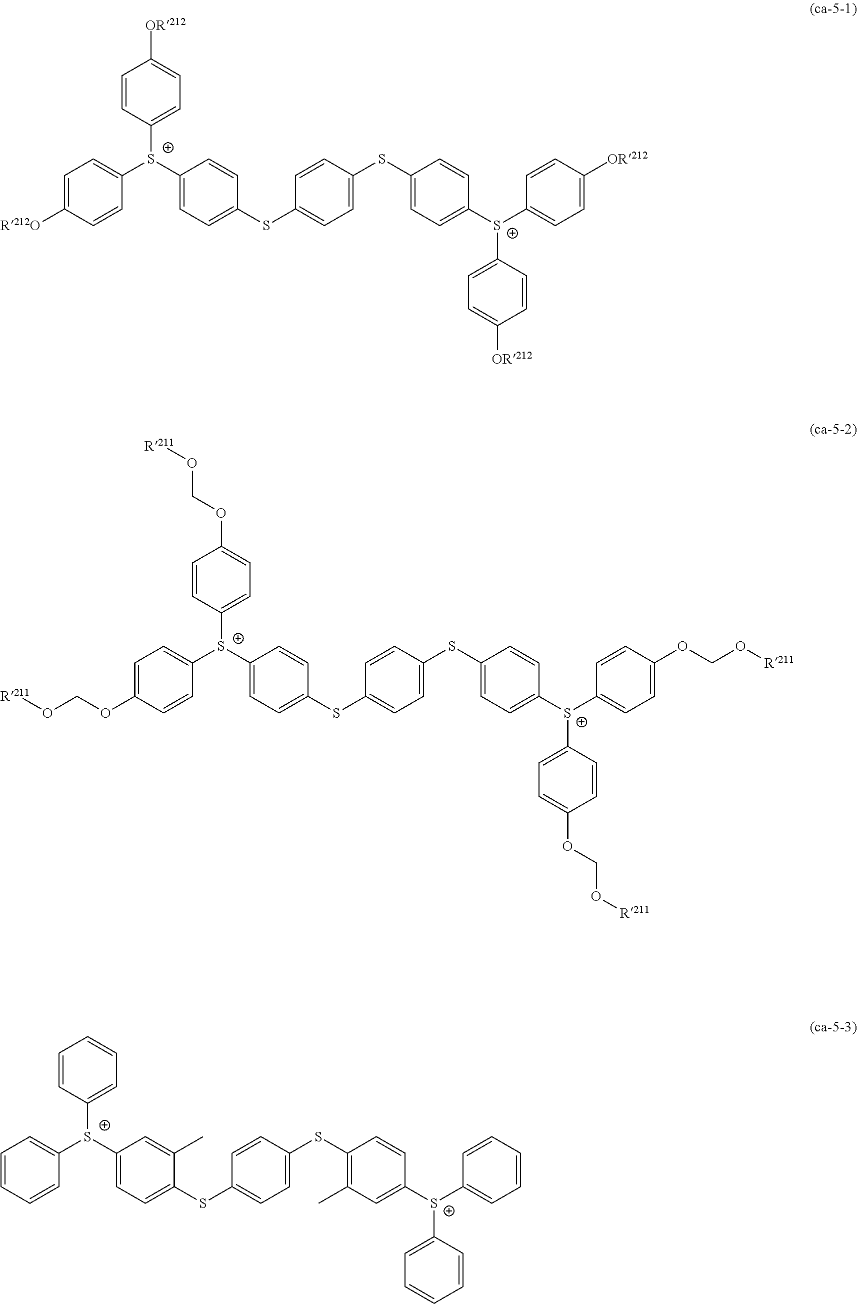

Among the examples, a cation represented by General Formula (ca-1) is preferable as the cation moiety ((M^{m+})_{1/m}).

In the resist composition according to the present embodiment, the component (B1) may be used alone or in combination of two or more kinds thereof.

In a case where the resist composition contains the component (B1), the content of the component (B1) in the resist composition is preferably less than 40 parts by mass, more preferably in a range of 1 to 30 parts by mass, and still more preferably in a range of 1 to 20 parts by mass with respect to 100 parts by mass of the component (A).

In a case where the content of the component (B1) is set to be in the above-described preferable ranges, defects are likely to be reduced. Further, each component of the resist composition is dissolved in an organic solvent from the viewpoint that a uniform solution is easily obtained and the storage stability of the resist composition is enhanced.

It is preferable that the resist composition according to the present embodiment not contain the component (B1).

### <Other Components>

The resist composition according to the present embodiment may further contain other components in addition to the component (A) and the component (B) described above. Examples of other components include a component (D), a component (E), a component (F), and a component (S) described below.

### <<Acid Diffusion Control Agent Component (D)>>

The resist composition according to the present embodiment may further contain, in addition to the component (A), an acid diffusion control agent component (component (D)) that traps an acid generated upon light exposure (that is, controls the diffusion of an acid). The component (D) acts as a quencher (an acid diffusion control agent) which traps the acid generated in the resist composition upon light exposure.

Examples of the component (D) include a photo-decomposable base (D1) having an acid diffusion controllability (hereinafter, referred to as “component (D1)”) which is lost by the decomposition upon light exposure and a nitrogen-containing organic compound (D2) (hereinafter, referred to as “component (D2)”) which does not correspond to the component (D1). Among these, the photo-decomposable base (component (D1)) is preferable from the viewpoint of easily increasing the sensitivity, reducing the roughness, and improving the characteristic of suppressing the occurrence of coating defects.

### Regarding Component (D1)

In a case where a resist composition containing the component (D1) is obtained, the contrast between an exposed portion and an unexposed portion of the resist film can be further improved in a case of forming a resist pattern.

The component (D1) is not particularly limited as long as the component is decomposed upon light exposure and loses an acid diffusion controllability, and one or more compounds selected from the group consisting of a compound represented by General Formula (d1-1) (hereinafter, referred to as “component (d1-1)”), a compound represented by General Formula (d1-2) (hereinafter, referred to as “component (d1-2)”), and a compound represented by General Formula (d1-3) (hereinafter, referred to as “component (d1-3)”) are preferable.

Since the components (d1-1) to (d1-3) are decomposed and lose their acid diffusion controllability (basicity), the components (d1-1) to (d1-3) do not function as a quencher at the exposed portion of the resist film, but function as a quencher at the unexposed portion of the resist film.

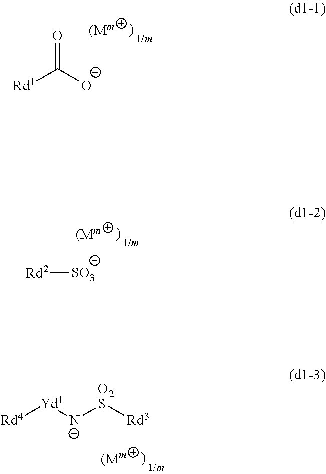

[In the formulae, Rd¹ to Rd⁴ represent a cyclic group which may have a substituent, a chain-like alkyl group which may have a substituent, or a chain-like alkenyl group which may have a substituent. Here, no fluorine atom is bonded to the carbon atom adjacent to the S atom in Rd² of Formula (d1-2). Yd¹ represents a single bond or a divalent linking group. m represents an integer of 1 or greater, and M^{m+}'s each independently represents an m-valent organic cation.]

{Component (d1-1)}

### Anion Moiety

In Formula (d1-1), Rd¹ represents a cyclic group which may have a substituent, a chain-like alkyl group which may have a substituent, or a chain-like alkenyl group which may have a substituent, and examples thereof include the same groups as those for R′²⁰¹.

Among these, it is preferable that Rd¹ represent an aromatic hydrocarbon group which may have a substituent, an aliphatic cyclic group which may have a substituent, or a chain-like alkyl group which may have a substituent. Examples of the substituent that may be included in these groups include a hydroxyl group, an oxo group, an alkyl group, an aryl group, a fluorine atom, a fluorinated alkyl group, a lactone-containing cyclic group represented by any of General Formulae (a2-r-1) to (a2-r-7), an ether bond, an ester bond, and a combination thereof. In a case where an ether bond or an ester bond is included as the substituent, the substituent may be bonded through an alkylene group, and a linking group represented by any of Formulae (y-al-1) to (y-al-5) is preferable as the substituent. Further, in a case where the aromatic hydrocarbon group, the aliphatic cyclic group, or the chain-like alkyl group as Rd¹ contain a linking group represented by any of General Formulae (y-al-1) to (y-al-7) as a substituent, V′¹⁰¹ in General Formulae (y-al-1) to (y-al-7) is bonded to the carbon atom constituting the aromatic hydrocarbon group, the aliphatic cyclic group, or the chain-like alkyl group as Rd¹ in Formula (d3-1), in General Formulae (y-al-1) to (y-al-7).

Suitable examples of the aromatic hydrocarbon group include a phenyl group, a naphthyl group, and a polycyclic structure having a bicyclooctane skeleton (for example, a polycyclic structure formed of a bicyclooctane skeleton and a ring structure other than the bicyclooctane skeleton).

As the aliphatic cyclic group, a group in which one or more hydrogen atoms have been removed from a polycycloalkane such as adamantane, norbornane, isobornane, tricyclodecane or tetracyclododecane is more preferable.

It is preferable that the chain-like alkyl group have 1 to 10 carbon atoms, and specific examples thereof include a linear alkyl group such as a methyl group, an ethyl group, a propyl group, a butyl group, a pentyl group, a hexyl group, a heptyl group, an octyl group, a nonyl group, or a decyl group; and a branched alkyl group such as a 1-methylethyl group, a 1-methylpropyl group, a 2-methylpropyl group, a 1-methylbutyl group, a 2-methylbutyl group, a 3-methylbutyl group, a 1-ethylbutyl group, a 2-ethylbutyl group, a 1-methylpentyl group, a 2-methylpentyl group, a 3-methylpentyl group, or a 4-methylpentyl group.

In a case where the chain-like alkyl group is a fluorinated alkyl group having a fluorine atom or a fluorinated alkyl group as a substituent, the fluorinated alkyl group has preferably 1 to 11 carbon atoms, more preferably 1 to 8 carbon atoms, and still more preferably 1 to 4 carbon atoms. The fluorinated alkyl group may have an atom other than a fluorine atom. Examples of the atom other than a fluorine atom include an oxygen atom, a sulfur atom, and a nitrogen atom.

Specific preferred examples of the anion moiety in the component (d1-1) are described below.

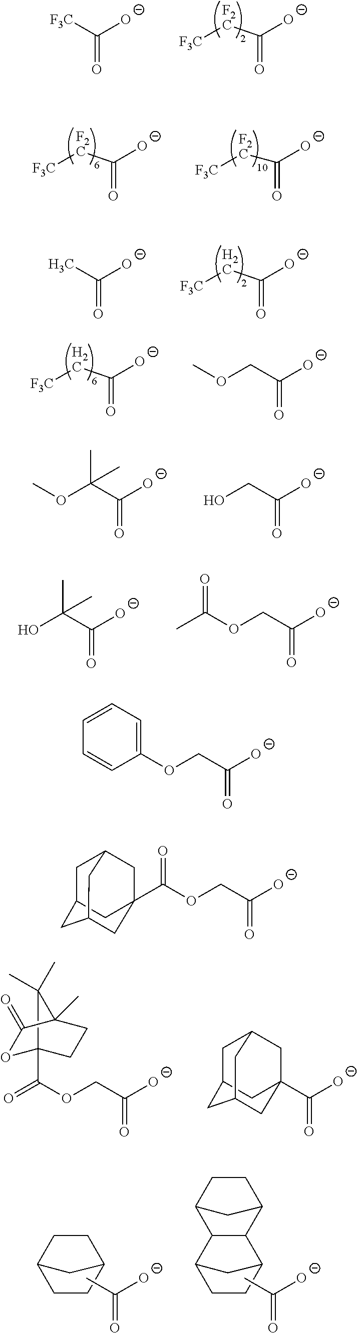

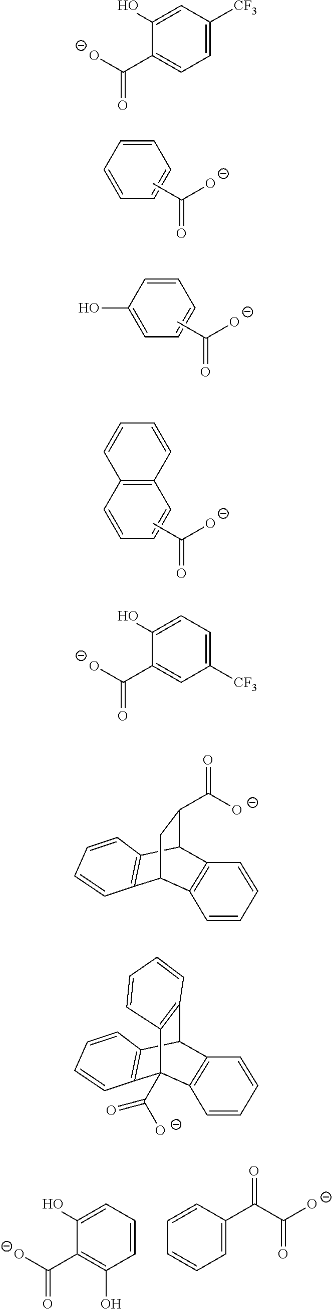

### Cation Moiety

In Formula (d1-1), M^{m+} represents an m-valent organic cation.

Suitable examples of the organic cation as M^{m+} include the same as those each represented by General Formulae (ca-1) to (ca-5). Among these, a cation represented by General Formula (ca-1) is more preferable, and cations each represented by Formulae (ca-1-1) to (ca-1-70) are still more preferable.

The component (d1-1) may be used alone or in combination of two or more kinds thereof.

{Component (d1-2)}

### Anion Moiety

In Formula (d1-2), Rd² represents a cyclic group which may have a substituent, a chain-like alkyl group which may have a substituent, or a chain-like alkenyl group which may have a substituent, and examples thereof include the same groups as those for R′²⁰¹.

Here, no fluorine atom is bonded to the carbon atom adjacent to the S atom in Rd² (the carbon atom is not substituted with fluorine). In this manner, the anion of the component (d1-2) is an appropriately weak acid anion, thereby improving the quenching ability of the component (D).

It is preferable that Rd² represent a chain-like alkyl group which may have a substituent or an aliphatic cyclic group which may have a substituent. The chain-like alkyl group has preferably 1 to 10 carbon atoms and more preferably 3 to 10 carbon atoms. As the aliphatic cyclic group, a group in which one or more hydrogen atoms have been removed from adamantane, norbornane, isobornane, tricyclodecane, or tetracyclododecane (a group which may have a substituent); and a group in which one or more hydrogen atoms have been removed from camphor are more preferable.

The hydrocarbon group as Rd² may have a substituent, and examples of the substituent include the same groups as those for the substituent that the hydrocarbon group (such as an aromatic hydrocarbon group, an aliphatic cyclic group, or a chain-like alkyl group) as Rd¹ in Formula (d1-1) may have.

Specific preferred examples of the anion moiety in the component (d1-2) are described below.

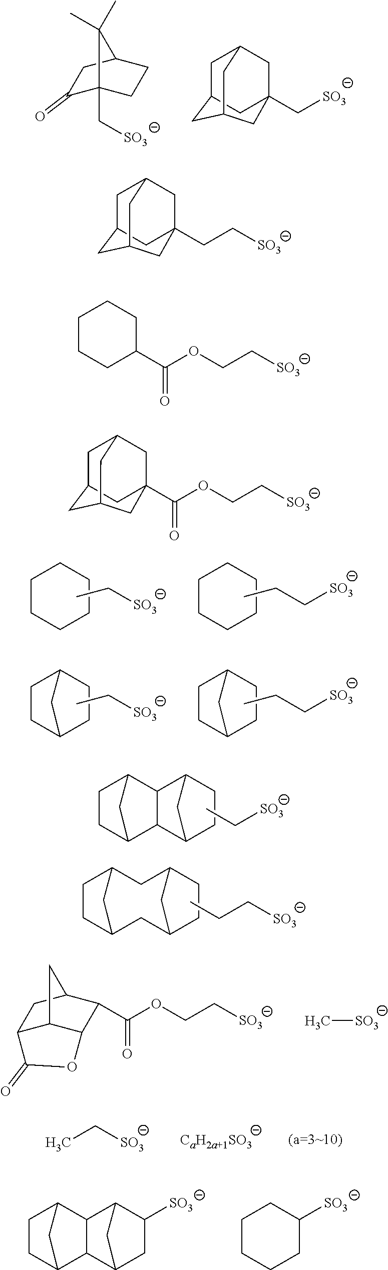

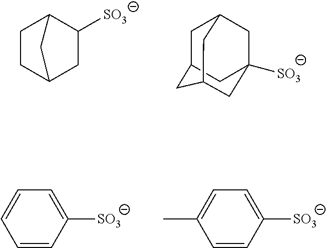

### Cation Moiety

In Formula (d1-2), M^{m+} represents an m-valent organic cation and is the same as that for M^{m+} in Formula (d1-1).

The component (d1-2) may be used alone or in combination of two or more kinds thereof.

{Component (d1-3)}

### Anion Moiety

In Formula (d1-3), Rd³ represents a cyclic group which may have a substituent, a chain-like alkyl group which may have a substituent, or a chain-like alkenyl group which may have a substituent, and examples thereof include the same groups as those for R′²⁰¹. Among these, a cyclic group having a fluorine atom, a chain-like alkyl group, or a chain-like alkenyl group is preferable. Among these, a fluorinated alkyl group is preferable, and the same groups as those for the fluorinated alkyl group represented by Rd¹ are more preferable.

In Formula (d1-3), Rd⁴ represents a cyclic group which may have a substituent, a chain-like alkyl group which may have a substituent, or a chain-like alkenyl group which may have a substituent, and examples thereof include the same groups as those for R′²⁰¹.

Among these, an alkyl group which may have a substituent, an alkoxy group which may have a substituent, an alkenyl group which may have a substituent, or a cyclic group which may have a substituent is preferable.

It is preferable that the alkyl group as Rd⁴ be a linear or branched alkyl group having 1 to 5 carbon atoms, and specific examples thereof include a methyl group, an ethyl group, a propyl group, an isopropyl group, an n-butyl group, an isobutyl group, a tert-butyl group, a pentyl group, an isopentyl group, and a neopentyl group. Some hydrogen atoms in the alkyl group as Rd⁴ may be substituted with a hydroxyl group, a cyano group, or the like.

It is preferable that the alkoxy group as Rd⁴ be an alkoxy group having 1 to 5 carbon atoms, and specific examples of the alkoxy group having 1 to 5 carbon atoms include a methoxy group, an ethoxy group, an n-propoxy group, an iso-propoxy group, an n-butoxy group, and a tert-butoxy group. Among these, a methoxy group and an ethoxy group are preferable.

Examples of the alkenyl group as Rd⁴ include the same groups as those for the alkenyl group as R′²⁰¹. Among these, a vinyl group, a propenyl group (an allyl group), a 1-methylpropenyl group, and a 2-methylpropenyl group are preferable. These groups may have an alkyl group having 1 to 5 carbon atoms or a halogenated alkyl group having 1 to 5 carbon atoms as a substituent.

Examples of the cyclic group as Rd⁴ include the same groups as those for the cyclic group as R′²⁰¹. Among these, an alicyclic group in which one or more hydrogen atoms have been removed from a cycloalkane such as cyclopentane, cyclohexane, adamantane, norbornane, isobornane, tricyclodecane or tetracyclododecane or an aromatic group such as a phenyl group or a naphthyl group is preferable. In a case where Rd⁴ represents an alicyclic group, the resist composition is satisfactorily dissolved in an organic solvent so that the lithography characteristics are enhanced. Further, in a case where Rd⁴ represents an aromatic group, the resist composition has excellent light absorption efficiency in lithography using EUV or the like as an exposure light source, and thus the sensitivity and lithography characteristics are enhanced.

In Formula (d1-3), Yd¹ represents a single bond or a divalent linking group. The divalent linking group as Yd¹ is not particularly limited, and examples thereof include a divalent hydrocarbon group (an aliphatic hydrocarbon group or an aromatic hydrocarbon group) which may have a substituent and a divalent linking group having a hetero atom. Examples of the divalent linking groups are the same as those for the divalent hydrocarbon group which may have a substituent and the divalent linking group having a hetero atom as described above for the divalent linking group as Ya²¹ in Formula (a2-1).

It is preferable that Yd¹ represent a carbonyl group, an ester bond, an amide bond, an alkylene group, or a combination thereof. As the alkylene group, a linear or branched alkylene group is more preferable, and a methylene group or an ethylene group is still more preferable.

Specific preferred examples of the anion moiety in the component (d1-3) are described below.

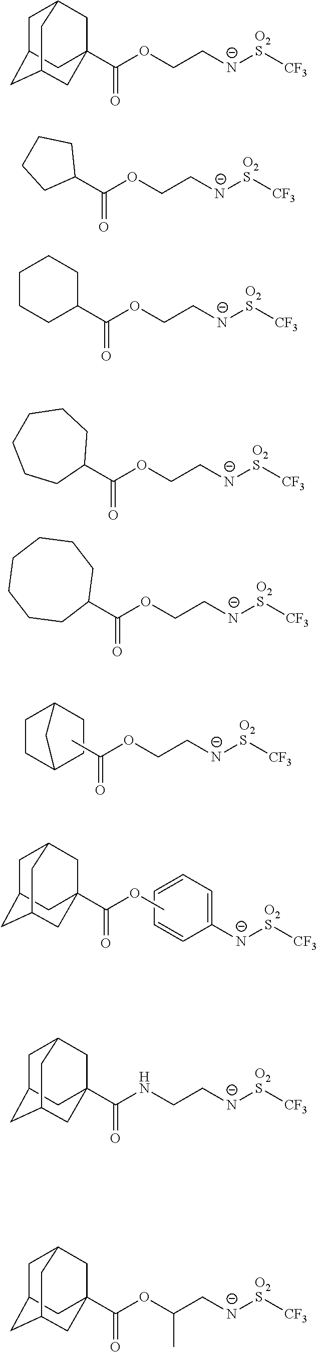

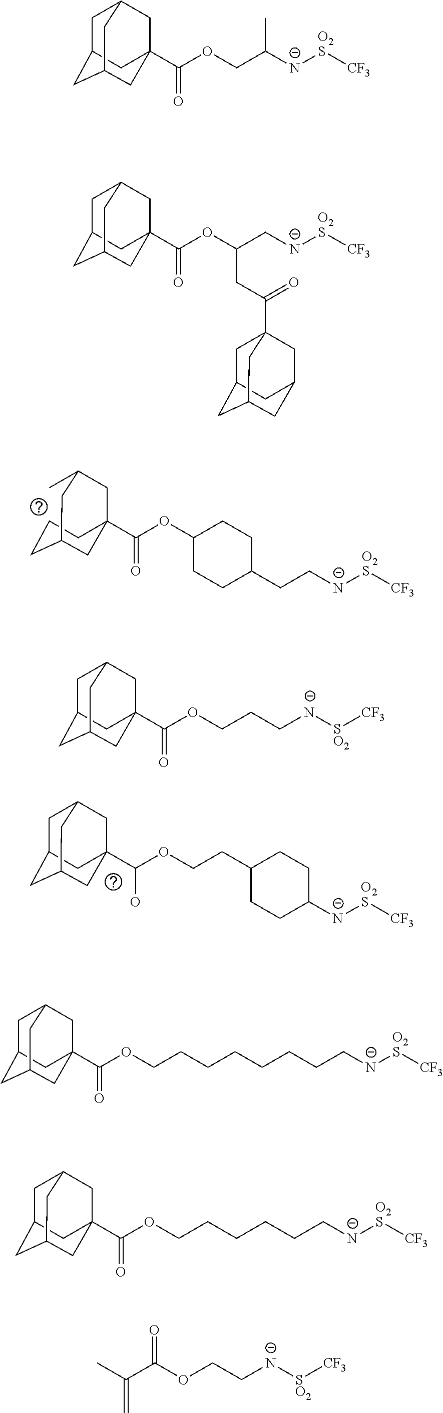

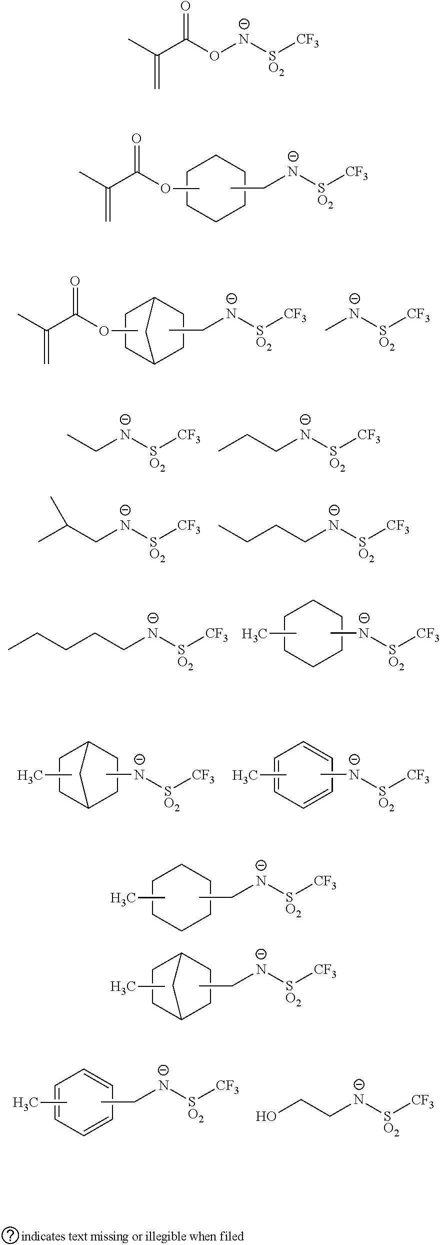

### Cation Moiety

In Formula (d1-3), M^{m+} represents an m-valent organic cation and is the same as that for M^{m+} in Formula (d1-1).

The component (d1-3) may be used alone or in combination of two or more kinds thereof.

As the component (D1), only one of the above-described components (d1-1) to (d1-3) or a combination of two or more kinds thereof may be used.

In a case where the resist composition contains the component (D1), the content of the component (D1) in the resist composition is preferably in a range of 0.5 to 20 parts by mass, more preferably in a range of 1 to 15 parts by mass, and still more preferably in a range of 2 to 8 parts by mass with respect to 100 parts by mass of the component (A1).

In a case where the content of the component (D1) is greater than or equal to the lower limits of the above-described ranges, particularly excellent lithography characteristics and an excellent resist pattern shape are easily obtained. On the contrary, in a case where the content is less than or equal to the upper limits of the above-described ranges, the sensitivity can be satisfactorily maintained and the throughput is also excellent.

### Method of Producing Component (D1):

The methods of producing the component (d1-1) and the component (d1-2) are not particularly limited, and these components can be produced by known methods.

Further, the method of producing the component (d1-3) is not particularly limited, and the component is produced by the same method as disclosed in United States Patent Application, Publication No. 2012-0149916.

### Regarding Component (D2)

The component (D) may contain a nitrogen-containing organic compound component (hereinafter, referred to as “component (D2)”) that does not correspond to the component (D1) described above.

The component (D2) is not particularly limited as long as the component functions as an acid diffusion control agent and does not correspond to the component (D1), and any component may be selected from known components and then used. Among the examples, an aliphatic amine is preferable, and particularly a secondary aliphatic amine and a tertiary aliphatic amine are more preferable.

The aliphatic amine is an amine containing one or more aliphatic groups, and the number of carbon atoms in the aliphatic group is preferably in a range of 1 to 12.

Examples of the aliphatic amine include amines in which at least one hydrogen atom of ammonia NH₃ has been substituted with an alkyl group or hydroxyalkyl group having 12 or less carbon atoms (alkylamines or alkylalcoholamines), and cyclic amines.

Specific examples of the alkylamines and the alkylalcoholamines include monoalkylamines such as n-hexylamine, n-heptylamine, n-octylamine, n-nonylamine, and n-decylamine; dialkylamines such as diethylamine, di-n-propylamine, di-n-heptylamine, di-n-octylamine, and dicyclohexylamine; trialkylamines such as trimethylamine, triethylamine, tri-n-propylamine, tri-n-butylamine, tri-n-pentylamine, tri-n-hexylamine, tri-n-heptylamine, tri-n-octylamine, tri-n-nonylamine, tri-n-decylamine, and tri-n-dodecylamine; and alkylalcoholamines such as diethanolamine, triethanolamine, diisopropanolamine, triisopropanolamine, di-n-octanolamine, and tri-n-octanolamine. Among these, a trialkylamine having 5 to 10 carbon atoms is preferable, and tri-n-pentylamine and tri-n-octylamine are particularly preferable.

Examples of the cyclic amine include a heterocyclic compound having a nitrogen atom as a hetero atom. The heterocyclic compound may be a monocyclic compound (aliphatic monocyclic amine) or a polycyclic compound (aliphatic polycyclic amine).

Specific examples of the aliphatic monocyclic amine include piperidine and piperazine.

The aliphatic polycyclic amine preferably has 6 to 10 carbon atoms, and specific examples thereof include 1, 5-diazabicyclo[4.3.0]-5-nonene, 1,8-diazabicyclo[5.4.0]-7-undecene, hexamethylenetetramine, and 1,4-diazabicyclo[2.2.2]octane.

Examples of other aliphatic amines include tris(2-methoxymethoxyethyl)amine, tris{2-(2-methoxyethoxy)ethyl}amine, tris{2-(2-methoxyethoxymethoxy)ethyl}amine, tris{2-(1-methoxyethoxy)ethyl}amine, tris{2-(1-ethoxyethoxy)ethyl}amine, tris{2-(1-ethoxypropoxy)ethyl}amine, tris[2-{2-(2-hydroxyethoxy)ethoxy}ethyl]amine, and triethanolamine triacetate. Among these, triethanolamine triacetate is preferable.

As the component (D2), an aromatic amine may be used.

Examples of aromatic amines include 4-dimethylaminopyridine, pyrrole, indole, pyrazole, imidazole, and derivatives thereof, tribenzylamine, 2,6-diisopropylaniline, N-tert-butoxycarbonylpyrrolidine, and 2,6-di-tert-butylpyridine.

The component (D2) may be used alone or in combination of two or more kinds thereof.

In a case where the resist composition contains the component (D2), the content of the component (D2) in the resist composition is typically in a range of 0.01 to 5 parts by mass with respect to 100 parts by mass of the component (A1). In a case where the content thereof is set to be in the above-described range, the resist pattern shape, the post exposure temporal stability, and the like are improved.

<<At Least One Compound (E) Selected from Group Consisting of Organic Carboxylic Acids, Phosphorus Oxo Acids, and Derivatives Thereof>>

For the purpose of preventing any deterioration in sensitivity and improving the resist pattern shape and the post-exposure temporal stability, the resist composition according to the present embodiment may contain, as an optional component, at least one compound (E) (hereinafter referred to as “component (E)”) selected from the group consisting of an organic carboxylic acid, and a phosphorus oxo acid and a derivative thereof.

Specific examples of the organic carboxylic acid include acetic acid, malonic acid, citric acid, malic acid, succinic acid, benzoic acid, and salicylic acid. Among these, salicylic acid is preferable.

Examples of the phosphorus oxo acid include phosphoric acid, phosphonic acid, and phosphinic acid. Among these, phosphonic acid is particularly preferable.

Examples of the phosphorus oxo acid derivative include an ester obtained by substituting a hydrogen atom in the above-described oxo acid with a hydrocarbon group. Examples of the hydrocarbon group include an alkyl group having 1 to 5 carbon atoms and an aryl group having 6 to 15 carbon atoms.

Examples of the phosphoric acid derivatives include phosphoric acid esters such as phosphoric acid di-n-butyl ester and phosphoric acid diphenyl ester.

Examples of the phosphonic acid derivatives include phosphonic acid esters such as phosphonic acid dimethyl ester, phosphonic acid di-n-butyl ester, phenylphosphonic acid, phosphonic acid diphenyl ester, and phosphonic acid dibenzyl ester.

Examples of the phosphinic acid derivatives include phosphinic acid ester and phenylphosphinic acid.

In the resist composition of the present embodiment, the component (E) may be used alone or in combination of two or more kinds thereof.

In a case where the resist composition contains the component (E), the content of the component (E) is preferably in a range of 0.01 to 5 parts by mass and more preferably in a range of 0.05 to 3 parts by mass with respect to 100 parts by mass of the component (A). In a case where the content thereof is in the above-described range, the sensitivity, lithography characteristics, and the like are improved.

### <<Fluorine Additive Component (F)>>

The resist composition according to the present embodiment may further contain a fluorine additive component (hereinafter, referred to as “component (F)”) as a hydrophobic resin. The component (F) is used to impart water repellency to the resist film and used as a resin different from the component (A), whereby the lithography characteristics can be improved.

As the component (F), for example, the fluorine-containing high-molecular-weight compounds described in Japanese Unexamined Patent Application, First Publication Nos. 2010-002870, 2010-032994, 2010-277043, 2011-13569, and 2011-128226 can be used.

Specific examples of the component (F) include a polymer having a constitutional unit (f1) represented by General Formula (f1-1). As the polymer, a polymer (homopolymer) formed of only the constitutional unit (f1) represented by Formula (f1-1); a copolymer of the constitutional unit (f1) and the constitutional unit (a1); or a copolymer of the constitutional unit (f1), a constitutional unit derived from acrylic acid or methacrylic acid, and the constitutional unit (a1) is preferable, and a copolymer of the constitutional unit (f1) and the constitutional unit (a1) is more preferable. Here, as the constitutional unit (a1) copolymerized with the constitutional unit (f1), a constitutional unit derived from 1-ethyl-1-cyclooctyl (meth)acrylate or a constitutional unit derived from 1-methyl-1-adamantyl (meth)acrylate is preferable, and a constitutional unit derived from 1-ethyl-1-cyclooctyl (meth)acrylate is more preferable.

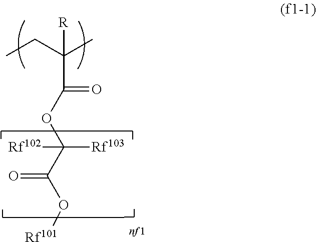

[In the formula, R is the same as described above, Rf¹⁰² and Rf¹⁰³ each independently represents a hydrogen atom, a halogen atom, an alkyl group having 1 to 5 carbon atoms, or a halogenated alkyl group having 1 to 5 carbon atoms, and Rf¹⁰² and Rf¹⁰³ may be the same as or different from each other. nf¹ represents an integer of 0 to 5, and Rf¹⁰¹ represents an organic group having a fluorine atom.]

In Formula (f1-1), R bonded to the carbon atom at the α-position is the same as described above. It is preferable that R represent a hydrogen atom or a methyl group. In Formula (f1-1), a fluorine atom is preferable as the halogen atom as Rf¹⁰² and Rf¹⁰³. Examples of the alkyl group having 1 to 5 carbon atoms as Rf¹⁰² and Rf¹⁰³ include the same groups as those for the alkyl group having 1 to 5 carbon atoms as R. Among the examples, a methyl group or an ethyl group is preferable. Specific examples of the halogenated alkyl group having 1 to 5 carbon atoms as Rf¹⁰² and Rf¹⁰³ include groups in which a part or all of the hydrogen atoms of an alkyl group having 1 to 5 carbon atoms have been substituted with halogen atoms. Among these, a fluorine atom is preferable as the halogen atom. Among these, Rf¹⁰² and Rf¹⁰³ represent preferably a hydrogen atom, a fluorine atom, or an alkyl group having 1 to 5 carbon atoms, more preferably a hydrogen atom, a fluorine atom, a methyl group, or an ethyl group, and still more preferably a hydrogen atom.

In Formula (f1-1), nf¹ represents an integer of 0 to 5, preferably an integer of 0 to 3, and more preferably 1 or 2.

In Formula (f1-1), Rf¹⁰¹ represents an organic group having a fluorine atom and preferably a hydrocarbon group having a fluorine atom.

The hydrocarbon group having a fluorine atom may be linear, branched, or cyclic, and the number of carbon atoms thereof is preferably in a range of 1 to 20, more preferably in a range of 1 to 15, and particularly preferably in a range of 1 to 10.

In the hydrocarbon group having a fluorine atom, preferably 25% or more of the hydrogen atoms in the hydrocarbon group are fluorinated, more preferably 50% or more thereof are fluorinated, and particularly preferably 60% or more thereof are fluorinated from the viewpoint of increasing the hydrophobicity of the resist film during immersion exposure.

Among the examples, Rf¹⁰¹ represents more preferably a fluorinated hydrocarbon group having 1 to 6 carbon atoms and particularly preferably a trifluoromethyl group, —CH₂—CF₃, —CH₂—CF₂—CF₃, —CH(CF₃)₂, —CH₂—CH₂—CF₃, or —CH₂—CH₂—CF₂—CF₂—CF₂—CF₃.

The weight-average molecular weight (Mw) (in terms of polystyrene according to gel permeation chromatography) of the component (F) is preferably in a range of 1,000 to 50,000, more preferably in a range of 5,000 to 40,000, and most preferably in a range of 10,000 to 30,000. In a case where the weight-average molecular weight thereof is less than or equal to the upper limits of the above-described ranges, the resist composition exhibits sufficient solubility in a solvent for a resist enough to be used as a resist. Meanwhile, in a case where the weight-average molecular weight thereof is greater than or equal to the lower limits of the above-described ranges, water repellency of the resist film is improved.

Further, the dispersity (Mw/Mn) of the component (F) is preferably in a range of 1.0 to 5.0, more preferably in a range of 1.0 to 3.0, and most preferably in a range of 1.0 to 2.5.

In the resist composition according to the present embodiment, the component (F) may be used alone or in combination of two or more kinds thereof.

In a case where the resist composition contains the component (F), the content of the component (F) is preferably in a range of 0.5 to 10 parts by mass and more preferably in a range of 1 to 10 parts by mass with respect to 100 parts by mass of the component (A).

### <<Organic Solvent Component (S)>>

The resist composition of the present embodiment can be produced by dissolving the resist materials in an organic solvent component (hereinafter, referred to as “component (S)”).

The component (S) may be any organic solvent which can dissolve each component to be used to obtain a uniform solution, and any organic solvent can be appropriately selected from those which have been known as solvents of a chemically amplified resist composition and then used.

Examples of the component (S) include lactones such as γ-butyrolactone; ketones such as acetone, methyl ethyl ketone, cyclohexanone, methyl-n-pentyl ketone, methyl isopentyl ketone, and 2-heptanone; polyhydric alcohols such as ethylene glycol, diethylene glycol, propylene glycol, and dipropylene glycol; compounds having an ester bond, such as ethylene glycol monoacetate, diethylene glycol monoacetate, propylene glycol monoacetate, and dipropylene glycol monoacetate; polyhydric alcohol derivatives of compounds having an ether bond such as monoalkyl ether or monophenyl ether, such as monomethylether, monoethylether, monopropylether, or monobutylether of polyhydric alcohols or compounds having an ester bond [among these, propylene glycol monomethyl ether acetate (PGMEA) and propylene glycol monomethyl ether (PGME) are preferable]; cyclic ethers such as dioxane; esters such as methyl lactate, ethyl lactate (EL), methyl acetate, ethyl acetate, butyl acetate, methyl pyruvate, ethyl pyruvate, methyl methoxypropionate, and ethyl ethoxypropionate; aromatic organic solvents such as anisole, ethylbenzylether, cresylmethylether, diphenylether, dibenzylether, phenetole, butylphenylether, ethylbenzene, diethylbenzene, pentylbenzene, isopropylbenzene, toluene, xylene, cymene, and mesitylene; and dimethylsulfoxide (DMSO).

In the resist composition of the present embodiment, the component (S) may be used alone or in the form of a mixed solvent of two or more kinds thereof. Among these, PGMEA, PGME, γ-butyrolactone, EL, or cyclohexanone is preferable.

Further, a mixed solvent obtained by mixing PGMEA with a polar solvent is also preferable as the component (S). The blending ratio (mass ratio) of the mixed solvent can be appropriately determined in consideration of the compatibility between PGMEA and the polar solvent, but is preferably in the range of 1:9 to 9:1 and more preferably in a range of 2:8 to 8:2.

More specifically, in a case where EL or cyclohexanone is blended as the polar solvent, the mass ratio of PGMEA to EL or cyclohexanone is preferably in a range of 1:9 to 9:1 and more preferably in a range of 2:8 to 8:2. Further, in a case where PGME is blended as the polar solvent, the mass ratio of PGMEA to PGME is preferably in a range of 1:9 to 9:1, more preferably in a range of 2:8 to 8:2, and still more preferably in a range of 3:7 to 7:3. Further, a mixed solvent of PGMEA, PGME, and cyclohexanone is also preferable.

Further, a mixed solvent of γ-butyrolactone and at least one selected from PGMEA and EL is also preferable as the component (S). In this case, as the mixing ratio, the mass ratio between the former and the latter is preferably in a range of 70:30 to 95:5.

The amount of the component (S) to be used is not particularly limited and is appropriately set to have a concentration which enables coating a substrate or the like depending on the thickness of the coated film. The component (S) is typically used in an amount such that the solid content concentration of the resist composition is set to be in a range of 0.1% to 20% by mass and preferably in a range of 0.2% to 15% by mass.

As desired, miscible additives such as additive resins, dissolution inhibitors, plasticizers, stabilizers, colorants, halation prevention agents, and dyes for improving the performance of the resist film can be added to the resist composition of the present embodiment, as appropriate.

After the resist material is dissolved in the component (S), impurities may be removed from the resist composition of the present embodiment using a porous polyimide film, a porous polyamideimide film, or the like. For example, the resist composition may be filtered using a filter formed of a porous polyimide film, a filter formed of a porous polyamideimide film, a filter formed of a porous polyimide film and a porous polyamideimide film, or the like. Examples of the porous polyimide film and the porous polyamideimide film include those described in Japanese Unexamined Patent Application, First Publication No. 2016-155121.

The resist composition according to the present embodiment described above contains the compound (B0) containing the cation (C0) as the component (B).

The cation (C0) is a Low LUMO cation having a planar structure. In a case where the compound (B0) contains the cation (C0), the acid generation efficiency upon light exposure is improved. In this manner, the sensitivity of the resist composition containing the compound (B0) is improved.

Further, the solubility of the cation (C0) in the component (S) is improved by allowing the phenyl group to contain two or more electron-withdrawing groups. Further, the solubility in a developing solution is improved in the development after light exposure. In this manner, the resist composition containing the compound (B0) can reduce defects.

As described above, according to the resist composition of the present embodiment, it is possible to achieve both high sensitivity and reduction of defects.

### Second Embodiment

The resist composition according to a second embodiment contains the compound (C) as the component (D). The resist composition according to the second embodiment contains the component (A) whose solubility in a developing solution is changed due to the action of the acid and an acid diffusion control agent component (D) that controls diffusion of an acid generated upon light exposure. The acid diffusion control agent component (D) contains a compound represented by General Formula (d0).

M^{d⊕}Xd^{⊖} (d0)

[In the formula, M^{d+} represents the cation (C0). Xd⁻ represents a counter anion.]

### <Component (A)>

As the component (A), the same component as the component (A) in the first embodiment can be used. It is preferable that the component (A) contain a resin component (A1) whose solubility in a developing solution is changed due to the action of an acid. As the component (A1), the same component as the component (A1) in the first embodiment can be used. The component (A1) may be used alone or in combination of two or more kinds thereof.

In the resist composition of the present embodiment, the content of the component (A) may be adjusted according to the thickness of the resist film intended to be formed.

### <(D) Component>

The resist composition according to the present embodiment contains an acid diffusion control agent component (D) that traps an acid generated upon light exposure (that is, controls the diffusion of an acid). The component (D) contains a compound (DO) (hereinafter, also referred to as “component (D0)”) represented by General Formula (d0). The compound (D0) is a photo-decomposable base that is decomposed upon light exposure and loses acid diffusion controllability.

### <<Component (D0)>>

The component (D0) is a compound represented by General Formula (d0).

M^{d⊕}Xd^{⊖} (d0)

[In the formula, M^{d+} represents the cation (C0). Xd⁻ represents a counter anion.]

### {Cation Moiety}

In General Formula (d0), M^{d+} represents the cation (C0).

### {Anion Moiety}

In General Formula (d0), Xd⁻ is a counter anion, and Xd⁻ is not particularly limited, and those which have been suggested as an anion moiety of a photo-decomposable base for a chemically amplified resist composition can be used.

Examples of Xd⁻ include an anion represented by General Formula (d-1-an), General Formula (d-2-an), or General Formula (d-3-an).

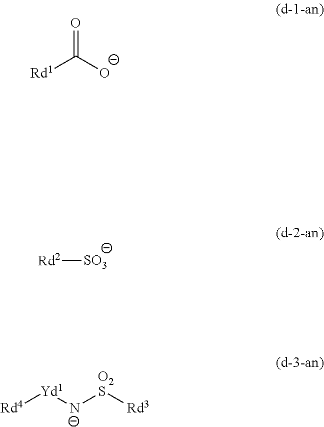

[In the formulae, Rd¹ to Rd⁴ represent a cyclic group which may have a substituent, a chain-like alkyl group which may have a substituent, or a chain-like alkenyl group which may have a substituent. Here, no fluorine atom is bonded to the carbon atom adjacent to the S atom in Rd² of Formula (d1-2). Yd¹ represents a single bond or a divalent linking group. m represents an integer of 1 or greater, and M^{m+}'s each independently represents an m-valent organic cation.]

Anion Represented by Formula (d-1-an)

The anion represented by Formula (d-1-an) is the same as the anion moiety of the component (d1-1).

Anion Represented by Formula (d-2-an)

The anion represented by Formula (d-2-an) is the same as the anion moiety of the component (d1-2).

Anion Represented by Formula (d-3-an)

The anion represented by Formula (d-3-an) is the same as the anion moiety of the component (d1-3).

Among the examples, it is preferable that Xd⁻ represent an anion represented by Formula (d-1-an).

Specific examples of the component (D0) are shown below, but the present invention is not limited thereto.

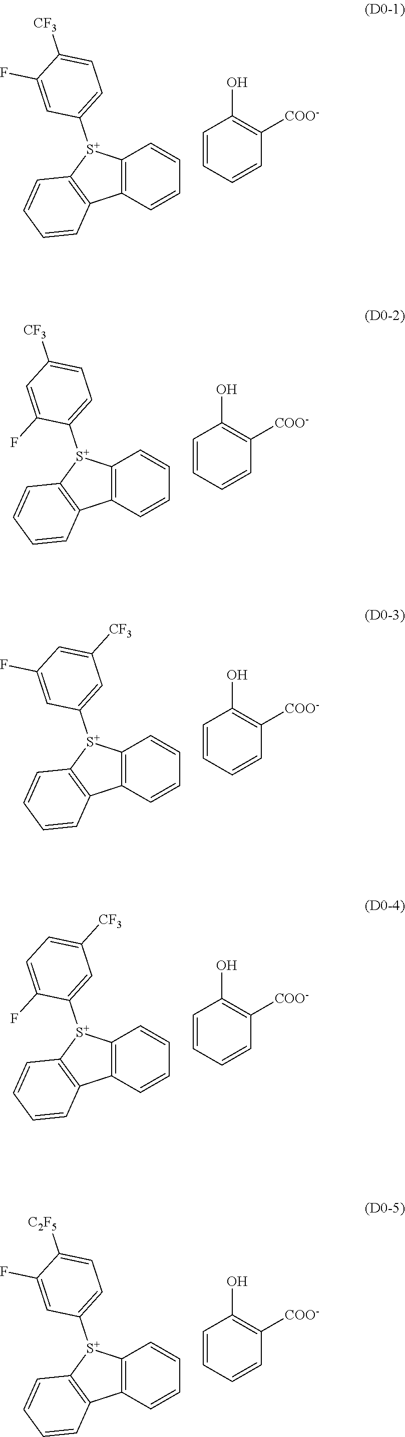

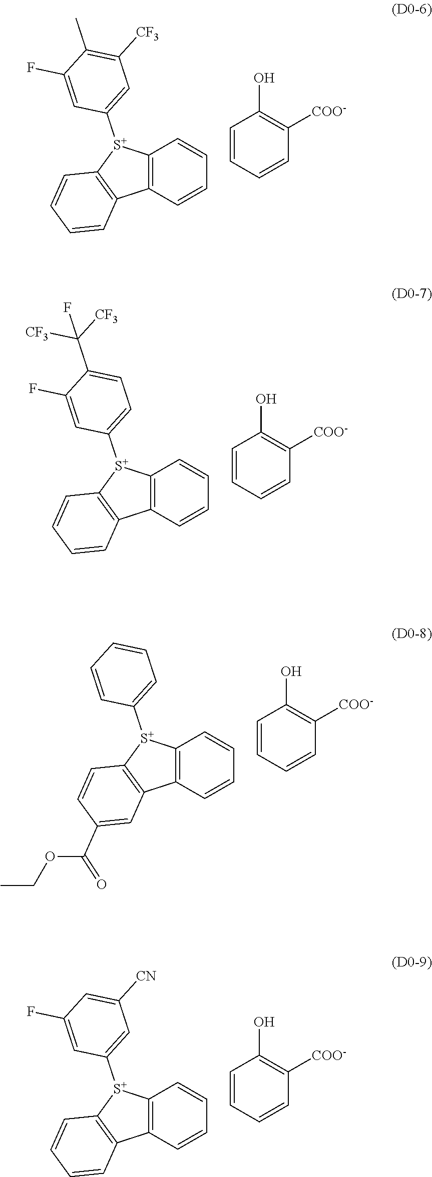

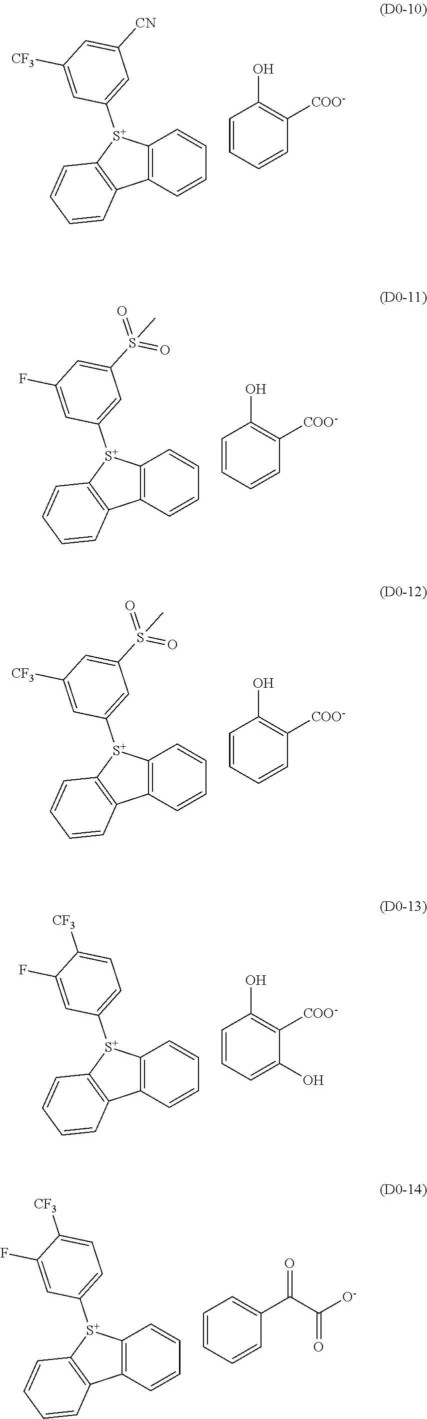

In the resist composition according to the present embodiment, the component (D0) may be used alone or may be used in a combination of two or more kinds thereof.

The content of the component (D0) in the resist composition according to the present embodiment is preferably in a range of 0.5 to 20 parts by mass, more preferably in a range of 1 to 15 parts by mass, and still more preferably in a range of 2 to 8 parts by mass with respect to 100 parts by mass of the component (A).

In a case where the content of the component (D0) is greater than or equal to the lower limits of the above-described preferable ranges, satisfactory lithography characteristics and a satisfactory resist pattern shape are likely to be obtained. On the contrary, in a case where the content is less than or equal to the upper limits of the above-described ranges, the sensitivity can be satisfactorily maintained and the throughput is also excellent.

In the resist composition according to the present embodiment, the proportion of the component (D0) in the total amount of the component (D) is, for example, 50% by mass or greater, preferably 70% by mass or greater, and more preferably 95% by mass or greater. The proportion of the component (D0) in the total amount of the component (D) may be 100% by mass.

The component (D) in the resist composition according to the present embodiment may contain an acid diffusion control agent component in addition to the above-described component (D0). Examples of the acid diffusion control agent component in addition to the component (D0) include a photo-decomposable base (D1) that is decomposed upon light exposure and loses acid diffusion controllability (hereinafter, referred to as “component (D1)”) (here, a component corresponding to the component (D0) is excluded) and a nitrogen-containing organic compound (D2) (hereinafter, referred to as “component (D2)”) that does not correspond to the component (D1).

### <<Component (D1)>>

As the component (D1), the same component as the component (D1) in the first embodiment can be used.

The component (D1) may be used alone or in a combination of two or more kinds thereof.

In a case where the resist composition according to the present embodiment contains the component (D1), the content of the component (D1) in the resist composition is preferably less than 20 parts by mass, more preferably less than 15 parts by mass, and still more preferably less than 8 parts by mass with respect to 100 parts by mass of the component (A1).

In a case where the content of the component (D1) is set to be in the above-described preferable ranges, defects are likely to be reduced.

It is preferable that the resist composition according to the present embodiment not contain the component (D1).

### <<Component (D2)>>

As the component (D2), the same component as the component (D2) in the first embodiment can be used.

The component (D2) may be used alone or in combination of two or more kinds thereof.

In a case where the resist composition according to the present embodiment contains the component (D2), the content of the component (D2) in the resist composition is preferably less than 10 parts by mass and more preferably less than 5 parts by mass with respect to 100 parts by mass of the component (A1).

In a case where the content of the component (D2) is set to be in the above-described preferable ranges, defects are likely to be reduced.

It is preferable that the resist composition according to the present embodiment not contain the component (D2).

### <Other Components>

The resist composition according to the present embodiment may further contain other components in addition to the component (A) and the component (D) described above. Examples of the other components include a component (B), a component (E), a component (F), and a component (S), which are described below.

### <<Component (B)>>

It is preferable that the resist composition according to the present embodiment contain the component (B).

As the component (B), the same component as the component (B1) in the first embodiment can be used.

In the resist composition according to the present embodiment, the component (B) may be used alone or in combination of two or more kinds thereof.

In the resist composition according to the present embodiment, the content of the component (B) is preferably in a range of 5 to 40 parts by mass, more preferably in a range of 10 to 40 parts by mass, still more preferably in a range of 12 to 40 parts by mass, and particularly preferably in a range of 15 to 35 parts by mass with respect to 100 parts by mass of the component (A).

In a case where the content of the component (B) is set to be in the above-described preferable range, pattern formation can be satisfactorily performed. Further, it is preferable that each component of the resist composition be dissolved in an organic solvent from the viewpoint that a uniform solution is easily obtained and the storage stability of the resist composition is improved.

### <<Component (E)>>

The resist composition according to the present embodiment may contain a component (E).

As the component (E), the same component as the component (E) in the first embodiment can be used.

In the resist composition of the present embodiment, the component (E) may be used alone or in combination of two or more kinds thereof.

In a case where the resist composition according to the present embodiment contains the component (E), the content of the component (E) is preferably in a range of 0.01 to 5 parts by mass and more preferably in a range of 0.05 to 3 parts by mass with respect to 100 parts by mass of the component (A). In a case where the content thereof is in the above-described range, the sensitivity, lithography characteristics, and the like are improved.

### <<Component (F)>>

The resist composition according to the present embodiment may contain a component (F).

As the component (F), the same component as the component (E) in the first embodiment can be used.

In the resist composition according to the present embodiment, the component (F) may be used alone or in combination of two or more kinds thereof.

In a case where the resist composition according to the present embodiment contains the component (F), the content of the component (F) is preferably in a range of 0.5 to 10 parts by mass and more preferably in a range of 1 to 10 parts by mass with respect to 100 parts by mass of the component (A).

### <<Component (S)>>

As the component (S), the same component as the component (S) in the first embodiment can be used.

The amount of the component (S) to be used is not particularly limited and is appropriately set to have a concentration which enables coating a substrate or the like depending on the thickness of the coated film. The component (S) is typically used in an amount such that the solid content concentration of the resist composition is set to be in a range of 0.1% to 20% by mass and preferably in a range of 0.2% to 15% by mass.

As desired, miscible additives such as additive resins, dissolution inhibitors, plasticizers, stabilizers, colorants, halation prevention agents, and dyes for improving the performance of the resist film can be added to the resist composition of the present embodiment, as appropriate.

Impurities and the like may be removed from the resist composition of the present embodiment using a porous polyimide film, a porous polyamideimide film, or the like after the resist material is dissolved in the component (S) in the same manner as that for the resist composition of the first embodiment.

The resist composition according to the present embodiment described above contains the compound (D0) containing the cation (C0) as the component (D).

The cation (C0) is a Low LUMO cation having a planar structure. In a case where the compound (D0) contains the cation (C0), the compound (D0) is decomposed upon light exposure and thus is likely to lose the acid diffusion controllability. In this manner, the sensitivity of the resist composition containing the compound (D0) is improved.

Further, the solubility of the cation (C0) in the component (S) is improved by allowing the phenyl group to contain two or more electron-withdrawing groups. Further, the solubility in a developing solution is improved in the development after light exposure. In this manner, the resist composition containing the compound (D0) can reduce defects.

As described above, according to the resist composition of the present embodiment, it is possible to achieve both high sensitivity and reduction of defects.

### Third Embodiment

The resist composition according to a third embodiment contains the compound (C) as the component (A). The resist composition according to the third embodiment contains the component (A) whose solubility in a developing solution is changed due to the action of an acid. The component (A) includes a resin component (A0) (hereinafter, also referred to as “component (A0)”) having a constitutional unit (a0) represented by General Formula (a0-1).

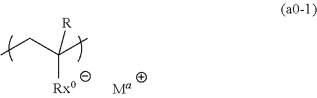

[In the formula, R represents a hydrogen atom, an alkyl group having 1 to 5 carbon atoms, or a halogenated alkyl group having 1 to 5 carbon atoms. M^{a+} represents the cation (C0). Rx⁰⁻ represents a group containing an anion.]

### <Component (A)>

The component (A) in the resist composition of the present embodiment contains the component (A0).

### Component (A0)

The component (A0) is a resin component having the constitutional unit (a0).

<<Constitutional unit (a0)>>

The constitutional unit (a0) is a constitutional unit represented by General Formula (a0-1). The constitutional unit (a0) may have an acid-generating agent function or an acid diffusion control agent function depending on the kind of Rx⁰⁻. Hereinafter, the constitutional unit (a0) having the acid-generating agent function will also be referred to as “constitutional unit (a0b). The constitutional unit (a0) having the acid diffusion control agent function will also be referred to as “constitutional unit (a0d).

Constitutional Unit (a0b):

### {Cation Moiety}

In General Formula (a0-1), M^{a+} represents the cation (C0).

### {Anion Moiety}

In General Formula (a0-1), R represents a hydrogen atom, an alkyl group having 1 to 5 carbon atoms, or a halogenated alkyl group having 1 to 5 carbon atoms. R is the same as that for R in Formula (a1-1).

In General Formula (a0-1), Rx⁰⁻ represents a group having an anion. In the constitutional unit (a0b), Rx⁰⁻ represents a group having an anion that generates an acid upon light exposure.

Examples of the anion contained in Rx⁰⁻ include an anion represented by Formula (b-1-an), an anion represented by Formula (b-2-an), and an anion represented by Formula (b-3-an).

Examples of the anion moiety of the constitutional unit (a0b) include an anion moiety represented by General Formula (a0-1-b).

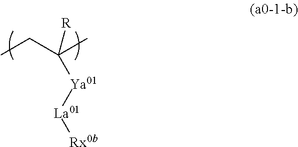

[In the formula, R represents a hydrogen atom, an alkyl group having 1 to 5 carbon atoms, or a halogenated alkyl group having 1 to 5 carbon atoms. Ya⁰¹ represents a single bond or a divalent linking group. La⁰¹ represents a single bond, —O—, —COO—, —CON(R′)—, —OCO—, —CONHCO—, or —CONHCS—, and R′ represents a hydrogen atom or a methyl group. Here, in a case where La⁰¹ represents —O—, Ya⁰¹ does not represent —CO—. Rx^{0b} represents a group represented by General Formula (b1-an), General Formula (b2-an), or General Formula (b3-an).]

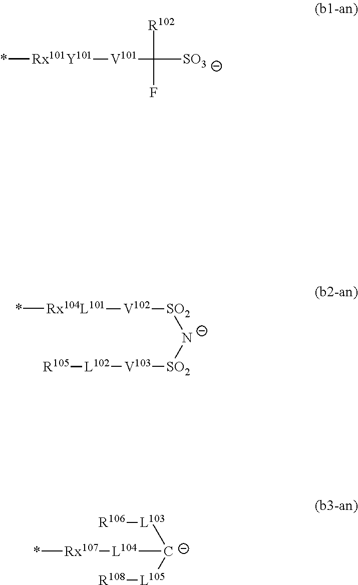

[In the formulae, Rx¹⁰¹, Rx¹⁰⁴ and Rx¹⁰⁷ each independently represents a cyclic group which may have a substituent, a chain-like alkylene group which may have a substituent, or a chain-like alkenediyl group which may have a substituent. R¹⁰⁵, R¹⁰⁶, and R¹⁰⁸ each independently represents a cyclic group which may have a substituent, a chain-like alkyl group which may have a substituent, or a chain-like alkenyl group which may have a substituent. Rx¹⁰⁴ and R¹⁰⁵ may be bonded to each other to form a ring structure. R¹⁰² represents a fluorinated alkyl group having 1 to 5 carbon atoms or a fluorine atom. Y¹⁰¹ represents a divalent linking group having an oxygen atom or a single bond. V¹⁰¹ to V¹⁰³ each independently represents a single bond, an alkylene group, or a fluorinated alkylene group. L¹⁰¹ and L¹⁰² each independently represents a single bond or an oxygen atom. L¹⁰³ to L¹⁰⁵ each independently represents a single bond, —CO—, or —SO₂—. * represents a bonding site bonded to La⁰¹ in Formula (a0-1-b).]

In Formula (a0-1-b), R represents a hydrogen atom, an alkyl group having 1 to 5 carbon atoms, or a halogenated alkyl group having 1 to 5 carbon atoms. R is the same as that for R in Formula (a1-1).

In Formula (a0-1-b), Ya⁰¹ represents a single bond or a divalent linking group. The divalent linking group is not particularly limited, and examples thereof include a divalent hydrocarbon group which may have a substituent and a divalent linking group having a hetero atom. Examples of the divalent hydrocarbon group which may have a substituent include the same groups as those for Ya²¹ in Formula (a2-1). Examples of the divalent linking group having a hetero atom include the same groups as those for Ya²¹ in Formula (a2-1).

Ya⁰¹ represents preferably a single bond, an ester bond [—C(═O)—O—], an ether bond (—O—), a linear or branched alkylene group, or a combination thereof and more preferably a single bond.

In Formula (a0-1-b), La⁰¹ represents —O—, —COO—, —CON(R′)—, —OCO—, —CONHCO—, or —CONHCS—. R′ represents a hydrogen atom or a methyl group. Here, in a case where La⁰¹ represents —O—, Ya⁰¹ does not represent —CO—. It is preferable that La⁰¹ represent —COO—.

In Formula (a0-1-b), Rx^{0b} represents a group represented by General Formula (b1-an), General Formula (b2-an), or General Formula (b3-an).

Group Represented by Formula (b1-an)

In Formula (b1-an), Rx¹⁰¹ represents a cyclic group which may have a substituent, a chain-like alkylene group which may have a substituent, or a chain-like alkenediyl group which may have a substituent.

Rx¹⁰¹ represents a group in which one hydrogen atom has been removed from R¹⁰¹ in Formula (b-1-an). Examples of Rx¹⁰¹ include a group in which one hydrogen atom has been removed from the group described as R¹⁰¹ in Formula (b-1-an). Rx¹⁰¹ represents preferably a cyclic group which may have a substituent and more preferably an alicyclic hydrocarbon group which may have a substituent. It is preferable that Rx¹⁰¹ represent a group in which two hydrogen atoms have been removed from a monocycloalkane or a polycycloalkane. It is preferable that the monocycloalkane have 3 to 6 carbon atoms, and specific examples thereof include cyclopentane and cyclohexane. It is preferable that the polycycloalkane have 7 to 30 carbon atoms, and specific examples include adamantane, norbornane, isobornane, tricyclodecane, and tetracyclododecane.

R¹⁰², Y¹⁰¹, and V¹⁰¹ each is the same as that for R¹⁰², Y¹⁰¹, and V¹⁰¹ in Formula (b-1-an).

Examples of the group represented by Formula (b1-an) include groups each represented by General Formulae (b1-an1) to (b1-an3).

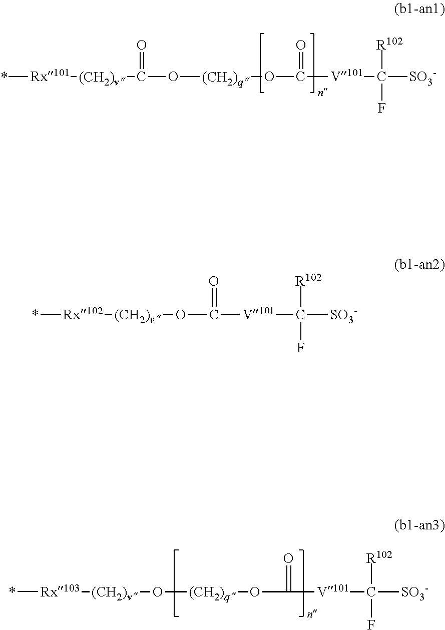

[In the formulae, Rx″¹⁰¹ represents an aliphatic cyclic group which may have a substituent, a group in which one hydrogen atom has been removed from a monovalent heterocyclic group represented by any of Chemical Formulae (r-hr-1) to (r-hr-6), a group in which one hydrogen atom has been removed from a fused cyclic group represented by Formula (r-br-1) or (r-br-2), and a chain-like alkylene group which may have a substituent. Rx″¹⁰² represents an aliphatic cyclic group which may have a substituent, a group in which one hydrogen atom has been removed from a fused cyclic group represented by Formula (r-br-1) or (r-br-2), a group in which one hydrogen atom has been removed from a lactone-containing cyclic group represented by any of General Formulae (a2-r-1) and (a2-r-3) to (a2-r-7), or a group in which one hydrogen atom has been removed from a —SO₂-containing cyclic group represented by any of General Formulae (a5-r-1) to (a5-r-4). Rx″¹⁰³ represents an aromatic cyclic group which may have a substituent, an aliphatic cyclic group which may have a substituent, or a chain-like alkenediyl group which may have a substituent. V″¹⁰¹ represents a single bond, an alkylene group having 1 to 4 carbon atoms, or a fluorinated alkylene group having 1 to 4 carbon atoms. R¹⁰² represents a fluorine atom or a fluorinated alkyl group having 1 to 5 carbon atoms. Each v″ independently represents an integer of 0 to 3, each q″ independently represents an integer of 0 to 20, and n″ represents 0 or 1.]

Rx″¹⁰¹ in Formula (α-1-an1) represents a group in which one hydrogen atom has been removed from R″¹⁰¹ in Formula (an-1).

Rx″¹⁰² in Formula (α-1-an2) represents a group in which one hydrogen atom has been removed from R″¹⁰² in Formula (an-2).

Rx″¹⁰³ in Formula (α-1-an3) represents a group in which one hydrogen atom has been removed from R″¹⁰³ in Formula (an-3).

V″¹⁰, R¹⁰², v″, q″, and n″ each is the same as that for V″¹⁰, R¹⁰², v″, q″, and n″ in Formula (an-1), Formula (an-2), and Formula (an-3).

Group Represented by Formula (b2-an)

In Formula (b2-an), Rx¹⁰⁴ represents a cyclic group which may have a substituent, a chain-like alkylene group which may have a substituent, or a chain-like alkenediyl group which may have a substituent.

Rx¹⁰⁴ represents a group in which one hydrogen atom has been removed from R¹⁰⁴ in Formula (b-2-an). Examples of Rx¹⁰⁴ include a group in which one hydrogen atom has been removed from the group described as R¹⁰⁴ in Formula (b-2-an).

R¹⁰⁵, L¹⁰¹, L¹⁰², V¹⁰², and V¹⁰³ each is the same as that for R¹⁰⁵, L¹⁰¹, L¹⁰², V¹⁰², and V¹⁰³ in Formula (b-2-an).

Group Represented by Formula (b3-an)

In Formula (b3-an), Rx¹⁰⁷ represents a cyclic group which may have a substituent, a chain-like alkylene group which may have a substituent, or a chain-like alkenediyl group which may have a substituent.

Rx¹⁰⁷ represents a group in which one hydrogen atom has been removed from R¹⁰⁷ in Formula (b-3-an). Examples of Rx¹⁰⁷ include a group in which one hydrogen atom has been removed from the group described as R¹⁰⁷ in Formula (b-3-an).

R¹⁰⁶, R¹⁰⁸, L¹⁰³, L¹⁰⁴, and L¹⁰⁵ each is the same as that for R¹⁰⁶, R¹⁰⁸, L¹⁰³, L¹⁰⁴, and L¹⁰⁵ in Formula (b-3-an).

It is preferable that Rx^{0b} represent a group represented by Formula (b1-an). Among the examples, Rx^{0b} represents more preferably a group represented by any of Formulae (b1-an1) to (b1-an3), still more preferably a group represented by Formula (b1-an1) or (b-an2), and particularly preferably a group represented by Formula (b1-an1).

Specific examples of the anion moiety of the constitutional unit (a0) in a case where Rx^{0b} represents a group represented by Formula (b1-an) are shown below, but the present invention is not limited thereto. In the formulae, R^{α} represents a hydrogen atom, a methyl group, or a trifluoromethyl group.

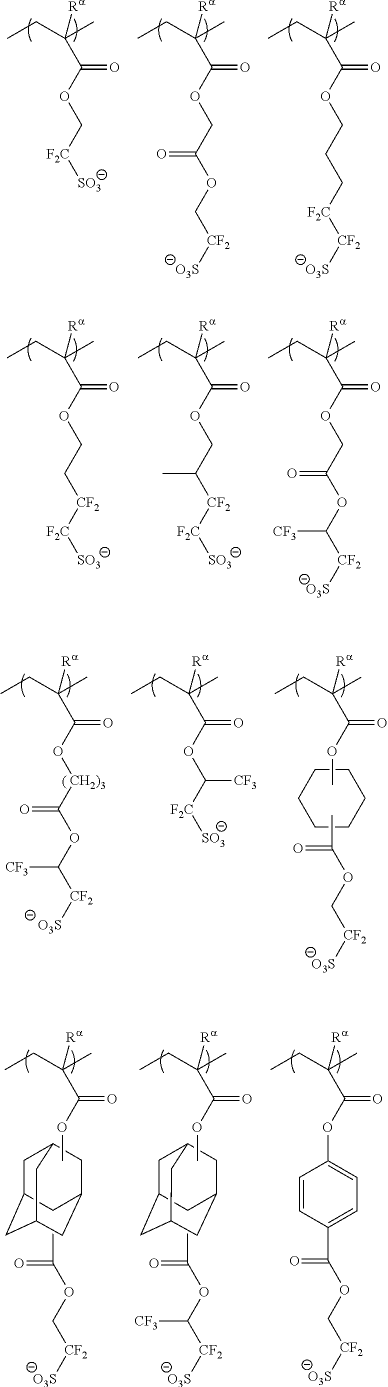

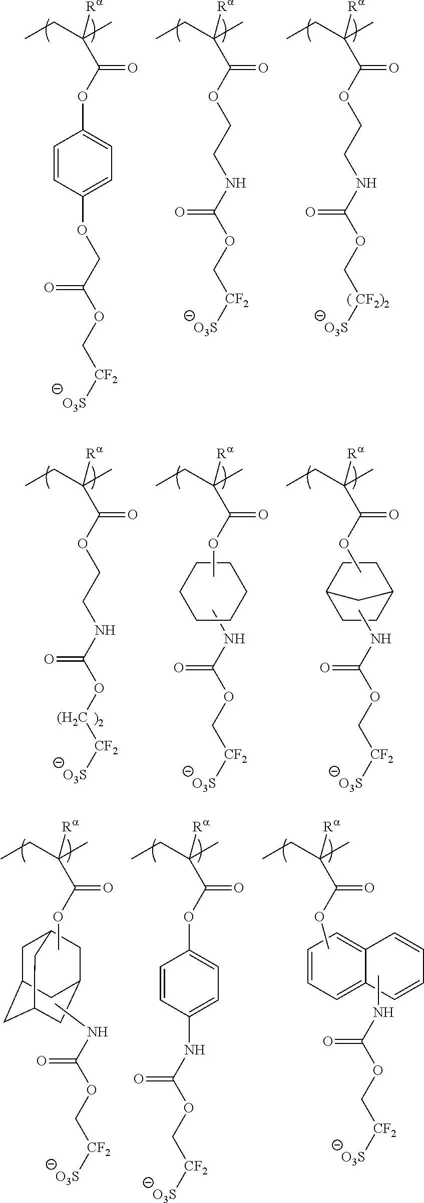

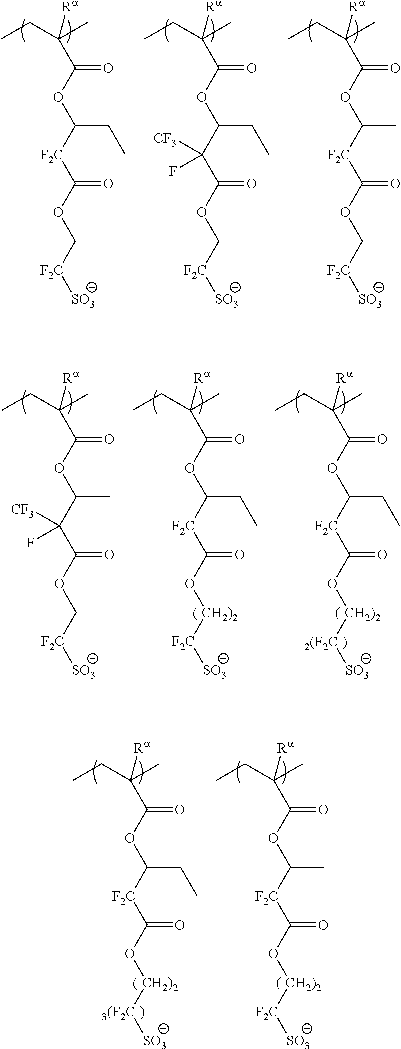

Specific examples of the anion moiety of the constitutional unit (a0) in a case where Rx^{0b} represents a group represented by Formula (b2-an) are shown below, but the present invention is not limited thereto. In the formulae, R^{α} represents a hydrogen atom, a methyl group, or a trifluoromethyl group.

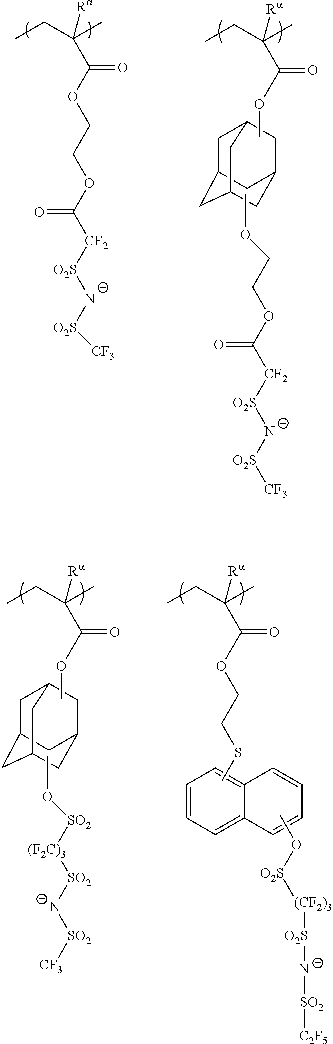

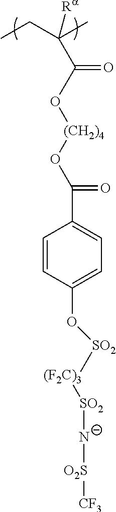

Specific examples of the anion moiety of the constitutional unit (a0) in a case where Rx^{0b} represents a group represented by Formula (b3-an) are shown below, but the present invention is not limited thereto. In the formulae, R^{α} represents a hydrogen atom, a methyl group, or a trifluoromethyl group.

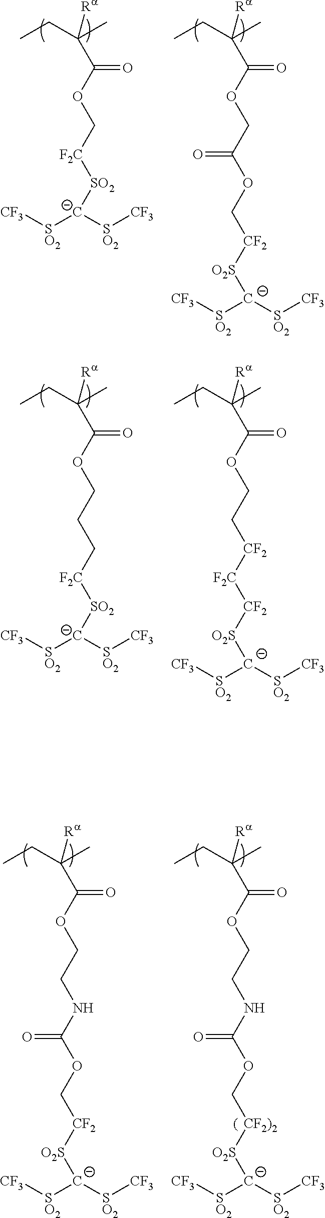

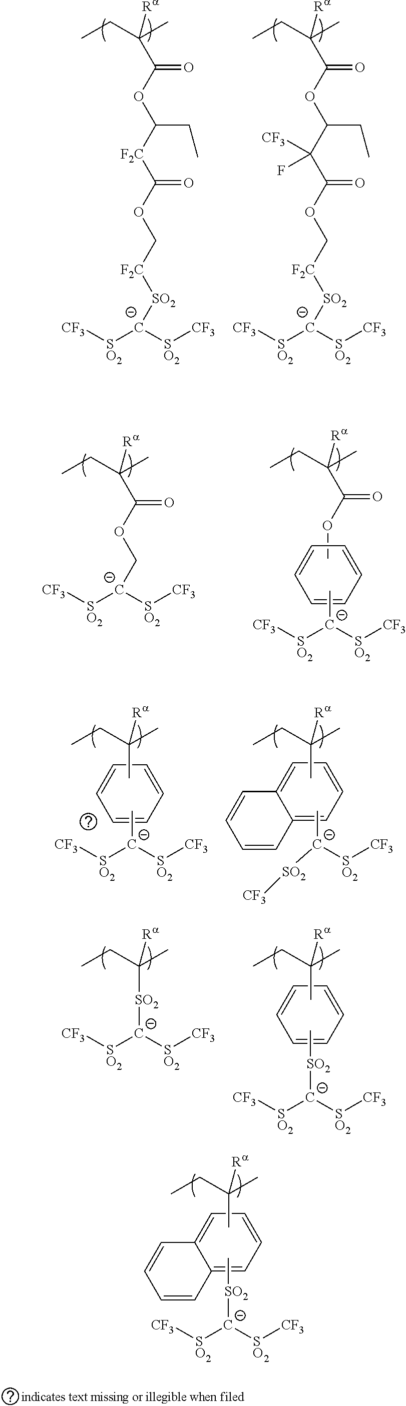

<<Constitutional Unit (a0d)>>

### {Cation Moiety}

In General Formula (a0-1), M^{a+} represents the cation (C0).

### {Anion Moiety}

In General Formula (a0-1), Rx⁰⁻ represents a group having an anion. In the constitutional unit (a0d), Rx⁰⁻ represents a group having an anion that traps an acid generated upon light exposure.

Examples of the anion contained in Rx⁰⁻ include an anion represented by Formula (d-1-an), an anion represented by Formula (d-2-an), and an anion represented by Formula (d-3-an).

Examples of the anion moiety of the constitutional unit (a0d) include an anion moiety represented by General Formula (a0-1-d).

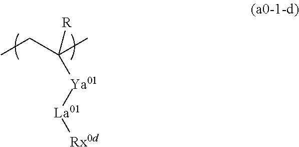

[In the formula, R represents a hydrogen atom, an alkyl group having 1 to 5 carbon atoms, or a halogenated alkyl group having 1 to 5 carbon atoms. Ya⁰¹ represents a single bond or a divalent linking group. La⁰¹ represents a single bond, —O—, —COO—, —CON(R′)—, —OCO—, —CONHCO—, or —CONHCS—, and R′ represents a hydrogen atom or a methyl group. Here, in a case where La⁰¹ represents —O—, Ya⁰¹ does not represent —CO—. Rx^{0d} represents a group represented by General Formula (d1-an), General Formula (d2-an), or General Formula (d3-an). * represents a bonding site bonded to La⁰¹ in Formula (a0-1-d).]

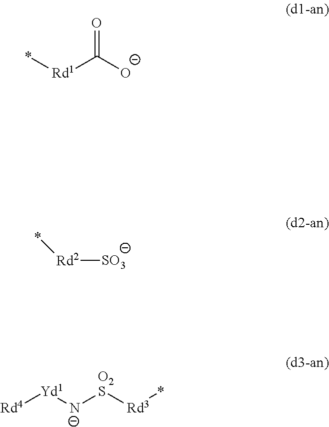

[In the formulae, Rd⁰¹ to Rd⁰³ represent a single bond, a cyclic group which may have a substituent, a chain-like alkylene group which may have a substituent, or a chain-like alkenediyl group which may have a substituent. Here, no fluorine atom is bonded to the carbon atom adjacent to the S atom in Rd⁰² of Formula (d1-2). Rd⁴ represents a cyclic group which may have a substituent, a chain-like alkyl group which may have a substituent, or a chain-like alkenyl group which may have a substituent. Yd¹ represents a single bond or a divalent linking group.]

In Formula (a0-1-d), R, Ya⁰¹, and La⁰¹ each is the same as that for R, Ya⁰¹, and La⁰¹ in Formula (a0-1-b).

In Formula (a0-1-d), Rx^{0d} represents a group represented by General Formula (d1-an), General Formula (d2-an), or General Formula (d3-an).

Group Represented by Formula (d1-an)

In Formula (d1-an), Rd⁰¹ represents a single bond, a cyclic group which may have a substituent, a chain-like alkylene group which may have a substituent, or a chain-like alkenediyl group which may have a substituent.

It is preferable that Rd⁰¹ represent a group in which one hydrogen atom has been removed from Rd¹ in Formula (d-1-an). Examples of Rd⁰¹ include a group in which one hydrogen atom has been removed from the group described as Rd¹ in Formula (d-1-an). It is preferable that Rd⁰¹ represent a cyclic group which may have a substituent. It is preferable that Rx¹⁰¹ represent a group in which two hydrogen atoms have been removed from a monocycloalkane or a polycycloalkane or an aromatic hydrocarbon group (for example, an arylene group). It is preferable that the monocycloalkane have 3 to 6 carbon atoms, and specific examples thereof include cyclopentane and cyclohexane. It is preferable that the polycycloalkane have 7 to 30 carbon atoms, and specific examples include adamantane, norbornane, isobornane, tricyclodecane, and tetracyclododecane. Examples of the aromatic hydrocarbon group include a phenylene group, a naphthylene group, and a polycyclic structure having a bicyclooctane skeleton (a polycyclic structure formed of a bicyclooctane skeleton and a ring structure other than the bicyclooctane skeleton).

Group Represented by Formula (d2-an)

In Formula (d2-an), Rd⁰² represents a single bond, a cyclic group which may have a substituent, a chain-like alkylene group which may have a substituent, or a chain-like alkenediyl group which may have a substituent.

It is preferable that Rd⁰² represent a group in which one hydrogen atom has been removed from Rd² in Formula (d-2-an). Examples of Rd⁰² include a group in which one hydrogen atom has been removed from the group described as Rd² in Formula (d-2-an).

Group Represented by Formula (d3-an)

In Formula (d3-an), Rd⁰³ represents a single bond, a cyclic group which may have a substituent, a chain-like alkylene group which may have a substituent, or a chain-like alkenediyl group which may have a substituent.

It is preferable that Rd⁰³ represent a group in which one hydrogen atom has been removed from Rd² in Formula (d-3-an). Examples of Rd⁰³ include a group in which one hydrogen atom has been removed from the group described as Rd² in Formula (d-3-an).

Rd⁴ and Yd¹ each is the same as that for Rd⁴ and Yd¹ in Formula (d-3-an).

Rx^{0d} represents preferably a group represented by Formula (d1-an) or Formula (d2-an) and more preferably a group represented by Formula (d1-an).

Specific examples of the anion moiety of the constitutional unit (a0) in a case where Rx^{0d} represents a group represented by Formula (d1-an) are shown below, but the present invention is not limited thereto. In the formulae, R^{α} represents a hydrogen atom, a methyl group, or a trifluoromethyl group. m″ represents an integer of 0 to 3.

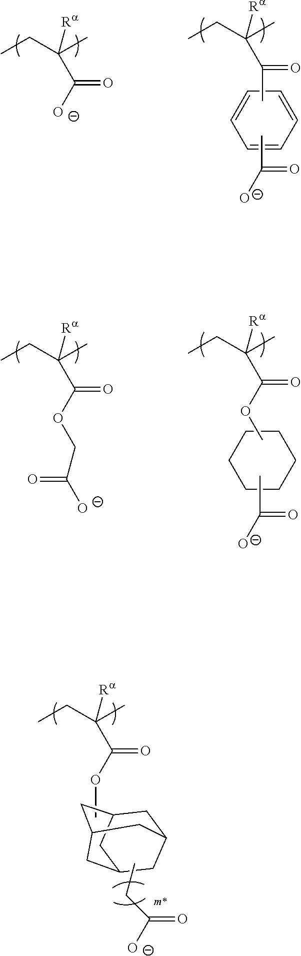

Specific examples of the anion moiety of the constitutional unit (a0) in a case where Rx^{0d} represents a group represented by Formula (d2-an) are shown below, but the present invention is not limited thereto. In the formulae, R^{α} represents a hydrogen atom, a methyl group, or a trifluoromethyl group.

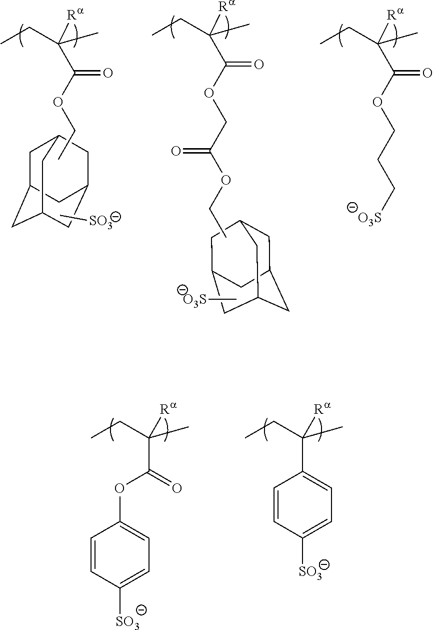

Specific examples of the anion moiety of the constitutional unit (a0) in a case where Rx^{0d} represents a group represented by Formula (d2-an) are shown below, but the present invention is not limited thereto. In the formulae, R^{α} represents a hydrogen atom, a methyl group, or a trifluoromethyl group.

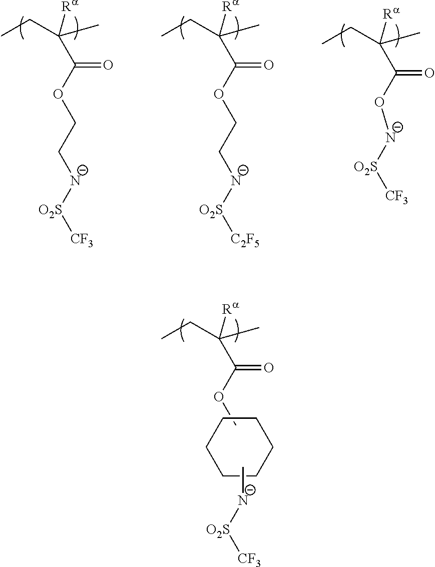

Specific examples of the constitutional unit (a0) are shown below, but the present invention is not limited thereto.

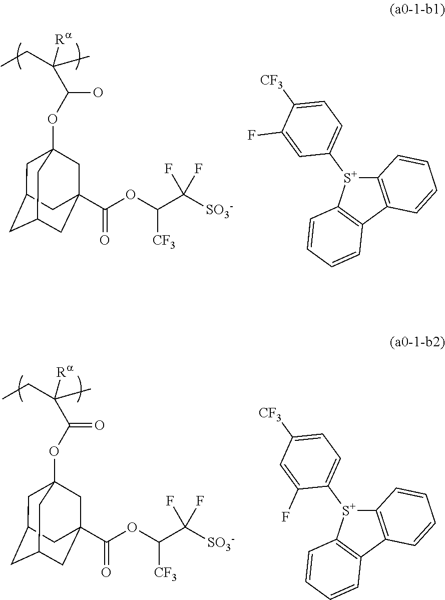

The constitutional unit (a0) included in the component (A0) may be used alone or two or more kinds thereof.

The proportion of the constitutional unit (a0) in the component (A0) is preferably in a range of 3% to 90% by mole, more preferably in a range of 5% to 75% by mole, still more preferably in a range of 10% to 70% by mole, and particularly preferably in a range of 10% to 60% by mole with respect to the total amount (100% by mole) of all constitutional units constituting the component (A0). The proportion of the constitutional unit (a0) in the component (A0) may be 100% by mole.

The component (A0) may be used alone or in combination of two or more kinds thereof.

The weight-average molecular weight (Mw) (in terms of polystyrene according to gel permeation chromatography (GPC)) of the component (A0) is not particularly limited, but is preferably in a range of 1,000 to 50,000, more preferably in a range of 2,000 to 30,000, and still more preferably in a range of 3,000 to 20,000.

In a case where the Mw of the component (A0) is less than or equal to the upper limits of the above-described preferable ranges, the resist composition exhibits sufficient solubility in a resist solvent for a resist enough to be used as a resist. Meanwhile, in a case where the Mw thereof is greater than or equal to the lower limits of the above-described preferable ranges, the dry etching resistance and the cross-sectional shape of the resist pattern are satisfactory.

Further, the dispersity (Mw/Mn) of the component (A0) is not particularly limited, but is preferably in a range of 1.0 to 4.0, more preferably in a range of 1.0 to 3.0, and particularly preferably in a range of 1.0 to 2.0.

### Component (A1)

It is preferable that the resist composition according to the present embodiment contain a resin component (A1) (component (A1)) whose solubility in a developing solution is changed due to the action of an acid. As the component (A1), the same component as the component (A1) in the first embodiment can be used. The component (A1) may be used alone or in combination of two or more kinds thereof.

### Component (A1) Having Constitutional Unit (a0) (Component (A1-0))

The component (A1) may have a constitutional unit (a0). In this case, the component (A1) is the component (A0). Hereinafter, the component (A1) having the constitutional unit (a0) will also be referred to as “component (A1-0)”.

The constitutional unit (a0) included in the component (A1-0) may be used alone or two or more kinds thereof.

In a case where the component (A1-0) has the constitutional unit (a0), the proportion of the constitutional unit (a0) in the component (A1-0) is preferably in a range of 3% to 50% by mole, more preferably in a range of 5% to 40% by mole, still more preferably in a range of 10% to 30% by mole, and particularly preferably in a range of 15% to 20% by mole with respect to the total amount (100% by mole) of all constitutional units constituting the component (A1-0).

In a case where the proportion of the constitutional unit (a0) is greater than or equal to the lower limits of the above-described preferable ranges, the lithography characteristics such as the sensitivity and reduction of defects are improved. In addition, in a case where the proportion of the constitutional unit (a0) is less than or equal to the upper limits of the above-described preferable ranges, the balance between the constitutional unit (a0) and other constitutional units can be achieved, and various lithography characteristics are enhanced.

<<Constitutional Unit (a1)>>

It is preferable that the component (A1-0) have a constitutional unit (a1) containing an acid-decomposable group whose polarity is increased due to the action of an acid. As the constitutional unit (a1), the same constitutional unit as the constitutional unit (a1) in the first embodiment can be used.

The constitutional unit (a1) of the component (A1-0) may be used alone or two or more kinds thereof.

The proportion of the constitutional unit (a1) in the component (A1-0) is preferably in a range of 5% to 80% by mole, more preferably in a range of 10% to 75% by mole, still more preferably in a range of 20% to 70% by mole, and particularly preferably in a range of 25% to 60% by mole with respect to the total amount (100% by mole) of all constitutional units constituting the component (A1).

In a case where the proportion of the constitutional unit (a1) is greater than or equal to the lower limits of the above-described preferable ranges, lithography characteristics such as the sensitivity, the resolution, and improved roughness are improved. Further, in a case where the proportion of the constitutional unit (a1) is less than or equal to the upper limits of the above-described preferable ranges, the balance between the constitutional unit (a1) and other constitutional units can be achieved, and various lithography characteristics are enhanced.

### <<Other Constitutional Units<<

The component (A1-0) may have other constitutional units as necessary in addition to the constitutional unit (a0) and the constitutional unit (a1). Examples of the other constitutional units include the constitutional unit (a2), the constitutional unit (a3), the constitutional unit (a4), the constitutional unit (a10), and the constitutional unit (st).

### Constitutional Unit (a2):

The component (A1-0) may have a constitutional unit (a2). As the constitutional unit (a2), the same constitutional unit (a2) as in the first embodiment can be used.

The constitutional unit (a2) of the component (A1-0) may be used alone or two or more kinds thereof.

In a case where the component (A1-0) has the constitutional unit (a2), the proportion of the constitutional unit (a2) is preferably in a range of 5% to 60% by mole, more preferably in a range of 10% to 60% by mole, still more preferably in a range of 20% to 60% by mole, and particularly preferably in a range of 30% to 60% by mole with respect to the total amount (100% by mole) of all constitutional units constituting the component (A1).

In a case where the proportion of the constitutional unit (a2) is greater than or equal to the lower limits of the above-described preferable ranges, the effect of containing the constitutional unit (a2) is sufficiently obtained. Further, in a case where the proportion of the constitutional unit (a2) is less than or equal to the upper limits of the above-described preferable ranges, the balance between the constitutional unit (a2) and other constitutional units can be achieved, and various lithography characteristics are enhanced.

### Constitutional Unit (a3):

The component (A1-0) may have the constitutional unit (a3). As the constitutional unit (a3), the same constitutional unit (a3) as in the first embodiment can be used.

The constitutional unit (a3) of the component (A1-0) may be used alone or two or more kinds thereof.

In a case where the component (A1-0) has the constitutional unit (a3), the proportion of the constitutional unit (a3) is preferably in a range of 1% to 30% by mole, more preferably in a range of 2% to 25% by mole, and still more preferably in a range of 5% to 20% by mole with respect to the total amount (100% by mole) of all constitutional units constituting the component (A1-0).

In a case where the proportion of the constitutional unit (a3) is greater than or equal to the lower limits of the above-described preferable ranges, the effect of containing the constitutional unit (a3) is sufficiently obtained. Further, in a case where the proportion of the constitutional unit (a3) is less than or equal to the upper limits of the above-described preferable ranges, the balance between the constitutional unit (a3) and other constitutional units can be achieved, and various lithography characteristics are enhanced.

### Constitutional Unit (a4):

The component (A1-0) may have the constitutional unit (a4). As the constitutional unit (a4), the same constitutional unit as the constitutional unit (a4) in the first embodiment can be used.

The constitutional unit (a4) of the component (A1-0) may be used alone or two or more kinds thereof.

In a case where the component (A1-0) contains the constitutional unit (a4), the proportion of the constitutional unit (a4) is preferably in a range of 1% to 40% by mole and more preferably in a range of 5% to 20% by mole with respect to the total amount (100% by mole) of all constitutional units constituting the component (A1-0). In a case where the proportion of the constitutional unit (a4) is greater than or equal to the lower limits of the above-described preferable ranges, the effect of containing the constitutional unit (a4) is sufficiently obtained. Further, in a case where the proportion of the constitutional unit (a4) is less than or equal to the upper limits of the above-described preferable ranges, the balance between the constitutional unit (a4) and other constitutional units can be achieved, and various lithography characteristics are enhanced.

Constitutional Unit (a10):

It is preferable that the component (A1-0) have the constitutional unit (a10). As the constitutional unit (a10), the same constitutional unit as the constitutional unit (a10) in the first embodiment can be used.

The constitutional unit (a10) of the component (A1-0) may be used alone or two or more kinds thereof.

In a case where the component (A1-0) has the constitutional unit (a10), the proportion of the constitutional unit (a10) is preferably in a range of 10% to 80% by mole, more preferably in a range of 20% to 70% by mole, still more preferably in a range of 30% to 60% by mole, and particularly preferably in a range of 30% to 55% by mole with respect to the total amount (100% by mole) of all constitutional units constituting the component (A1-0).

In a case where the proportion of the constitutional unit (a10) is greater than or equal to the lower limits of the above-described preferable ranges, the sensitivity is more likely to be increased. Further, in a case where the proportion of the constitutional unit (a10) is less than or equal to the upper limits of the above-described preferable ranges, the balance between the constitutional unit (a10) and other constitutional units can be achieved, and various lithography characteristics are enhanced.

### Constitutional Unit (st):

The component (A1-0) may have the constitutional unit (st). As the constitutional unit (st), the same constitutional unit as the constitutional unit (st) in the first embodiment can be used.

The constitutional unit (st) of the component (A1-0) may be used alone or two or more kinds thereof.

In a case where the component (A1-0) has the constitutional unit (st), the proportion of the constitutional unit (st) is preferably in a range of 1% to 30% by mole and more preferably in a range of 3% to 20% by mole with respect to the total amount (100% by mole) of all constitutional units constituting the component (A1-0).

In a case where the proportion of the constitutional unit (st) is greater than or equal to the lower limits of the above-described preferable ranges, the effect of containing the constitutional unit (st) is sufficiently obtained. Further, in a case where the proportion of the constitutional unit (st) is less than or equal to the upper limits of the above-described preferable ranges, the balance between the constitutional unit (st) and other constitutional units can be achieved, and various lithography characteristics are enhanced.

The component (A1-0) may be used alone or in combination of two or more kinds thereof.

Examples of the component (A1-0) include a high-molecular-weight compound having a repeating structure of the constitutional unit (a0) and the constitutional unit (a1), and preferred examples thereof include a high-molecular-weight compound having a repeating structure of the constitutional unit (a0), the constitutional unit (a1), and the constitutional unit (a10).

Among these, suitable examples of the component (A1-0) include a high-molecular-weight compound formed of a repeating structure of the constitutional unit (a0) and the constitutional unit (a1), and a high-molecular-weight compound formed of a repeating structure of the constitutional unit (a0), the constitutional unit (a1), and the constitutional unit (a10).

The weight-average molecular weight (Mw) (in terms of polystyrene according to gel permeation chromatography (GPC)) of the component (A1-0) is not particularly limited, but is preferably in a range of 1,000 to 50,000, more preferably in a range of 2,000 to 30,000, and still more preferably in a range of 3,000 to 20,000.

In a case where the Mw of the component (A1-0) is less than or equal to the upper limits of the above-described preferable ranges, the resist composition exhibits sufficient solubility in a resist solvent for a resist enough to be used as a resist. Meanwhile, in a case where the Mw of the component (A1-0) is greater than or equal to the lower limits of the above-described preferable ranges, the dry etching resistance and the cross-sectional shape of the resist pattern are enhanced.

Further, the dispersity (Mw/Mn) of the component (A1) is not particularly limited, but is preferably in a range of 1.0 to 4.0, more preferably in a range of 1.0 to 3.0, and particularly preferably in a range of 1.0 to 2.0.

### Component (A2)

The resist composition according to the present embodiment may contain the component (A2) as the component (A). As the component (A2), the same component as the component (A2) in the first embodiment can be used.

The proportion of the component (A1-0) in the component (A) is preferably 25% by mass or greater, more preferably 50% by mass or greater, and still more preferably 75% by mass or greater, and may be 100% by mass with respect to the total mass of the component (A). In a case where the proportion thereof is 25% by mass or greater, a resist pattern having excellent various lithography characteristics such as high sensitivity, high resolution, improved roughness, and reduction of defects is likely to be formed.

In the resist composition of the present embodiment, the content of the component (A) may be adjusted according to the thickness of the resist film intended to be formed.

### <Other Components>

The resist composition according to the present embodiment may further contain other components in addition to the component (A) described above. Examples of the other components include the component (B), the component (D), the component (E), the component (F), and the component (S), which are described below.

### <<Component (B)>>

The resist composition according to the present embodiment may contain the component (B).

As the component (B), the same component as the component (B1) in the first embodiment can be used.

In the resist composition according to the present embodiment, the component (B) may be used alone or in combination of two or more kinds thereof.

In a case where the resist composition according to the present embodiment contains the component (B), the content of the component (B) is preferably in a range of 5 to 40 parts by mass, more preferably in a range of 10 to 40 parts by mass, still more preferably in a range of 12 to 40 parts by mass, and particularly preferably in a range of 15 to 35 parts by mass with respect to 100 parts by mass of the component (A).

In a case where the content of the component (B) is set to be in the above-described preferable range, pattern formation can be satisfactorily performed. Further, it is preferable that each component of the resist composition be dissolved in an organic solvent from the viewpoint that a uniform solution is easily obtained and the storage stability of the resist composition is improved.

### <<Component (D)>>

It is preferable that the resist composition according to the present embodiment contain the component (D).

As the component (D), the same component as the component (D) in the first embodiment can be used.

In the resist composition according to the present embodiment, the component (D) may be used alone or in combination of two or more kinds thereof.

In a case where the resist composition according to the present embodiment contains the component (D), the content of the component (D) is preferably in a range of 0.5 to 20 parts by mass, more preferably in a range of 1 to 15 parts by mass, and still more preferably in a range of 2 to 8 parts by mass with respect to 100 parts by mass of the component (A).

In a case where the content of the component (D) is greater than or equal to the lower limits of the above-described preferable ranges, satisfactory lithography characteristics and a satisfactory resist pattern shape are likely to be obtained. Meanwhile, in a case where the content of the component (D) is less than or equal to the upper limits of the above-described preferable ranges, the sensitivity can be satisfactorily maintained.

### <<Component (E)>>

The resist composition according to the present embodiment may contain a component (E).

As the component (E), the same component as the component (E) in the first embodiment can be used.

In the resist composition of the present embodiment, the component (E) may be used alone or in combination of two or more kinds thereof.

In a case where the resist composition according to the present embodiment contains the component (E), the content of the component (E) is preferably in a range of 0.01 to 5 parts by mass and more preferably in a range of 0.05 to 3 parts by mass with respect to 100 parts by mass of the component (A). In a case where the content thereof is in the above-described range, the sensitivity, lithography characteristics, and the like are improved.

### <<Component (F)>>

The resist composition according to the present embodiment may contain a component (F).

As the component (F), the same component as the component (E) in the first embodiment can be used.

In the resist composition according to the present embodiment, the component (F) may be used alone or in combination of two or more kinds thereof.

In a case where the resist composition according to the present embodiment contains the component (F), the content of the component (F) is preferably in a range of 0.5 to 10 parts by mass and more preferably in a range of 1 to 10 parts by mass with respect to 100 parts by mass of the component (A).

### <<Component (S)>>

As the component (S), the same component as the component (S) in the first embodiment can be used.

The amount of the component (S) to be used is not particularly limited and is appropriately set to have a concentration which enables coating a substrate or the like depending on the thickness of the coated film. The component (S) is typically used in an amount such that the solid content concentration of the resist composition is set to be in a range of 0.1% to 20% by mass and preferably in a range of 0.2% to 15% by mass.

As desired, miscible additives such as additive resins, dissolution inhibitors, plasticizers, stabilizers, colorants, halation prevention agents, and dyes for improving the performance of the resist film can be added to the resist composition of the present embodiment, as appropriate.

Impurities and the like may be removed from the resist composition of the present embodiment using a porous polyimide film, a porous polyamideimide film, or the like after the resist material is dissolved in the component (S) in the same manner as that for the resist composition of the first embodiment.

The resist composition according to the present embodiment described above contains, as the component (A), the resin component (A0) containing the cation (C0).

The cation (C0) is a Low LUMO cation having a planar structure. The resin component (A0) has a constitutional unit (a0) that generates acid upon light exposure. In a case where the constitutional unit (a0) contains the cation (C0), the acid generation efficiency upon light exposure is improved, and thus the sensitivity of the resist composition containing the resin component (A0) is improved.

Further, the solubility of the cation (C0) in the component (S) is improved by allowing the phenyl group to contain two or more electron-withdrawing groups. Further, the solubility in a developing solution is improved in the development after light exposure. In this manner, the resist composition containing the resin component (A0) can reduce defects.

As described above, according to the resist composition of the present embodiment, it is possible to achieve both high sensitivity and reduction of defects.

### Fourth to Seventh Embodiments

The resist composition according to a fourth embodiment contains a compound (C) as the component (B) and the component (D). That is, the resist composition according to the fourth embodiment contains the component (B0) and the component (D0). It is preferable that the resist composition according to the fourth embodiment contain the component (A1), the component (B0), and the component (D0).

The resist composition according to the fourth embodiment may contain the component (E), the component (F), and the component (S) as other components.

In the resist composition according to the fourth embodiment, the content of each component can be set to the same as in the above-described first to third embodiments.

The resist composition according to a fifth embodiment contains the compound (C) as the component (B) and the component (A). That is, the resist composition according to the fifth embodiment contains the component (B0) and the component (A0). The component (A0) may have any one or both of the constitutional unit (a0b) and the constitutional unit (a0d), but it is preferable that the component (A0) have the constitutional unit (a0d). It is preferable that the resist composition according to the fifth embodiment contain the component (A1-0) and the component (B0).

The resist composition according to the fifth embodiment may contain the component (D), the component (E), the component (F), and the component (S) as other components.

In the resist composition according to the fifth embodiment, the content of each component can be set to the same as in the above-described first to third embodiments.

The resist composition according to a sixth embodiment contains the compound (C) as the component (D) and the component (A). That is, the resist composition according to the sixth embodiment contains the component (D0) and the component (A0). The component (A0) may have any one or both of the constitutional unit (a0b) and the constitutional unit (a0d), but it is preferable that the component (A0) have the constitutional unit (a0b). It is preferable that the resist composition according to the sixth embodiment contain the component (A1-0) and the component (D0).

The resist composition according to the sixth embodiment may contain the component (B), the component (E), the component (F), and the component (S) as other components.

In the resist composition according to the sixth embodiment, the content of each component can be set to the same as in the above-described first to third embodiments.

The resist composition according to a seventh embodiment contains the compound (C) as the component (B), the component (D), and the component (A). That is, the resist composition according to the seventh embodiment contains the component (B0), the component (D0), and the component (A0). The component (A0) may have any one or both of the constitutional unit (a0b) and the constitutional unit (a0d). It is preferable that the resist composition according to the seventh embodiment contain the component (B0), the component (A1-0), and the component (D0).

The resist composition according to the seventh embodiment may contain the component (E), the component (F), and the component (S) as other components.

In the resist composition according to the seventh embodiment, the content of each component can be set to the same as in the above-described first to third embodiments.

### (Resist Pattern Formation Method)

The resist pattern formation method according to the second aspect of the present invention is a method including a step of forming a resist film on a support using the resist composition according to the first aspect described above, a step of exposing the resist film to light, and a step of developing the resist film exposed to light to form a resist pattern.

According to the embodiment of the resist pattern formation method, a resist pattern formation method by performing processes as described below is an exemplary example.

First, a support is coated with the resist composition of the present embodiment using a spinner or the like, and a bake (post applied bake (PAB)) treatment is performed under a temperature condition of 80° C. to 150° C. for 40 to 120 seconds and preferably 60 to 90 seconds to form a resist film.

Following the selective exposure carried out on the resist film by, for example, exposure through a mask (mask pattern) having a predetermined pattern formed on the mask by using an exposure apparatus such as an electron beam lithography apparatus or an ArF exposure apparatus, or direct irradiation of the resist film for drawing with an electron beam without using a mask pattern, baking treatment (post-exposure baking (PEB)) is carried out, for example, under a temperature condition in a range of 80° C. to 150° C. for 40 to 120 seconds and preferably 60 to 90 seconds.

Next, the resist film is subjected to a developing treatment. The developing treatment is conducted using an alkali developing solution in a case of an alkali developing process and using a developing solution containing an organic solvent (organic developing solution) in a case of a solvent developing process.

After the developing treatment, it is preferable to conduct a rinse treatment. As the rinse treatment, water rinsing using pure water is preferable in a case of the alkali developing process, and rinsing using a rinse solution containing an organic solvent is preferable in a case of the solvent developing process.

In a case of the solvent developing process, after the developing treatment or the rinse treatment, the developing solution or the rinse solution attached onto the pattern may be removed by a treatment using a supercritical fluid.

After the developing treatment or the rinse treatment, drying is conducted. As desired, a bake treatment (post bake) may be conducted after the developing treatment.

In this manner, a resist pattern can be formed.

The support is not particularly limited and a known support of the related art can be used, and examples thereof include a substrate for an electronic component and a substrate on which a predetermined wiring pattern has been formed. Specific examples thereof include a metal substrate such as a silicon wafer, copper, chromium, iron, or aluminum; and a glass substrate. As the materials of the wiring pattern, copper, aluminum, nickel, or gold can be used.

Further, as the support, any one of the above-described supports provided with an inorganic and/or organic film on the above-described substrate may be used. As the inorganic film, an inorganic antireflection film (inorganic BARC) can be used. As the organic film, an organic film such as an organic antireflection film (organic BARC) or a lower-layer organic film used in a multilayer resist method can be used.

Here, the multilayer resist method is a method of providing at least one layer of an organic film (lower-layer organic film) and at least one layer of a resist film (upper-layer resist film) on a substrate and performing patterning of the lower-layer organic film using a resist pattern formed on the upper-layer resist film as a mask, and this method is considered to enable formation of a pattern with a high aspect ratio. That is, according to the multilayer resist method, since a desired thickness can be ensured by the lower-layer organic film, the thickness of the resist film can be reduced, and a fine pattern with a high aspect ratio can be formed.

The multilayer resist method is basically classified into a method in which a double-layer structure consisting of an upper-layer resist film and a lower-layer organic film is formed (two-layer resist method), and a method in which a multilayer structure having three or more layers consisting of an upper-layer resist film, a lower-layer organic film, and one or more intermediate layers (thin metal film and the like) provided between the upper-layer resist film and the lower-layer organic film (three-layer resist method).

The wavelength used for light exposure is not particularly limited and the exposure can be conducted using radiation such as an ArF excimer laser, a KrF excimer laser, an F₂ excimer laser, extreme ultraviolet rays (EUV), vacuum ultraviolet rays (VUV), electron beams (EB), X-rays, and soft X-rays. The resist composition is highly useful for a KrF excimer laser, an ArF excimer laser, EB, or EUV, and is more useful for an ArF excimer laser.

The exposure of the resist film to light can be a general exposure (dry exposure) conducted in air or an inert gas such as nitrogen, or liquid immersion exposure (liquid immersion lithography).

The liquid immersion lithography is an exposure method in which the region between the resist film and the lens at the lowermost position of the exposure apparatus is pre-filled with a solvent (liquid immersion medium) that has a larger refractive index than the refractive index of air, and the exposure (immersion exposure) is carried out in this state.

As the liquid immersion medium, a solvent which has a refractive index larger than the refractive index of air but smaller than the refractive index of the resist film to be exposed to light is preferable. The refractive index of such a solvent is not particularly limited as long as the refractive index is in the above-described range. Examples of the solvent which has a refractive index that is larger than the refractive index of air but smaller than the refractive index of the resist film include water, a fluorine-based inert liquid, a silicon-based solvent, and a hydrocarbon-based solvent.

Specific examples of the fluorine-based inert liquid include a liquid containing a fluorine-based compound such as C₃HCl₂F₅, C₄F₉OCH₃, C₄F₉OC₂H₅, or C₅H₃F₇ as a main component, and a liquid with a boiling point of 70° C. to 180° C. is preferable and a liquid with a boiling point of 80° C. to 160° C. is more preferable. A fluorine-based inert liquid having a boiling point in the above-described range is preferable from the viewpoint that a medium used for liquid immersion can be removed using a simple method after completion of light exposure.

As the fluorine-based inert liquid, a perfluoroalkyl compound in which all hydrogen atoms in the alkyl group have been substituted with fluorine atoms is particularly preferable. Specific examples of the perfluoroalkyl compound include a perfluoroalkylether compound and a perfluoroalkylamine compound.

Further, specific examples of the perfluoroalkylether compound include perfluoro(2-butyl-tetrahydrofuran) (boiling point of 102° C.), and specific examples of the perfluoroalkylamine compound include perfluorotributylamine (boiling point of 174° C.).

As the liquid immersion medium, water is preferable from the viewpoints of the cost, the safety, the environmental issues, and the versatility.

As the alkali developing solution used for the developing treatment in the alkali developing process, a 0.1 to 10 mass % tetramethylammonium hydroxide (TMAH) aqueous solution is an exemplary example.

The organic solvent contained in the organic developing solution used for the developing treatment in the solvent developing process may be any solvent that is capable of dissolving the component (A) (the component (A) before light exposure) and can be appropriately selected from known organic solvents. Specific examples thereof include a polar solvent such as a ketone-based solvent, an ester-based solvent, an alcohol-based solvent, a nitrile-based solvent, an amide-based solvent, and an ether-based solvent, and a hydrocarbon-based solvent.

The ketone-based solvent is an organic solvent containing C—C(═O)—C in the structure thereof. The ester-based solvent is an organic solvent containing C—C(═O)—O—C in the structure thereof. The alcohol-based solvent is an organic solvent containing an alcoholic hydroxyl group in the structure thereof. The term “alcoholic hydroxyl group” indicates a hydroxyl group bonded to a carbon atom of an aliphatic hydrocarbon group. The nitrile-based solvent is an organic solvent containing a nitrile group in the structure thereof. The amide-based solvent is an organic solvent containing an amide group in the structure thereof. The ether-based solvent is an organic solvent containing C—O—C in the structure thereof.

Some organic solvents have a plurality of the functional groups which characterize each of the solvents in the structure thereof. In such a case, the organic solvents are considered to correspond to all the solvents containing the functional groups. For example, diethylene glycol monomethylether corresponds to both the alcohol-based solvent and the ether-based solvent which have been classified above.

The hydrocarbon-based solvent is a hydrocarbon solvent which is formed of a hydrocarbon that may be halogenated and does not have a substituent other than halogen atoms. Among these, a fluorine atom is preferable as the halogen atom.

Among the examples, as the organic solvent contained in the organic developing solution, a polar solvent is preferable. Further, a ketone-based solvent, an ester-based solvent, and a nitrile-based solvent are preferable.

Examples of the ketone-based solvent include 1-octanone, 2-octanone, 1-nonanone, 2-nonanone, acetone, 4-heptanone, 1-hexanone, 2-hexanone, diisobutyl ketone, cyclohexanone, methylcyclohexanone, phenylacetone, methyl ethyl ketone, methyl isobutyl ketone, acetylacetone, acetonylacetone, ionone, diacetonylalcohol, acetylcarbinol, acetophenone, methyl naphthyl ketone, isophorone, propylenecarbonate, γ-butyrolactone, and methyl amyl ketone (2-heptanone). Among these examples, methyl amyl ketone (2-heptanone) is preferable as the ketone-based solvent.

Examples of the ester-based solvent include methyl acetate, butyl acetate, ethyl acetate, isopropyl acetate, amyl acetate, isoamyl acetate, ethyl methoxyacetate, ethyl ethoxyacetate, ethylene glycol monoethyl ether acetate, ethylene glycol monopropyl ether acetate, ethylene glycol monobutyl ether acetate, ethylene glycol monophenyl ether acetate, diethylene glycol monomethyl ether acetate, diethylene glycol monopropyl ether acetate, diethylene glycol monophenyl ether acetate, diethylene glycol monobutyl ether acetate, diethylene glycol monoethyl ether acetate, 2-methoxybutyl acetate, 3-methoxybutyl acetate, 4-methoxybutyl acetate, 3-methyl-3-methoxybutyl acetate, 3-ethyl-3-methoxybutyl acetate, propylene glycol monomethyl ether acetate, propylene glycol monoethyl ether acetate, propylene glycol monopropyl ether acetate, 2-ethoxybutyl acetate, 4-ethoxybutyl acetate, 4-propoxybutyl acetate, 2-methoxypentyl acetate, 3-methoxypentyl acetate, 4-methoxypentyl acetate, 2-methyl-3-methoxypentyl acetate, 3-methyl-3-methoxypentyl acetate, 3-methyl-4-methoxypentyl acetate, 4-methyl-4-methoxypentyl acetate, propylene glycol diacetate, methyl formate, ethyl formate, butyl formate, propyl formate, ethyl lactate, butyl lactate, propyl lactate, ethyl carbonate, propyl carbonate, butyl carbonate, methyl pyruvate, ethyl pyruvate, propyl pyruvate, butyl pyruvate, methyl acetoacetate, ethyl acetoacetate, methyl propionate, ethyl propionate, propyl propionate, isopropyl propionate, methyl 2-hydroxypropionate, ethyl 2-hydroxypropionate, methyl-3-methoxypropionate, ethyl-3-methoxypropionate, ethyl-3-ethoxypropionate, and propyl-3-methoxypropionate. Among these examples, butyl acetate is preferable as the ester-based solvent.

Examples of the nitrile-based solvent include acetonitrile, propionitrile, valeronitrile, and butyronitrile.

Known additives can be blended into the organic developing solution as necessary. Examples of the additive include a surfactant. The surfactant is not particularly limited, and for example, an ionic or non-ionic fluorine-based and/or silicon-based surfactant can be used. As the surfactant, a non-ionic surfactant is preferable, and a non-ionic fluorine-based surfactant or a non-ionic silicon-based surfactant is more preferable.

In a case where a surfactant is blended into the solution, the amount of the surfactant to be blended is typically in a range of 0.001% to 5% by mass, preferably in a range of 0.005% to 2% by mass, and more preferably in a range of 0.01% to 0.5% by mass with respect to the total amount of the organic developing solution.

The developing treatment can be performed according to a known developing method, and examples thereof include a method of immersing a support in a developing solution for a certain time (a dip method), a method of raising a developing solution on the surface of a support using the surface tension and maintaining the state for a certain time (a puddle method), a method of spraying a developing solution to the surface of a support (spray method), and a method of continuously ejecting a developing solution onto a support rotating at a certain rate while scanning a developing solution ejection nozzle at a certain rate (dynamic dispense method).

As the organic solvent contained in the rinse solution used for the rinse treatment after the developing treatment in the solvent developing process, a solvent that is unlikely to dissolve a resist pattern can be appropriately selected from the organic solvents described as the organic solvent used in the organic developing solution and then used. Typically, at least one solvent selected from a hydrocarbon-based solvent, a ketone-based solvent, an ester-based solvent, an alcohol-based solvent, an amide-based solvent, and an ether-based solvent is used. Among these, at least one solvent selected from a hydrocarbon-based solvent, a ketone-based solvent, an ester-based solvent, an alcohol-based solvent, and an amide-based solvent is preferable, at least one solvent selected from an alcohol-based solvent and an ester-based solvent is more preferable, and an alcohol-based solvent is particularly preferable.

As the alcohol-based solvent used in the rinse solution, a monohydric alcohol having 6 to 8 carbon atoms is preferable, and the monohydric alcohol may be linear, branched, or cyclic. Specific examples thereof include 1-hexanol, 1-heptanol, 1-octanol, 2-hexanol, 2-heptanol, 2-octanol, 3-hexanol, 3-heptanol, 3-octanol, 4-octanol, and benzyl alcohol. Among these, 1-hexanol, 2-heptanol, and 2-hexanol are preferable, and 1-hexanol and 2-hexanol are more preferable.

These organic solvents may be used alone or in combination of two or more kinds thereof. Further, an organic solvent other than the above-described solvents and water may be mixed and used. However, in consideration of the development characteristics, the amount of water to be blended into the rinse solution is preferably 30% by mass or less, more preferably 10% by mass or less, still more preferably 5% by mass or less, and particularly preferably 3% by mass or less with respect to the total amount of the rinse solution.

A known additive can be blended into the rinse solution as necessary. Examples of the additive include a surfactant. As the surfactant, the same surfactants as those described above are exemplary examples. Among these, a non-ionic surfactant is preferable, and a non-ionic fluorine-based surfactant or a non-ionic silicon-based surfactant is more preferable.

In a case where a surfactant is blended into the solution, the amount of the surfactant to be blended is typically in a range of 0.001% to 5% by mass, preferably in a range of 0.005% to 2% by mass, and more preferably in a range of 0.01% to 0.5% by mass with respect to the total amount of the rinse solution.

The rinse treatment carried out using a rinse solution (washing treatment) can be performed according to a known rinse method. Examples of the method of performing the rinse treatment include a method of continuously ejecting a rinse solution onto a support rotating at a certain rate (rotary coating method), a method of immersing a support in a rinse solution for a certain time (dip method), and a method of spraying a rinse solution to the surface of a support (spray method).

According to the resist pattern formation method of the present embodiment described above, since the resist composition of the first aspect described above is used, a resist pattern with reduced defects can be formed.

### (Compound)

The compound according to the third aspect of the present invention is a compound represented by General Formula (M0) (hereinafter, also referred to as “compound (M0)”).

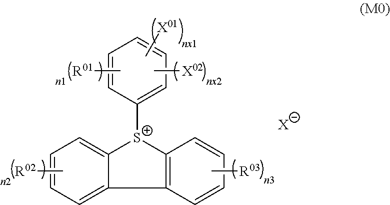

[In the formula, X⁰¹ represents an electron-withdrawing group, X⁰² represents an electron-withdrawing group different from X⁰¹, and R⁰¹ represents an electron-donating group. R⁰² and R⁰³ each independently represents a substituent. nx1 and nx2 each independently represents an integer of 1 to 4, n1 represents an integer of 0 to 3, and nx1+nx2+n1≤5 is satisfied. n2 and n3 each independently represents an integer of 0 to 4. In a case where nx1 represents 2 or greater, a plurality of X¹'s may be the same as or different from each other. In a case where nx2 represents 2 or greater, a plurality of X⁰²'s may be the same as or different from each other. In a case where n1 represents 2 or greater, a plurality of R⁰¹'s may be the same as or different from each other. In a case where n2 represents 2 or greater, a plurality of R⁰²'s may be the same as or different from each other. In a case where n3 represents 2 or greater, a plurality of R⁰³'s may be the same as or different from each other. X⁻ represents a counter anion.]

### {Cation Moiety}

The cation moiety is the same as the cation (C0) represented by General Formula (c0).

### {Anion Moiety}

X⁻ represents a counter anion. Examples of X⁻ include those described as the anion moiety of the compound represented by General Formula (b0), those described as the anion moiety of the compound represented by General Formula (d0), and those described as the anion moiety of the constitutional unit represented by General Formula (a0-1).

### <Method of Producing Compound>

The compound (M0) can be produced by a known method.

Examples of a method of producing the compound (M0) include a production method including a first step and a second step described below.

As the compound used in each step, a commercially available product or a compound obtained by synthesis may be used.

As a solvent in each reaction of the first step and the second step, a solvent which can dissolve the compounds used in each step and does not react with the compounds can be used. Examples of the solvent include dichloromethane, dichloroethane, chloroform, tetrahydrofuran, N,N-dimethylformamide, dimethylacetamide, dimethyl sulfoxide, acetonitrile, and propionitrile.

### First Step:

In the first step, a compound (pre) is obtained by mixing a compound (I), a compound (II), and TMS-X″ in a solvent and carrying out a reaction.

Examples of the solvent in the first step include tetrahydrofuran.

The reaction temperature may be, for example, in a range of 0° C. to 50° C. and is preferably in a range of 10° C. to 40° C. The reaction time can be appropriately set according to the reactivity, the amount, and the like of the compound to be used. The reaction time may be, for example, 10 minutes or longer and 24 hours or shorter and is preferably 10 minutes or longer and 12 hours or shorter.

After any reaction time, the reaction may be stopped by adding water to the reaction solution. After completion of the reaction, the compound (pre) may be purified by solvent extraction, vacuum concentration, crystallization, or the like. Examples of the solvent used for the solvent extraction include dichloromethane. Examples of the solvent used for crystallization include a mixed solvent of dichloromethane and tert-butyl ethyl ether.

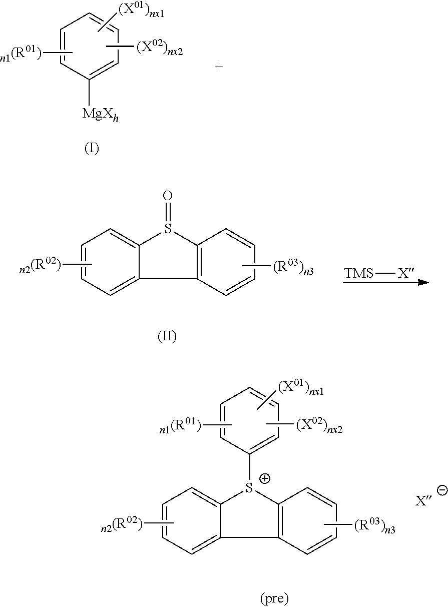

[In the formulae, X⁰¹, X⁰², R⁰¹ to R⁰³, nx1, nx2, and n1 to n3 each is the same as in Formula (M0). Xₕ represents a halogen atom. TMS represents a trimethylsilyl group. X″⁻ represents a counter anion in the compound (pre).]

Examples of the halogen atom as Xₕ in the reaction formula include a fluorine atom, a chlorine atom, a bromine atom, and an iodine atom.

Examples of the counter anion as X″⁻ include Br⁻, Cl⁻, and CF₃SO₃⁻. Examples of the compound represented by TMS-X″ include trimethylsilyl trifluoromethanesulfonate (TMSOTf).

### Second Step:

In the second step, a compound (M0) is obtained by mixing the compound (pre) and a compound (III) for salt exchange in a solvent to carry out a salt exchange reaction. Examples of the solvent in the second step include dichloromethane.

The reaction temperature may be, for example, in a range of 0° C. to 100° C. and is preferably in a range of 0° C. to 50° C. The reaction time can be appropriately set according to the reactivity, the amount, and the like of the compound to be used. The reaction time may be, for example, 10 minutes or longer and 24 hours or shorter and is preferably 10 minutes or longer and 12 hours or shorter.

After completion of the salt exchange reaction, the compound (M0) may be isolated and/or purified. Known methods can be used for the isolation and/or purification of the compound (M0). Examples of the isolation and/or purification method include concentration, solvent extraction, distillation, crystallization, recrystallization, and chromatography, and a combination of these methods can be used as appropriate.

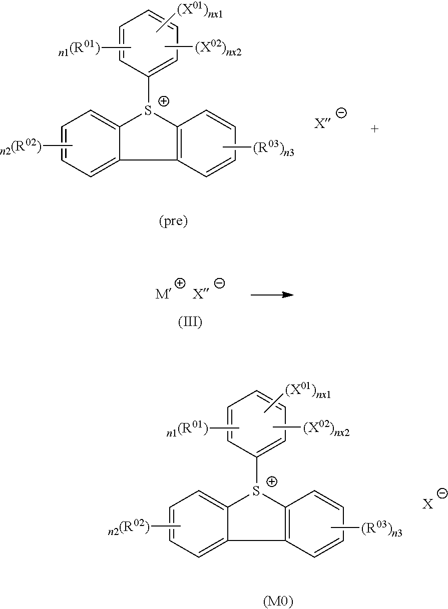

[In the formula, X⁰¹, X⁰², R⁰¹ to R⁰³, nx1, nx2, n1 to n3, and X⁻ each is the same as in Formula (M0). X″ represents a counter anion in the compound (pre). M′⁺ represents an ammonium cation.]

In the reaction formula, examples of the ammonium cation as M′⁺ include organic ammonium cations. Among these, a quaternary ammonium cation is preferable. The ammonium cation as M′⁺ can be appropriately selected depending on the counter anion in the compound (III).

The structure of the compound obtained as described above can be identified by typical organic analysis methods such as ¹H-nuclear magnetic resonance (NMR) spectroscopy, ¹³C-NMR spectroscopy, ¹⁹F-NMR spectroscopy, infrared (IR) absorption spectroscopy, mass spectrometry (MS), elemental analysis, and X-ray crystal diffraction.

Specific examples of the compound (M0) include the same compounds described as the specific examples of the compound (B0) or the compound (D0).

### (Acid-Generating Agent)

The acid-generating agent according to a fourth aspect of the present invention contains a compound represented by General Formula (B0).

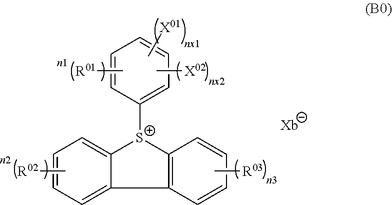

[In the formula, X⁰¹ represents an electron-withdrawing group, X⁰² represents an electron-withdrawing group different from X⁰¹, and R⁰¹ represents an electron-donating group. R⁰² and R⁰³ each independently represents a substituent. nx1 and nx2 each independently represents an integer of 1 to 4, n1 represents an integer of 0 to 3, and nx1+nx2+n1≤5 is satisfied. n2 and n3 each independently represents an integer of 0 to 4. In a case where nx1 represents 2 or greater, a plurality of X⁰¹'s may be the same as or different from each other. In a case where nx2 represents 2 or greater, a plurality of X⁰²'s may be the same as or different from each other. In a case where n1 represents 2 or greater, a plurality of R⁰¹'s may be the same as or different from each other. In a case where n2 represents 2 or greater, a plurality of R⁰²'s may be the same as or different from each other. In a case where n3 represents 2 or greater, a plurality of R⁰³'s may be the same as or different from each other. Xb⁻ represents a counter anion.]

### {Cation Moiety}

The cation moiety is the same as the cation (C0) represented by General Formula (c0).

### {Anion Moiety}

Xb⁻ represents a counter anion. Xb⁻ is the same as that for Xb⁻ in General Formula (b0).

Specific examples of the compound represented by General Formula (B0) include the same compounds described as the specific examples of the compound (B0).

The acid-generating agent of the present embodiment contains a compound represented by General Formula (B0). The cation moiety of the compound is a planar Low LUMO cation and has high acid generation efficiency. Therefore, the sensitivity of a resist composition containing the acid-generating agent of the present embodiment is improved.

Further, since the compound has a phenyl group containing two or more electron-withdrawing groups in the cation moiety, the solubility in the component (S) is improved in a case where the compound is used in a resist composition. Further, the solubility in a developing solution is improved in the development after light exposure. In this manner, the resist composition containing the acid-generating agent according to the present embodiment can reduce defects.

As described above, according to the acid-generating agent of the present embodiment, both high sensitivity of the resist composition and reduction of defects can be achieved.

### (Acid Diffusion Control Agent)

The acid diffusion control agent according to a fifth aspect of the present invention contains a compound represented by General Formula (D0).

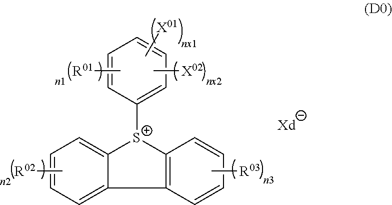

[In the formula, X⁰¹ represents an electron-withdrawing group, X⁰² represents an electron-withdrawing group different from X⁰¹, and R⁰¹ represents an electron-donating group. R⁰² and R⁰³ each independently represents a substituent. nx1 and nx2 each independently represents an integer of 1 to 4, n1 represents an integer of 0 to 3, and nx1+nx2+n1≤5 is satisfied. n2 and n3 each independently represents an integer of 0 to 4. In a case where nx1 represents 2 or greater, a plurality of X⁰¹'s may be the same as or different from each other. In a case where nx2 represents 2 or greater, a plurality of X⁰²'s may be the same as or different from each other. In a case where n1 represents 2 or greater, a plurality of R⁰¹'s may be the same as or different from each other. In a case where n2 represents 2 or greater, a plurality of R⁰²'s may be the same as or different from each other. In a case where n3 represents 2 or greater, a plurality of R⁰³'s may be the same as or different from each other. Xd⁻ represents a counter anion.]

### {Cation Moiety}

The cation moiety is the same as the cation (C0) represented by General Formula (c0).

### {Anion Moiety}

Xd⁻ represents a counter anion. Xd⁻ is the same as that for Xd⁻ in General Formula (d0).

Specific examples of the compound represented by General Formula (D0) include the same compounds described as the specific examples of the compound (D0).

The acid diffusion control agent according to the present embodiment contains a compound represented by General Formula (D0). The cation moiety of the compound is a planar Low LUMO cation, and is decomposed upon light exposure and easily loses the acid diffusion controllability. As a result, the resist composition containing the acid diffusion control agent of the present embodiment has improved sensitivity.

Further, since the compound has a phenyl group containing two or more electron-withdrawing groups in the cation moiety, the solubility in the component (S) is improved in a case where the compound is used in a resist composition. Further, the solubility in a developing solution is improved in the development after light exposure. In this manner, the resist composition containing the acid diffusion control agent according to the present embodiment can reduce defects.

As described above, according to the acid diffusion control agent of the present embodiment, both high sensitivity of the resist composition and reduction of defects can be achieved.

### (High-Molecular-Weight Compound)

The high-molecular-weight compound according to a sixth aspect of the present invention has a constitutional unit represented by General Formula (A0-1).

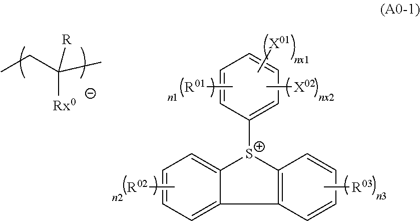

[In the formula, R represents a hydrogen atom, an alkyl group having 1 to 5 carbon atoms, or a halogenated alkyl group having 1 to 5 carbon atoms. Rx⁰ represents a group containing an anion. X⁰¹ represents an electron-withdrawing group, X⁰² represents an electron-withdrawing group different from X⁰¹, and R⁰¹ represents an electron-donating group. R⁰² and R⁰³ each independently represents a substituent. nx1 and nx2 each independently represents an integer of 1 to 4, n1 represents an integer of 0 to 3, and nx1+nx2+n1≤5 is satisfied. n2 and n3 each independently represents an integer of 0 to 4. In a case where nx1 represents 2 or greater, a plurality of X¹'s may be the same as or different from each other. In a case where nx2 represents 2 or greater, a plurality of X⁰²'s may be the same as or different from each other. In a case where n1 represents 2 or greater, a plurality of R⁰¹'s may be the same as or different from each other. In a case where n2 represents 2 or greater, a plurality of R⁰²'s may be the same as or different from each other. In a case where n3 represents 2 or greater, a plurality of R⁰³'s may be the same as or different from each other.]

### {Cation Moiety}

The cation moiety is the same as the cation (C0) represented by General Formula (c0).

### {Anion Moiety}

Rx⁰ represents a group containing an anion. Rx⁰ is the same as that for Rx⁰ in General Formula (a0-1).

Specific examples of the constitutional unit represented by General Formula (A0-1) include the same compounds described as the specific examples of the constitutional unit (a0).

Examples of the high-molecular-weight compound according to the present embodiment include the same compounds as those for the resin component (A-0) or the resin component (A1-0).

The high-molecular-weight compound according to the present embodiment has a constitutional unit represented by General Formula (A0-1). The cation moiety of the constitutional unit is a planar Low LUMO cation and is likely to be decomposed upon light exposure. Therefore, in a case where the high-molecular-weight compound has an acid-generating agent function, the high-molecular-weight compound has high acid generation efficiency. Further, in a case where the high-molecular-weight compound has an acid diffusion control agent function, the acid diffusion controllability is likely to be lost upon light exposure. Therefore, the sensitivity of the resist composition containing the high-molecular-weight compound according to the present embodiment is improved.

Further, since the constitutional unit has a phenyl group containing two or more electron-withdrawing groups in the cation moiety, the solubility in the component (S) is improved in a case where the constitutional unit is used in a resist composition. Further, the solubility in a developing solution is improved in the development after light exposure. In this manner, the resist composition containing the acid diffusion control agent according to the present embodiment can reduce defects.

As described above, according to the high-molecular-weight compound of the present embodiment, both high sensitivity of the resist composition and reduction of defects can be achieved.

### <Method of Producing High-Molecular-Weight Compound>

The high-molecular-weight compound according to the present embodiment can be obtained by polymerizing a monomer from which the constitutional unit (a0) is derived and, as necessary, a monomer from which other constitutional units (constitutional units (a1) to (a4), (a10), (st), and the like) are derived, for example, by a known method such as radical polymerization using a radical polymerization initiator such as azobisisobutyronitrile (AIBN).

Various materials that are used in the resist composition according to the above-described embodiment and the resist pattern forming method according to the above-described embodiment (for example, a resist solvent, a developing solution, a rinse liquid, a composition for forming an antireflection film, and a composition for forming a top coat) preferably do not contain impurities such as a metal, a metal salt containing halogen, an acid, an alkali, and a component containing a sulfur atom or phosphorus atom.

Here, examples of the impurities containing metal atoms include Na, K, Ca, Fe, Cu, Mn, Mg, Al, Cr, Ni, Zn, Ag, Sn, Pb, Li, and salts thereof. The content of the impurities contained in these materials is preferably 200 ppb or less, more preferably 1 ppb or less, still more preferably 100 parts per trillion (ppt) or less, particularly preferably 10 ppt or less, and most preferably substantially zero (less than or equal to the detection limit of the measuring device).

### EXAMPLES

Hereinafter, the present invention will be described in more detail based on examples, but the present invention is not limited to these examples.

### <Production of Compounds>

### Production Example B1

### Production Example B1-1

Magnesium (4.2 g) and tetrahydrofuran (19 g) were stirred at 50° C., and a solution of the compound (1) (38 g) in tetrahydrofuran (THF) (75 g) was added dropwise thereto at the same temperature. After completion of the dropwise addition, the mixture was stirred for 2 hours and cooled to room temperature, and tetrahydrofuran (75 g) was added thereto, thereby obtaining a THE solution (solution 1) of a compound (1′). Dibenzothiophene 5-oxide (10 g) and THE (75 g) were placed in another container, and the mixture was stirred at room temperature. Trimethylsilyl trifluoromethanesulfonate (TMSOTf) (63 g) and the solution 1 were added dropwise thereto. After completion of the dropwise addition, the reaction was continued at room temperature for 1 hour, and the reaction was completed. Thereafter, dichloromethane (100 g) and water (100 g) were added thereto, the mixture was stirred for 30 minutes, and the aqueous layer was removed. The organic layer was washed three times with ultrapure water (100 g) and concentrated under reduced pressure. The concentrated residues were crystallized with dichloromethane/tert-butyl methyl ether, thereby obtaining a precursor (pre-1) (6.2 g) as a white solid. In the following formula, “OTf⁻” represents trifluoromethanesulfonate.

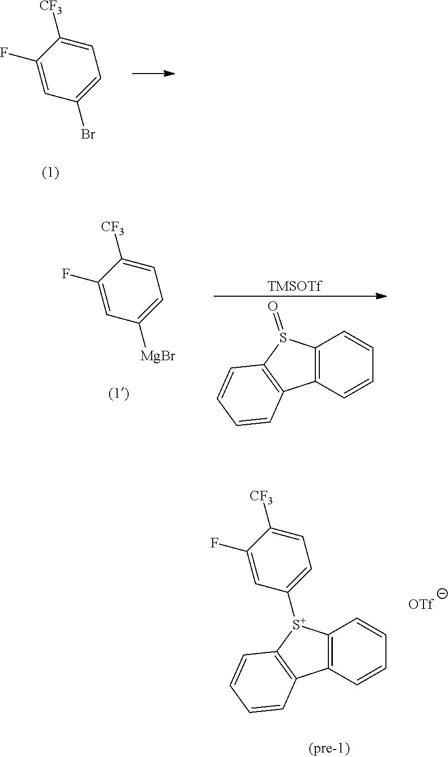

### Production Example B1-2

Dichloromethane (50 g) and water (50 g) were added to the precursor (pre-1) (6.2 g) and the compound (B-an1) (5.8 g), and the mixture was sufficiently stirred for liquid separation. The organic layer was washed with 1% hydrochloric acid (50 g) and washed three times with pure water (50 g). Diethyl ether (150 g) was added to the organic layer, and the precipitated crystals were collected by filtration under reduced pressure. The obtained crystals were dried under reduced pressure, thereby obtaining a compound (0-1) (17.1 g).

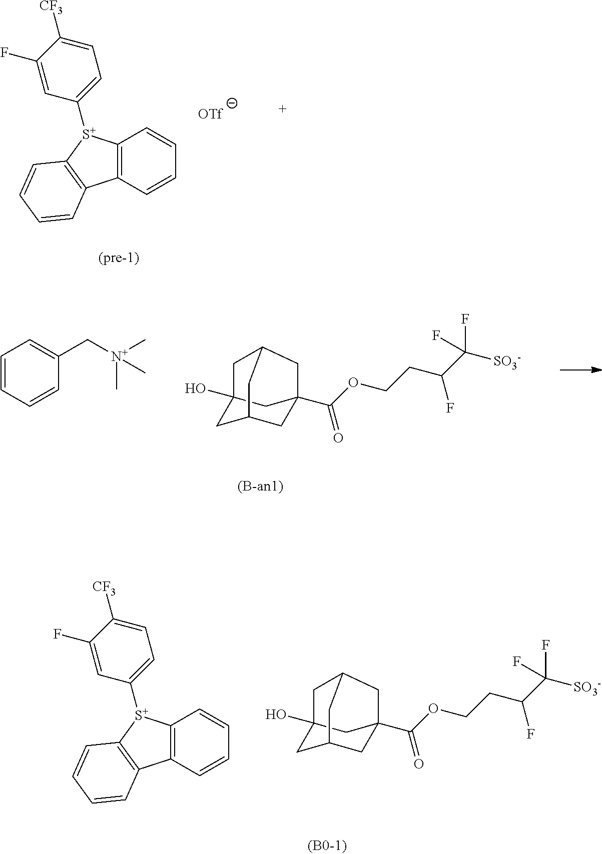

### Production Examples B2 to B12

Each of precursors (pre-2) to (pre-12) was obtained in the same manner as in Production Example B1-1 except that the compound (1) was changed to each of compounds (2) to (12).

Each of compounds (B0-2) to (B0-12) was obtained in the same manner as in Production Example B1-2 except that the precursor (pre-1) was changed to each of precursors (pre-2) to (pre-12).

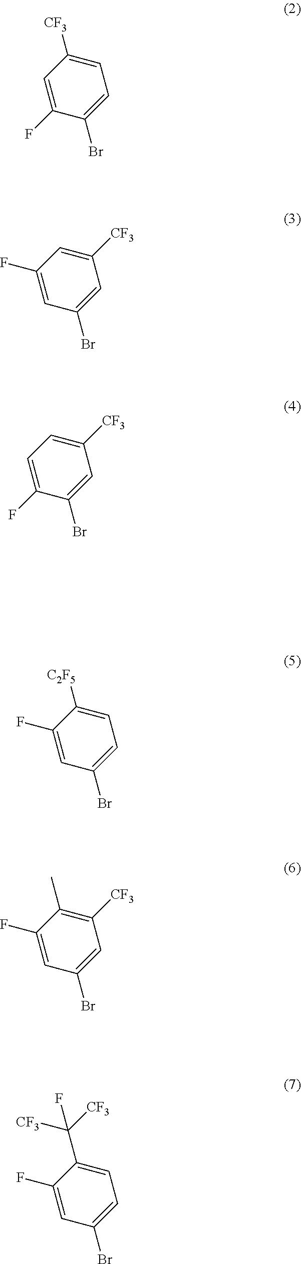

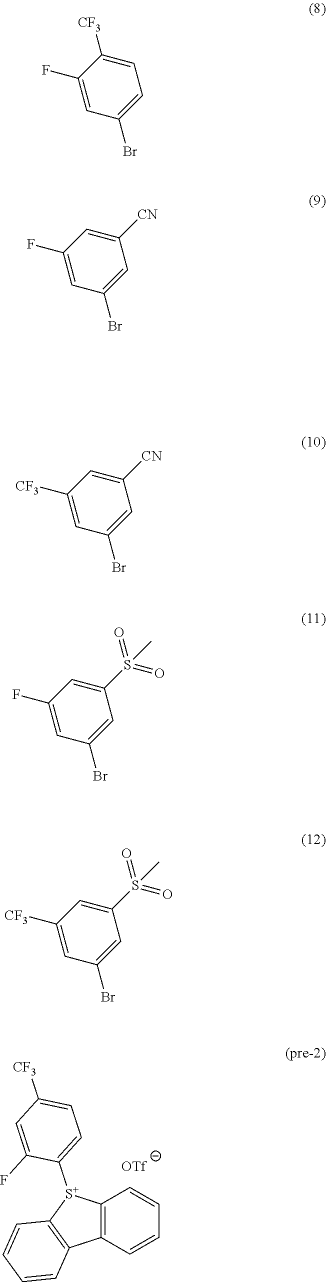 [SMILES: Fc1cc(Br)ccc1C(F)(F)F ; N#Cc1cc(F)cc(Br)c1 ; N#Cc1cc(Br)cc(C(F)(F)F)c1 ; CS(=O)(=O)c1cc(F)cc(Br)c1 ; CS(=O)(=O)c1cc(Br)cc(C(F)(F)F)c1 ; Fc1cc(C(F)(F)F)ccc1-[s+]1c2ccccc2c2ccccc21]

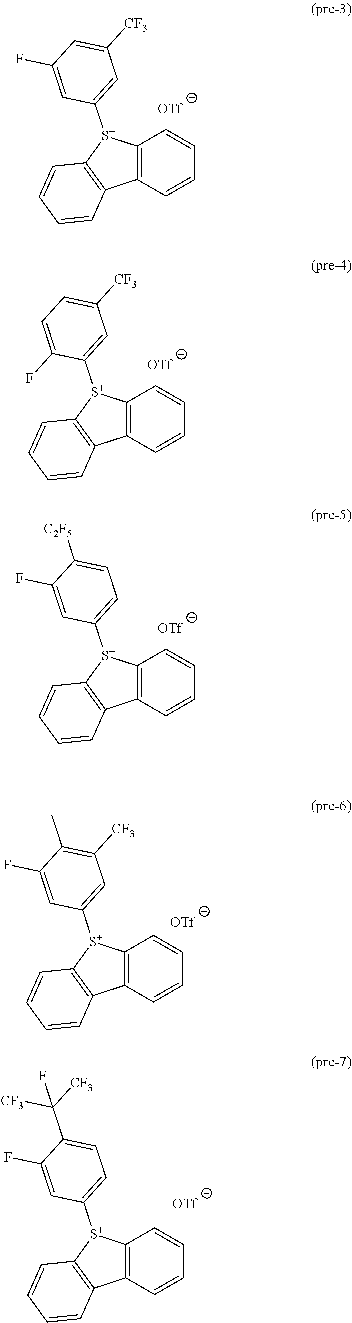

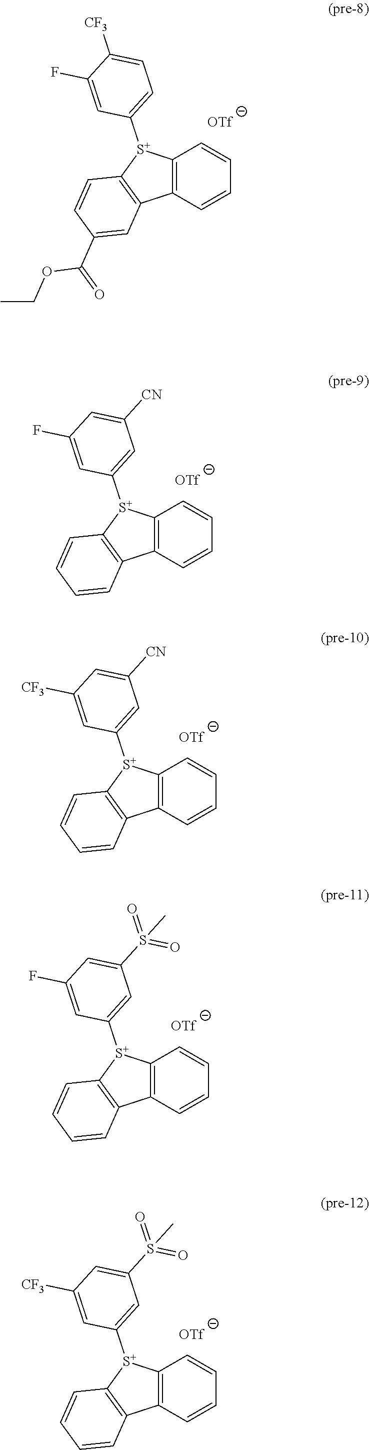 [SMILES: CCOC(=O)c1ccc2c(c1)c1ccccc1[s+]2-c1ccc(C(F)(F)F)c(F)c1 ; N#Cc1cc(F)cc(-[s+]2c3ccccc3c3ccccc32)c1 ; N#Cc1cc(-[s+]2c3ccccc3c3ccccc32)cc(C(F)(F)F)c1 ; CS(=O)(=O)c1cc(F)cc(-[s+]2c3ccccc3c3ccccc32)c1 ; CS(=O)(=O)c1cc(-[s+]2c3ccccc3c3ccccc32)cc(C(F)(F)F)c1]

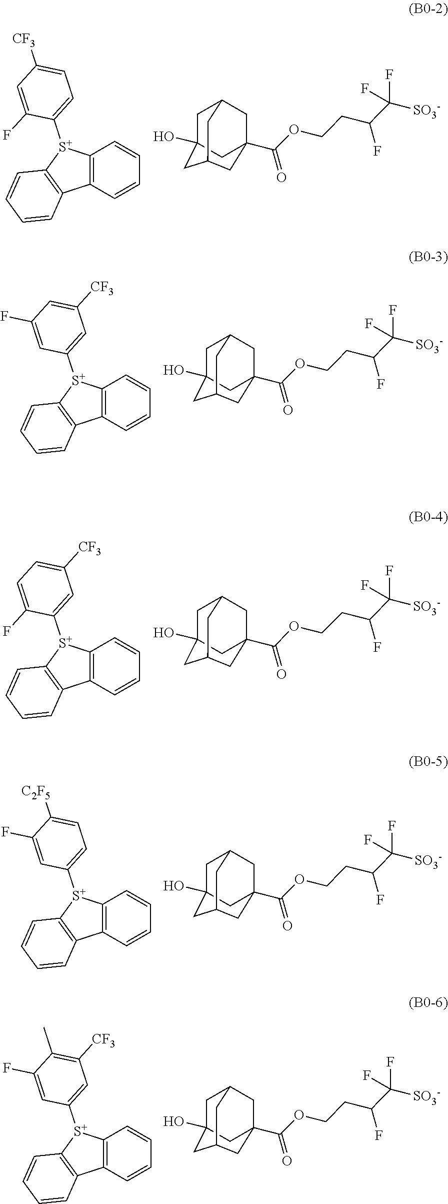

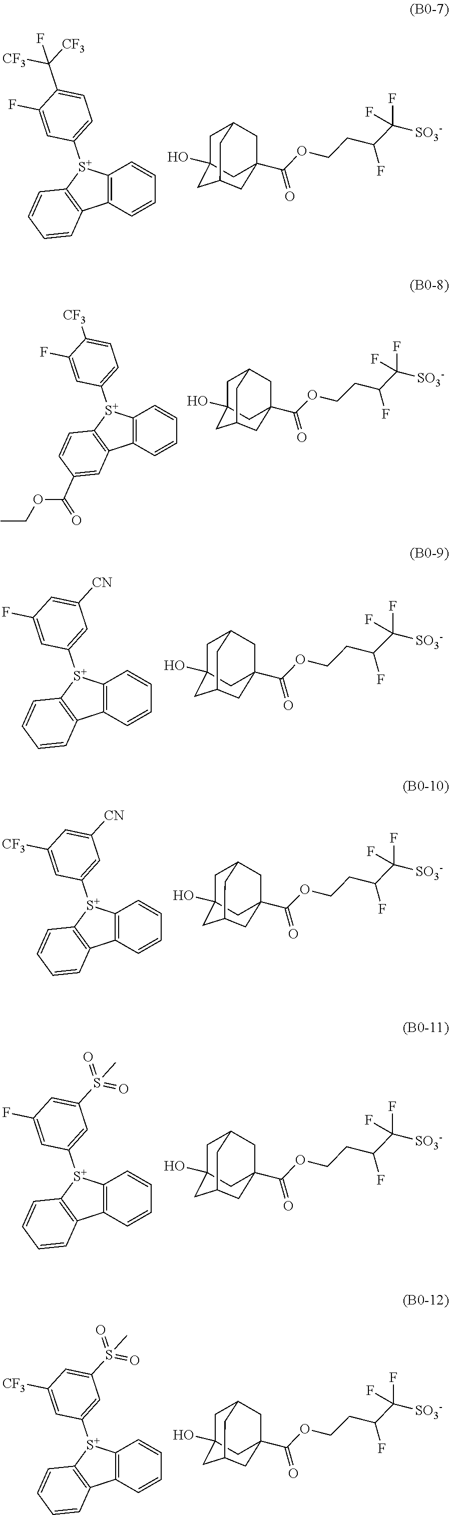

### Production Examples B13 to B16

Each of compounds (B0-13) to (B0-16) was obtained in the same manner as in Production Example B1 except that the compound (B-an1) was changed to each of compounds (B-an13) to (B-an16).

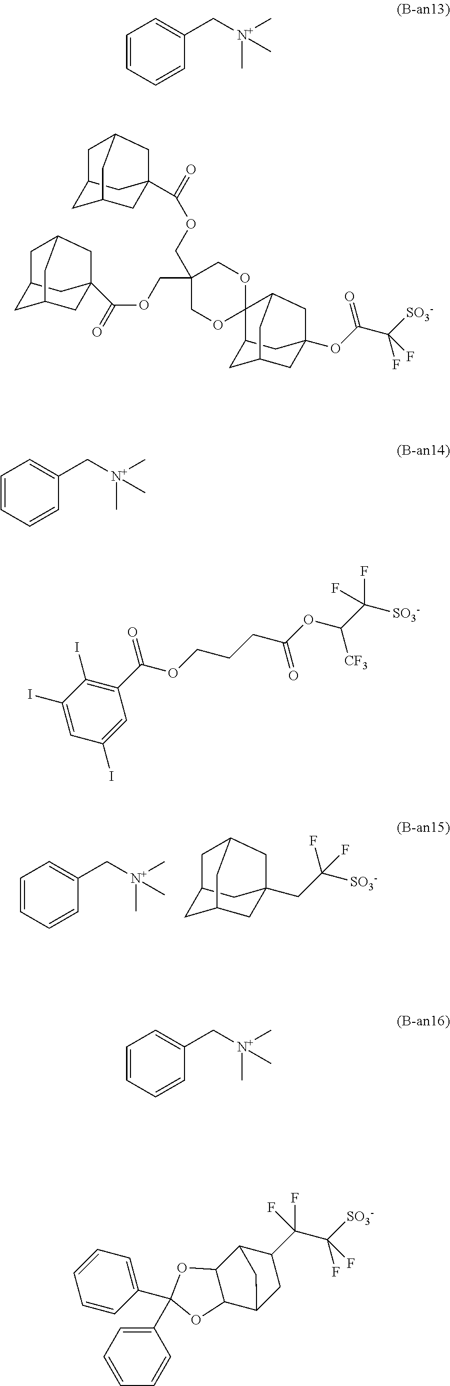

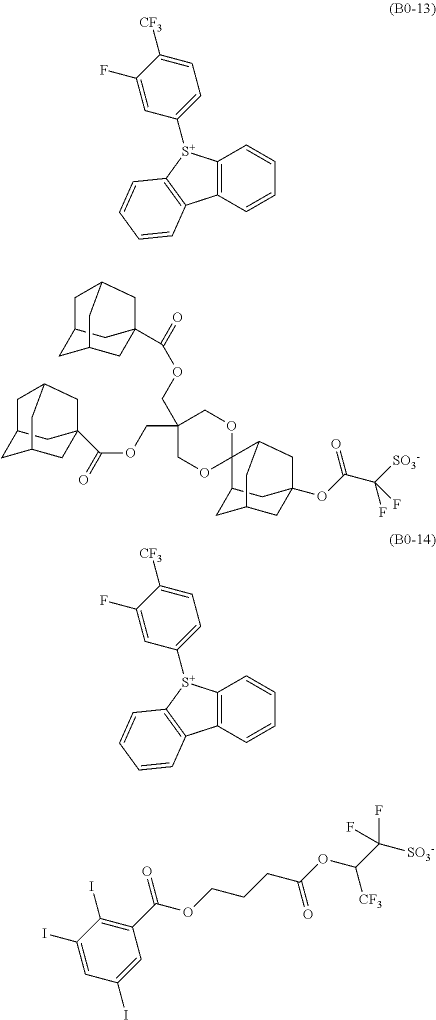

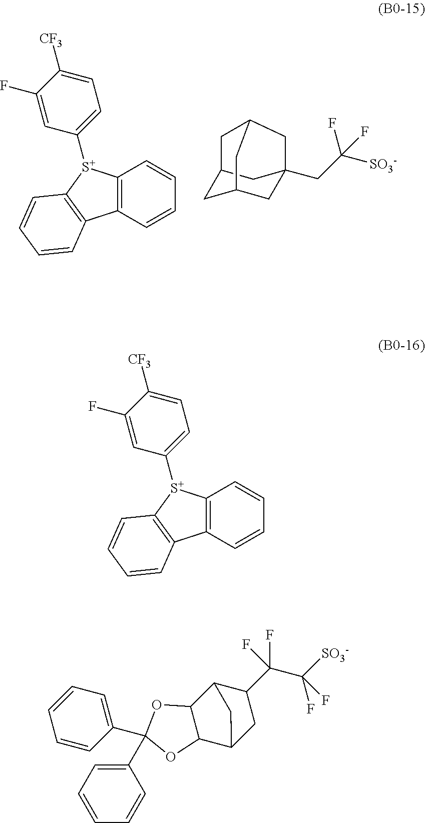

### Production Example D1

Dichloromethane (50 g) and water (50 g) were added to the precursor (pre-1) (36.2 g) and the compound (D-an1) (5.2 g), and the mixture was sufficiently stirred for liquid separation. The organic layer was washed with 1% hydrochloric acid (50 g) and washed three times with pure water (50 g). Diethyl ether (150 g) was added to the organic layer, and the precipitated crystals were collected by filtration under reduced pressure. The obtained crystals were dried under reduced pressure, thereby obtaining a compound (D0-1) (5.9 g).

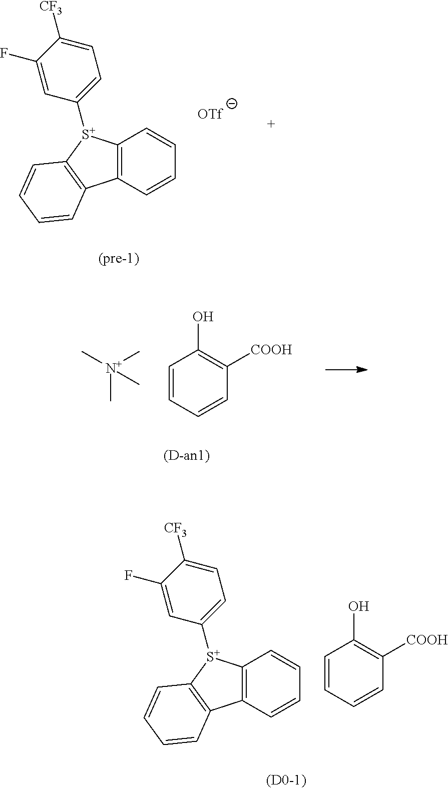

### Production Examples D2 to D12

Each of compounds (D0-2) to (D0-12) was obtained in the same manner as in Production Example D1 except that the precursor (pre-1) was changed to each of precursors (pre-2) to (pre-12).

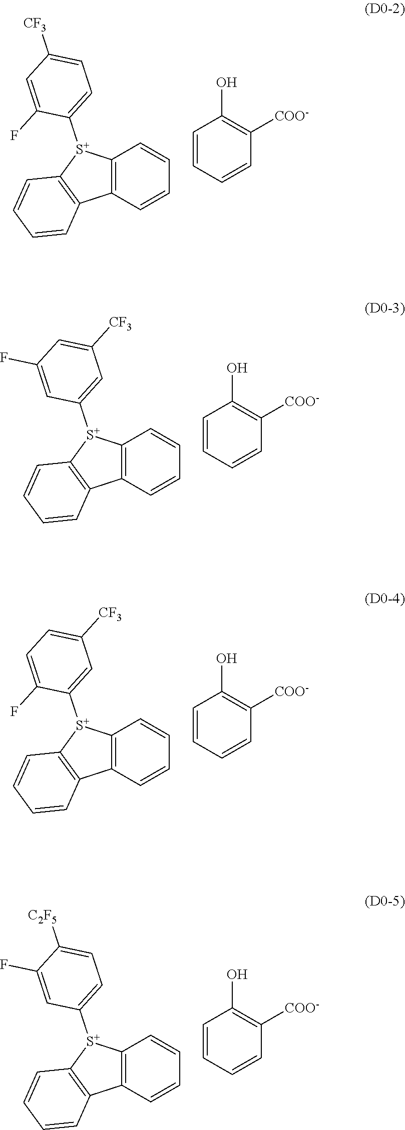

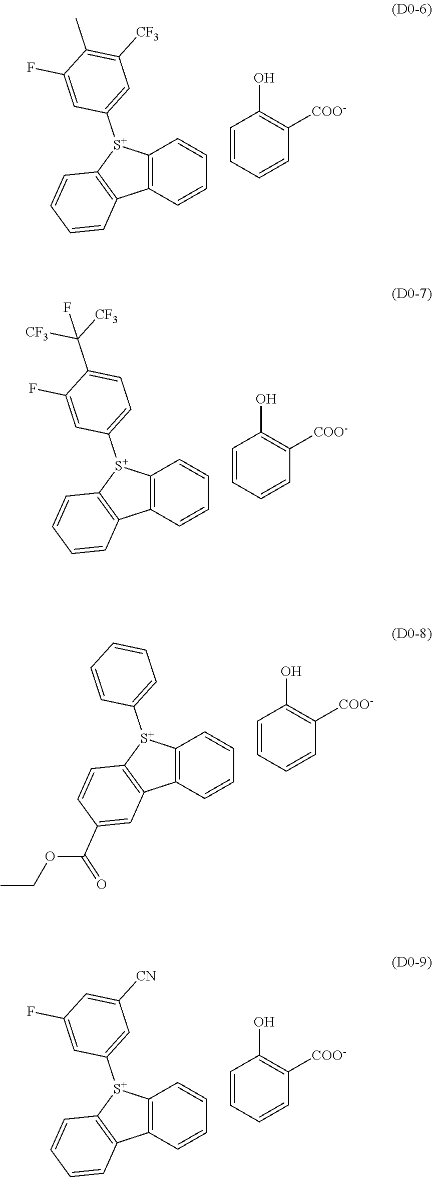

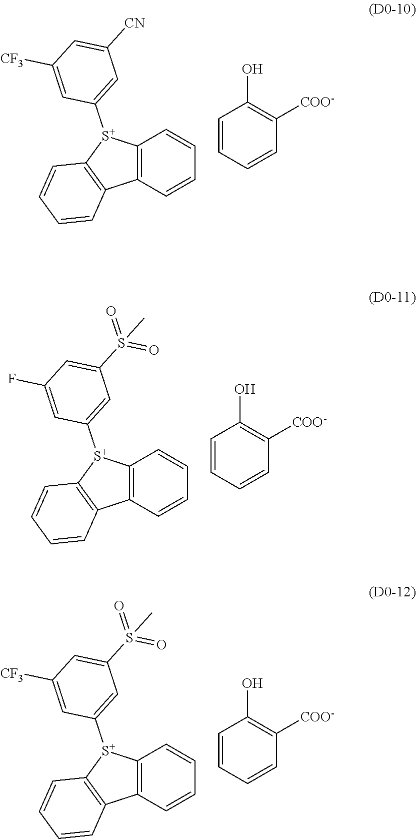

### Production Examples D13 and D14

Each of compounds (D0-13) to (D0-14) was obtained in the same manner as in Production Example D1 except that the compound (D-an1) was changed to each of compounds (D-an13) and (D-an14).

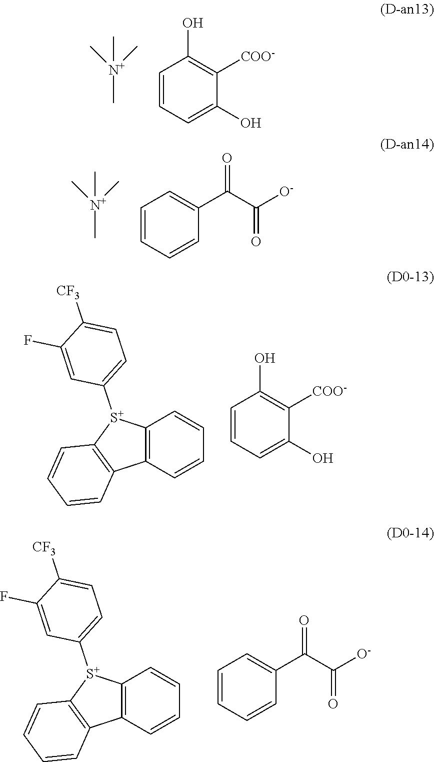

The NMR measurement results of the compounds (B0-1) to (B0-16) and the compounds (D0-1) to (D0-14) produced in each of the above-described production examples are listed in Tables 1 to 7.

TABLE 1

| | ¹H-NMR | ¹⁹F-NMR |
|---|---|---|
| Compound | (DMSO, 400 MHz) | (DMSO, 376 MHz) |
| B0-1 | 1.60-1.72 (2H, m), 1.79-2.06 (14H, m), 1.98 (2H, m), 2.01 (dd, | −56.4, −99.5, |
| | J = 13.3, 2.7 Hz), 1.95 (3H, m), 1.94 (d, J = 16.3 Hz), 1.86 (dd, | −111.8, −122.3, |
| | J = 13.0, 2.8 Hz), 1.83 (3H, m), 2.01 (sept, J = 2.8 Hz)), | −203.4 |
| | 4.29-4.37 (2H, m), 4.45 (1H, t, J = 7.4 Hz), 7.33-7.56 (5H, m), 7.54 | |
| | (dd, J = 8.2, 1.6 Hz)), 7.72-7.80 (3H, 7.77 (ddd, J = 7.7, 1.5, 0.5 | |
| | Hz), 7.72 (dd, J = 8.2, 0.5 Hz)), 8.02 (2H, ddd, J = 8.6, 1.5, 0.5 Hz) | |
| B0-2 | 1.60-1.72 (2H, m), 1.79-2.06 (14H, m), 1.98 (2H, m), 2.01 (dd, | −54.2, −98.5, |
| | J = 13.3, 2.7 Hz), 1.95 (3H, m), 1.94 (d, J = 16.3 Hz), 1.86 (dd, | −111.8, −122.3, |
| | J = 13.0, 2.8 Hz), 1.83 (3H, m), 2.01 (sept, J = 2.8 Hz)), | −203.4 |
| | 4.29-4.37 (2H, m), 4.45 (1H, t, J = 7.4 Hz),, 7.27 (1H, dd, J = 8.2, | |
| | 1.7 Hz), 7.33-7.51 (5H, 7.46 (ddd, J = 8.6, 8.4, 1.5 Hz), 7.38 | |
| | (ddd, J = 8.4, 7.7, 1.5 Hz), 7.44 (dd, J = 8.2, 0.4 Hz)), 7.58 (1H, | |
| | dd, J = 1.7, 0.4 Hz), 7.76 (2H, ddd, J = 7.7, 1.5, 0.5 Hz), 8.02 | |
| | (2H, ddd, J = 8.6, 1.5, 0.5 Hz) | |
| B0-3 | 1.60-1.72 (2H, m), 1.79-2.06 (14H, m), 1.98 (2H, m), 2.01 (dd, | −58.1, −101.2, |
| | J = 13.3, 2.7 Hz), 1.95 (3H, m), 1.94 (d, J = 16.3 Hz), 1.86 (dd, | −111.8, −122.3, |
| | J = 13.0, 2.8 Hz), 1.83 (3H, m), 2.01 (sept, J = 2.8 Hz)), | −203.4 |
| | 4.29-4.37 (2H, m), 4.45 (1H, t, J = 7.4 Hz),, 7.20-7.25 (2H, 7.24 (dd, | |
| | J = 1.9, 1.6 Hz), 7.21 (dd, J = 2.2, 1.6 Hz)), 7.33-7.50 (4H, 7.45 | |
| | (ddd, J = 8.6, 8.4, 1.5 Hz), 7.38 (ddd, J = 8.4, 7.7, 1.5 Hz)), 7.56 | |
| | (1H, dd, J = 2.2, 1.9 Hz), 7.77 (2H, ddd, J = 7.7, 1.5, 0.5 Hz), | |
| | 8.01 (2H, ddd, J = 8.6, 1.5, 0.5 Hz) | |
| B0-4 | 1.60-1.72 (2H, m), 1.79-2.06 (14H, m), 1.98 (2H, m), 2.01 (dd, | −59.8, −103.5, |
| | J = 13.3, 2.7 Hz), 1.95 (3H, m), 1.94 (d, J = 16.3 Hz), 1.86 (dd, | −111.8, −122.3, |
| | J = 13.0, 2.8 Hz), 1.83 (3H, m), 2.01 (sept, J = 2.8 Hz)), | −203.4 |
| | 4.29-4.37 (2H, m), 4.45 (1H, t, J = 7.4 Hz),, 4.45 (1H, t, J = 7.4 | |
| | Hz)7.15 (1H, dd, J = 1.6, 0.5 Hz), 7.24 (1H, dd, J = 8.4, 1.6 Hz), | |
| | 7.33-7.51 (4H, 7.46 (ddd, J = 8.6, 8.4, 1.5 Hz), 7.37 (ddd, J = | |
| | 8.4, 7.7, 1.5 Hz)), 7.60 (1H, dd, J = 8.4, 0.5 Hz), 7.77 (2H, ddd, | |
| | J = 7.7, 1.5, 0.5 Hz), 8.02 (2H, ddd, J = 8.6, 1.5, 0.5 Hz). | |
| B0-5 | 1.60-1.72 (2H, m), 1.79-2.06 (14H, m), 1.98 (2H, m), 2.01 (dd, | −79,2, −112.1, |
| | J = 13.3, 2.7 Hz), 1.95 (3H, m), 1.94 (d, J = 16.3 Hz), 1.86 (dd, | −104.0, −111.8, |
| | J = 13.0, 2.8 Hz), 1.83 (3H, m), 2.01 (sept, J = 2.8 Hz)), | −122.3, −203.4 |
| | 4.29-4.37 (2H, m), 4.45 (1H, t, J = 7.4 Hz),, 7.13 (1H, dd, J = 1.6, | |
| | 0.5 Hz), 7.33-7.54 (5H, 7.45 (ddd, J = 8.6, 8.4, 1.5 Hz), 7.38 | |
| | (ddd, J = 8.4, 7.7, 1.5 Hz), 7.51 (dd, J = 8.2, 1.6 Hz)), 7.74-7.82 | |
| | (3H, 7.77 (ddd, J = 7.7, 1.5, 0.5 Hz), 7.80 (dd, J = 8.2, 0.5 Hz)), | |
| | 8.01 (2H, ddd, J = 8.6, 1.5, 0.5 Hz). | |

TABLE 2

| | ¹H-NMR | ¹⁹F-NMR |
|---|---|---|
| Compound | (DMSO, 400 MHz) | (DMSO, 376 MHz) |
| B0-6 | 7.20 (1H, dd, J = 1.6, 0.5 Hz), 1.60-1.72 (2H, m), 1.79-2.06 | −62.1, −105.2, |
| | (14H, m), 1.98 (2H, m), 2.01 (dd, J = 13.3, 2.7 Hz), 1.95 | −111.8, −122.3, |
| | (3H, m), 1.94 (d, J = 16.3 Hz), 1.86 (dd, J = 13.0, 2.8 Hz), | −203.4 |
| | 1.83 (3H, m), 2.01 (sept, J = 2.8 Hz)), 4.29-4.37 (2H, m), | |
| | 4.45 (1H, t, J = 7.4 Hz),, 7.13 (1H, dd, J = 1.6, 0.5 Hz), | |
| | 7.33-7.54 (5H, 7.45 (ddd, J = 8.6, 8.4, 1.5 Hz), 7.38 (ddd, | |
| | J = 8.4, 7.7, 1.5 Hz), 7.51 (dd, J = 8.2, 1.6 Hz)), 7.74-7.82 | |
| | (3H, 7.77 (ddd, J = 7.7, 1.5, 0.5 Hz), 7.80 (dd, J = 8.2, | |
| | 0.5 Hz)), 8.01 (2H, ddd, J = 8.6, 1.5, 0.5 Hz). | |
| B0-7 | 1.60-1.72 (2H, m), 1.79-2.06 (14H, m), 1.98 (2H, m), 2.01 | −70.1, 123.1, |
| | (dd, J = 13.3, 2.7 Hz), 1.95 (3H, m), 1.94 (d, J = 16.3 Hz), | −112., −111.8, |
| | 1.86 (dd, J = 13.0, 2.8 Hz), 1.83 (3H, m), 2.01 (sept, J = | −122.3, −203.4 |
| | 2.8 Hz)), 4.29-4.37 (2H, m), 4.45 (1H, t, J = 7.4 Hz),, 7.12 | |
| | (1H, dd, J = 1.6, 0.5 Hz), 7.33-7.53 (6H, 7.45 (ddd, J = 8.6, | |
| | 8.4, 1.5 Hz), 7.44 (dd, J = 8.2, 0.5 Hz), 7.37 (ddd, J = 8.4, | |
| | 7.7, 1.5 Hz), 7.50 (dd, J = 8.2, 1.6 Hz)), 7.77 (2H, ddd, J = | |
| | 7.7, 1.5, 0.5 Hz), 8.01 (2H, ddd, J = 8.6, 1.5, 0.5 Hz). | |
| B0-8 | 1.60-1.72 (2H, m), 1.79-2.06 (14H, m), 1.98 (2H, m), 2.01 | −56.1, −99.9, |
| | (dd, J = 13.3, 2.7 Hz), 1.95 (3H, m), 1.94 (d, J = 16.3 Hz), | −111.8, −122.3, |
| | 1.86 (dd, J = 13.0, 2.8 Hz), 1.83 (3H, m), 2.01 (sept, J = | −203.4 |
| | 2.8 Hz)), 4.29-4.37 (2H, m), 4.45 (1H, t, J = 7.4 Hz),, 7.21 | |
| | (1H, dd, J = 1.6, 0.5 Hz), 7.42 (1H, ddd, J = 8.3, 7.7, 1.5 Hz), | |
| | 7.53 (1H, dd, J = 8.2, 1.6 Hz), 7.57-7.74 (3H, 7.67 (dd, J = | |
| | 8.5, 0.5 Hz), 7.71 (dd, J = 8.2, 0.5 Hz), 7.62 (ddd, J = 8.6, | |
| | 8.3, 1.5 Hz)), 7.82 (1H, ddd, J = 7.7, 1.5, 0.5 Hz), 7.93 (1H, | |
| | ddd, J = 8.6, 1.5, 0.5 Hz), 8.07 (1H, dd, J = 8.5, 1.8 Hz), | |
| | 8.32 (1H, dd, J = 1.8, 0.5 Hz). | |
| B0-9 | 1.60-1.72 (2H, m), 1.79-2.06 (14H, m), 1.98 (2H, m), 2.01 | −86.7, −111.8, |
| | (dd, J = 13.3, 2.7 Hz), 1.95 (3H, m), 1.94 (d, J = 16.3 Hz), | −122.3, −203.4 |
| | 1.86 (dd, J = 13.0, 2.8 Hz), 1.83 (3H, m), 2.01 (sept, J = | |
| | 2.8 Hz)), 4.29-4.37 (2H, m), 4.45 (1H, t, J = 7.4 Hz),, | |
| | 7.31-7.49 (6H, 7.41 (dd, J = 1.9, 1.6 Hz), 7.36 (ddd, J = | |
| | 8.4, 8.3, 1.5 Hz), 7.37 (t, J = 1.9 Hz), 7.44 (ddd, J = 8.6, | |
| | 8.4, 1.5 Hz)), 7.76-7.84 (3H, 7.79 (ddd, J = 8.3, 1.5, | |
| | 0.5 Hz), 7.83 (dd, J = 1.9, 1.6 Hz)), 7.86 (2H, ddd, | |
| | J = 8.6, 1.5, 0.5 Hz). | |
| B0-10 | 1.60-1.72 (2H, m), 1.79-2.06 (14H, m), 1.98 (2H, m), | −47.9, −111.8, |
| | 2.01 (dd, J = 13.3, 2.7 Hz), 1.95 (3H, m), 1.94 (d, J = | −122.3, −203.4 |
| | 16.3 Hz), 1.86 (dd, J = 13.0, 2.8 Hz), 1.83 (3H, m), 2.01 | |
| | (sept, J = 2.8 Hz)), 4.29-4.37 (2H, m), 4.45 (1H, t, J = 7.4 | |
| | Hz), 7.33-7.52 (4H, 7.47 (ddd, J = 8.6, 8.4, 1.5 Hz), 7.38 | |
| | (ddd, J = 8.4, 8.3, 1.5 Hz)), 7.54 (1H, t, J = 1.8 Hz), | |
| | 7.79-7.92 (5H, 7.90 (t, J = 1.8 Hz), 7.88 (ddd, J = 8.6, | |
| | 1.5, 0.5 Hz), 7.82 (ddd, J = 8.3, 1.5, 0.5 Hz)), 8.18 | |
| | (1H, t, J = 1.8 Hz). | |

TABLE 3

| | ¹H-NMR | ¹⁹F-NMR |
|---|---|---|
| Compound | (DMSO, 400 MHz) | (DMSO, 376 MHz) |
| B0-11 | 1.60-1.72 (2H, m), 1.79-2.06 (14H, m), 1.98 (2H, m), | −92.3, −111.8, |
| | 2.01 (dd, J = 13.3, 2.7 Hz), 1.95 (3H, m), 1.94 (d, J = | −122.3, −203.4 |
| | 16.3 Hz), 1.86 (dd, J = 13.0, 2.8 Hz), 1.83 (3H, m), | |
| | 2.01 (sept, J = 2.8 Hz)), 4.29-4.37 (2H, m), 4.45 (1H, | |
| | t, J = 7.4 Hz)), 7.36 (ddd, J = 8.4, 7.7, 1.5 Hz), 7.44 | |
| | (t, J = 1.9 Hz)), 7.74 (1H, dd, J = 1.9, 1.8 Hz), 7.77-7.89 | |
| | (4H, 7.80 (ddd, J = 7.7, 1.5, 0.5 Hz), 7.86 (ddd, J = | |
| | 8.6, 1.5, 0.5 Hz)). | |
| B0-12 | 1.60-1.72 (2H, m), 1.79-2.06 (14H, m), 1.98 (2H, m), | −52.8, −111.8, |
| | 2.01 (dd, J = 13.3, 2.7 Hz), 1.95 (3H, m), 1.94 (d, J = | −122.3, −203.4 |
| | 16.3 Hz), 1.86 (dd, J = 13.0, 2.8 Hz), 1.83 (3H, m), | |
| | 2.01 (sept, J = 2.8 Hz)), 4.29-4.37 (2H, m), 4.45 , | |
| | (1H, t, J = 7.4 Hz), 3.06 (3H, s), 7.28 (1H, t, J = 1.8 Hz), | |
| | 7.38 (2H, ddd, J = 8.4, 7.7, 1.5 Hz), 7.58 (2H, ddd, | |
| | J = 8.6, 8.4, 1.5 Hz), 7.73-7.78 (2H, 7.74 (t, J = 1.8 Hz), | |
| | 7.76 (t, J = 1.8 Hz)), 7.80-7.91 (4H, 7.83 (ddd, J = 7.7, | |
| | 1.5, 0.5 Hz), 7.88 (ddd, J = 8.6, 1.5, 0.5 Hz). | |
| B0-13 | 1.60-1.69 (12H, m), 1.78-1.93 (22H, m), 1.84 (dd, J = | −56.4, −99.5, |
| | 12.8, 2.8 Hz), 1.89 (sept, J = 2.8 Hz), 1.83 (dt, J = 12.9, | −110.1 |
| | 2.8 Hz)), 1.96-2.16 (9H, m), 2.06 (dd, J = 13.2, 2.8 Hz), | |
| | 2.00 (dd, J = 13.2, 2.8 Hz), 2.11 (dd, J = 13.6, 2.8 Hz), | |
| | 2.11 (dd, J = 13.6, 2.8 Hz)), 3.71 (1H, d, J = 16.1 Hz), | |
| | 3.67-3.82 (3H, 3.78 (d, J = 16.1 Hz), 3.71 (d, J = 14.8 | |
| | Hz), 3.78 (d, J = 14.8 Hz)), 4.20-4.21 (4H, 4.20 (s), | |
| | 4.20 (s)), 7.23 (1H, dd, J = 1.6, 0.5 Hz), 7.33-7.56 | |
| | (5H, 7.46 (ddd, J = 8.6, 8.4, 1.5 Hz), 7.38 (ddd, J = | |
| | 8.4, 7.7, 1.5 Hz), 7.54 (dd, J = 8.2, 1.6 Hz)), | |
| | 7.69-7.80 (3H, 7.77 (ddd, J = 7.7, 1.5, 0.5 Hz), 7.72 | |
| | (dd, J = 8.2, 0.5 Hz)), 8.02 (2H, ddd, J = 8.6, 1.5, | |
| | 0.5 Hz) | |
| B0-14 | 1.93-2.09 (2H, 2.01 (quint, J = 7.4 Hz), 2.01 (quint, J = | −56.4, −99.5, |
| | 7.4 Hz)), 2.35-2.44 (2H, 2.40 (t, J = 7.4 Hz), 2.40 (t, , | −78.2, −121.7 |
| | J = 7.4 Hz)) 4.12-4.20 (2H, 4.16 (t, J = 7.4 Hz), 4.16 (t, | |
| | J = 7.4 Hz)), 5.61 (1H, s), 7.22 (1H, d, J = 1.6 Hz), 8.07 | |
| | (1H, d, J = 1.6 Hz), 7.23 (1H, dd, J = 1.6, 0.5 Hz), , | |
| | 7.33-7.56 (5H, 7.46 (ddd, J = 8.6, 8.4 1.5 Hz), 7.38 | |
| | (ddd, J = 8.4, 7.7, 1.5 Hz), 7.54 (dd, J = 8.2, 1.6 Hz)), | |
| | 7.69-7.80 (3H, 7.77 (ddd, J = 7.7, 1.5, 0.5 Hz), 7.72 (dd, | |
| | J = 8.2, 0.5 Hz)), 8.02 (2H, ddd, J = 8.6, 1.5, 0.5 Hz) | |
| B0-15 | 1.58-1.68 (6H, 1.63 (dt, J = 13.2, 2.8 Hz), 1.63 (dt, J = | −56.4, −99.5, |
| | 13.2, 2.8 Hz)), 1.69-1.79 (6H, 1.74 (dd, J = 13.2, 2.8 Hz), | −114.5 |
| | 1.74 (dd, J = 13.2, 2.8 Hz)), 1.88 (3H, sept, J = 2.8 Hz), | |
| | 2.06 (2H, s), 7.23 (1H, dd, J = 1.6, 0.5 Hz), 7.33-7.56 | |
| | (5H, 7.46 (ddd, J = 8.6, 8.4, 1.5 Hz), 7.38 (ddd, J = | |
| | 8.4, 7.7, 1.5 Hz), 7.54 (dd, J = 8.2, 1.6 Hz)), 7.69-7.80 | |
| | (3H, 7.77 (ddd, J = 7.7, 1.5, 0.5 Hz), 7.72 (dd, J = | |
| | 8.2, 0.5 Hz)), 8.02 (2H, ddd, J = 8.6, 1.5, 0.5 Hz) | |

TABLE 4

| | ¹H-NMR | ¹⁹F-NMR |
|---|---|---|
| Compound | (DMSO, 400 MHz) | (DMSO, 376 MHz) |
| B0-16 | 1.61-1.92 (4H, m), 1.71 (ddd, J = 17.7, 4.9, 4.2 Hz), 1.85 | −56.4, −99.5, |
| | (ddd, J = 17.7, 8.1, 1.4 Hz), 1.70 (ddd, J = 15.4, 2.5, 2.4 | −109.4, −124.0 |
| | Hz)), 2.06 (1H, ddd, J = 8.1, 4.2, 1.4 Hz), 2.25-2.33 (2H, | |
| | 2.28 (dtdd, J = 4.9, 2.5, 1.6, 1.4 Hz), 2.30 (1H, m), 4.23 | |
| | (1H, dd, J = 8.1, 1.6 Hz), 4.40 (1H, dd, J = 8.1, 1.6 Hz), | |
| | 7.35-7.49 (6H, 7.44 (dddd, J = 8.0, 7.5, 1.6, 0.5 Hz), 7.38 | |
| | (tt, J = 7.5, 1.6 Hz), 7.38 (tt, J = 7.5, 1.6 Hz), 7.44 (dddd, | |
| | J = 8.0, 7.5, 1.6, 0.5 Hz)), 7.75-7.82 (4H, 7.78 (dddd, J = | |
| | 8.0, 1.6, 1.4, 0.5 Hz), 7.78 (dddd, J = 8.0, 1.6, 1.4, 0.5 | |
| | Hz)), 7.23 (1H, dd, J = 1.6, 0.5 Hz), 7.33-7.56 (5H, 7.46 | |
| | (ddd, J = 8.6, 8.4, 1.5 Hz), 7.38 (ddd, J = 8.4, 7.7, 1.5 | |
| | Hz), 7.54 (dd, J = 8.2, 1.6 Hz)), 7.69-7.80 (3H, 7.77 | |
| | (ddd, J = 7.7, 1.5, 0.5 Hz), 7.72 (dd, J = 8.2, 0.5 Hz)), | |
| | 8.02 (2H, ddd, J = 8.6, 1.5, 0.5 Hz) | |

TABLE 5

| | ¹H-NMR | ¹⁹F-NMR |
|---|---|---|
| Compound | (DMSO, 400 MHz) | (DMSO, 376 MHz) |
| D0-1 | 7.05 (1H, ddd, J = 8.3, 1.2, 0.5 Hz), 7.26 (1H, ddd, J = | −56.4, −99.5 |
| | 8.1, 7.3, 1.2 Hz), 7.51 (1H, ddd, J = 8.3, 7.3, 1.4 Hz), 7.94 | |
| | (1H, ddd, J = 8.1, 1.4, 0.5 Hz), 7.23 (1H, dd, J = 1.6, 0.5 | |
| | Hz), 7.33-7.56 (5H, 7.46 (ddd, J = 8.6, 8.4, 1.5 Hz), 7.38 | |
| | (ddd, J = 8.4, 7.7, 1.5 Hz), 7.54 (dd, J = 8.2, 1.6 Hz)), | |
| | 7.69-7.80 (3H, 7.77 (ddd, J = 7.7, 1.5, 0.5 Hz), 7.72 | |
| | (dd, J = 8.2, 0.5 Hz)), 8.02 (2H, ddd, J = 8.6, 1.5, 0.5 Hz) | |
| D0-2 | 7.05 (1H, ddd, J = 8.3, 1.2, 0.5 Hz), 7.26 (1H, ddd, J = 8.1, | −54.2, −98.5 |
| | 7.3, 1.2 Hz), 7.51 (1H, ddd, J = 8.3, 7.3, 1.4 Hz), 7.94 (1H, | |
| | ddd, J = 8.1, 1.4, 0.5 Hz), 7.27 (1H, dd, J = 8.2, 1.7 Hz), | |
| | 7.33-7.51 (5H, 7.46 (ddd, J = 8.6, 8.4, 1.5 Hz), 7.38 (ddd, | |
| | J = 8.4, 7.7, 1.5 Hz), 7.44 (dd, J = 8.2, 0.4 Hz)), 7.58 (1H, | |
| | dd, J = 1.7, 0.4 Hz), 7.76 (2H, ddd, J = 7.7, 1.5, 0.5 Hz), | |
| | 8.02 (2H, ddd, J = 8.6, 1.5, 0.5 Hz) | |
| D0-3 | 7.05 (1H, ddd, J = 8.3, 1.2, 0.5 Hz), 7.26 (1H, ddd, J = 8.1, | −58.1, −101.2 |
| | 7.3, 1.2 Hz), 7.51 (1H, ddd, J = 8.3, 7.3, 1.4 Hz), 7.94 (1H, | |
| | ddd, J = 8.1, 1.4, 0.5 Hz), 7.20-7.25 (2H, 7.24 (dd, J = 1.9, | |
| | 1.6 Hz), 7.21 (dd, J = 2.2, 1.6 Hz)), 7.33-7.50 (4H, 7.45 | |
| | (ddd, J = 8.6, 8.4, 1.5 Hz), 7.38 (ddd, J = 8.4, 7.7, 1.5 Hz)), | |
| | 7.56 (1H, dd, J = 2.2, 1.9 Hz), 7.77 (2H, ddd, J = 7.7, 1.5, | |
| | 0.5 Hz), 8.01 (2H, ddd, J = 8.6, 1.5, 0.5 Hz) | |
| D0-4 | 7.05 (1H, ddd, J = 8.3, 1.2, 0.5 Hz), 7.26 (1H, ddd, J = 8.1, | −59.8, −103.5 |
| | 7.3, 1.2 Hz), 7.51 (1H, ddd, J = 8.3, 7.3, 1.4 Hz), 7.94 (1H, | |
| | ddd, J = 8.1, 1.4, 0.5 Hz), 7.15 (1H, dd, J = 1.6, 0.5 Hz), | |
| | 7.24 (1H, dd, J = 8.4, 1.6 Hz), 7.33-7.51 (4H, 7.46 (ddd, J = | |
| | 8.6, 8.4, 1.5 Hz), 7.37 (ddd, J = 8.4, 7.7, 1.5 Hz)), 7.60 | |
| | (1H, dd, J = 8.4, 0.5 Hz), 7.77 (2H, ddd, J = 7.7, 1.5, 0.5 | |
| | Hz), 8.02 (2H, ddd, J = 8.6, 1.5, 0.5 Hz). | |
| D0-5 | 7.05 (1H, ddd, J = 8.3, 1.2, 0.5 Hz), 7.26 (1H, ddd, J = 8.1, | −79,2, −112,1, |
| | 7.3, 1.2 Hz), 7.51 (1H, ddd, J = 8.3, 7.3, 1.4 Hz), 7.94 (1H, | −104.0 |
| | ddd, J = 8.1, 1.4, 0.5 Hz), 7.13 (1H, dd, J = 1.6, 0.5 Hz), | |
| | 7.33-7.54 (5H, 7.45 (ddd, J = 8.6, 8.4, 1.5 Hz), 7.38 (ddd, | |
| | J = 8.4, 7.7, 1.5 Hz), 7.51 (dd, J = 8.2, 1.6 Hz)), 7.74-7.82 | |
| | (3H, 7.77 (ddd, J = 7.7, 1.5, 0.5 Hz), 7.80 (dd, J = 8.2, | |
| | 0.5 Hz)), 8.01 (2H, ddd, J = 8.6, 1.5, 0.5 Hz). | |

TABLE 6

| | ¹H-NMR | ¹⁹F-NMR |
|---|---|---|
| Compound | (DMSO, 400 MHz) | (DMSO, 376 MHz) |
| D0-6 | 7.05 (1H, ddd, J = 8.3, 1.2, 0.5 Hz), 7.26 (1H, ddd, J = | −62.1, −105.2 |
| | 8.1, 7.3, 1.2 Hz), 7.51 (1H, ddd, J = 8.3, 7.3, 1.4 Hz), | |
| | 7.94 (1H, ddd, J = 8.1, 1.4, 0.5 Hz), 7.13 (1H, dd, | |
| | J = 1.6, 0.5 Hz), 7.33-7.54 (5H, 7.45 (ddd, J = 8.6, | |
| | 8.4, 1.5 Hz), 7.38 (ddd, J = 8.4, 7.7, 1.5 Hz), 7.51 | |
| | (dd, J = 8.2, 1.6 Hz)), 7.74-7.82 (3H, 7.77 (ddd, | |
| | J = 7.7, 1.5, 0.5 Hz), 7.80 (dd, J = 8.2, 0.5 Hz)), 8.01 | |
| | (2H, ddd, J = 8.6, 1.5, 0.5 Hz). | |
| D0-7 | 7.05 (1H, ddd, J = 8.3, 1.2, 0.5 Hz), 7.26 (1H, ddd, | −70.1, 123.1, |
| | J = 8.1, 7.3, 1.2 Hz), 7.51 (1H, ddd, J = 8.3, 7.3, | −112.1 |
| | 1.4 Hz), 7.94 (1H, ddd, J = 8.1, 1.4, 0.5 Hz), 7.12 | |
| | (1H, dd, J = 1.6, 0.5 Hz), 7.33-7.53 (6H, 7.45 | |
| | (ddd, J = 8.6, 8.4, 1.5 Hz), 7.44 (dd, J = 8.2, 0.5 Hz), | |
| | 7.37 (ddd, J = 8.4, 7.7, 1.5 Hz), 7.50 (dd, J = 8.2, | |
| | 1.6 Hz)), 7.77 (2H, ddd, J = 7.7, 1.5, 0.5 Hz), 8.01 | |
| | (2H, ddd, J = 8.6, 1.5, 0.5 Hz). | |
| D0-8 | 7.05 (1H, ddd, J = 8.3, 1.2, 0.5 Hz), 7.26 (1H, ddd, J = | −56.1, −99.9 |
| | 8.1, 7.3, 1.2 Hz), 7.51 (1H, ddd, J = 8.3, 7.3, 1.4 Hz), | |
| | 7.94 (1H, ddd, J = 8.1, 1.4, 0.5 Hz), 1.30 (3H, t, J = | |
| | 7.1 Hz), 4.08-4.13 (2H, 4.11 (q, J = 7.1 Hz), 4.11 | |
| | (q, J = 7.1 Hz)), 7.21 (1H, dd, J = 1.6, 0.5 Hz), 7.42 | |
| | (1H, ddd, J = 8.3, 7.7, 1.5 Hz), 7.53 (1H, dd, J = 8.2, | |
| | 1.6 Hz), 7.57-7.74 (3H, 7.67 (dd, J = 8.5, 0.5 Hz), | |
| | 7.71 (dd, J = 8.2, 0.5 Hz), 7.62 (ddd, J = 8.6, 8.3, | |
| | 1.5 Hz)), 7.82 (1H, ddd, J = 7.7, 1.5, 0.5 Hz), 7.93 | |
| | (1H, ddd, J = 8.6, 1.5, 0.5 Hz), 8.07 (1H, dd, J = 8.5, | |
| | 1.8 Hz), 8.32 (1H, dd, J = 1.8, 0.5 Hz). | |
| D0-9 | 7.05 (1H, ddd, J = 8.3, 1.2, 0.5 Hz), 7.26 (1H, ddd, | −86.7 |
| | J = 8.1, 7.3, 1.2 Hz), 7.51 (1H, ddd, J = 8.3, 7.3, | |
| | 1.4 Hz), 7.94 (1H, ddd, J = 8.1, 1.4, 0.5 Hz), 7.31-7.49 | |
| | (6H, 7.41 (dd, J = 1.9, 1.6 Hz), 7.36 (ddd, J = 8.4, 8.3, | |
| | 1.5 Hz), 7.37 (t, J = 1.9 Hz), 7.44 (ddd, J = 8.6, 8.4, | |
| | 1.5 Hz)), 7.76-7.84 (3H, 7.79 (ddd, J = 8.3, 1.5, 0.5 Hz), | |
| | 7.83 (dd, J = 1.9, 1.6 Hz)), 7.86 (2H, ddd, J = 8.6, | |
| | 1.5, 0.5 Hz). | |
| D0-10 | 7.05 (1H, ddd, J = 8.3, 1.2, 0.5 Hz), 7.26 (1H, ddd, | −47.9 |
| | J = 8.1, 7.3, 1.2 Hz), 7.51 (1H, ddd, J = 8.3, 7.3, 1.4 Hz), | |
| | 7.94 (1H, ddd, J = 8.1, 1.4, 0.5 Hz), 7.33-7.52 (4H, | |
| | 7.47 (ddd, J = 8.6, 8.4, 1.5 Hz), 7.38 (ddd, J = 8.4, 8.3, | |
| | 1.5 Hz)), 7.54 (1H, t, J = 1.8 Hz), 7.79-7.92 (5H, 7.90 | |
| | (t, J = 1.8 Hz), 7.88 (ddd, J = 8.6, 1.5, 0.5 Hz), 7.82 | |
| | (ddd, J = 8.3, 1.5, 0.5 Hz)), 8.18 (1H, t, J = 1.8 Hz). | |

TABLE 7

| | ¹H-NMR | ¹⁹F-NMR |
|---|---|---|
| Compound | (DMSO, 400 MHz) | (DMSO, 376 MHz) |
| D0-11 | 7.05 (1H, ddd, J = 8.3, 1.2, 0.5 Hz), 7.26 (1H, ddd, J = 8.1, | −92.3 |
| | 7.3, 1.2 Hz), 7.51 (1H, ddd, J = 8.3, 7.3, 1.4 Hz), 7.94 (1H, | |
| | ddd, J = 8.1, 1.4, 0.5 Hz), 3.06 (3H, s), 7.31-7.49 (6H, 7.47 | |
| | (dd, J = 1.9, 1.8 Hz), 7.44 (ddd, J = 8.6, 8.4, 1.5 Hz), 7.36 | |
| | (ddd, J = 8.4, 7.7, 1.5 Hz), 7.44 (t, J = 1.9 Hz)), 7.74 (1H, | |
| | dd, J = 1.9, 1.8 Hz), 7.77-7.89 (4H, 7.80 (ddd, J = 7.7, 1.5, | |
| | 0.5 Hz), 7.86 (ddd, J = 8.6, 1.5, 0.5 Hz)). | |
| D0-12 | 7.05 (1H, ddd, J = 8.3, 1.2, 0.5 Hz), 7.26 (1H, ddd, J = 8.1, | −52.8 |
| | 7.3, 1.2 Hz), 7.51 (1H, ddd, J = 8.3, 7.3, 1.4 Hz), 7.94 (1H, | |
| | ddd, J = 8.1, 1.4, 0.5 Hz), 3.06 (3H, s), 7.28 (1H, t, J = 1.8 | |
| | Hz), 7.38 (2H, ddd, J = 8.4, 7.7, 1.5 Hz), 7.58 (2H, ddd, J = | |
| | 8.6, 8.4, 1.5 Hz), 7.73-7.78 (2H, 7.74 (t, J = 1.8 Hz), 7.76 | |
| | (t, J = 1.8 Hz)), 7.80-7.91 (4H, 7.83 (ddd, J = 7.7, 1.5, 0.5 | |
| | Hz), 7.88 (ddd, J = 8.6, 1.5, 0.5 Hz)). | |
| D0-13 | 6.74 (2H, dd, J = 8.0, 1.6 Hz), 7.42 (1H, t, J = 8.0 Hz), 7.23 | −56.4, −99.5 |
| | (1H, dd, J = 1.6, 0.5 Hz), 7.33-7.56 (5H, 7.46 (ddd, J = 8.6, | |
| | 8.4, 1.5 Hz), 7.38 (ddd, J = 8.4, 7.7, 1.5 Hz), 7.54 (dd, J = | |
| | 8.2, 1.6 Hz)), 7.69-7.80 (3H, 7.77 (ddd, J = 7.7, 1.5, 0.5 | |
| | Hz), 7.72 (dd, J = 8.2, 0.5 Hz)), 8.02 (2H, ddd, J = 8.6, | |
| | 1.5, 0.5 Hz) | |
| D0-14 | 7.53-7.69 (3H, 7.66 (tt, J = 7.4, 1.5 Hz), 7.58 (dddd, J = 8.4, | −56.4, −99.5 |
| | 7.4, 1.4, 0.5 Hz)), 7.97 (2H, dddd, J = 8.4, 1.7, 1.5, 0.5 Hz), | |
| | 7.23 (1H, dd, J = 1.6, 0.5 Hz), 7.33-7.56 (5H, 7.46 (ddd, J = | |
| | 8.6, 8.4, 1.5 Hz), 7.38 (ddd, J = 8.4, 7.7, 1.5 Hz), 7.54 | |
| | (dd, J = 8.2, 1.6 Hz)), 7.69-7.80 (3H, 7.77 (ddd, J = 7.7, | |
| | 1.5, 0.5 Hz), 7.72 (dd, J = 8.2, 0.5 Hz)), 8.02 (2H, ddd, | |
| | J = 8.6, 1.5, 0.5 Hz) | |

### <Production of High-Molecular-Weight Compound>

Each of high-molecular-weight compounds (A1-01) to (A1-02) used in the present examples was obtained by carrying out radical polymerization using monomers from which constitutional units constituting each of the high-molecular-weight compounds were derived, at a predetermined molar ratio.

The weight-average molecular weight (Mw) and the molecular weight dispersity (Mw/Mn) of each of the obtained high-molecular-weight compounds were determined by GPC measurement (in terms of the standard polystyrene).

In addition, the copolymerization composition ratio (the proportion (molar ratio) of each constitutional unit in the structural formula) of each of the obtained high-molecular-weight compounds was determined from the carbon 13 nuclear magnetic resonance spectrum (600 MHz_¹³C-NMR).

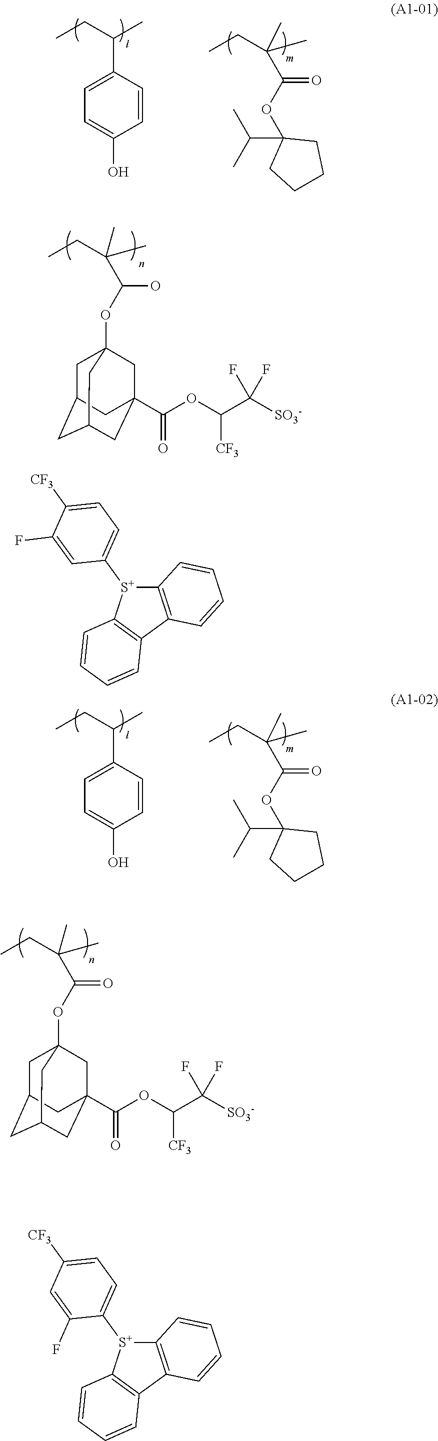

High-molecular-weight compound (A1-01): weight-average molecular weight (Mw) of 9,500, molecular weight dispersity (Mw/Mn) of 1.72, 1/m/n=55/30/15
High-molecular-weight compound (A1-02): weight-average molecular weight (Mw) of 9,500, molecular weight dispersity (Mw/Mn) of 1.75, 1/m/n=55/30/15

### <Preparation of Resist Composition>

### Examples 1 to 60 and Comparative Examples 1 to 24

The components listed in Tables 8 to 19 were mixed and dissolved to prepare a resist composition of each example.

TABLE 8

| | Component | Component | Component | Component |
|---|---|---|---|---|
| | (A) | (B) | (D) | (S) |
| Example | (A1)-1 | (B0)-1 | (D1)-11 | (S)-1 |
| 1 | [100] | [22] | [3.0] | [6400] |
| Example | (A1)-1 | (B0)-2 | (D1)-11 | (S)-1 |
| 2 | [100] | [22] | [3.0] | [6400] |
| Example | (A1)-1 | (B0)-3 | (D1)-11 | (S)-1 |
| 3 | [100] | [22] | [3.0] | [6400] |
| Example | (A1)-1 | (B0)-4 | (D1)-11 | (S)-1 |
| 4 | [100] | [22] | [3.0] | [6400] |
| Example | (A1)-1 | (B0)-5 | (D1)-11 | (S)-1 |
| 5 | [100] | [23.5] | [3.0] | [6400] |
| Example | (A1)-1 | (B0)-6 | (D1)-11 | (S)-1 |
| 6 | [100] | [22.4] | [3.0] | [6400] |
| Example | (A1)-1 | (B0)-7 | (D1)-11 | (S)-1 |
| 7 | [100] | [25] | [3.0] | [6400] |
| Example | (A1)-1 | (B0)-8 | (D1)-11 | (S)-1 |
| 8 | [100] | [24.1] | [3.0] | [6400] |
| Example | (A1)-1 | (B0)-9 | (D1)-11 | (S)-1 |
| 9 | [100] | [20.7] | [3.0] | [6400] |
| Example | (A1)-1 | (B0)-10 | (D1)-11 | (S)-1 |
| 10 | [100] | [22.2] | [3.0] | [6400] |
| Example | (A1)-1 | (B0)-11 | (D1)-11 | (S)-1 |
| 11 | [100] | [22.3] | [3.0] | [6400] |
| Example | (A1)-1 | (B0)-12 | (D1)-11 | (S)-1 |
| 12 | [100] | [23.8] | [3.0] | [6400] |

TABLE 9

| | Component | Component | Component | Component |
|---|---|---|---|---|
| | (A) | (B) | (D) | (S) |
| Example | (A1)-1 | (B0)-13 | (D1)-11 | (S)-1 |
| 13 | [100] | [33.4] | [3.0] | [6400] |
| Example | (A1)-1 | (B0)-14 | (D1)-11 | (S)-1 |
| 14 | [100] | [34.3] | [3.0] | [6400] |
| Example | (A1)-1 | (B0)-15 | (D1)-11 | (S)-1 |
| 15 | [100] | [18.8] | [3.0] | [6400] |
| Example | (A1)-1 | (B0)-16 | (D1)-11 | (S)-1 |
| 16 | [100] | [24.6] | [3.0] | [6400] |
| Example | (A1)-1 | (B0)-5 | (D1)-12 | (S)-1 |
| 17 | [100] | [23.5] | [3.1] | [6400] |
| Example | (A1)-1 | (B0)-5 | (D1)-13 | (S)-1 |
| 18 | [100] | [23.5] | [3.1] | [6400] |

TABLE 10

| | Component | Component | Component | Component |
|---|---|---|---|---|
| | (A) | (B) | (D) | (S) |
| Example | (A1)-2 | (B0)-5 | (D1)-11 | (S)-1 |
| 19 | [100] | [23.5] | [3.0] | [6400] |
| Example | (A1)-3 | (B0)-5 | (D1)-11 | (S)-1 |
| 20 | [100] | [23.5] | [3.0] | [6400] |
| Example | (A1)-01 | — | (D1)-11 | (S)-1 |
| 21 | [100] | | [3.0] | 6400] |
| Example | (A1)-02 | — | (D1)-11 | (S)-1 |
| 22 | [100] | | [3.0] | [6400] |

TABLE 11

| | Component | Component | Component | Component |
|---|---|---|---|---|
| | (A) | (B) | (D) | (S) |
| Example | (A1)-1 | (B1)-5 | (D0)-1 | (S)-1 |
| 23 | [100] | [22] | [3.6] | [6400] |
| Example | (A1)-1 | (B1)-5 | (D0)-2 | (S)-1 |
| 24 | [100] | [22] | [3.6] | [6400] |
| Example | (A1)-1 | (B1)-5 | (D0)-3 | (S)-1 |
| 25 | [100] | [22] | [3.6] | [6400] |
| Example | (A1)-1 | (B1)-5 | (D0)-4 | (S)-1 |
| 26 | [100] | [22] | [3.6] | [6400] |
| Example | (A1)-1 | (B1)-5 | (D0)-5 | (S)-1 |
| 27 | [100] | [22] | [4.0] | [6400] |
| Example | (A1)-1 | (B1)-5 | (D0)-6 | (S)-1 |
| 28 | [100] | [22] | [3.7] | [6400] |
| Example | (A1)-1 | (B1)-5 | (D0)-7 | (S)-1 |
| 29 | [100] | [22] | [4.4] | [6400] |
| Example | (A1)-1 | (B1)-5 | (D0)-8 | (S)-1 |
| 30 | [100] | [22] | [4.2] | [6400] |
| Example | (A1)-1 | (B1)-5 | (D0)-9 | (S)-1 |
| 31 | [100] | [22] | [3.3] | [6400] |
| Example | (A1)-1 | (B1)-5 | (D0)-10 | (S)-1 |
| 32 | [100] | [22] | [3.7] | [6400] |
| Example | (A1)-1 | (B1)-5 | (D0)-11 | (S)-1 |
| 33 | [100] | [22] | [3.7] | [6400] |
| Example | (A1)-1 | (B1)-5 | (D0)-12 | (S)-1 |
| 34 | [100] | [22] | [4.1] | [6400] |

TABLE 12

| | Component | Component | Component | Component |
|---|---|---|---|---|
| | (A) | (B) | (D) | (S) |
| Example | (A1)-1 | (B1)-7 | (D0)-1 | (S)-1 |
| 35 | [100] | [33.5] | [3.6] | [6400] |
| Example | (A1)-1 | (B1)-8 | (D0)-1 | (S)-1 |
| 36 | [100] | [34.4] | [3.6] | [6400] |
| Example | (A1)-1 | (B1)-9 | (D0)-1 | (S)-1 |
| 37 | [100] | [18.9] | [3.6] | [6400] |
| Example | (A1)-1 | (B1)-10 | (D0)-1 | (S)-1 |
| 38 | [100] | [24.6] | [3.6] | [6400] |
| Example | (A1)-1 | (B1)-5 | (D0)-13 | (S)-1 |
| 39 | [100] | [22] | [3.8] | [6400] |
| Example | (A1)-1 | (B1)-5 | (D0)-14 | (S)-1 |
| 40 | [100] | [22] | [3.7] | [6400] |

TABLE 13

| | Component | Component | Component | Component |
|---|---|---|---|---|
| | (A) | (B) | (D) | (S) |
| Example | (A1)-2 | (B1)-5 | (D0)-5 | (S)-1 |
| 41 | [100] | [22] | [4.0] | [6400] |
| Example | (A1)-3 | (B1)-5 | (D0)-5 | (S)-1 |
| 42 | [100] | [22] | [4.0] | [6400] |

TABLE 14

| | Component | Component | Component | Component |
|---|---|---|---|---|
| | (A) | (B) | (D) | (S) |
| Example | (A1)-1 | (B0)-1 | (D0)-1 | (S)-1 |
| 43 | [100] | [22] | [3.6] | [6400] |
| Example | (A1)-1 | (B0)-2 | (D0)-2 | (S)-1 |
| 44 | [100] | [22] | [3.6] | [6400] |
| Example | (A1)-1 | (B0)-3 | (D0)-3 | (S)-1 |
| 45 | [100] | [22] | [3.6] | [6400] |
| Example | (A1)-1 | (B0)-4 | (D0)-4 | (S)-1 |
| 46 | [100] | [22] | [3.6] | [6400] |
| Example | (A1)-1 | (B0)-5 | (D0)-5 | (S)-1 |
| 47 | [100] | [23.5] | [4.0] | [6400] |
| Example | (A1)-1 | (B0)-6 | (D0)-6 | (S)-1 |
| 48 | [100] | [22.4] | [3.7] | [6400] |
| Example | (A1)-1 | (B0)-7 | (D0)-7 | (S)-1 |
| 49 | [100] | [25] | [4.4] | [6400] |
| Example | (A1)-1 | (B0)-8 | (D0)-8 | (S)-1 |
| 50 | [100] | [24.1] | [4.2] | [6400] |
| Example | (A1)-1 | (B0)-9 | (D0)-9 | (S)-1 |
| 51 | [100] | [20.7] | [3.3] | [6400] |
| Example | (A1)-1 | (B0)-10 | (D0)-10 | (S)-1 |
| 52 | [100] | [22.2] | [3.7] | [6400] |
| Example | (A1)-1 | (B0)-11 | (D0)-11 | (S)-1 |
| 53 | [100] | [22.3] | [3.7] | [6400] |
| Example | (A1)-1 | (B0)-12 | (D0)-12 | (S)-1 |
| 54 | [100] | [23.8] | [4.1] | [6400] |

TABLE 15

| | Component | Component | Component | Component |
|---|---|---|---|---|
| | (A) | (B) | (D) | (S) |
| Example | (A1)-1 | (B0)-13 | (D0)-1 | (S)-1 |
| 55 | [100] | [33.4] | [3.6] | [6400] |
| Example | (A1)-1 | (B0)-14 | (D0)-1 | (S)-1 |
| 56 | [100] | [34.3] | [3.6] | [6400] |
| Example | (A1)-1 | (B0)-15 | (D0)-1 | (S)-1 |
| 57 | [100] | [18.8] | [3.6] | [6400] |
| Example | (A1)-1 | (B0)-16 | (D0)-1 | (S)-1 |
| 58 | [100] | [24.6] | [3.6] | [6400] |

TABLE 16

| | Component | Component | Component | Component |
|---|---|---|---|---|
| | (A) | (B) | (D) | (S) |
| Example | (A1)-2 | (B0)-5 | (D0)-5 | (S)-1 |
| 59 | [100] | [23.5] | [4.0] | [6400] |
| Example | (A1)-3 | (B0)-5 | (D0)-5 | (S)-1 |
| 60 | [100] | [23.5] | [4.0] | [6400] |

TABLE 17

| | Component | Component | Component | Component |
|---|---|---|---|---|
| | (A) | (B) | (D) | (S) |
| Comparative | (A1)-1 | (B1)-1 | (D1)-1 | (S)-1 |
| Example 1 | [100] | [19.9] | [3.1] | [6400] |
| Comparative | (A1)-1 | (B1)-2 | (D1)-2 | (S)-1 |
| Example 2 | [100] | [19.9] | [3.1] | [6400] |
| Comparative | (A1)-1 | (B1)-3 | (D1)-3 | (S)-1 |
| Example 3 | [100] | [20.4] | [3.2] | [6400] |
| Comparative | (A1)-1 | (B1)-4 | (D1)-4 | (S)-1 |
| Example 4 | [100] | [20.5] | [3.3] | [6400] |
| Comparative | (A1)-1 | (B1)-5 | (D1)-5 | (S)-1 |
| Example 5 | [100] | [22] | [4.0] | [6400] |
| Comparative | (A1)-1 | (B1)-6 | (D1)-6 | (S)-1 |
| Example 6 | [100] | [22.1] | [3.7] | [6400] |

TABLE 18

| | Component | Component | Component | Component |
|---|---|---|---|---|
| | (A) | (B) | (D) | (S) |
| Comparative | (A1)-1 | (B1)-7 | (D1)-5 | (S)-1 |
| Example 7 | [100] | [33.5] | [4.0] | [6400] |
| Comparative | (A1)-1 | (B1)-8 | (D1)-5 | (S)-1 |
| Example 8 | [100] | [34.4] | [4.0] | [6400] |
| Comparative | (A1)-1 | (B1)-9 | (D1)-5 | (S)-1 |
| Example 9 | [100] | [18.9] | [4.0] | [6400] |
| Comparative | (A1)-1 | (B1)-10 | (D1)-5 | (S)-1 |
| Example 10 | [100] | [24.6] | [4.0] | [6400] |
| Comparative | (A1)-1 | (B1)-11 | (D1)-4 | (S)-1 |
| Example 11 | [100] | [31.9] | [3.3] | [6400] |
| Comparative | (A1)-1 | (B1)-12 | (D1)-4 | (S)-1 |
| Example 12 | [100] | [32.8] | [3.3] | [6400] |
| Comparative | (A1)-1 | (B1)-13 | (D1)-4 | (S)-1 |
| Example 13 | [100] | [17.3] | [3.3] | [6400] |
| Comparative | (A1)-1 | (B1)-14 | (D1)-4 | (S)-1 |
| Example 14 | [100] | [23.1] | [3.3] | [6400] |
| Comparative | (A1)-1 | (B1)-1 | (D1)-7 | (S)-1 |
| Example 15 | [100] | [19.9] | [3.4] | [6400] |
| Comparative | (A1)-1 | (B1)-5 | (D1)-8 | (S)-1 |
| Example 16 | [100] | [22] | [3.8] | [6400] |
| Comparative | (A1)-1 | (B1)-1 | (D1)-9 | (S)-1 |
| Example 17 | [100] | [19.9] | [3.3] | [6400] |
| Comparative | (A1)-1 | (B1)-5 | (D1)-10 | (S)-1 |
| Example 18 | [100] | [22] | [3.7] | [6400] |

TABLE 19

| | Component | Component | Component | Component |
|---|---|---|---|---|
| | (A) | (B) | (D) | (S) |
| Comparative | (A1)-2 | (B1)-5 | (D1)-5 | (S)-1 |
| Example 19 | [100] | [22] | [4.0] | [6400] |
| Comparative | (A1)-3 | (B1)-5 | (D1)-5 | (S)-1 |
| Example 20 | [100] | [22] | [4.0] | [6400] |
| Comparative | (A1)-2 | (B1)-4 | (D1)-4 | (S)-1 |
| Example 21 | [100] | [20.5] | [3.3] | [6400] |
| Comparative | (A1)-3 | (B1)-4 | (D1)-4 | (S)-1 |
| Example 22 | [100] | [20.5] | [3.3] | [6400] |
| Comparative | (A1)-4 | — | (D1)-5 | (S)-1 |
| Example 23 | [100] | | [4.0] | [6400] |
| Comparative | (A1)-5 | — | (D1)-4 | (S)-1 |
| Example 24 | [100] | | [3.3] | [6400] |

In Tables 8 to 19, each abbreviation has the following meaning. The numerical values in the brackets are blending amounts (parts by mass).
(A1)-01 and (A1)-02: high-molecular-weight compounds (A1-01) to (A1-02) shown above
(A1)-1 to (A1)-5: high-molecular-weight compounds (A1-1) to (A1-5) shown above
High-molecular-weight compound (A1-1): weight-average molecular weight (Mw) of 5,500, molecular weight dispersity (Mw/Mn) of 1.76, 1/m=40/60
High-molecular-weight compound (A1-2): weight-average molecular weight (Mw) of 5,500, molecular weight dispersity (Mw/Mn) of 1.66, 1/m=40/60
High-molecular-weight compound (A1-3): weight-average molecular weight (Mw) of 5,500, molecular weight dispersity (Mw/Mn) of 1.72, 1/m/n=30/60/10
High-molecular-weight compound (A1-4): weight-average molecular weight (Mw) of 9,500, molecular weight dispersity (Mw/Mn) of 1.72, 1/m/n=55/30/15
High-molecular-weight compound (A1-5): weight-average molecular weight (Mw) of 9,500, molecular weight dispersity (Mw/Mn) of 1.75, 1/m/n=55/30/15

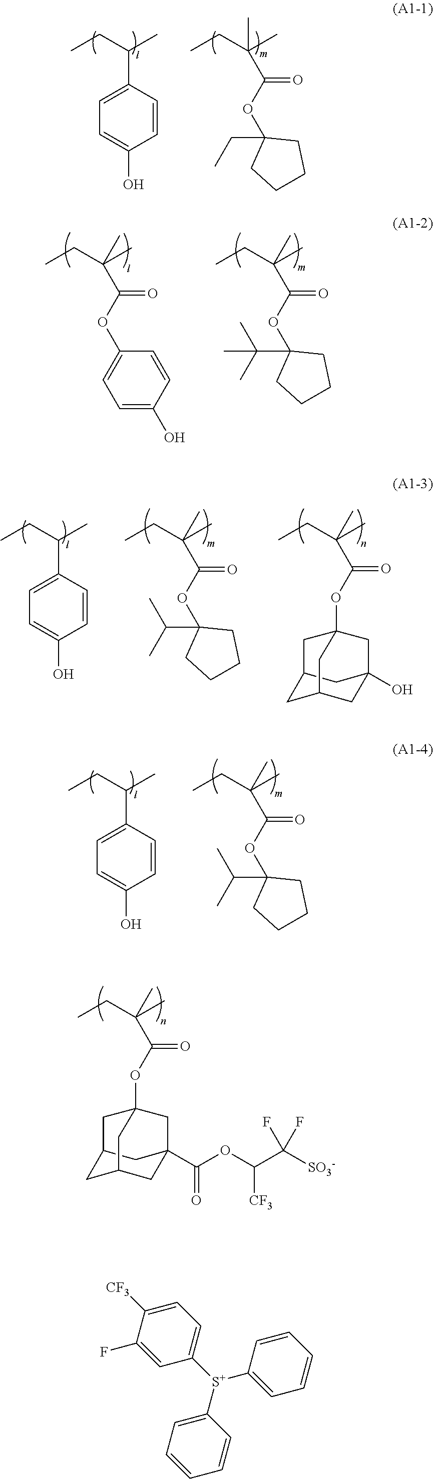

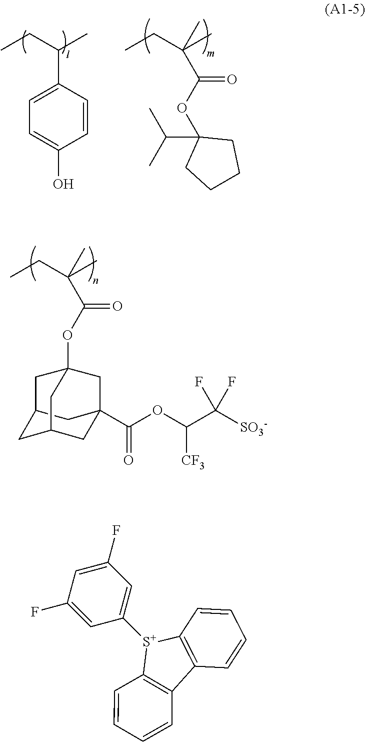

(B0)-1 to (B0)-16: acid-generating agents formed of compounds (B0-1) to (B0-16) shown above
(B1)-1 to (B1)-14: acid-generating agents formed of compounds (B1-1 to (B1-14) shown above

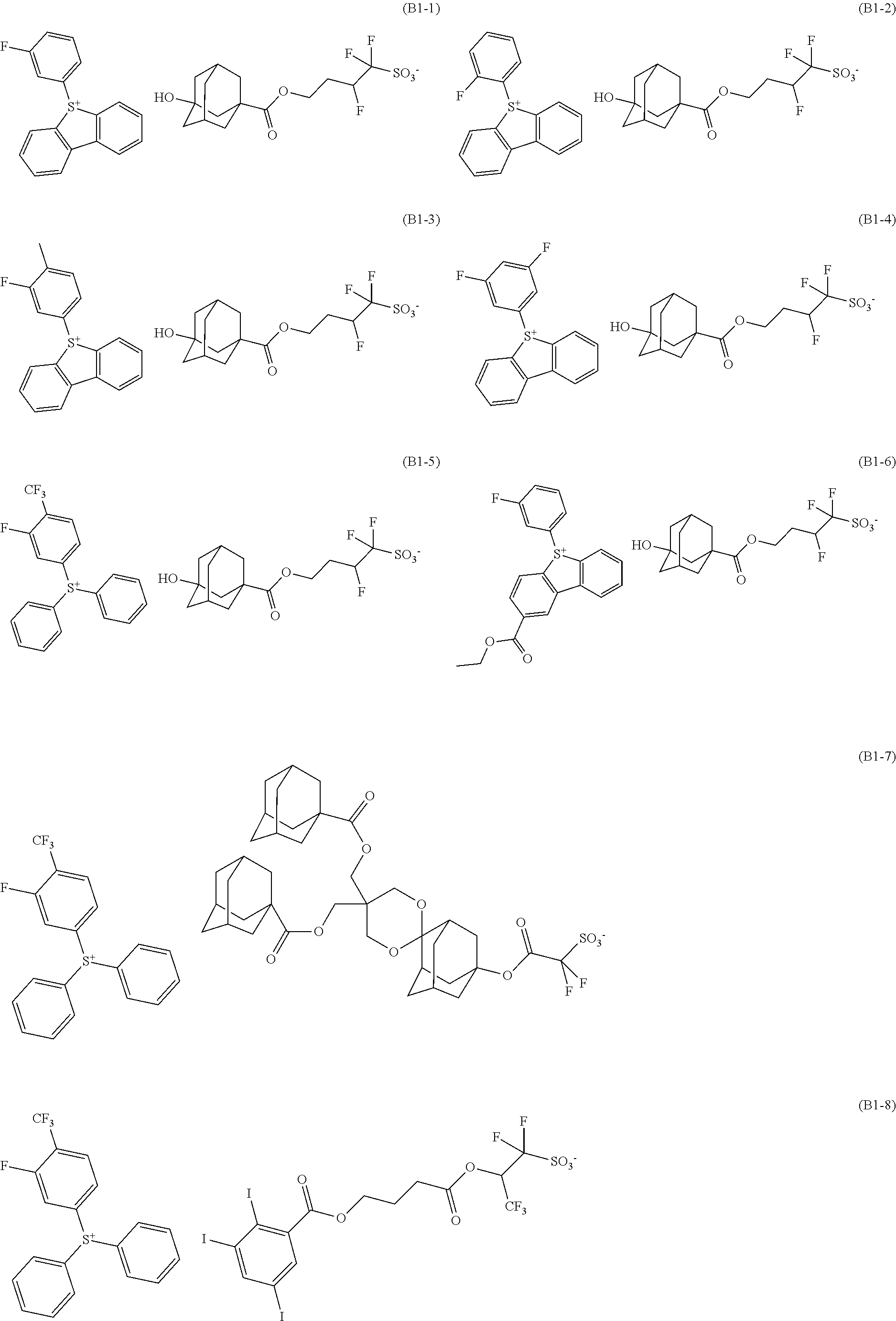

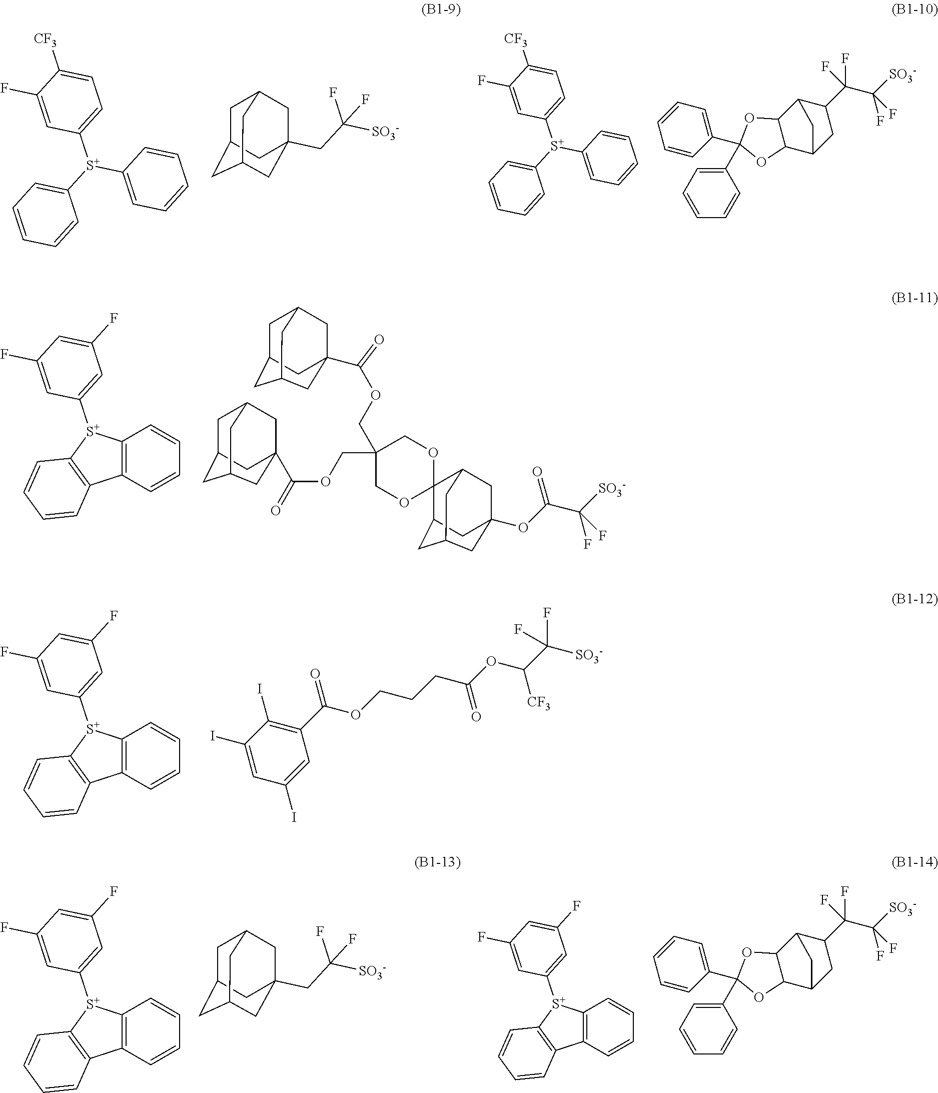

(D0)-1 to (D0)-14: photo-decomposable bases formed of compounds (D0-1) to (D0-14) shown above
(D1)-1 to (D1)-13: acid diffusion control agents formed of compounds each represented by Chemical Formulae (D1-1) to (D1-13).

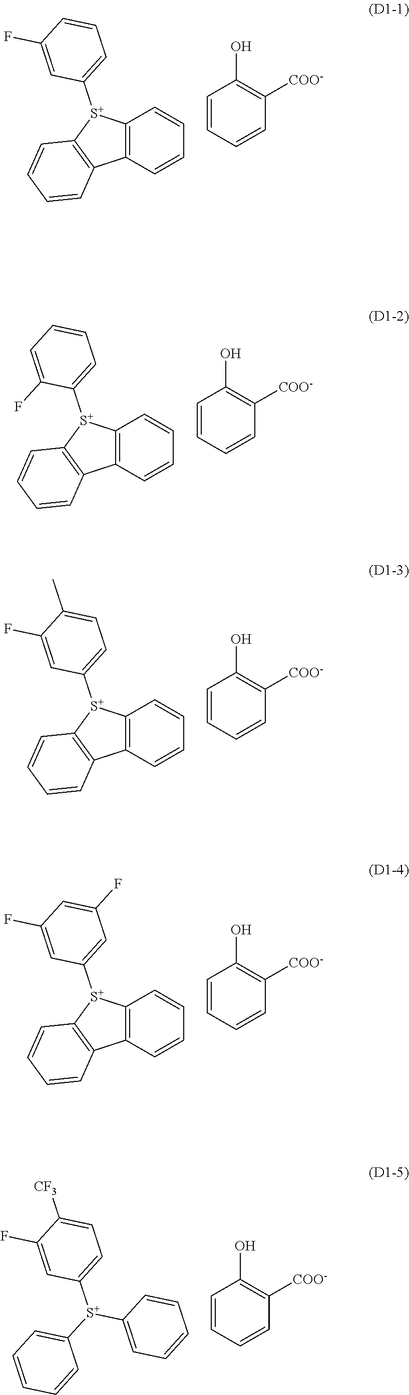

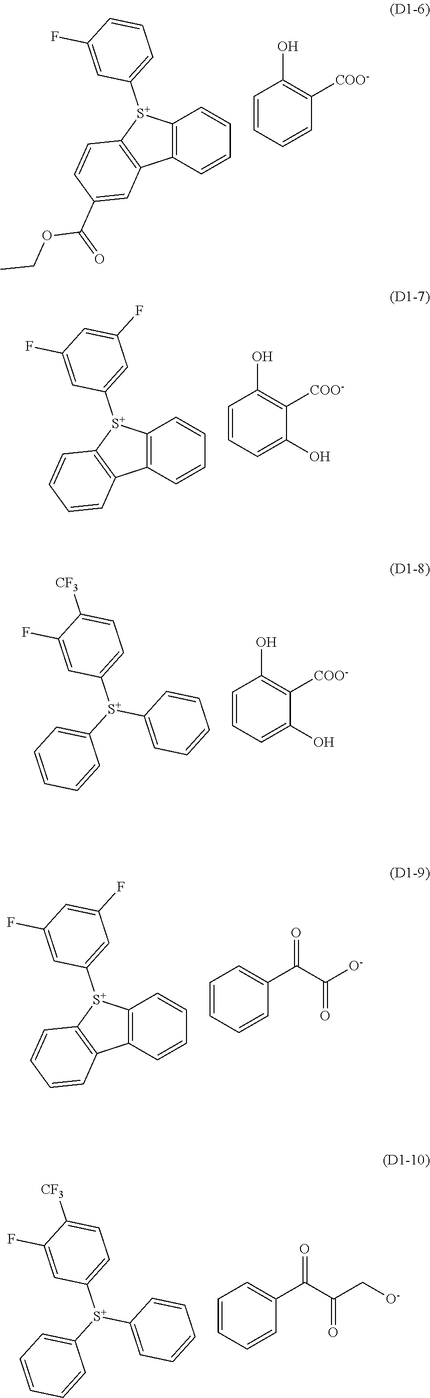

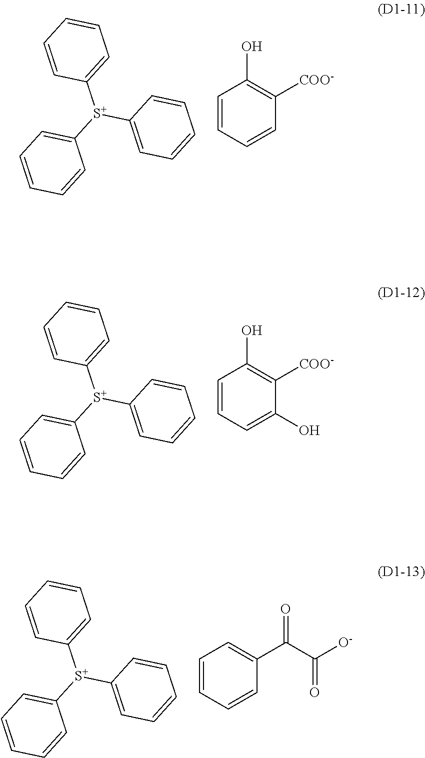

(S)-1: A mixed solvent of propylene glycol monomethyl ether acetate/propylene glycol monomethyl ether=60/40 (mass ratio)

### <Resist Pattern Formation>

An 8-inch silicon substrate which had been subjected to a hexamethyldisilazane (HMDS) treatment was coated with each resist composition of each example using a spinner, and subjected to a pre-baking (PAB) treatment on a hot plate at a temperature of 110° C. for 60 seconds so that the substrate was dried, thereby forming a resist film having a film thickness of 50 nm.

Next, drawing (exposure) was carried out on the resist film by using an electron beam lithography apparatus JEOL-JBX-9300FS (manufactured by JEOL Ltd.), with a target size set to a 1:1 line and space pattern (hereinafter, referred to as “LS pattern”) having a line width of 50 nm, at an acceleration voltage of 100 kV Thereafter, a post-exposure bake (PEB) treatment was performed thereon at 100° C. for 60 seconds.

Subsequently, alkali development was performed at 23° C. for 60 seconds using a 2.38 mass % tetramethylammonium hydroxide (TMAH) aqueous solution “NMD-3” (trade name, manufactured by TOKYO OHKA KOGYO CO., LTD.). Thereafter, water rinsing was performed for 15 seconds using pure water. As a result, a 1:1 LS pattern having a line width of 50 nm was formed.

### [Evaluation of Optimum Exposure Amount (Eop)]

An optimum exposure amount Eop (mJ/cm²) for forming the LS pattern having the target size was determined as described above in <Formation of resist pattern>. The results are listed in Tables 13 to 24 in the columns of “Eop (mJ/cm²)”.

### [Evaluation of Defects]

An 8-inch silicon substrate which had been subjected to a hexamethyldisilazane (HMDS) treatment was coated with each resist composition of each example using a spinner, and subjected to a pre-baking (PAB) treatment on a hot plate at a temperature of 110° C. for 60 seconds so that the resist composition was dried, thereby forming a resist coating film having a film thickness of 50 nm. The number of foreign substances/defects in this coating film was measured using Surfscan SP2 (product name, manufactured by KLA Corporation).

In a case where the number of foreign substances/defects with a size of 80 nm or less present on the surface of the coating film was set to 1.0 in Comparative Example 1, the relative number in each example was calculated, and the evaluation was performed according to the following evaluation standards. The results are listed in the columns of “defects” in Tables 20 to 31.

### (Evaluation Standards)

A: 0.2 or less
B: 0.2 to 0.5
C: 0.5 to 1.0
D: More than 1.0

TABLE 20

| | PAB | PEB | Eop | |
|---|---|---|---|---|
| | (° C.) | (° C.) | [μC/cm²] | Defects |
| Example 1 | 110 | 100 | 90 | A |
| Example 2 | 110 | 100 | 90 | A |
| Example 3 | 110 | 100 | 85 | A |
| Example 4 | 110 | 100 | 90 | A |
| Example 5 | 110 | 100 | 90 | B |
| Example 6 | 110 | 100 | 90 | B |
| Example 7 | 110 | 100 | 90 | B |
| Example 8 | 110 | 100 | 90 | B |
| Example 9 | 110 | 100 | 95 | B |
| Example 10 | 110 | 100 | 90 | B |
| Example 11 | 110 | 100 | 95 | B |
| Example 12 | 110 | 100 | 90 | B |

TABLE 21

| | PAB | PEB | Eop | |
|---|---|---|---|---|
| | (° C.) | (° C.) | [μC/cm²] | Defects |
| Example 13 | 110 | 100 | 100 | B |
| Example 14 | 110 | 100 | 90 | B |
| Example 15 | 110 | 100 | 90 | B |
| Example 16 | 110 | 100 | 90 | B |
| Example 17 | 110 | 100 | 90 | B |
| Example 18 | 110 | 100 | 90 | B |

TABLE 22

| | PAB | PEB | Eop | |
|---|---|---|---|---|
| | (° C.) | (° C.) | [μC/cm²] | Defects |
| Example 19 | 110 | 100 | 95 | B |
| Example 20 | 110 | 100 | 100 | B |
| Example 21 | 110 | 100 | 90 | B |
| Example 22 | 110 | 100 | 90 | B |

TABLE 23

| | PAB | PEB | Eop | |
|---|---|---|---|---|
| | (° C.) | (° C.) | [μC/cm²] | Defects |
| Example 23 | 110 | 100 | 90 | A |
| Example 24 | 110 | 100 | 90 | A |
| Example 25 | 110 | 100 | 85 | A |
| Example 26 | 110 | 100 | 90 | A |
| Example 27 | 110 | 100 | 90 | B |
| Example 28 | 110 | 100 | 90 | B |
| Example 29 | 110 | 100 | 90 | B |
| Example 30 | 110 | 100 | 90 | B |
| Example 31 | 110 | 100 | 95 | B |
| Example 32 | 110 | 100 | 90 | B |
| Example 33 | 110 | 100 | 95 | B |
| Example 34 | 110 | 100 | 90 | B |

TABLE 24

| | PAB | PEB | Eop | |
|---|---|---|---|---|
| | (° C.) | (° C.) | [μC/cm²] | Defects |
| Example 35 | 110 | 100 | 100 | B |
| Example 36 | 110 | 100 | 90 | B |
| Example 37 | 110 | 100 | 90 | B |
| Example 38 | 110 | 100 | 90 | B |
| Example 39 | 110 | 100 | 90 | B |
| Example 40 | 110 | 100 | 90 | B |

TABLE 25

| | PAB | PEB | Eop | |
|---|---|---|---|---|
| | (° C.) | (° C.) | [μC/cm²] | Defects |
| Example 41 | 110 | 100 | 95 | B |
| Example 42 | 110 | 100 | 100 | B |

TABLE 26

| | PAB | PEB | Eop | |
|---|---|---|---|---|
| | (° C.) | (° C.) | [μC/cm²] | Defects |
| Example 43 | 110 | 100 | 90 | A |
| Example 44 | 110 | 100 | 90 | A |
| Example 45 | 110 | 100 | 85 | A |
| Example 46 | 110 | 100 | 90 | A |
| Example 47 | 110 | 100 | 90 | B |
| Example 48 | 110 | 100 | 90 | B |
| Example 49 | 110 | 100 | 90 | B |
| Example 50 | 110 | 100 | 90 | B |
| Example 51 | 110 | 100 | 95 | B |
| Example 52 | 110 | 100 | 90 | B |
| Example 53 | 110 | 100 | 95 | B |
| Example 54 | 110 | 100 | 90 | B |

TABLE 27

| | PAB | PEB | Eop | |
|---|---|---|---|---|
| | (° C.) | (° C.) | [μC/cm²] | Defects |
| Example 55 | 110 | 100 | 100 | B |
| Example 56 | 110 | 100 | 90 | B |
| Example 57 | 110 | 100 | 90 | B |
| Example 58 | 110 | 100 | 90 | B |

TABLE 28

| | PAB | PEB | Eop | |
|---|---|---|---|---|
| | (° C.) | (° C.) | [μC/cm²] | Defects |
| Example 59 | 110 | 100 | 95 | B |
| Example 60 | 110 | 100 | 100 | B |

TABLE 29

| | PAB | PEB | Eop | |
|---|---|---|---|---|
| | (° C.) | (° C.) | [μC/cm²] | Defects |
| Comparative | 110 | 100 | 120 | C |
| Example 1 | | | | |
| Comparative | 110 | 100 | 120 | C |
| Example 2 | | | | |
| Comparative | 110 | 100 | 125 | C |
| Example 3 | | | | |
| Comparative | 110 | 100 | 120 | D |
| Example 4 | | | | |
| Comparative | 110 | 100 | 120 | C |
| Example 5 | | | | |
| Comparative | 110 | 100 | 110 | C |
| Example 6 | | | | |

TABLE 30

| | PAB | PEB | Eop | |
|---|---|---|---|---|
| | (° C.) | (° C.) | [μC/cm²] | Defects |
| Comparative | 110 | 100 | 120 | B |
| Example 7 | | | | |
| Comparative | 110 | 100 | 120 | B |
| Example 8 | | | | |
| Comparative | 110 | 100 | 115 | B |
| Example 9 | | | | |
| Comparative | 110 | 100 | 115 | B |
| Example 10 | | | | |
| Comparative | 110 | 100 | 110 | D |
| Example 11 | | | | |
| Comparative | 110 | 100 | 110 | D |
| Example 12 | | | | |
| Comparative | 110 | 100 | 105 | D |
| Example 13 | | | | |
| Comparative | 110 | 100 | 105 | D |
| Example 14 | | | | |
| Comparative | 110 | 100 | 120 | C |
| Example 15 | | | | |
| Comparative | 110 | 100 | 120 | C |
| Example 16 | | | | |
| Comparative | 110 | 100 | 120 | C |
| Example 17 | | | | |
| Comparative | 110 | 100 | 120 | C |
| Example 18 | | | | |

TABLE 31

| | PAB | PEB | Eop | |
|---|---|---|---|---|
| | (° C.) | (° C.) | [μC/cm²] | Defects |
| Comparative | 110 | 100 | 110 | D |
| Example 19 | | | | |
| Comparative | 110 | 100 | 115 | D |
| Example 20 | | | | |
| Comparative | 110 | 100 | 105 | C |
| Example 21 | | | | |
| Comparative | 110 | 100 | 110 | C |
| Example 22 | | | | |
| Comparative | 110 | 100 | 110 | C |
| Example 23 | | | | |
| Comparative | 110 | 100 | 110 | D |
| Example 24 | | | | |

As listed in Tables 20 to 31, it was confirmed that all the resist compositions of the examples were more satisfactory in terms of the sensitivity and the number of defects than the resist compositions of the comparative examples.

While preferred embodiments of the present invention have been described and illustrated above, it should be understood that these are exemplary of the present invention and are not to be considered as limiting. Additions, omissions, substitutions, and other modifications can be made without departing from the scope of the invention. Accordingly, the present invention is not to be considered as being limited by the foregoing description and is only limited by the scope of the appended claims.

What is claimed is:

1. A resist composition which generates an acid upon light exposure and whose solubility in a developing solution is changed due to an action of the acid, the resist composition comprising:
a compound which contains a cation (C0) represented by General Formula (c0), 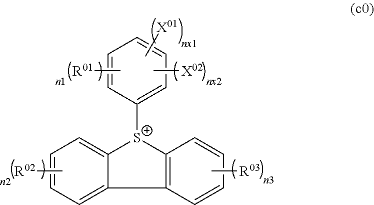
wherein X⁰¹ represents an electron-withdrawing group, X⁰² represents an electron-withdrawing group different from X⁰¹, R⁰¹ represents an electron-donating group, R⁰² and R⁰³ each independently represents a substituent, nx1 and nx2 each independently represents an integer of 1 to 4, n1 represents an integer of 0 to 3, provided that nx1+nx2+n1≤5 is satisfied, n2 and n3 each independently represents an integer of 0 to 4, a plurality of X⁰¹'s may be the same as or different from each other when nx1 represents 2 or greater, a plurality of X⁰²'s may be the same as or different from each other when nx2 represents 2 or greater, a plurality of R⁰¹'s may be the same as or different from each other when n1 represents 2 or greater, a plurality of R⁰²'s may be the same as or different from each other when n2 represents 2 or greater, and a plurality of R⁰³'s may be the same as or different from each other when n3 represents 2 or greater.

2. The resist composition according to claim 1, further comprising:
a resin component (A1) whose solubility in a developing solution is changed due to the action of an acid; and
an acid-generating agent component (B) which generates an acid upon light exposure,
wherein the acid-generating agent component (B) contains a compound represented by General Formula (b0),
M^{b⊕}Xb^{⊖} (b0)
wherein M^{b+} represents the cation (C0), and Xb⁻ represents a counter anion.

3. The resist composition according to claim 1, further comprising:
a resin component (A1) whose solubility in a developing solution is changed due to the action of an acid; and
an acid diffusion control agent component (D) which controls diffusion of an acid generated upon light exposure,
wherein the acid diffusion control agent component (D) contains a compound represented by General Formula (d0),
M^{d⊕}Xd^{⊕} (d0)
wherein M^{d+} represents the cation (C0), and Xd⁻ represents a counter anion.

4. The resist composition according to claim 1, further comprising:
a resin component (A0) which has a constitutional unit (a0) represented by General Formula (a0-1), 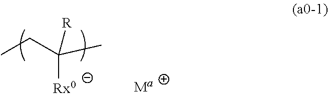
wherein R represents a hydrogen atom, an alkyl group having 1 to 5 carbon atoms, or a halogenated alkyl group having 1 to 5 carbon atoms, M^{a+} represents the cation (C0), and Rx⁰⁻ represents a group having an anion.

5. The resist composition according to claim 4, further comprising:
a resin component (A1) whose solubility in a developing solution is changed due to the action of an acid,
wherein the resin component (A1) has contains the constitutional unit (a0).

6. A resist pattern formation method, comprising:
forming a resist film on a support using the resist composition according to claim 1;
exposing the resist film to light; and
developing the resist film exposed to light to form a resist pattern.

7. A compound represented by General Formula (M0), 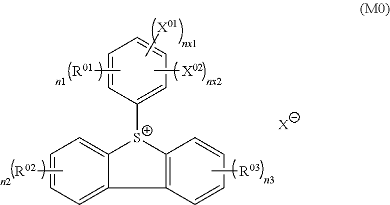
wherein X⁰¹ represents an electron-withdrawing group, X⁰² represents an electron-withdrawing group different from X⁰¹, R⁰¹ represents an electron-donating group, R⁰² and R⁰³ each independently represents a substituent, nx1 and nx2 each independently represents an integer of 1 to 4, n1 represents an integer of 0 to 3, provided that nx1+nx2+n1≤5 is satisfied, n2 and n3 each independently represents an integer of 0 to 4, a plurality of X⁰¹'s may be the same as or different from each other when nx1 represents 2 or greater, a plurality of X⁰²'s may be the same as or different from each other when nx2 represents 2 or greater, a plurality of R⁰¹'s may be the same as or different from each other when n1 represents 2 or greater, a plurality of R⁰²'s may be the same as or different from each other when n2 represents 2 or greater, a plurality of R⁰³'s may be the same as or different from each other when n3 represents 2 or greater, and X⁻ represents a counter anion.

8. An acid-generating agent comprising:
a compound represented by General Formula (B0), 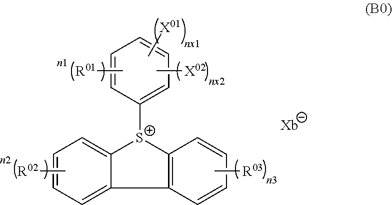
wherein X⁰¹ represents an electron-withdrawing group, X⁰² represents an electron-withdrawing group different from X⁰¹, R⁰¹ represents an electron-donating group, R⁰² and R⁰³ each independently represents a substituent, nx1 and nx2 each independently represents an integer of 1 to 4, n1 represents an integer of 0 to 3, provided that nx1+nx2+n1≤5 is satisfied, n2 and n3 each independently represents an integer of 0 to 4, a plurality of X⁰¹'s may be the same as or different from each other when nx1 represents 2 or greater, a plurality of X⁰²'s may be the same as or different from each other when nx2 represents 2 or greater, a plurality of R⁰¹'s may be the same as or different from each other when n1 represents 2 or greater, a plurality of R⁰²'s may be the same as or different from each other when n2 represents 2 or greater, a plurality of R⁰³'s may be the same as or different from each other when n3 represents 2 or greater, and Xb⁻ represents a counter anion.

9. An acid diffusion control agent, comprising:
a compound represented by General Formula (D0), 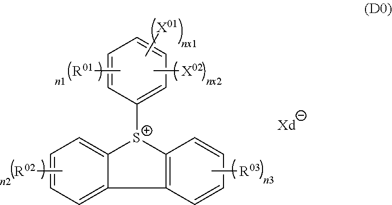
wherein X⁰¹ represents an electron-withdrawing group, X⁰² represents an electron-withdrawing group different from X⁰¹, R⁰¹ represents an electron-donating group, R⁰² and R⁰³ each independently represents a substituent, nx1 and nx2 each independently represents an integer of 1 to 4, n1 represents an integer of 0 to 3, provided that nx1+nx2+n1≤5 is satisfied, n2 and n3 each independently represents an integer of 0 to 4, a plurality of X⁰¹'s may be the same as or different from each other when nx1 represents 2 or greater, a plurality of X⁰²'s may be the same as or different from each other when nx2 represents 2 or greater, a plurality of R⁰¹'s may be the same as or different from each other when n1 represents 2 or greater, a plurality of R⁰²'s may be the same as or different from each other when n2 represents 2 or greater, a plurality of R⁰³'s may be the same as or different from each other when n3 represents 2 or greater, and Xd⁻ represents a counter anion.

10. A high-molecular-weight compound which has a constitutional unit (a0) represented by General Formula (A0-1), 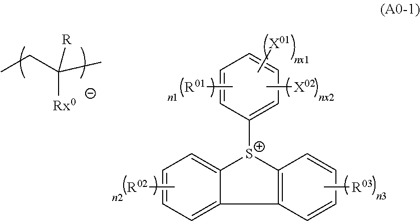
wherein R represents a hydrogen atom, an alkyl group having 1 to 5 carbon atoms, or a halogenated alkyl group having 1 to 5 carbon atoms, Rx⁰⁻ represents a group having an anion, X⁰¹ represents an electron-withdrawing group, X⁰² represents an electron-withdrawing group different from X⁰¹, R⁰¹ represents an electron-donating group, R⁰² and R⁰³ each independently represents a substituent, nx1 and nx2 each independently represents an integer of 1 to 4, n1 represents an integer of 0 to 3, provided that nx1+nx2+n1≤5 is satisfied, n2 and n3 each independently represents an integer of 0 to 4, a plurality of X⁰¹'s may be the same as or different from each other when nx1 represents 2 or greater, a plurality of X⁰²'s may be the same as or different from each other when nx2 represents 2 or greater, a plurality of R⁰¹'s may be the same as or different from each other when n1 represents 2 or greater, a plurality of R⁰²'s may be the same as or different from each other when n2 represents 2 or greater, and a plurality of R⁰³'s may be the same as or different from each other when n3 represents 2 or greater.
